(19) United States
(12) Patent Application Publication
(10) Pub. No.: US 2007/0015149 A1
(43) Pub. Date: Jan. 18, 2007

(54) Prlz regulatory elements in the treatment of disease and the discovery of therapeutics

(76) Inventors: Ruoxing Wang, Norcross, GA (US); Leland W. K. Chung, Atlanta, GA (US)

Correspondence Address: PATENT ADMINISTRATOR;KATTEN MUCHIN ROSENMAN LLP, 1025 THOMAS JEFFERSON STREET, N.W., EAST LOBBY: SUITE 700, WASHINGTON, DC, 20007-5201, US

(21) Appl. No.: 10/510,148

(22) PCT Filed: Apr. 7, 2003
(86) PCT No.: PCT/US03/10536

§ 371 Date: Oct. 5, 2004

Related U.S. Application Data
(60) Provisional application No. 60/370,557, filed on Apr. 5, 2002.

(51) Int. Cl. C12Q 1/68; G01N 33/574; C07H 21/04; C12P 21/06; C07K 14/82; C07K 16/30; C40B 30/06

(52) U.S. Cl. 435/6; 536/23.5; 530/350; 530/388.8; 435/69.1; 435/320.1; 435/325; 435/7.23

(57) ABSTRACT

The compositions and methods of the present invention are based, in part, on a gene designated prostate leucine zipper (PrLZ) and the sequences that mediate its expression. The presence of the leucine zipper indicates that PrLZ encodes a protein that interacts with other proteins. Methods to inhibit the activity of PrLZ by identifying and inhibiting the interaction between PrLZ and such binding partners are within the scope of the invention, as are methods of inhibiting PrLZ in other ways (by, for example, inhibiting its expression with antisense molecules or siRNAs or inhibiting its activity with, for example, anti-PrLZ antibodies). Such inhibition is useful in the treatment of cancers or dysplasias affecting PrLZ-positive tissues, such as those in the prostate.

### RELATED APPLICATIONS

This application claims priority from a United States provisional application (U.S. Ser. No. 60/370,557), which was filed on Apr. 5, 2002. For the purpose of any United States application that may issue as a U.S. patent from the present application, the contents of the prior provisional application is hereby incorporated by reference in its entirety.

### TECHNICAL FIELD

This invention relates to the use of prostate-expressed genes and the sequences that regulate their expression to diagnose and treat disease, and to identify therapeutic agents. More particularly, the invention relates to the use of the Prostate Leucine Zipper (PrLZ) gene and the sequence that directs its expression.

### BACKGROUND

The human prostate is the largest accessory sex organ, and it is frequently a site of disease in aging males. Developmentally, a series of morphogenetic and organogenetic events, which involve growth, branching, canalization, and cytodifferentiation, occur prior to the formation of functional prostatic ducts (Hayward et al., Radiol. Clin. North Am. 38:1-14, 2000). During neonatal growth and in adolescence, the developing prostate undergoes extensive growth and is subject to hormonal “imprinting” or “programming” (Rajfer et al., Invest. Urol. 16:186-190, 1978, Chung et al., Invest. Urol. 17:337-342, 1980, Singh et al., Biol. Reprod. 61:200-208, 1999). Many transcription factors, such as those from the sex steroid receptor superfamilies (Gelmann, J. Clin. Oncol. 20:3001-3015, 2002) and homeobox genes (Sciavolino et al, Ann. Med. 30:357-368, 1998), regulate the development and maturation of the prostate gland. Aberrant regulation of these genes may and contribute to prostate diseases, including benign prostatic hypertrophy (13PH), prostate intraepithelial neoplasia (PIN), and prostate cancer. Prostate cancer is one of the most prevalent cancers in men. In the early stages, most prostate cancers are responsive to anti-androgen therapy because proliferation of the cancer cells depends on the presence of androgens. However, many prostate cancers ultimately become androgen independent and can then spread to other tissues.

### SUMMARY

The compositions and methods of the present invention are based, in part, on a gene designated prostate leucine zipper (PrLZ) and the sequences that mediate its expression (in U.S. Ser. No. 60/370,557, the priority document for the present application, this gene was designated PC-1; the name has been changed to PrLZ to avoid confusion with other genes that were also designated “C-1”). The presence of the leucine zipper indicates that PrLZ encodes a protein that interacts with other proteins. Methods to inhibit the activity of PrLZ by identifying and inhibiting the interaction between PrLZ and such binding partners are within the scope of the invention and are described further below, as are methods of inhibiting PrLZ in other ways (by, for example, inhibiting its expression with antisense molecules or siRNAs or inhibiting its activity with, for example, anti-PrLZ antibodies). As we explain further below, such inhibition is useful in the treatment of cancers or dysplasias affecting PrLZ-positive tissues, such as those in the prostate.

PrLZ is expressed in healthy and cancerous cells, including prostate cancer cells. More specifically, PrLZ expression is enhanced upon malignant transformation but low in normal prostate epithelia and in the event of benign prostatic hypertrophy (BPH). In fact, PrLZ is elevated significantly in prostate cancer cells and those affected by high-grade prostate intraepithelial neoplasia (PIN). In view of this discovery, we propose evaluating PrLZ as a marker of prostate cancer progression. Moreover, as enhanced expression of PrLZ in the prostate, either by genomic amplification or transcriptional activation, appears to promote malignant transformation of prostate epithelial cells, the invention features methods of inhibiting PrLZ expression or activity.

The genomic sequence of PrLZ, including the sequence of a PrLZ promoter, has been elucidated (see FIG. 2). As PrLZ is expressed, and often highly expressed, in the prostate in the event of either androgen-dependent or androgen-independent prostate cancer, one or more of the sequences we have found that mediate PrLZ expression can be used (alone or in combination with each other and/or other regulatory sequences (e.g., regulatory sequences that mediate the expression of other prostate-specific genes, such as PSA (prostate specific antigen)) to specifically express one or more genes in prostate cancer cells. For example, the 12 kb sequence shown in FIG. 1 (SEQ ID NO:1) can be used to express diagnostic or therapeutic agents (e.g., therapeutic proteins), as can shorter, operable sequences within SEQ ID NO:1. An “operable” portion of SEQ ID NO:1 is a portion of the sequence that is sufficient to drive gene expression at a level that is useful in diagnosis (e.g., to a detectable, preferably readily detectable level under physiological conditions) or treatment (e.g., to a level that confers a therapeutic benefit on a patient). An operable portion of SEQ ID NO:1 may be a fragment of contiguous nucleotides of SEQ ID NO:1 or a discontinuous assembly of sequences containing one or more of the regulatory elements identifiable within SEQ ID NO:1.

The gene products that can be expressed include therapeutically beneficial proteins, including cellular toxins or agents that inhibit cellular proliferation (e.g., proteins encoded by tumor suppressor genes or sequences that inhibit PrLZ expression), and diagnostic markers (therapeutic and diagnostic agents are described further below).

Where a regulatory sequence of the invention is used to express a marker, the compositions of the invention can be used to facilitate molecular imaging and cancer detection. While markers are discussed further below, we note here that a regulatory element (e.g., a PrLZ regulatory element (e.g., SEQ ID NO:1 or an operable portion thereof)) can be used to express a fluorescent or luminescent (e.g., bioluminescent or chemiluminescent) compound, a radioactively-tagged agent, an enzyme, or metal chelator. The enzyme can be one that catalyzes a reaction that produces a detectable reaction product (e.g., horseradish peroxidase, alkaline phosphatase, beta-galactosidase (β-gal), acetylcholinesterase, or chloramphenicol acetyltransferase (CAT)). The marker can also be a protein that can be subsequently detected with an antibody (e.g., an anti-myc antibody, an anti-FLAG antibody, or any other such antibody).

As noted above, PrLZ is expressed in cancerous cells other than those of the prostate, and it is expressed (albeit at lower levels) in normal epithelial cells, including epithelial cells in the prostate (e.g., PrLZ is involved in the growth and development of prostate glands in embryonic and adolescent mammals). Accordingly, one or more PrLZ regulatory elements (e.g. the promoter represented in SEQ ID NO:1 or a minimal promoter within that sequence with, optionally, one or more of the regulatory elements described below) can be used to direct gene expression in these cells and any other PrLZ-positive cell type. The expression of PrLZ in normal prostate cells does not preclude the use of PrLZ regulatory sequences in transgene-based cancer diagnostics or therapies, nor does it limit the application of compounds that decrease PrLZ expression (another aspect of the invention; discussed further below). This is due, at least in part, to the fact that the prostate is a non-essential organ; the prostate can be removed, or PrLZ-positive cells within the prostate can be destroyed or otherwise affected (e.g., prevented from dividing), without threatening the overall well being of the patient.

The invention is not limited, however, to compositions in which PrLZ regulatory sequences are used to inhibit cancerous cells. These sequences can also be used, for example, to express gene products that help maintain the health of healthy cells (i.e., the compositions of the invention can be used in methods of cancer prevention) or to aid in the repair or regeneration of tissue. Thus, the regulatory sequence described herein can be used to express structural proteins, receptors, ion channels, “housekeeping” genes, or any other cellular element found deficient.

PrLZ can also be used as a diagnostic marker. While it can be detected in the prostate (e.g., in a biopsy obtained from the prostate), it can also be detected in bodily fluids such as blood (or a component thereof, such as serum), urine, or ejaculate. Diagnostic methods and the compositions, including diagnostic kits, required to carry them out are within the scope of the present invention.

One or more of the PrLZ regulatory elements can also be used to identify factors (e.g., transcription factors and upstream signaling molecules) that mediate the expression of PrLZ and other genes involved in prostate cancer and other cancers. While compositions and methods for detecting cancer-related factors are described further below, we note here that a PrLZ regulatory element (or a combination of elements, up to and including the sequences described herein that flank the PrLZ gene (including all or part of SEQ D NO:1)) can be operably linked to a gene that encodes a detectable product (i.e., a reporter gene). Cells expressing such a construct can then be exposed to any number, type, or combination of molecules (e.g., those available within small molecule libraries or expressed from cDNA libraries), and the activity of the reporter gene can then be assessed. Molecules that upregulate the reporter stimulate PrLZ expression (and may, thereby, stimulate unwanted cellular proliferation (accordingly, the methods of the invention can be used to identify carcinogenic substances)). On the other hand, molecules that suppress reporter gene expression, suppress PrLZ expression (and may, thereby, suppress cellular proliferation (accordingly, the methods of the invention can be used to identify chemotherapeutic agents)). While reporter gene expression can be conveniently detected, one can also assess PrLZ expression per se.

The nucleic acid molecules of the invention (particularly those that include an operative portion of SEQ ID NO:1, can include one or more of the following regulatory elements, which are known in the art and identifiable by simple search: a TATA box, a CCAAT box, an Sp1 site, a TRE (TPA (12-O-tetradecanoyl-phorbol-13-acetate) response element), a CRE (cyclic AMP response element), an Ets-binding site, an ERE (estrogen response element), a Myc binding site or a binding motif for Myc-Max dimers, an Nf-l binding site (a binding site for NF1-A, NF1-B, NF1-C, or NF1-X), a GATA binding site (a binding site for one of GATA-1 to GATA-6), an E2F binding site, an LSF binding site, a Mef-2 binding site, a CarG box (also known as an SRF (serum response factor) binding site), a Myf binding site (a binding site for MyoD, myogenin, myf5 or MRF4), or a TEA/ATTS domain (a Tef (transcription enhancer factor) binding site).

The PrLZ gene structure and genomic sequence are also useful in allelotyping (e.g., haplotyping) patients to identify those at increased risk for prostate cancer and other cancers.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a representation of a genomic sequence that regulates the expression of PrLZ (SEQ ID NO:1)

FIG. 2 is a representation of a genomic sequence that includes SEQ ID NO:1. It also includes additional 5′ sequence, the six exons of the PrLZ gene, the intervening introns, and 3′ flanking sequence. The sequence of FIG. 2 is represented by SEQ ID NO:2.

FIG. 3 is a representation of the amino acid sequences of human PrLZ (SEQ ID NO:5), murine PrLZ (SEQ ID NO:4), and other TPD52 family protins. Compared with human PrLZ, an identical residue is shown by a dash. Spaces were introduced to facilitate the alignment to the highest identity. Within the PrLZ sequence, bold-lettered S and T residues indicate putative phosphorylation sites, while bold N on a black background represents a putative N-glycosylation site. In addition, the citron homology domain is shown with a double underline. The two putative PEST domains are indicated with a single underline. The coiled coil leucine zipper region is highlighted, with the L and I residues shown in bold with thick underlines.

FIG. 4 is a schematic representation of the relationship between PrLZ and TPD52. The shaded area indicates the coding regions and the slashed areas share identical sequence among different cDNA clones. PrLZ- represented the TPD52 transcript identified in this study, from the screening of the C4-2 cDNA library. PrLZ-502 is a fragment containing the gene-specific region of the PrLZ and was used as a probe in hybridization experiments.

### DETAILED DESCRIPTION

We have performed a variety of studies that focus on the genomic sequence of PrLZ, and we have used lineage-related human prostate cancer cell lines to study the onset and progression of human prostate cancer. More specifically, we used an androgen-independent LNCaP subline, C4-2, which was established through tumor-stroma interaction and xenograft selection (Thalmann et al., Cancer Res. 54:2577-2581, 1994, Wu et al., Int. J. Cancer 57:406-412, 1994). While growth of the parental LNCAP cell line is androgen-dependent (AD, defined as capable of proliferation in intact but not in castrated male hosts), the derivative C4-2 subline is androgen-independent (AI, capable of proliferation in castrated male hosts). C4-2 cells express androgen receptor (AR), secrete PSA in the absence of androgenic hormones, and have metastatic potential to lymph node and bone. Numerous studies have shown that lineage-related LNCaP and C4-2 cells mimic the progression of clinical prostate cancers. The C4-2 cells exhibit bone-like properties, expressing a series of non-collagenous bone matrix proteins, such as osteocalcin, osteopontin, osteonectin and bone sialoprotein. We also found that C4-2 cells express osteoblastic factors and receptors, such as RANKL (receptor activator of NF-kB ligand) and CXCR4, a receptor for stromal factor-1, and are capable of forming mineralized bone in cell culture. We have extensively studied these acquired phenotypic properties of C4-2 cells for clues that may define genetic and gene expressional changes associated with the AD to AI transition.

We examined differential gene expression between LNCaP and C4-2 cells by a microarry screen. Total RNA was isolated from LNCaP and C4-2 cells, and we used a reverse transcription process to prepare fluorescence-labeled cDNA probes (Lin et al., Cancer Res. 60:858-863, 2000). An array of 1,500 unique cDNA sequences, which were derived from the prostate expression database (Nelson et al., Nucleic Acids Res. 28:212-213, 2000) were examined in the hybridization. Hybridization signals were processed and compared between LNCaP and C4-2. This comparative analysis and the subsequent Northern blot hybridization identified eight cDNA clones that were seen with more than two fold higher expression in C4-2 than in the LNCAP cell line.

One of the eight clones showed a nine fold higher expression in C4-2 than in LNCAP cells, as determined by Northern blot hybridization. On the original microarray, this clone was represented with a 330 base pair (bp) cDNA insert (Genbank accession number 1113969). Using this insert as a probe, we screened a constructed cDNA library of the C4-2 cells and isolated fourteen positive clones. Subsequent restriction mapping and DNA sequencing analyses revealed that these clones were from two transcripts that shared identical sequence in the 3′ half, but contained distinctive sequences in their 5′regions. One of the two transcripts represented a previously reported TPD52 (Byrne et al., Cancer Res. 55:2896-2903, 1995), except that the new isolate contained a longer 5′ untranslated region (UTR) and a shorter 3′UTR. The other transcript contained a unique 502 bp sequence in its 5′region, completely divergent from that of TPD52. Consequently, it encoded a polypeptide with a 41 residue unique N-terminus, 35 residues longer than the PTD52 protein. It represented a new transcript and was a new member of the recently identified TPD52 gene family (Byrne et al., Genomics 35:523-32, 1996). We named it PrLZ and have since focused on the characterization of this novel gene and its product.

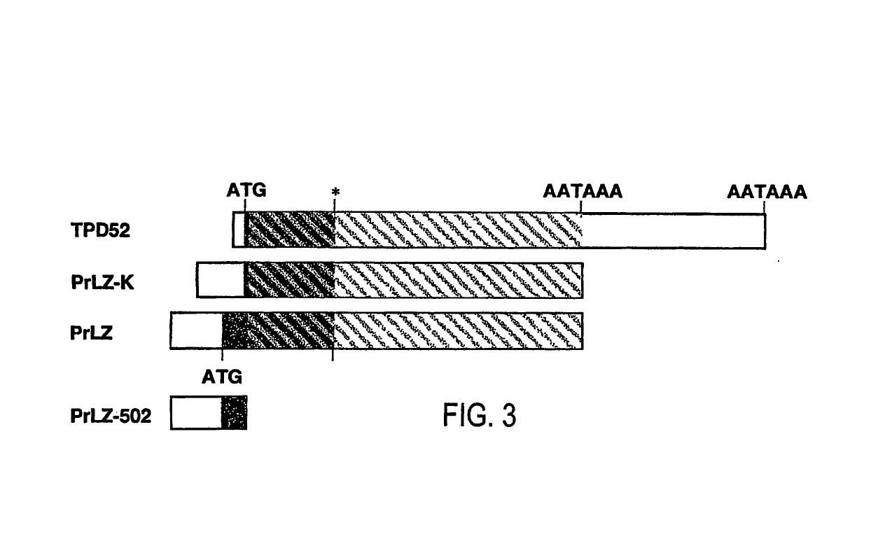

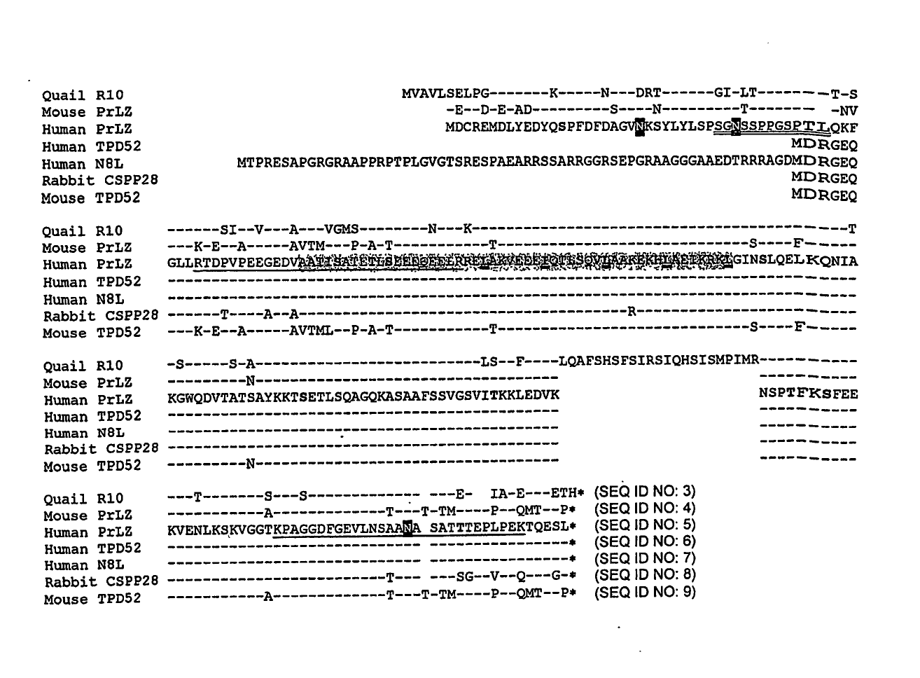

The full cDNA sequence for PrLZ was 2573 bp in size, capable of encoding a 224 amino acid residue polypeptide, with a predicted molecular weight of 24.4 kDa. Many structural features of the TPD52 family were conserved in PrLZ. These included a coiled coil leucine zipper in the central region, suggesting an involvement of PrLZ in protein-protein interactions (Sathasivam et al., Biochem. Biophys. Res. Commun. 288:56-61, 2001). Flanking the central domain were two PEST domains (Rogers et al., Science 234:364-368, 1986), indicative of proteolytic modification of the PrLZ protein. There were several canonical serine/threonine phosphorylation sites, substrate for casein II kinase, protein kinase C, cAMP- and cGMP-dependent kinases. This signifies that several signal transduction pathways could modulate function of the PrLZ protein. In addition, compared to other members of the TPD52 family, PrLZ protein harbored two additional N-glycosylation sites and a N-terminal citron motif in its unique N-terminus, suggesting that PrLZ was a glycosylated protein (see FIG. 3).

To determine subcellular localization of the PrLZ protein, we transfected PrLZ as a GFP fusion protein into prostate cancer cell lines. Fluorescence microscopy and confocal microscopic analysis demonstrated that, like other TPD52 family members (Proux et al., J. Biol. Chem. 271:30790-30797, 1996), PrLZ was mostly a cytoplasmic protein.

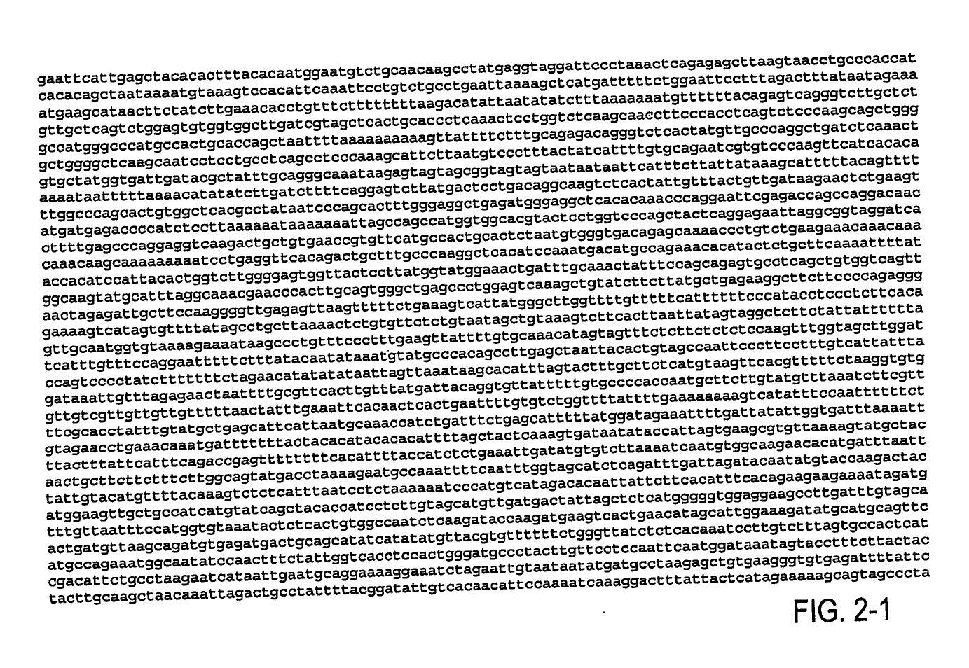

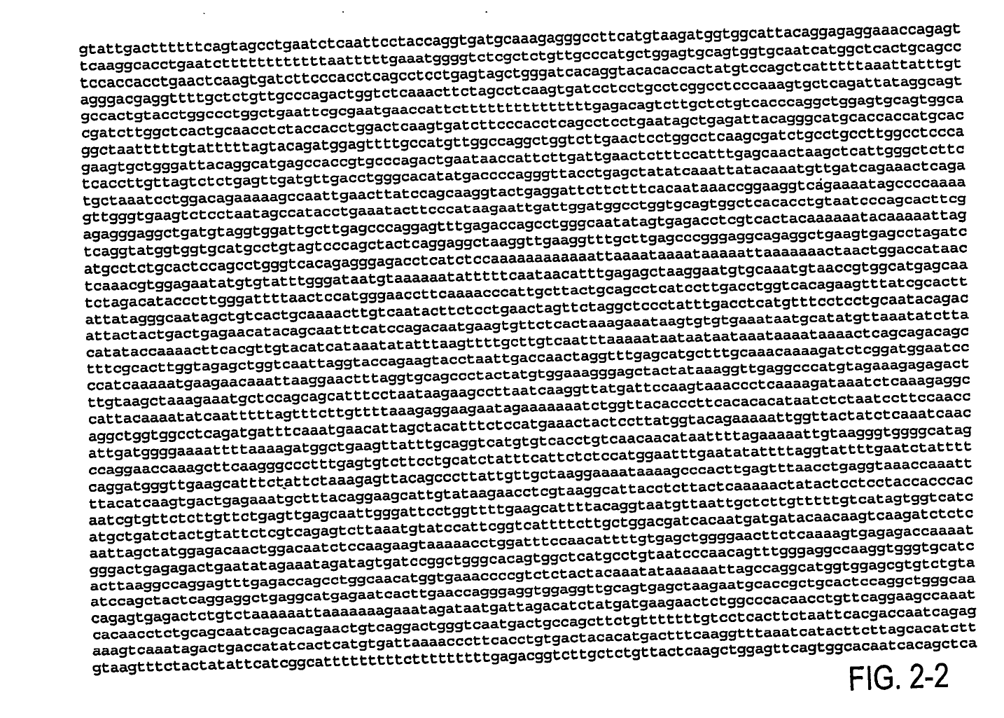

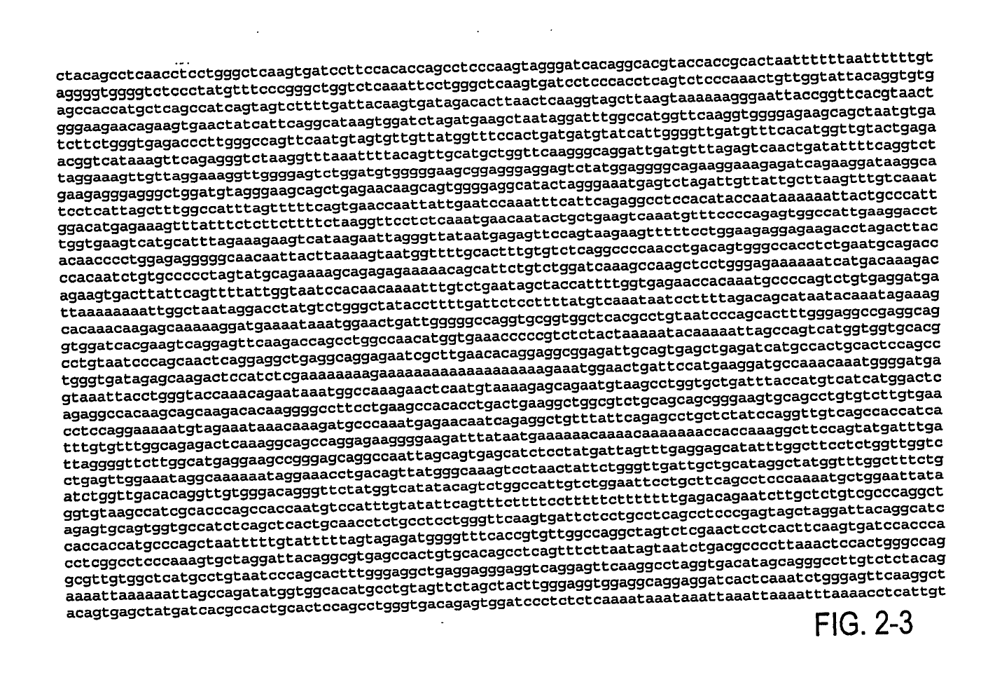

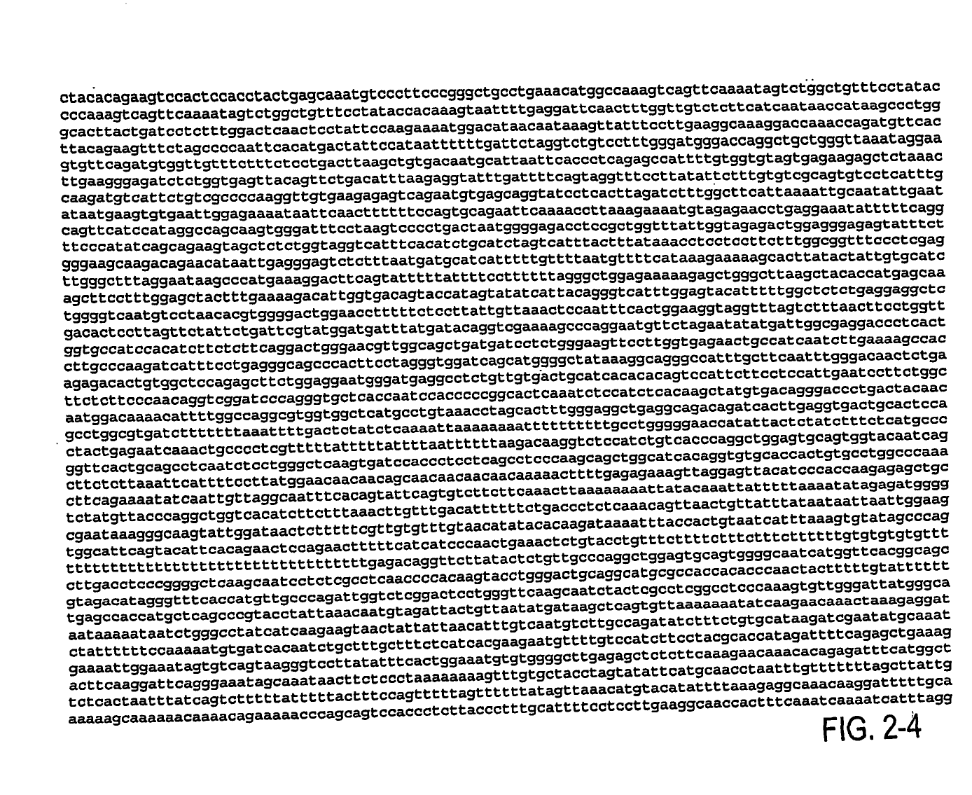

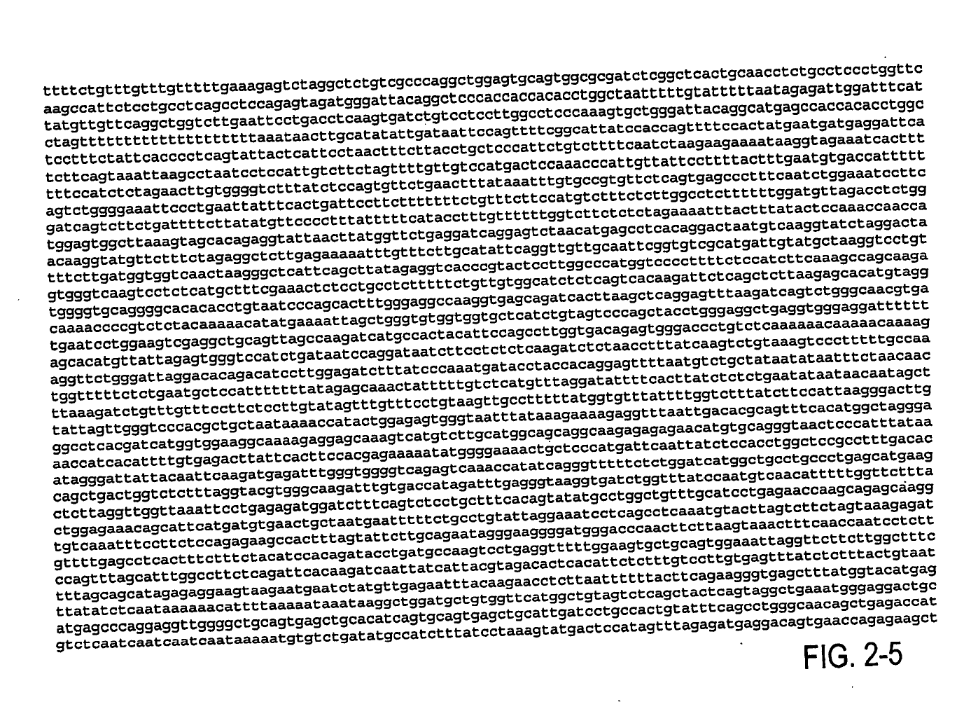

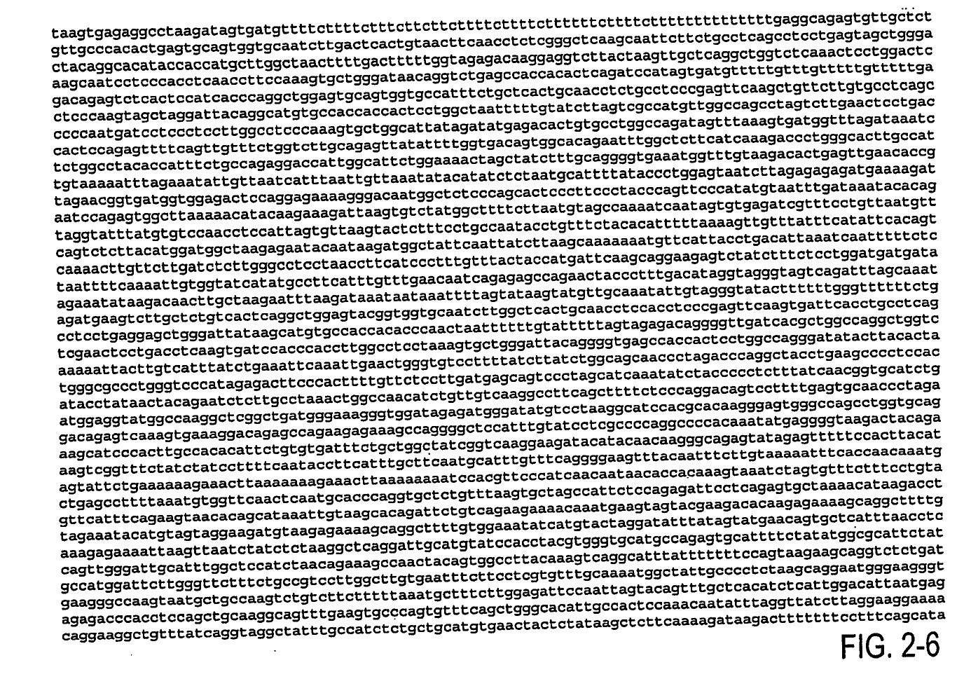

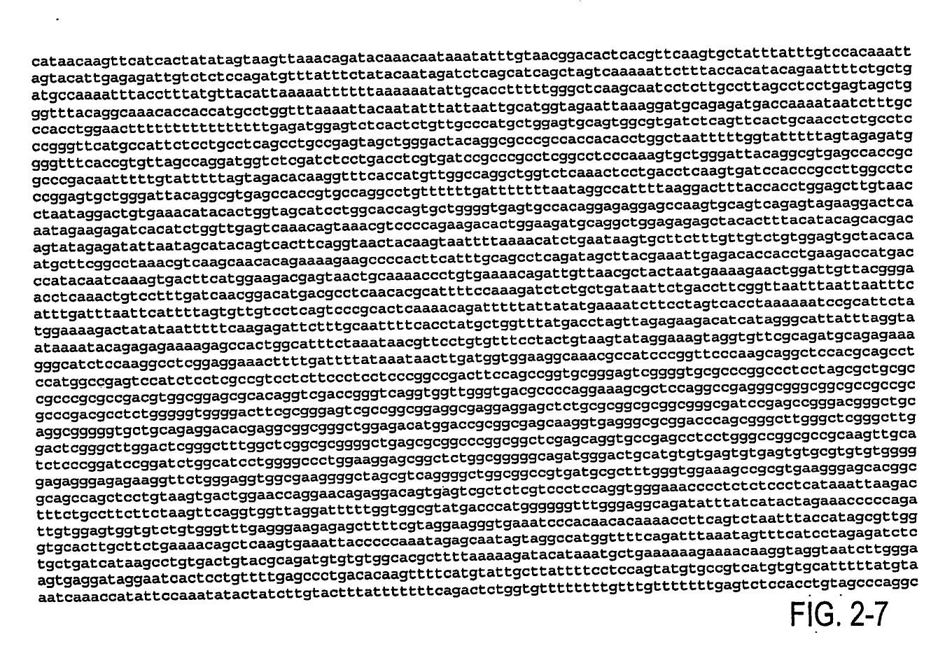

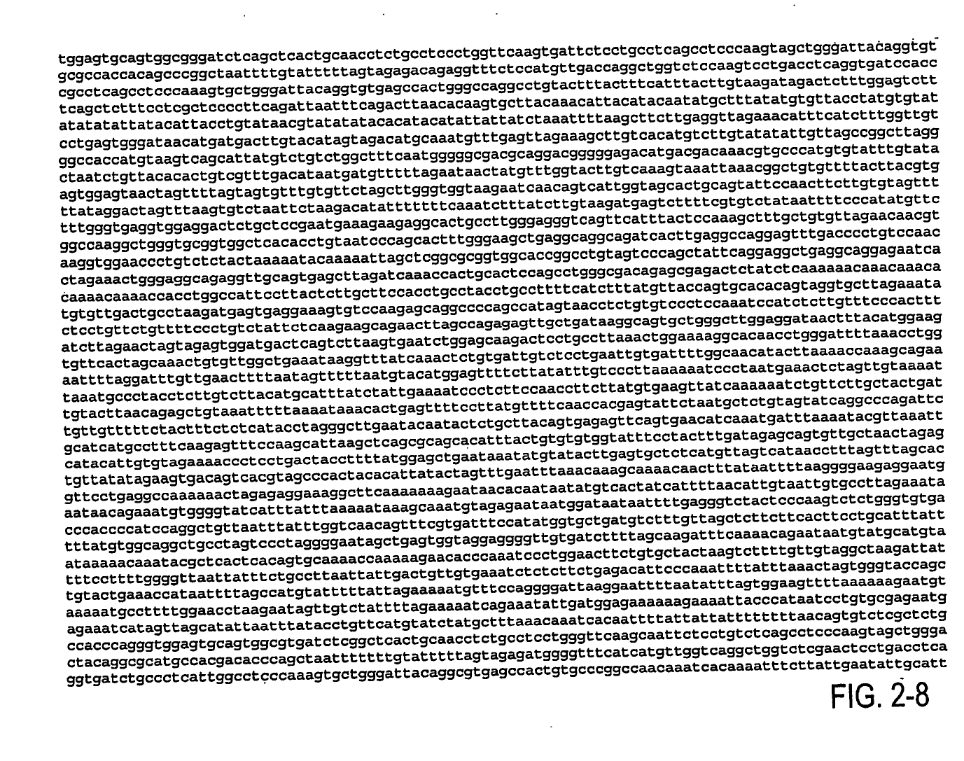

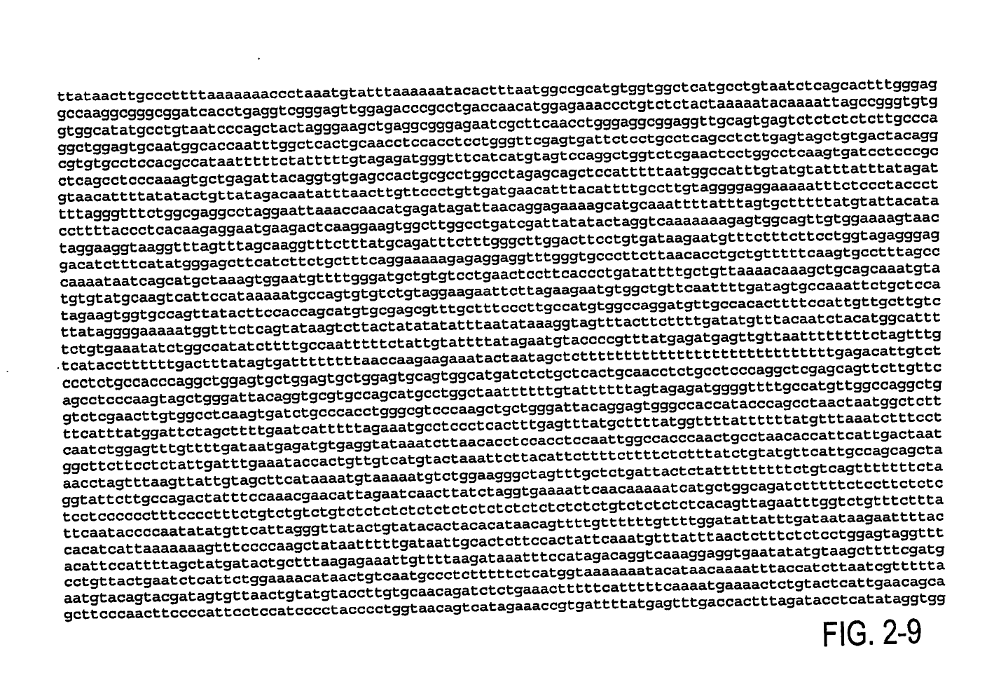

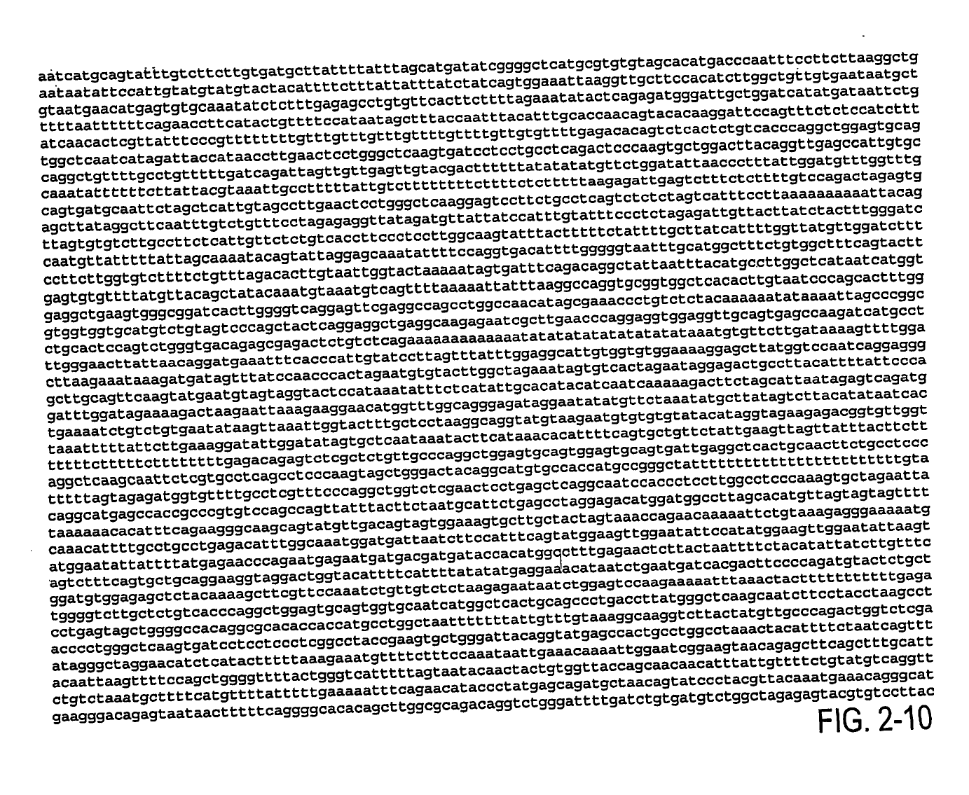

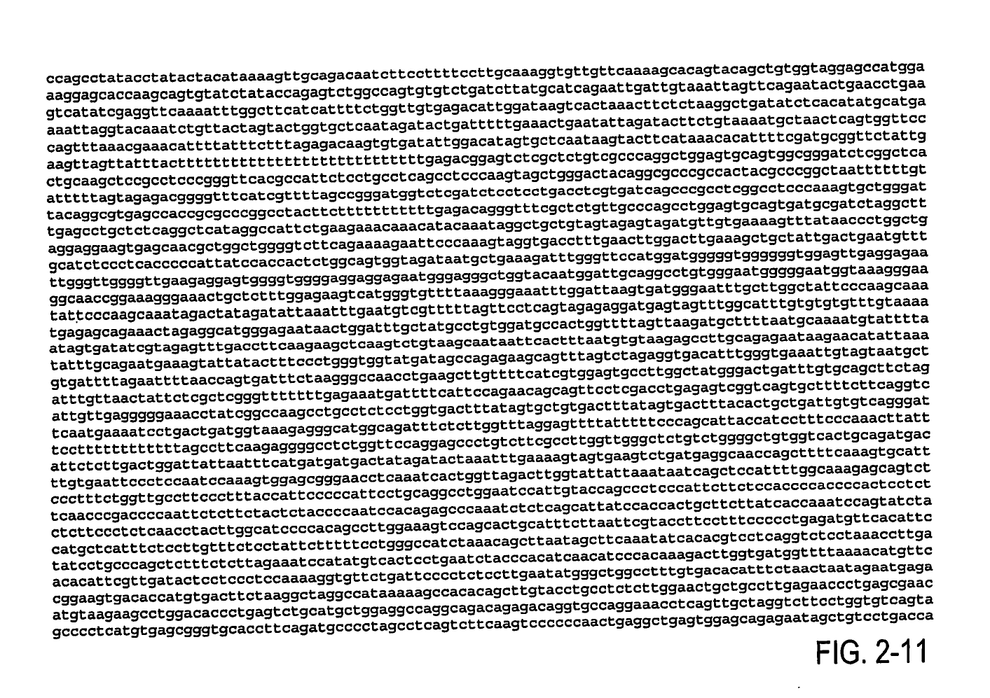

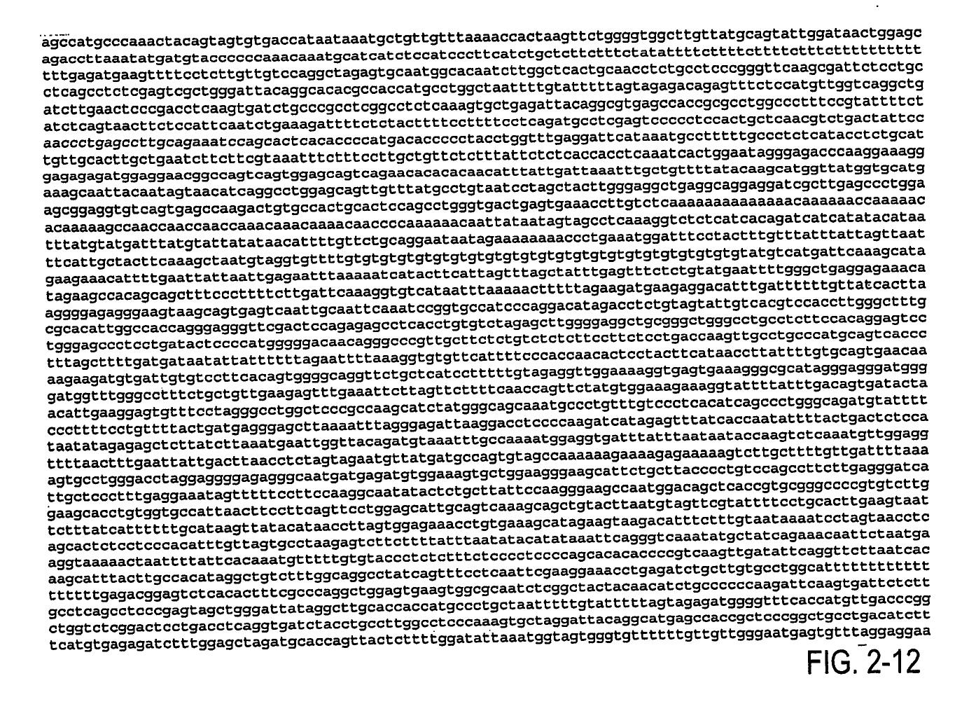

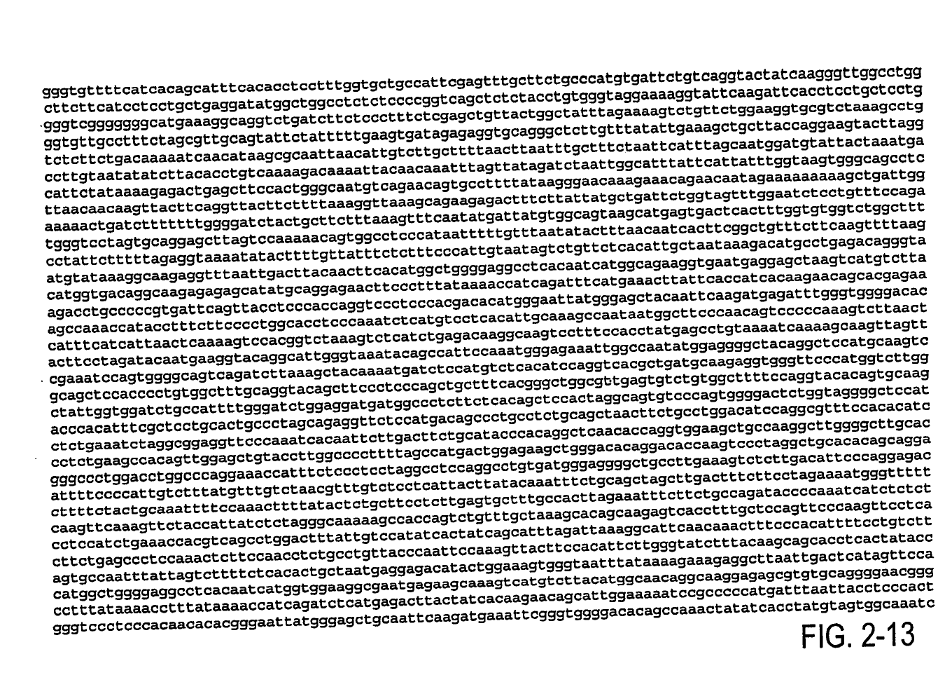

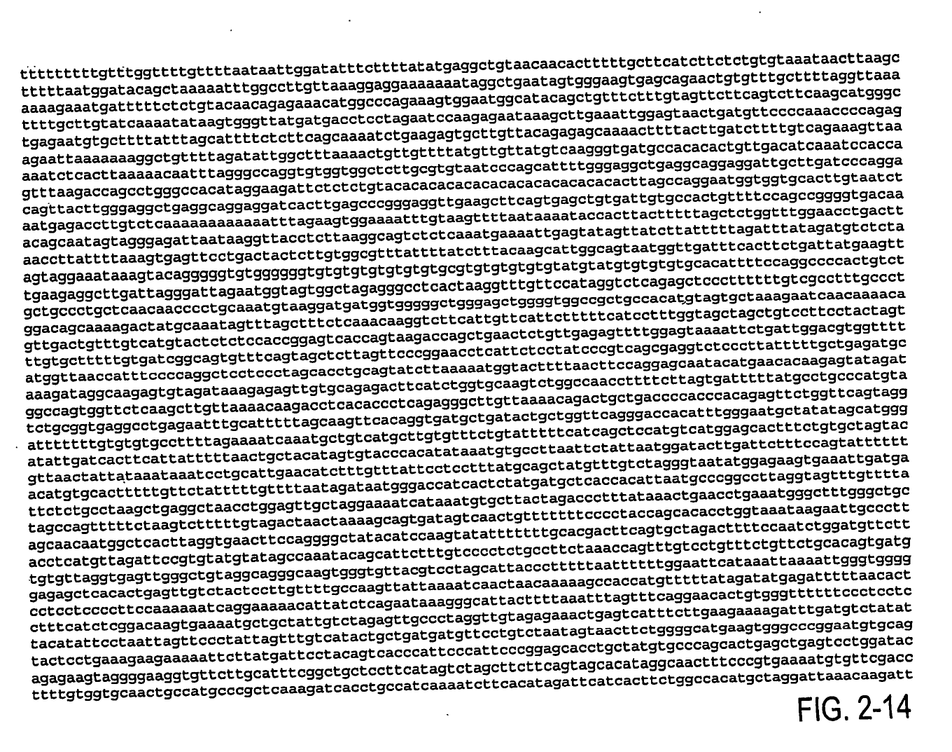

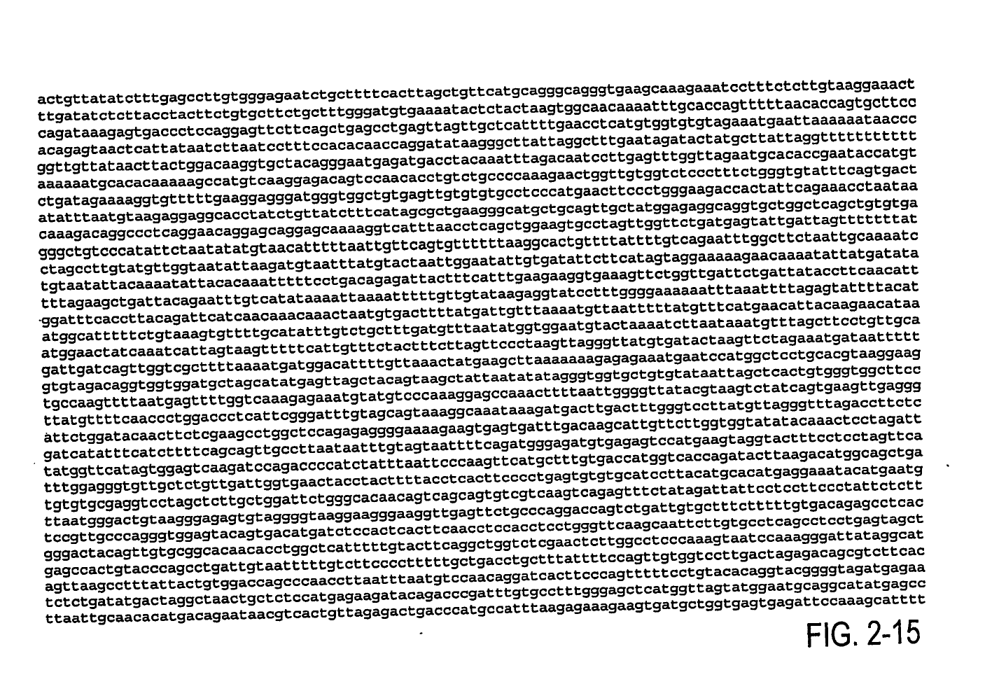

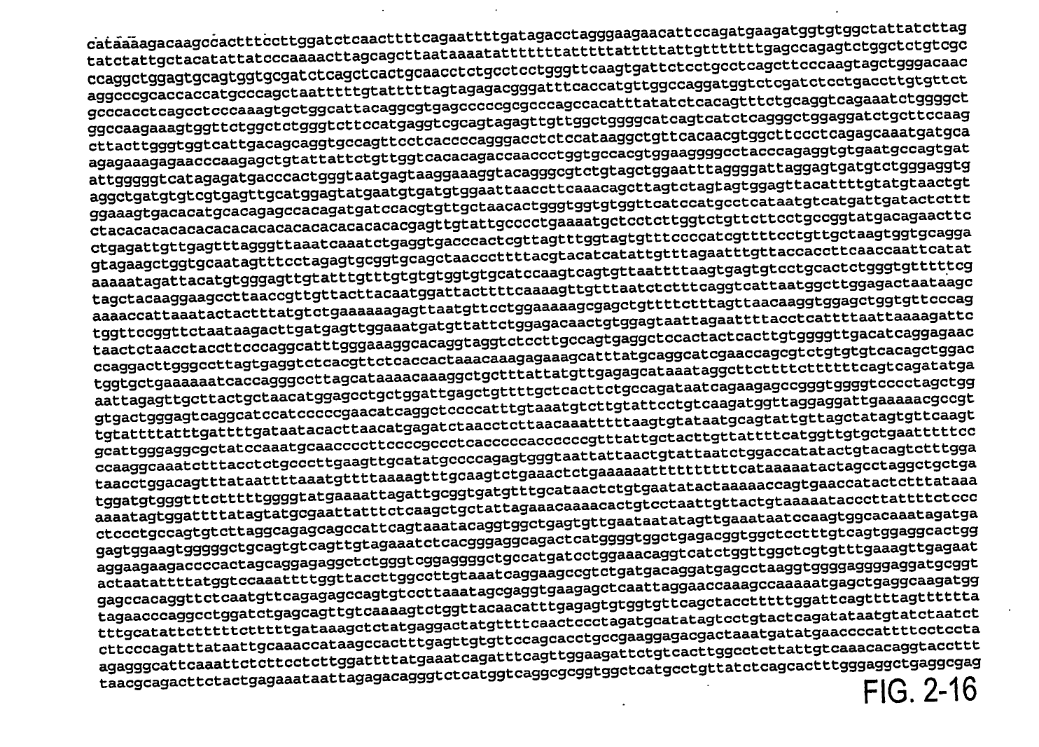

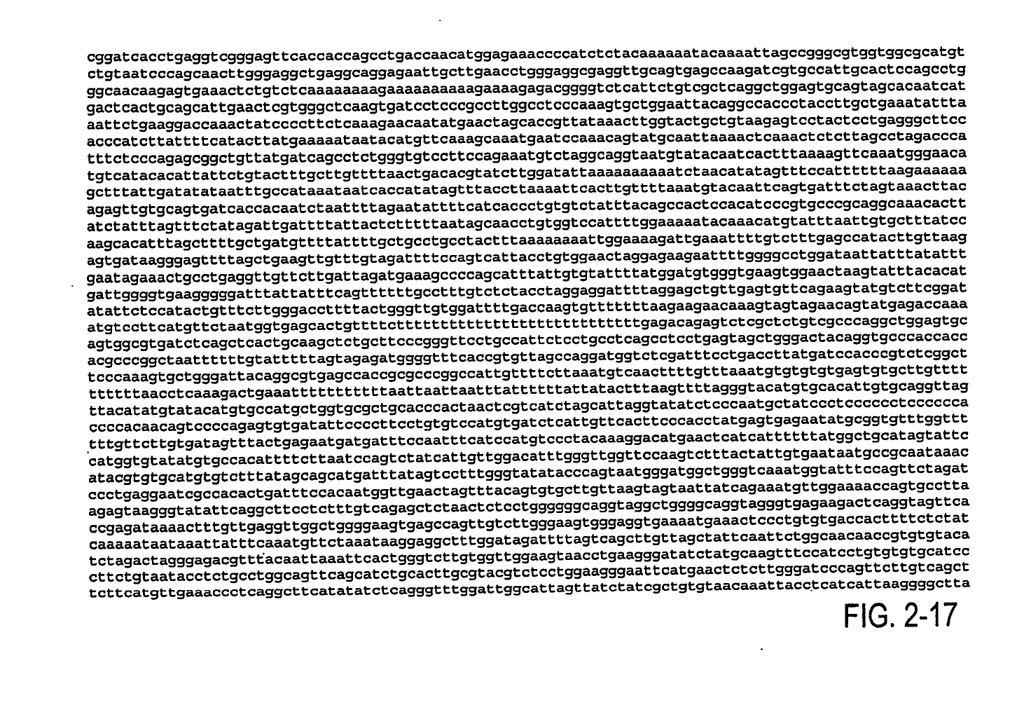

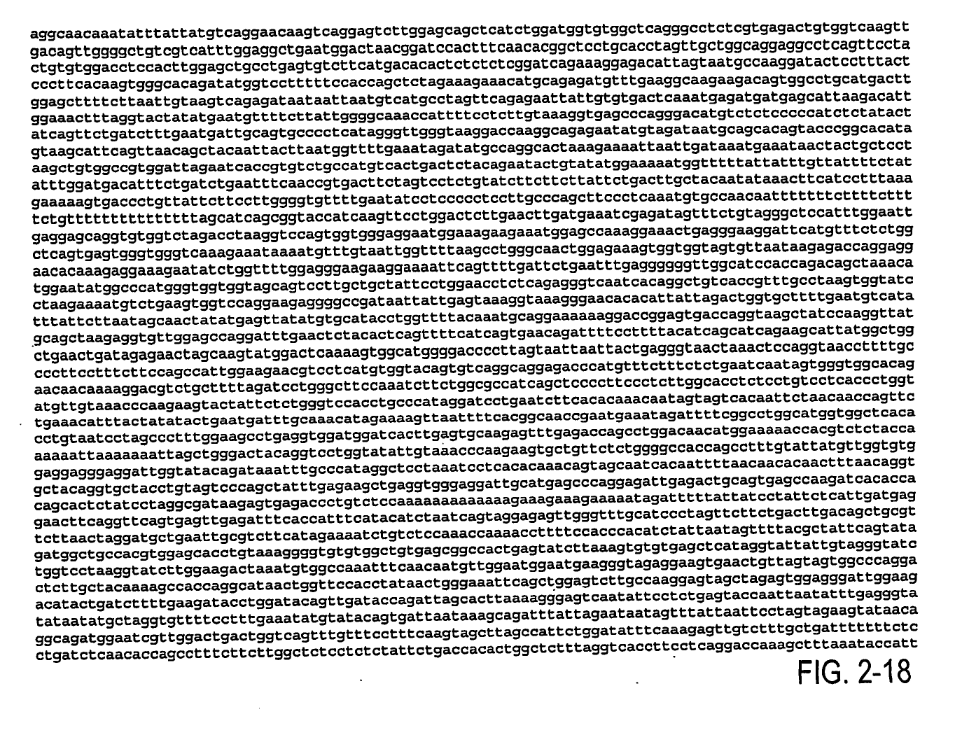

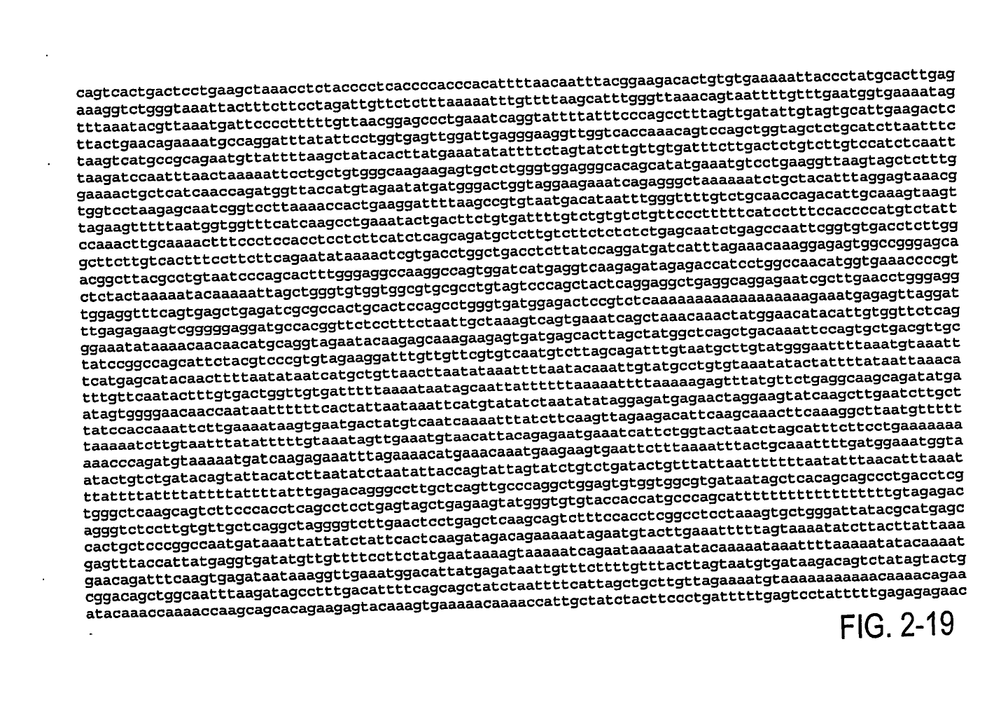

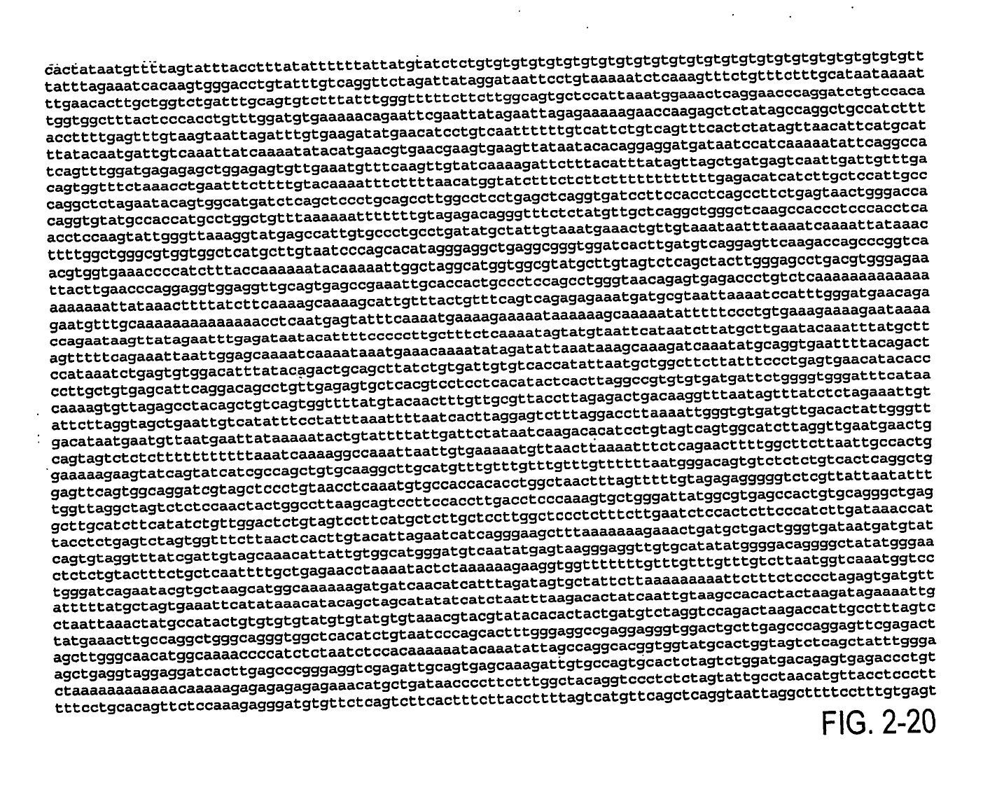

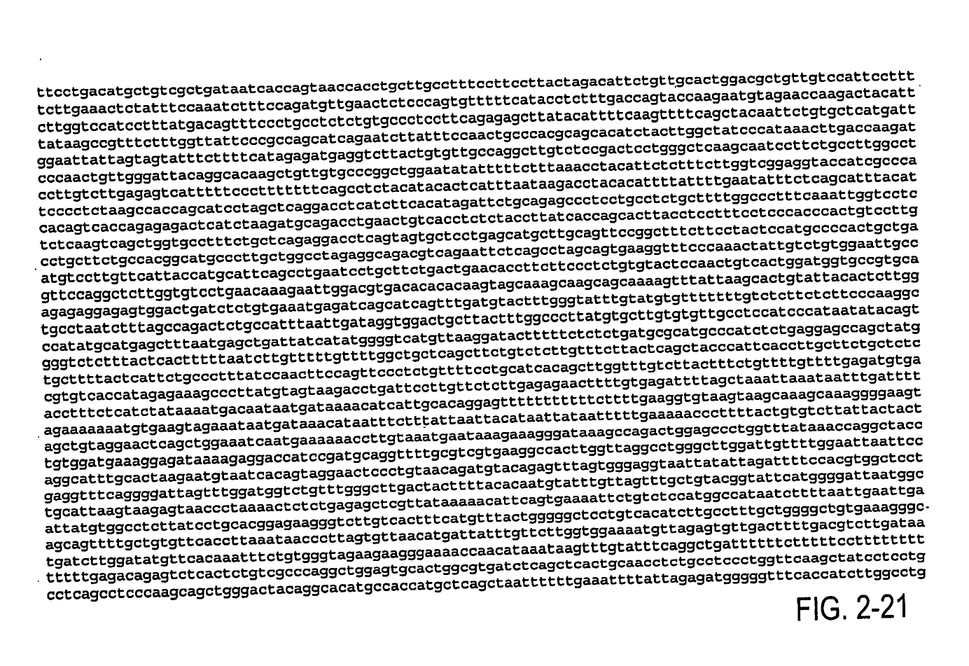

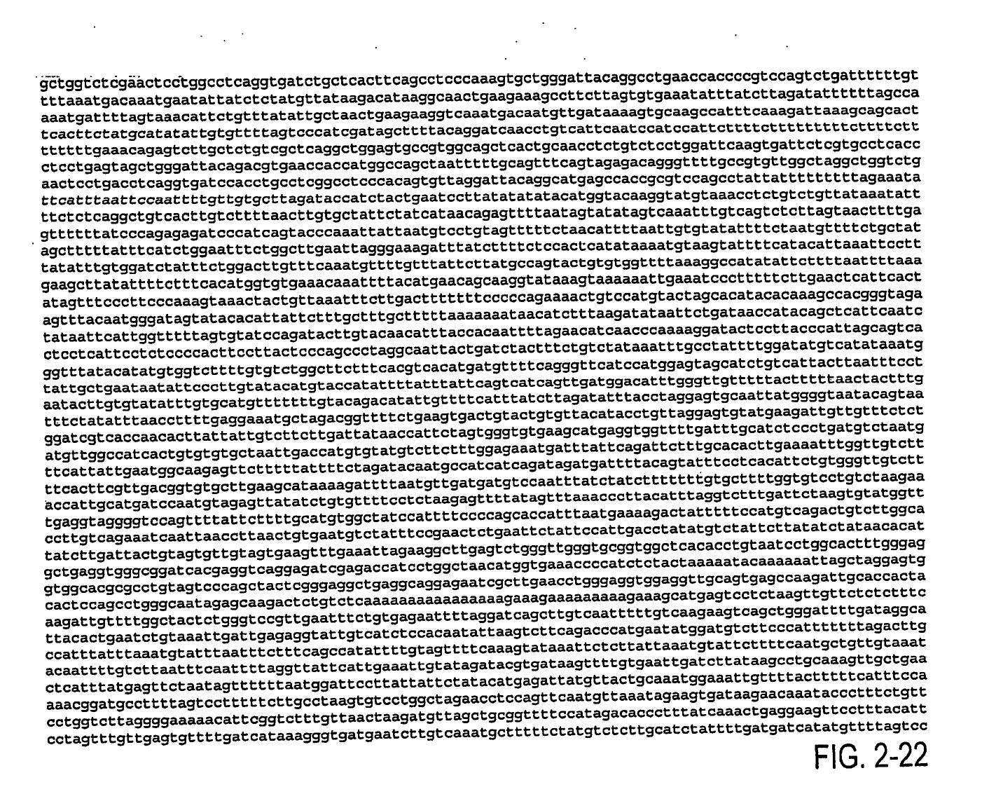

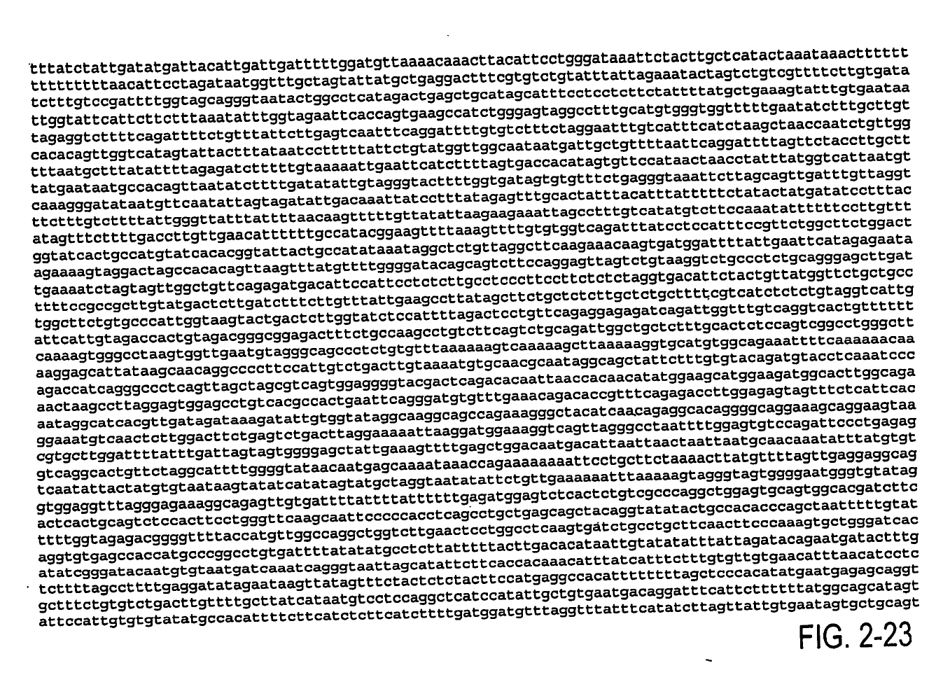

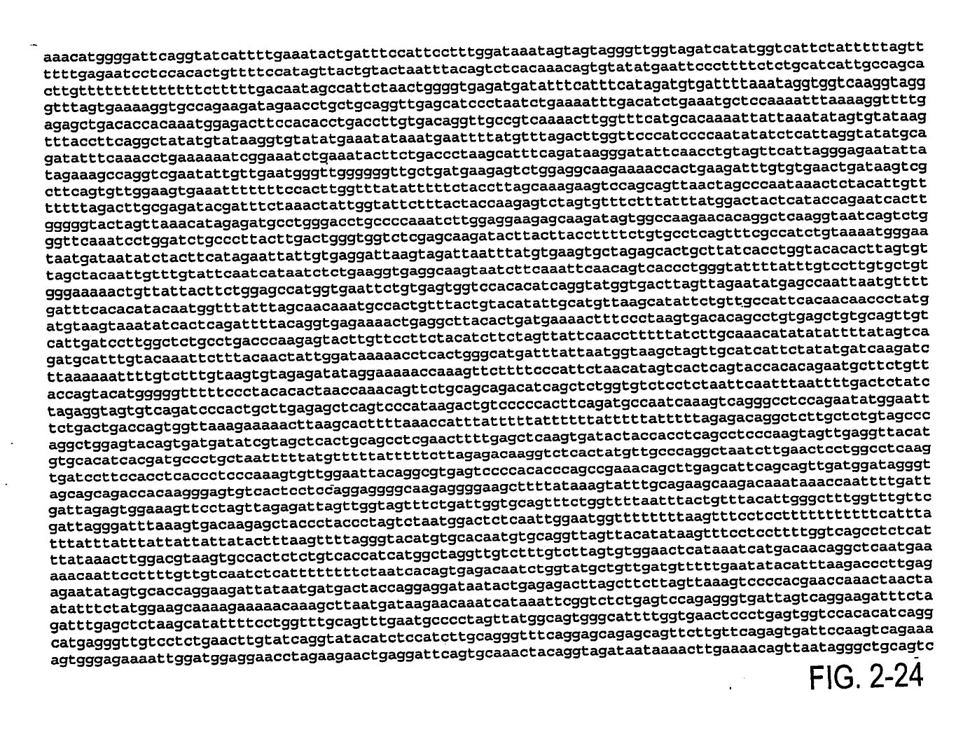

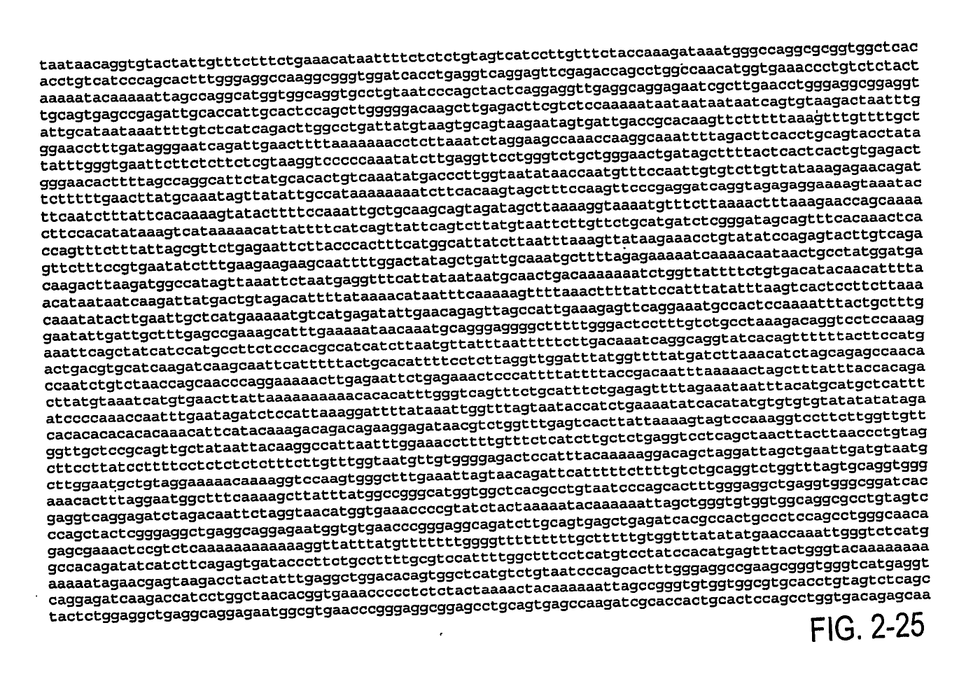

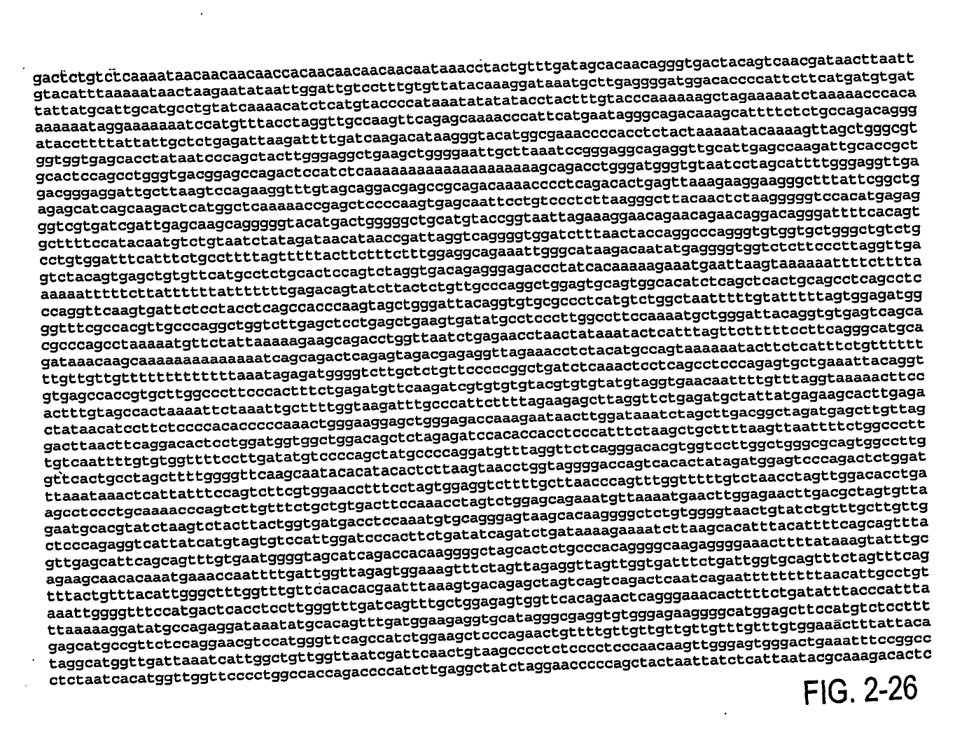

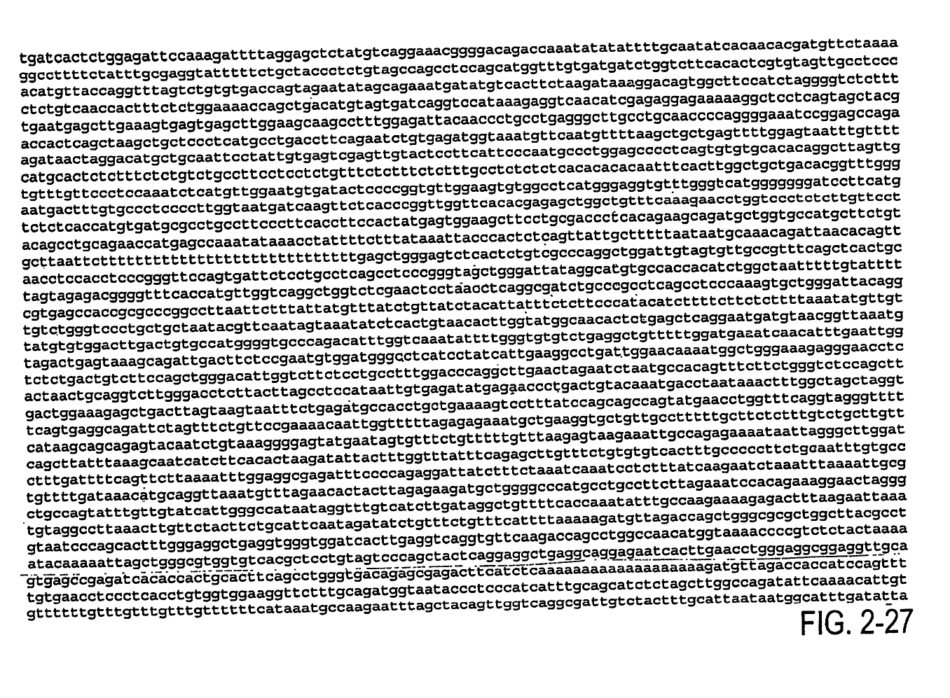

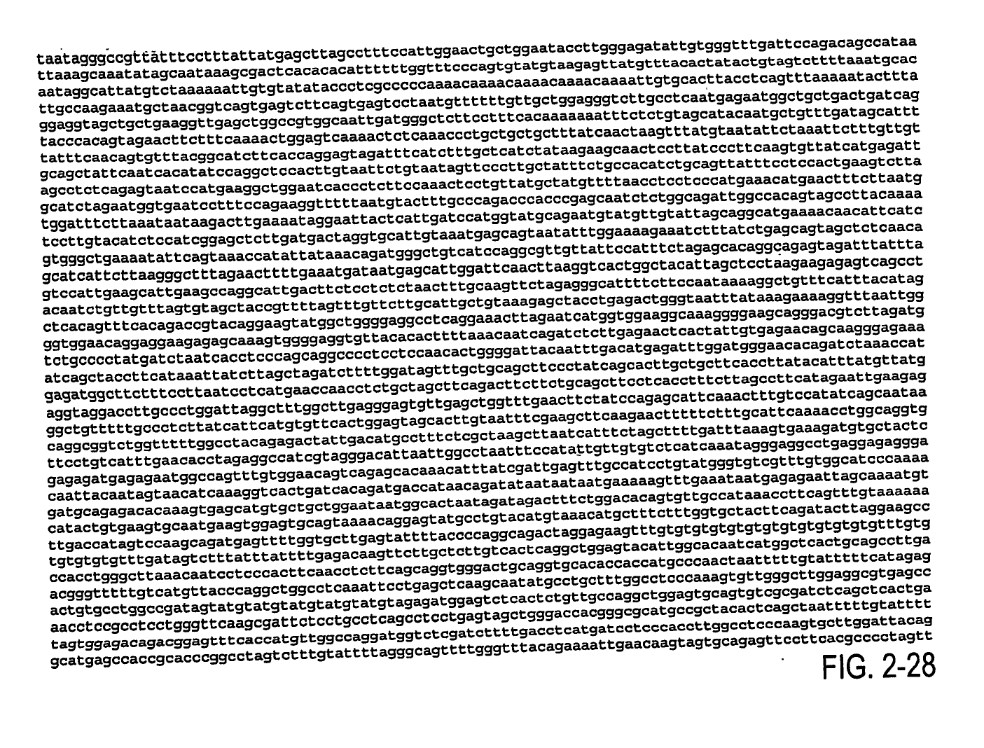

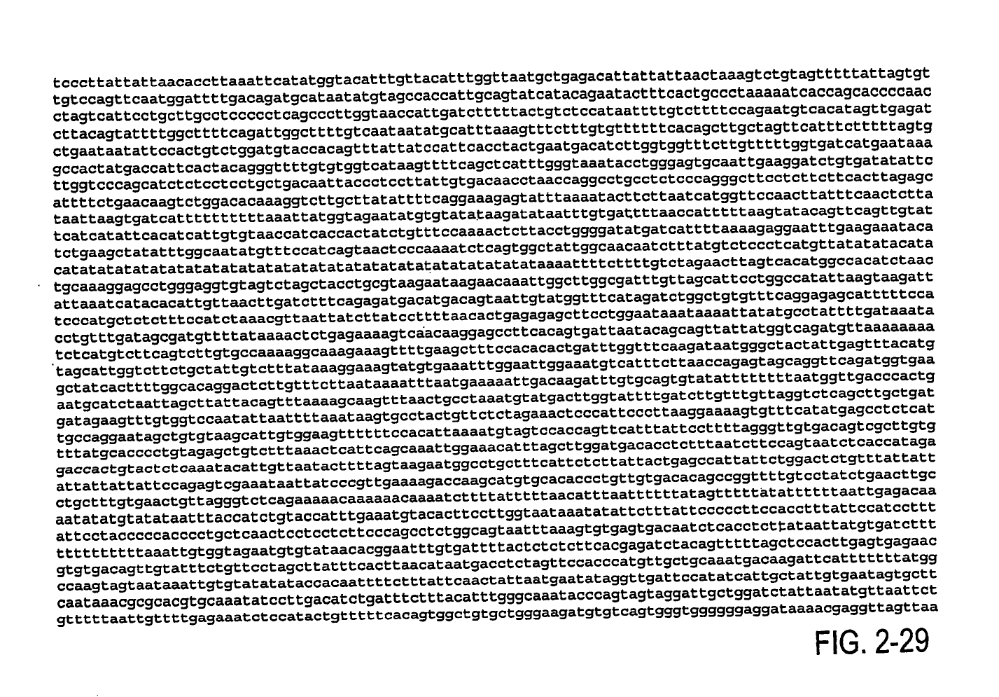

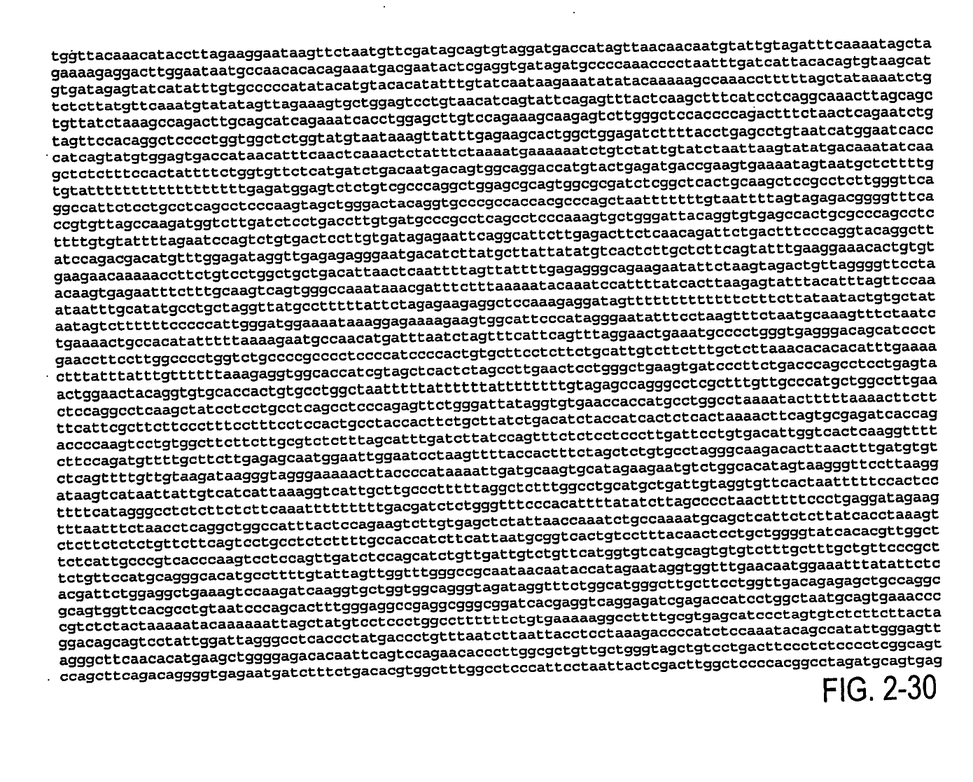

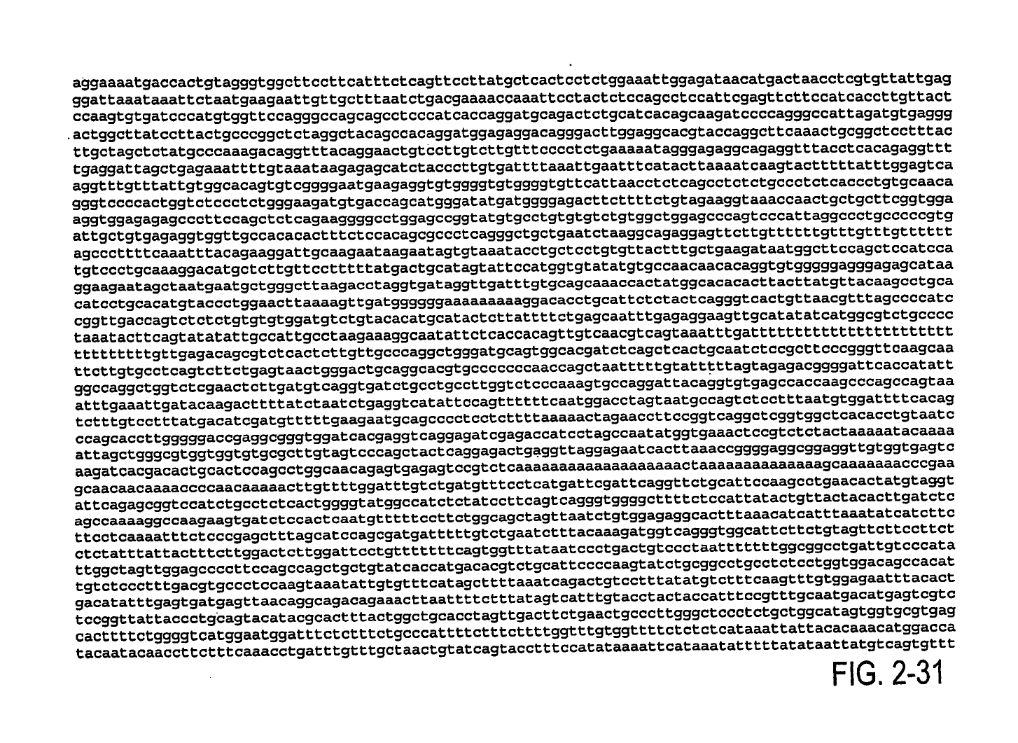

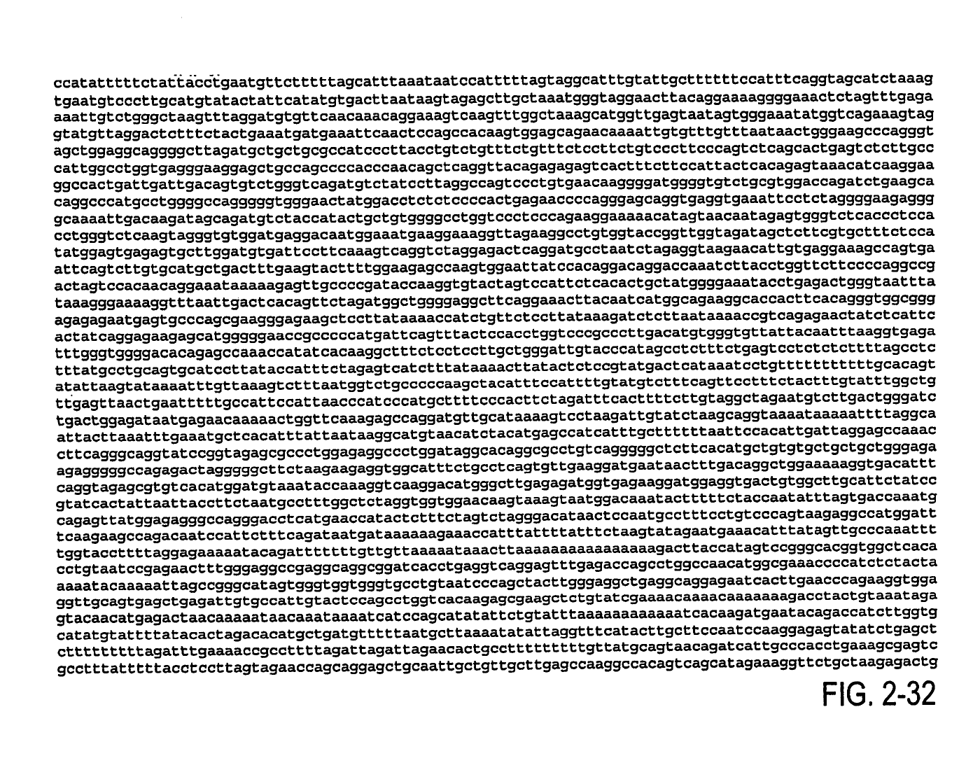

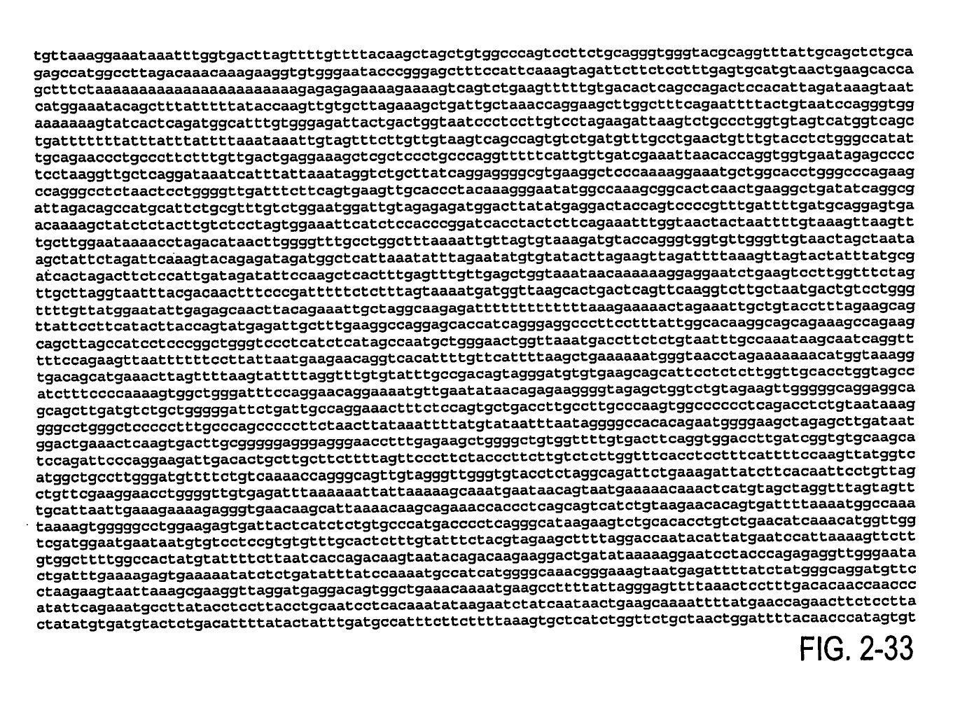

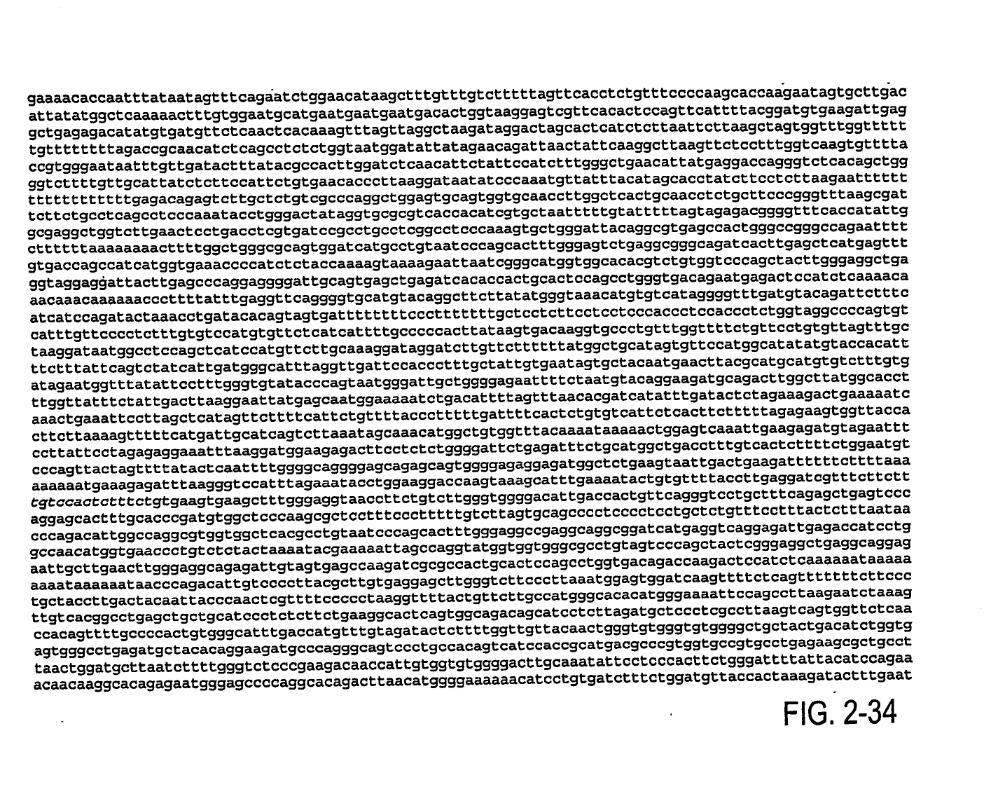

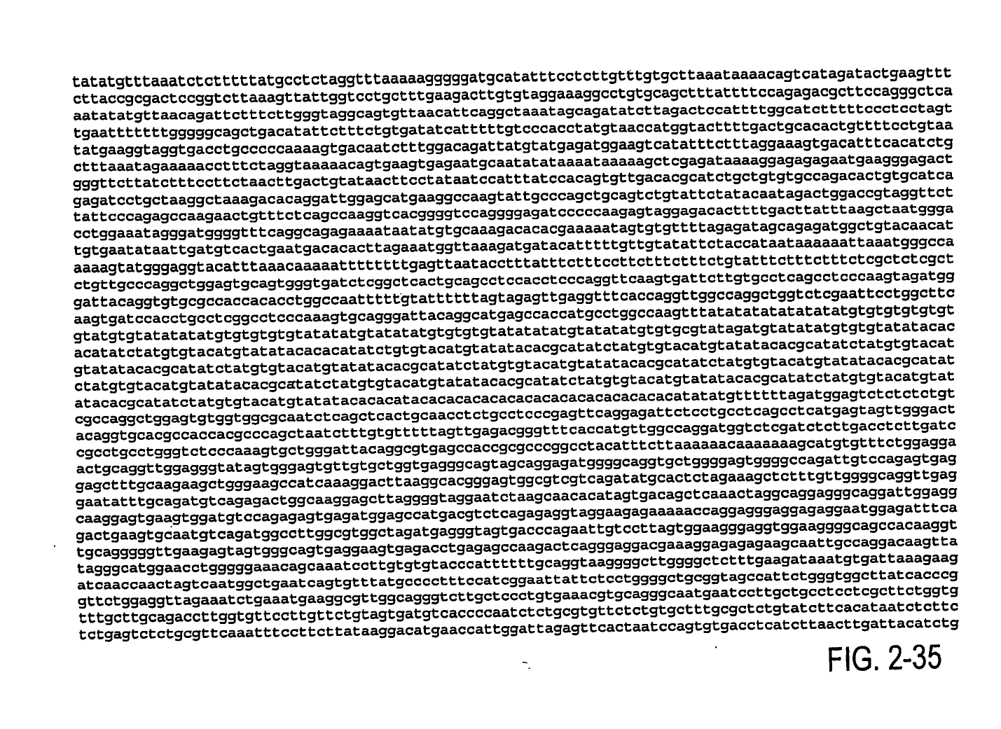

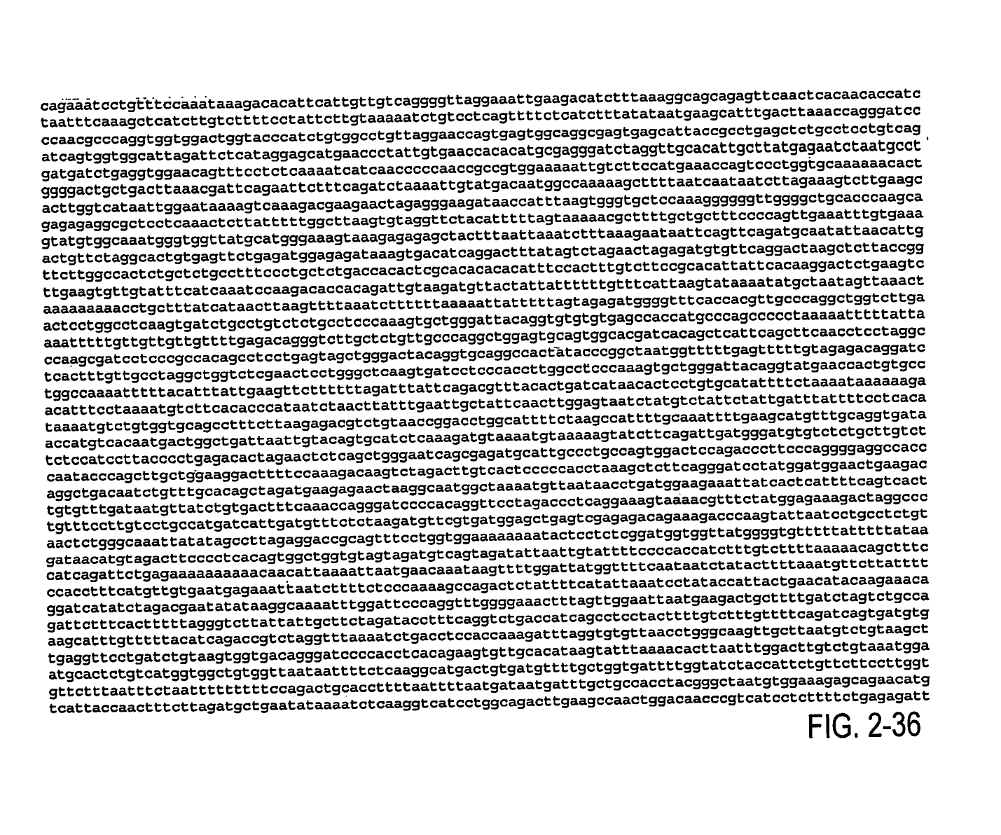

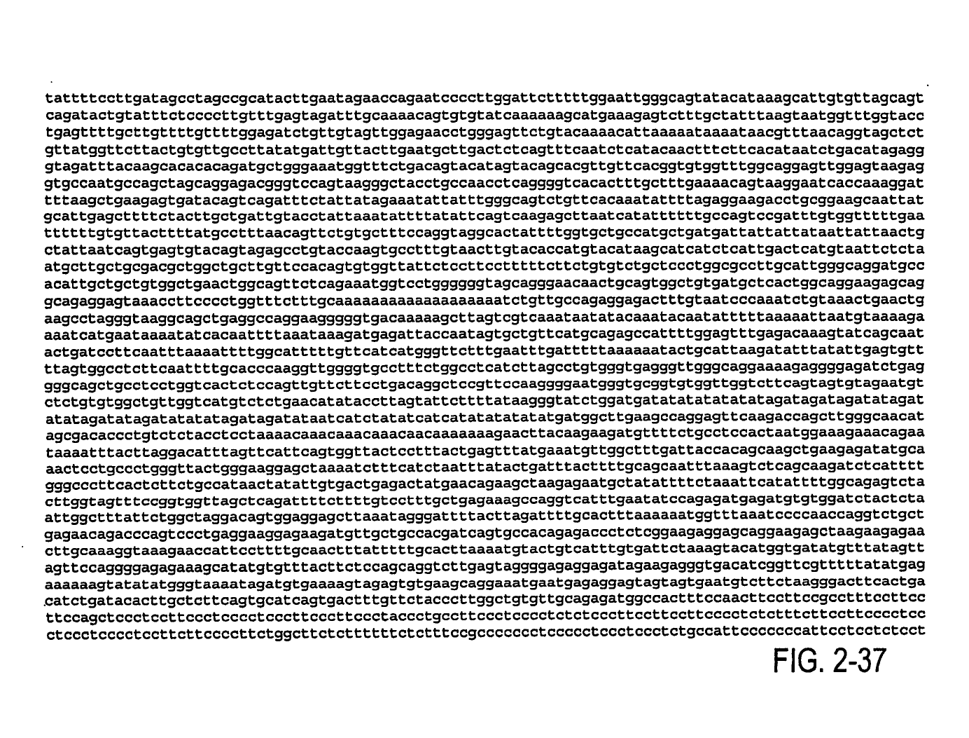

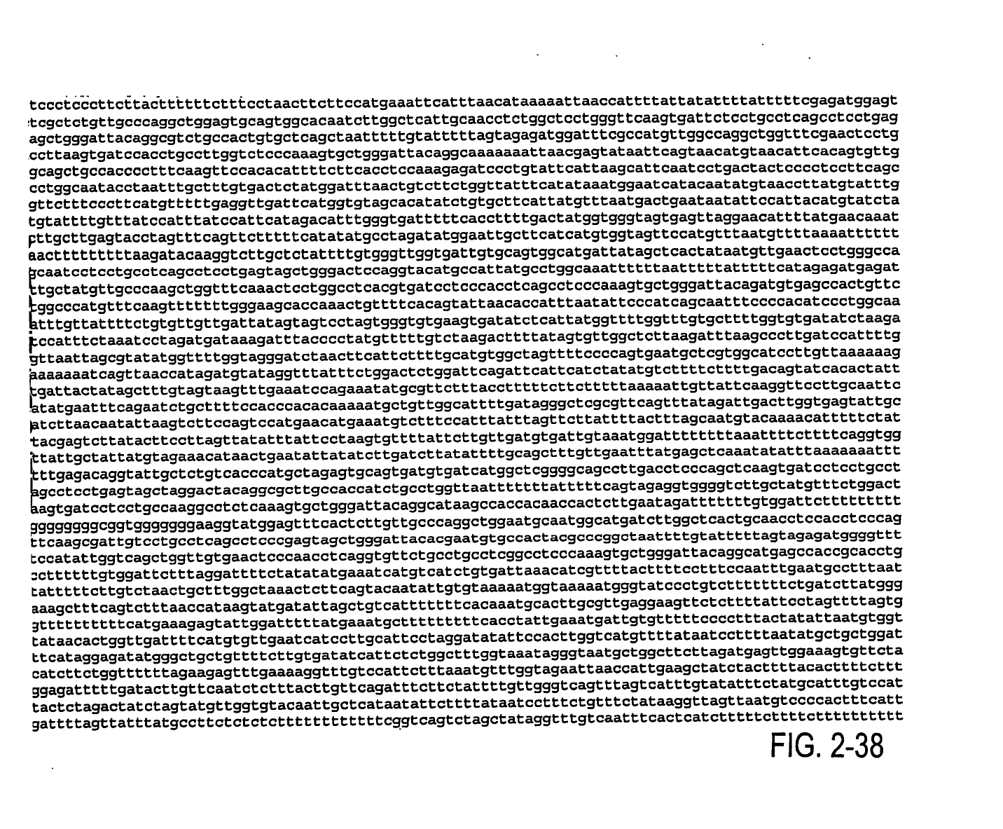

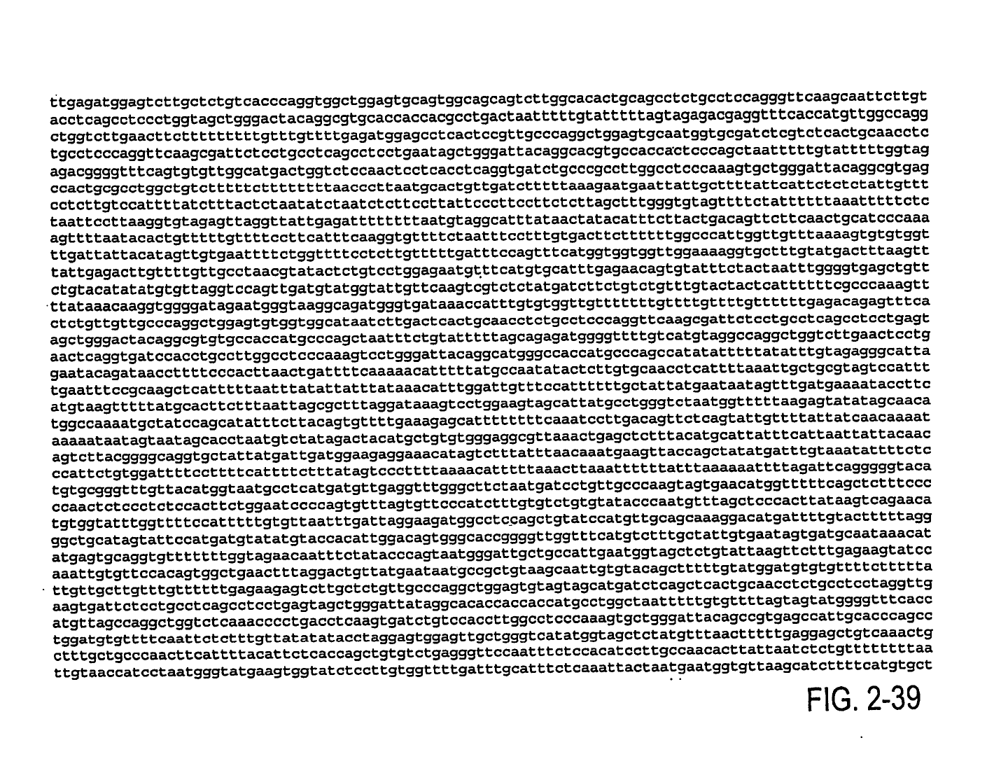

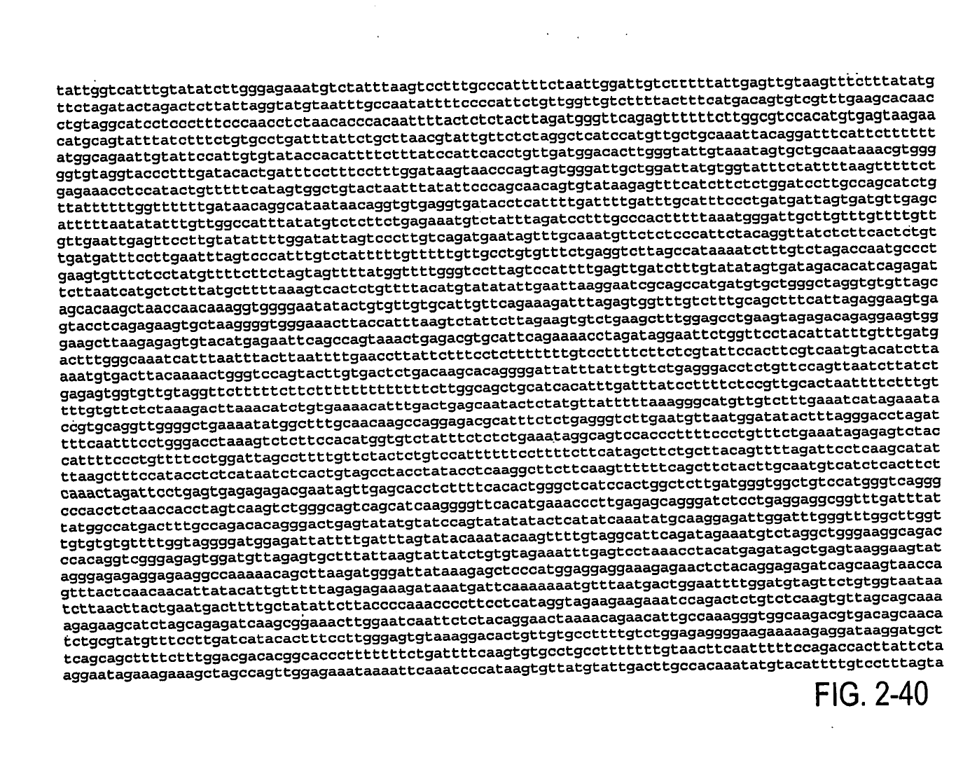

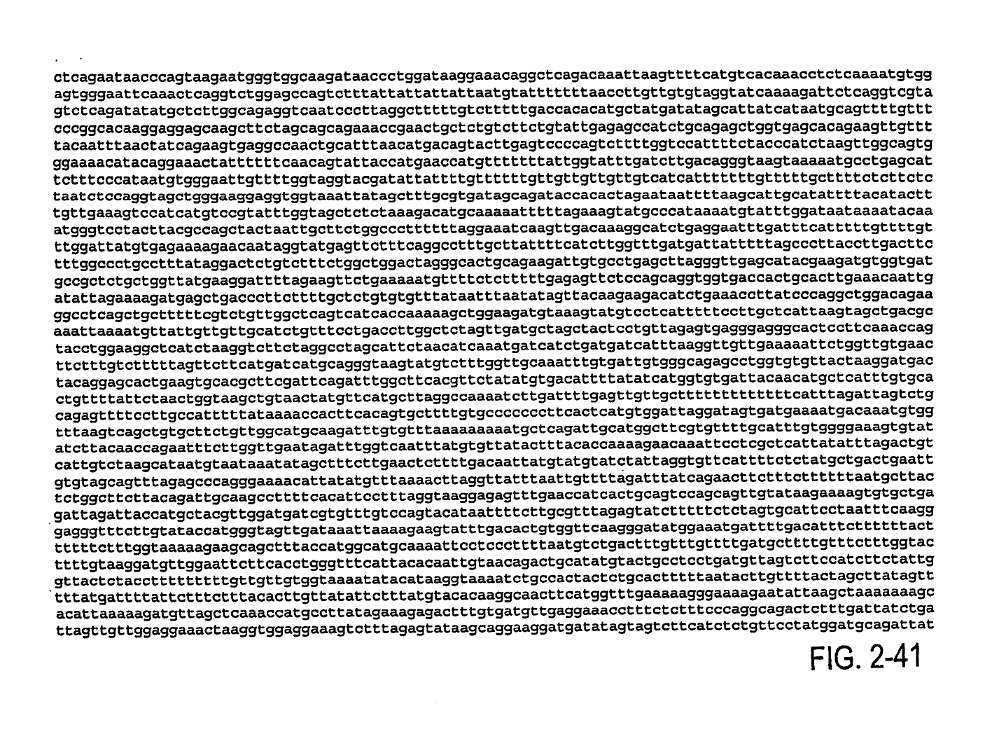

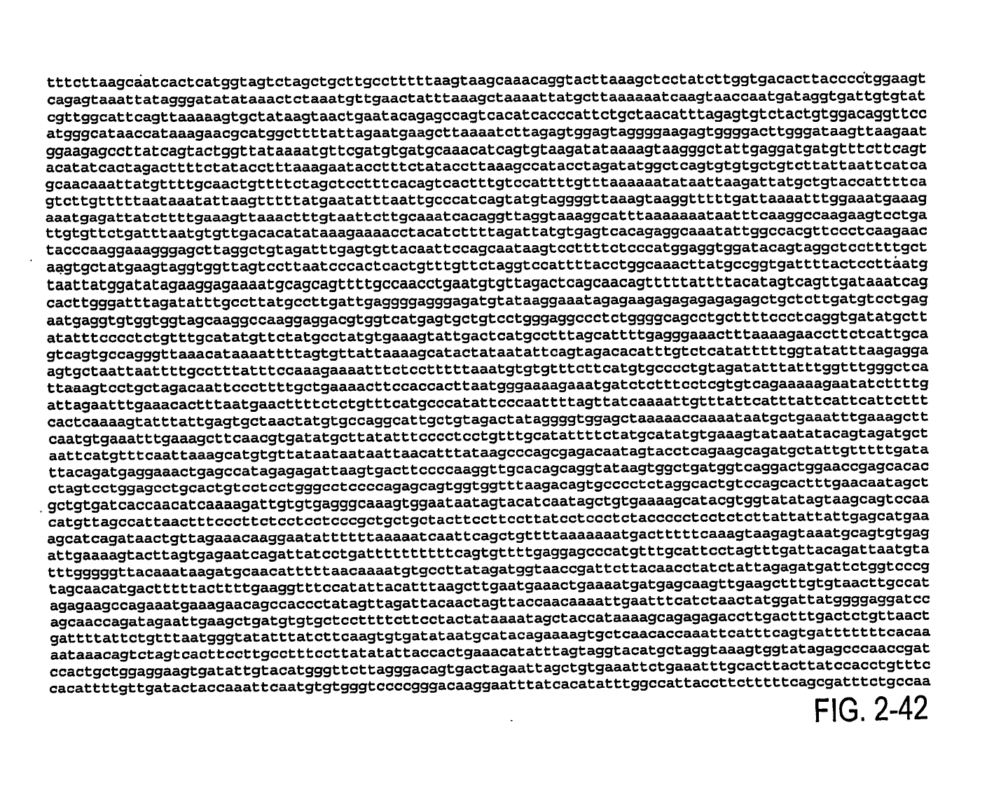

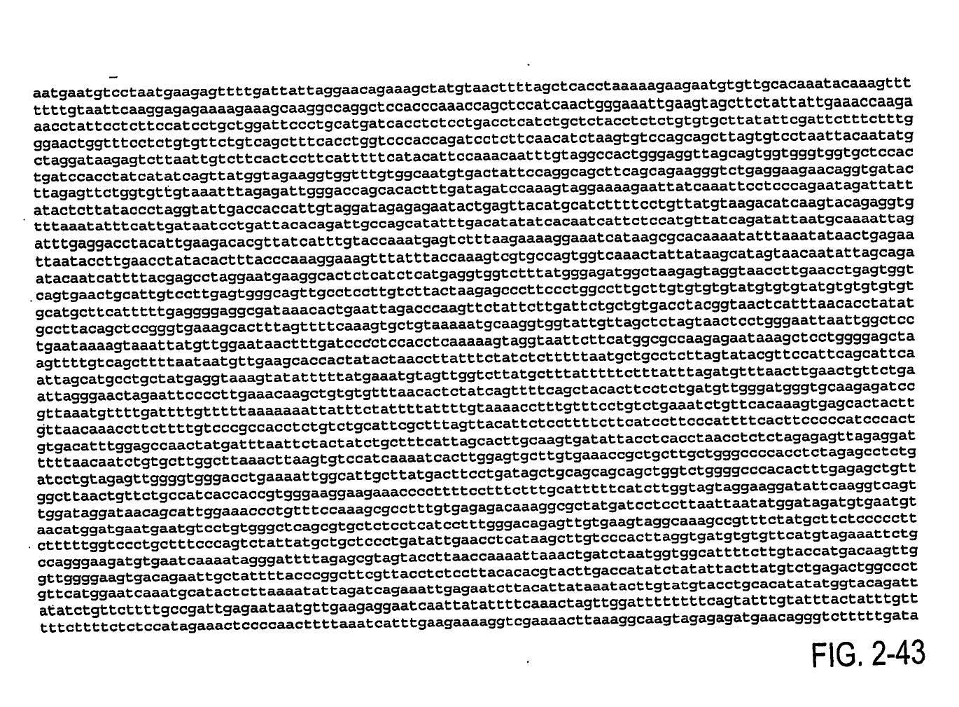

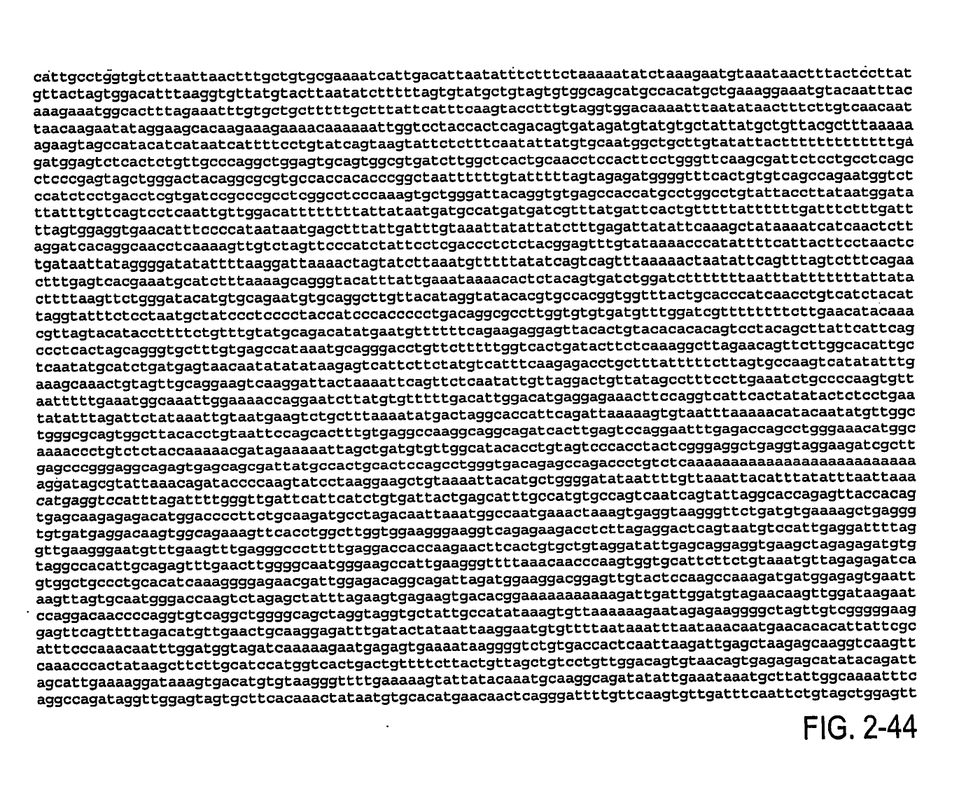

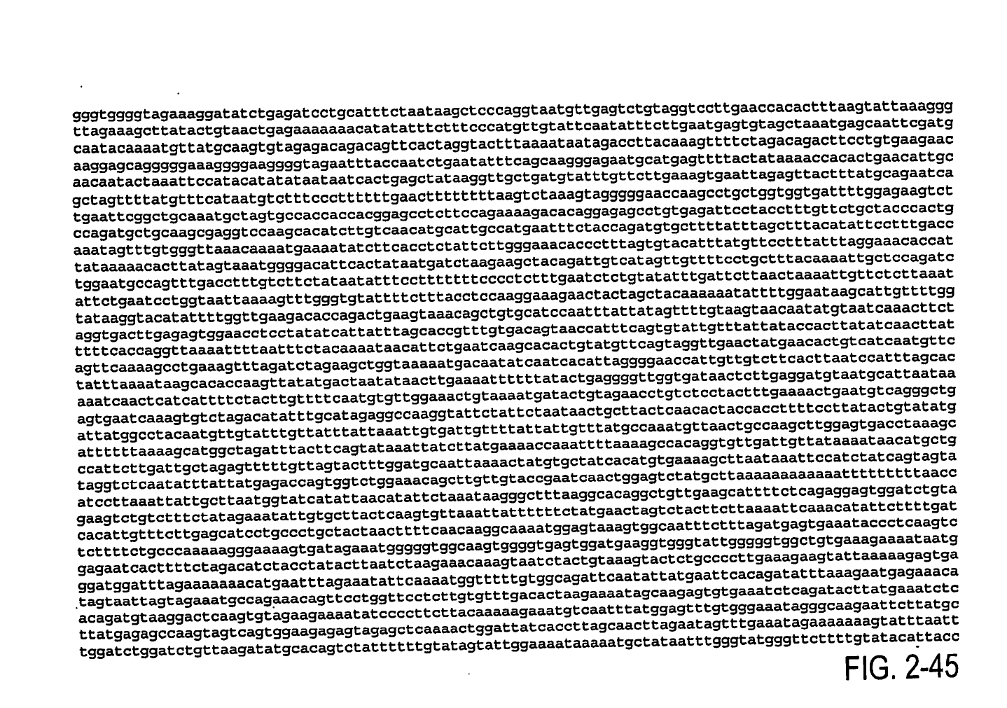

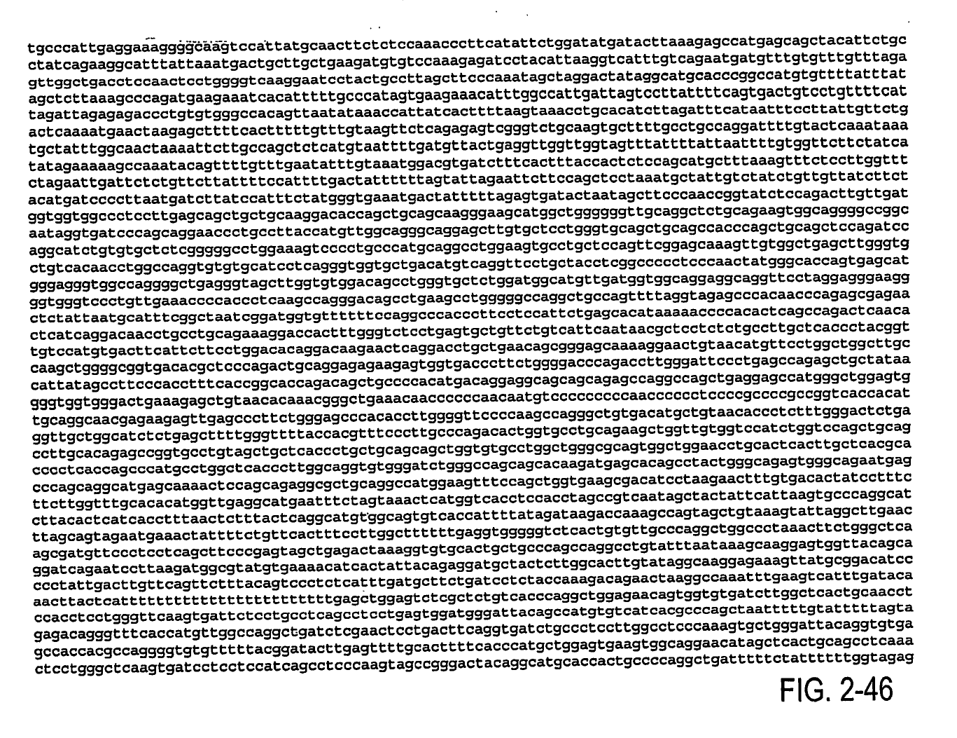

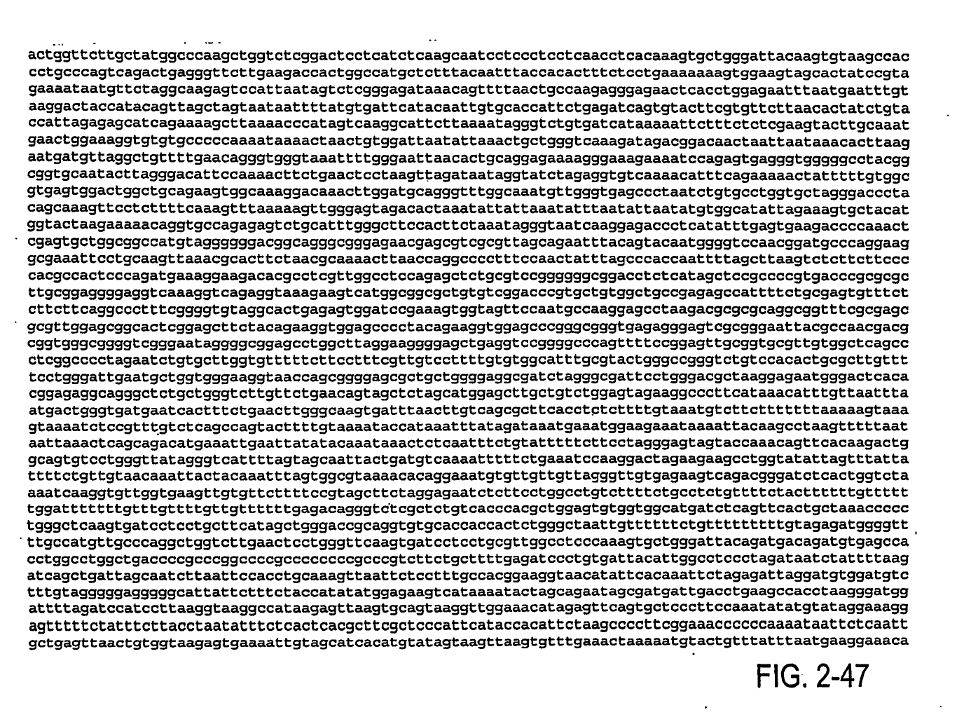

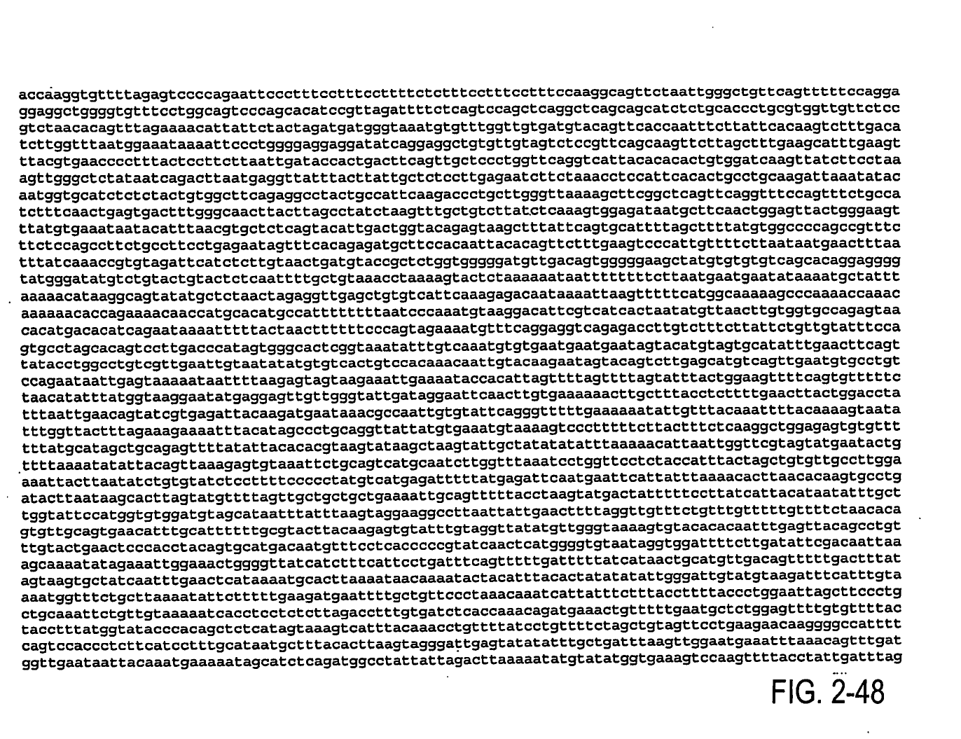

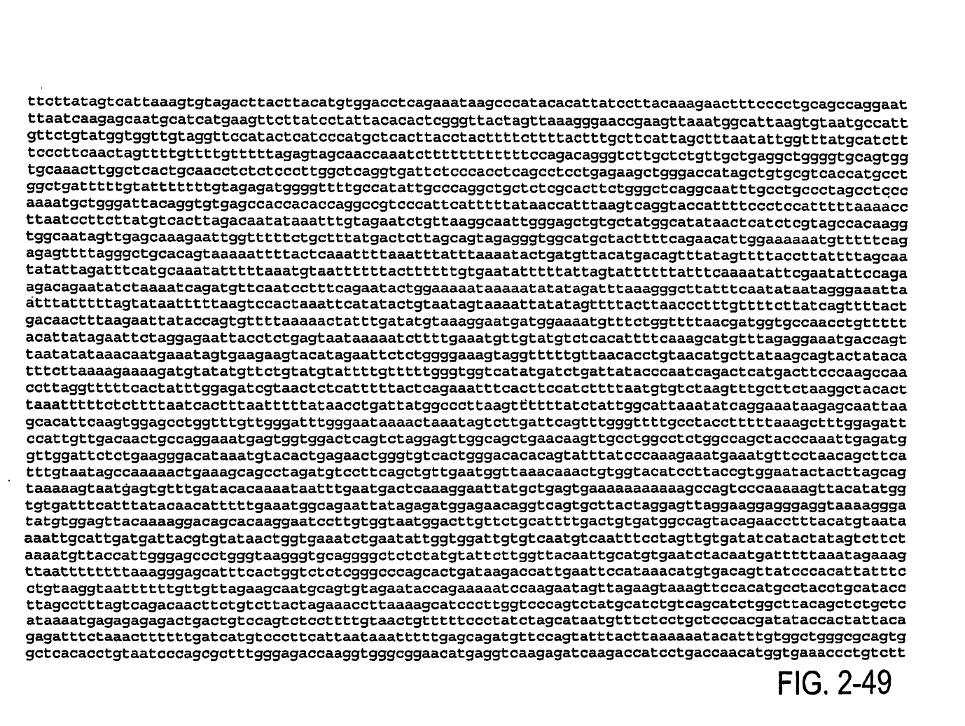

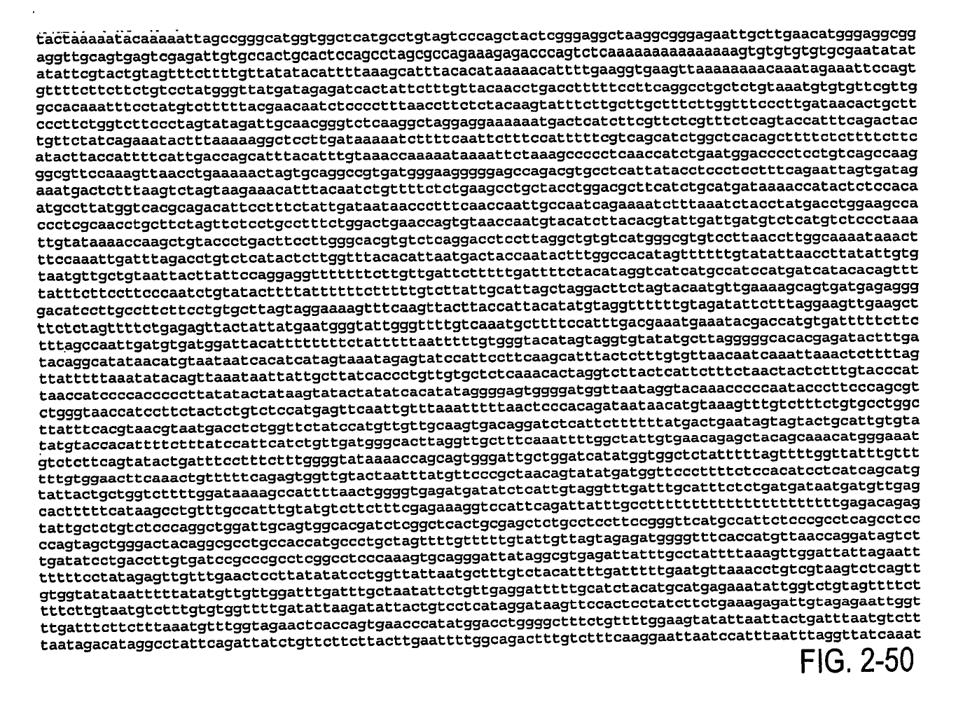

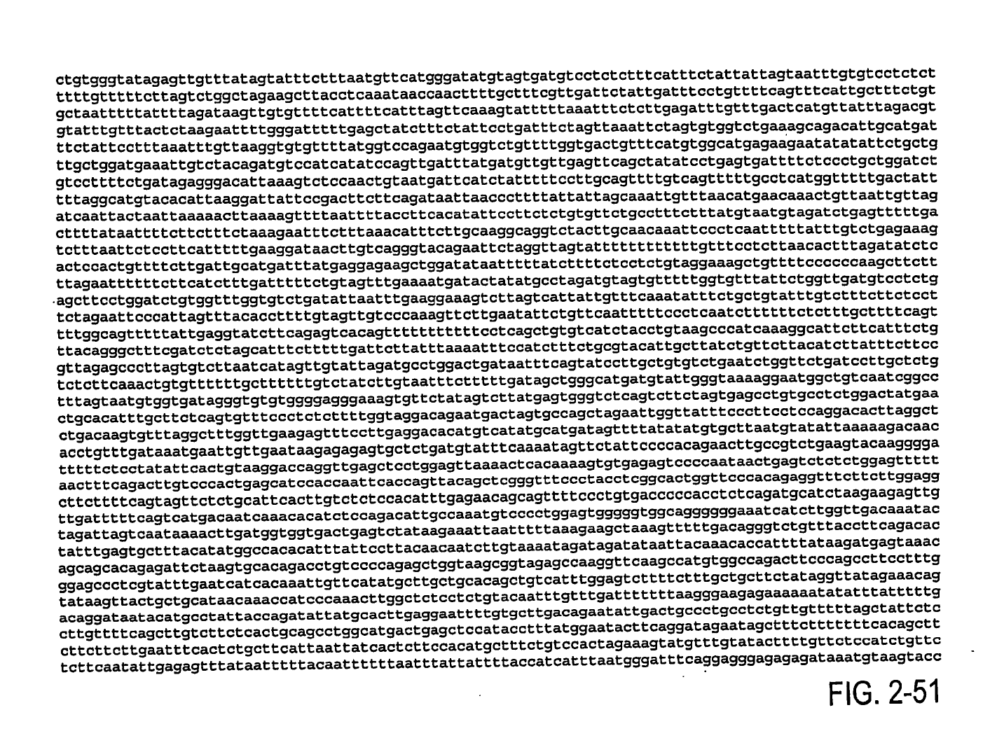

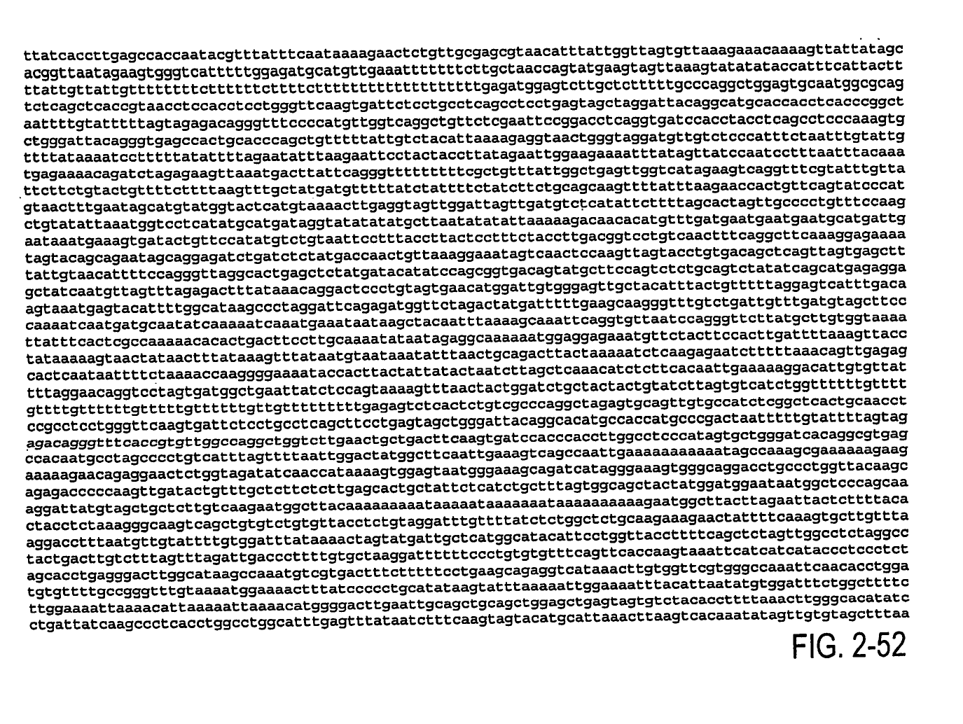

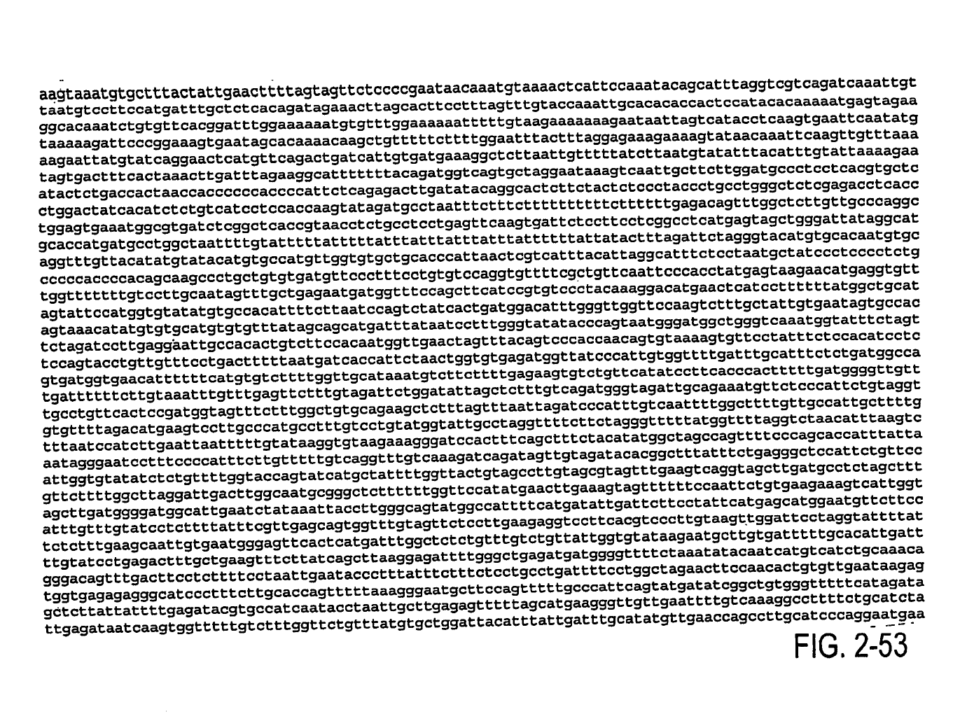

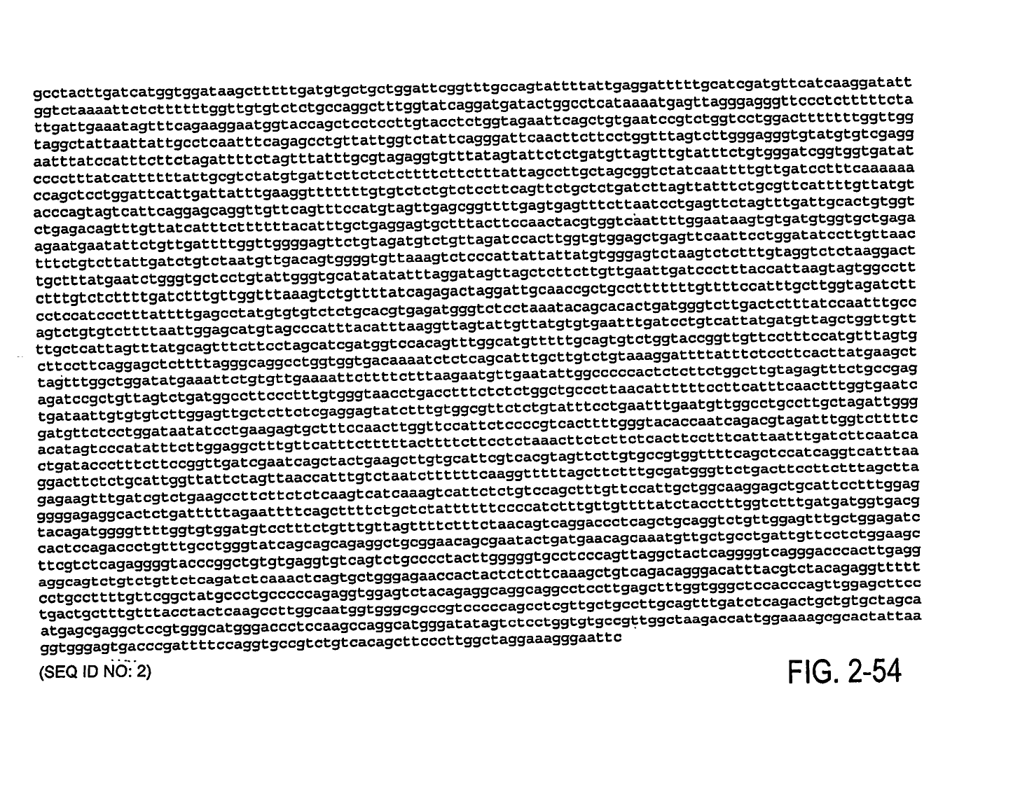

In the 5′region of the PrLZ cDNA, there was a unique 502 bp sequence containing the entire 5′UTR and the sequence coding for the N-terminal 41 amino acid residues (from 1 to 502 in Genbank accession number AF202897). In addition, we performed Southern blot hybridization to confirm that there was only one copy of this sequence in the human genome. Taking advantage of its gene-specificity, we used this fragment as a probe and localized the PrLZ gene to human chromosome 8q21.1 by fluorescence in situ hybridization. This gene-specific fragment was also used in the isolation of 270F20, a 180 kilobase pair (kb) genomic fragment cloned in the bacterial artificial chromosome (SAC), which encompassed the PrLZ gene and was mapped to the same chromosomal region. Exons of the PrLZ gene were mapped and corresponding restriction fragments subcloned for DNA sequencing analysis. Recently, the Human Genome Sequence Project released the draft sequence of the 8q21.1 region (Genbank accession number NT_{—}0233700), which contained the PrLZ gene. Our genomic studies indicated that the PrLZ mRNA was transcribed from six exons, which were scattered in a 45 kb region (the exons are present in FIG. 2).

We used the 502 bp gene-specific fragment as a probe to study the distribution of PrLZ in human tissues and cell lines. In studies with the human Multiple Tissue Expression array (Clontech, Palo Alto, CA), we found that PrLZ was predominantly expressed in human prostate, with minimal expression found only in the gastrointestinal tract, and a few other glandular tissues with secretive function (ie., salivary and mammary glands, pancreas, and kidney). These tissues would, therefore, be among those referred to herein as PrLZ-positive tissues (such tissues are amenable to the diagnostic and therapeutic methods of the present invention). We note here that, while we expect human cells and tissues to be used extensively in the methods of the invention, the invention is not so limited; any PrLZ-positive cells can be used, including those of mammals such as dogs, cats, monkey, pigs, cows, horses, sheep, goats, mice, rats, guinea pigs and other domesticated or laboratory-studied animals. In addition, cells from these tissues or cell lines derived from them can be used in the screening methods described herein to identify carcinogenic and chemotherapeutic agents.

PrLZ was highly expressed in prostate, but not in other paired normal or tumor samples of extra-prostatic organs and tissues. By analogy to the expression patterns of other prostate-specific genes, including the prostate specific antigen (PSA), the prostate specific membrane antigen (PSMA), and NKX3.1, we concluded that PrLZ was a prostate-specific gene.

Under normal culture condition (T-medium with 10% fetal bovine serum; Invitrogen, Carlsbad, Calif.), PrLZ was found expressed in all the tested prostate cancer cell lines, including both the AR positive LNCaP, C4-2, C4-2B4 cells, the ARCaP (low AR) cells, and the AR-negative PC3 and DU145 cells; these cell lines are among those having “PrLZ-positive” cells and, as such, are suitable for use in the screening methods of the present invention. Among these cell lines, the C4-2 cell line exhibited the highest constitutive expression. The expression pattern of PrLZ was in sharp contrast to those of the PSA and PSMA, which were expressed exclusively in the AR-positive INCAP and its lineage-derived sublines (i.e., C4-2, and C4-2B4).

The invention provides isolated or purified nucleic acid molecules that encode a PrLZ polypeptide described herein (e.g., a full-length PrLZ protein or a fragment thereof, such as those encoding a fragment of (e.g., an antigenic or immunologically detectable fragment of) SEQ ID NO:5 (e.g., the N-terminal 41 amino acids of PrLZ protein)). Also included are nucleic acid fragments suitable for use as primers (e.g., for the amplification or mutation of nucleic acid molecules) or hybridization probes; for use as antisense reagents (e.g., a ssRNA, dsRNA, siRNA, dsDNA, or mRNA-cDNA hybrid fragment); or for use in the production of anti-PrLZ antibodies (e.g., monoclonal, polyclonal, recombinant, chimeric, or humanized antibodies, or fragments thereof (e.g., single chain antibodies; such antibodies are within the scope of the present invention; see also, below).

An isolated nucleic acid molecule of the invention can include the nucleotide sequence shown in SEQ ID NO:1 or 2 or a portion of these sequences (e.g., an operable portion of the regulatory sequence of SEQ ID NO:1). Isolated nucleic acid molecules of the invention can include nucleic acid molecules that are complementary of the nucleotide sequence shown in SEQ ID NO:1 or 2 (or one or more portions thereof); such molecules can be used in the methods requiring PrLZ inhibition (these molecules are referred to above as “antisense” reagents), and they can also be used to detect PrLZ sequences in diagnostic assays. Nucleic acid molecules that are sufficiently complementary to the nucleotide sequence shown in SEQ ID NO:1 or SEQ ID NO:2 (or to a portion thereof) that they hybridize to SEQ ID NO:1 or SEQ ID NO:2 (thereby forming a stable duplex) under conditions defined in the art of molecular biology as “stringent” conditions are also within the scope of the present invention. For example, stringent conditions can refer to hybridization in 6× sodium chloride/sodium citrate (SSC) buffer at about 45° C., followed by two washes in 0.2×SSC buffer, 0.1% SDS at 60° C or 65° C. As used herein, the term “hybridizes under low stringency conditions” refers to conditions for hybridization in 6×SSC buffer at about 45° C., followed by two washes in 6×SSC buffer, 0.1% (w/v) SDS at 50° C.

We recognize that the sequence of the nucleic acid need not be identical to, or an identical complement of, SEQ ID NO:1 or SEQ ID NO:2 in order to be useful in the diagnostic, therapeutic, and screening methods of the present invention. More specifically, the invention encompasses nucleic acids molecules that are at least about: 60%, 65%, 70%, 75%, 80%, 85%, 90%, 91%, 92%, 93%, 94%, 95%, 96%, 97%, 98%, 99%, or more homologous to the entire length of the nucleotide sequences shown in SEQ ID NO:1 or SEQ ID NO:2 or to a portion, preferably of the same length, of any of these nucleotide sequences. Other nucleic acids of the invention differ by at least 1, but less than 5, 10, 20, 50, or 100 nucleotides relative to the nucleotide sequences represented by SEQ ID NO:1 or SEQ ID NO:2. Where the intended use of an antisense oligonucleotide is to inhibit gene transcription, the oligonucleotide can be made complementary to the region surrounding the translation start site of PrLZ mRNA (e.g., between the −10 and +10 regions of the target gene nucleotide sequence of interest—see, for example, the last 10 nucleotides of SEQ ID NO:1 (5′ ctgatgtttg 3′ and the corresponding position in SEQ ID NO:2)). If alignment is needed for this comparison the sequences should be aligned for maximum homology.

As implied by the preceding description, the invention encompasses nucleic acid molecules that include only a portion of the nucleic acid sequences of SEQ ID NO:1 or 2. For example, such a nucleic acid molecule can include a fragment which can be used as a probe or primer or a fragment encoding a portion of a PrLZ protein (e.g., an immunogenic or biologically active portion of a PrLZ protein). For example, a fragment can comprise those nucleotides of SEQ ID NO:1 or 2 that encode a leucine rich (e.g., a leucine zipper (for example, a leucine zipper shown in FIG. 3) of PrLZ, a serine-rich domain of PrLZ, or another domain described herein. While the present methods focus primarily on PrLZ, the probes and primers described herein can be used to identify and/or clone other TPD52 family members (or fragments thereof), as well as PrLZ homologues (or fragments thereof) from other species. The oligonucleotides of the invention can contain at least (or about) 7, 12 or 15 nucleotides; they may also be longer (e.g., at least (or about) 20 or 25 nucleotides (e.g., 30, 35, 40, 45, 50, 55, 60, 65, or 75 consecutive nucleotides of a sense or antisense sequence of SEQ ID NO:1 or SEQ ID NO:2. In one or more of the methods of the invention, a PrLZ-specific probe or primer may be attached to a solid support (e.g., a bead, chip, or slide).

The nucleic acids of the invention (whether comprising sense or antisense sequence; whether comprising non-coding regulatory sequence (e.g., 5′ regulatory elements (e.g. SEQ ID NO:1)); or whether further comprising a vector for expression) can be included in a kit that is packaged with instructions for its use. Where the use is detection of a PrLZ gene, nucleic acid primers can be packaged in pairs to amplify the PrLZ gene or an exon within it (see the Examples herein). For example, the kit can contain a “forward” primer that anneals to the coding strand and a “reverse” primer that anneals to the non-coding strand. Methods for amplifying sequences with primers, or otherwise detecting sequences by hybridization, are now well known in the art.

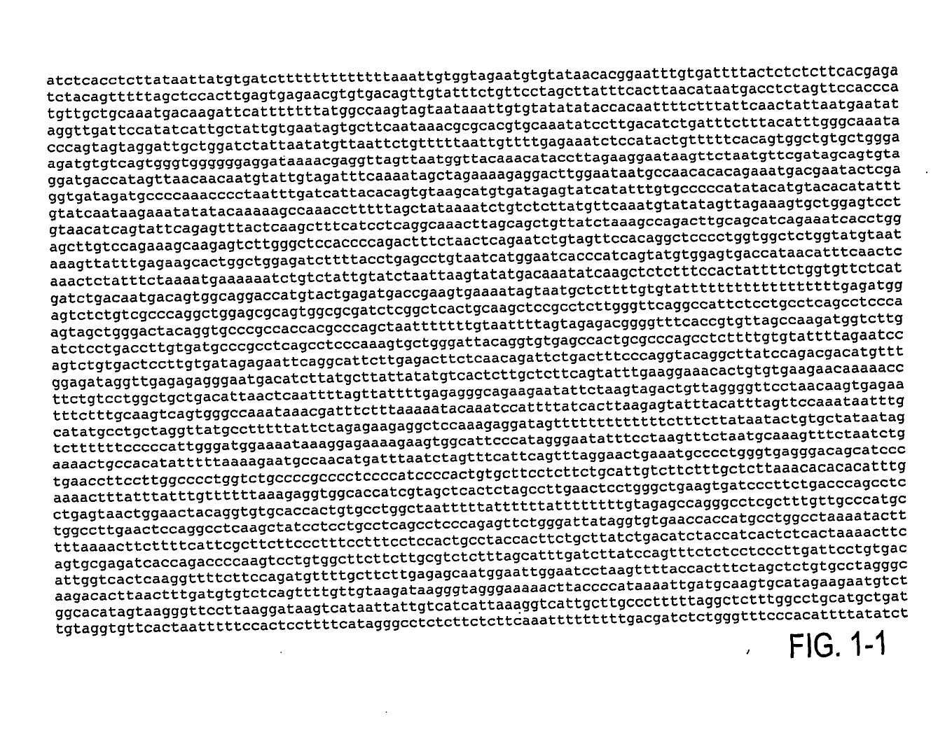

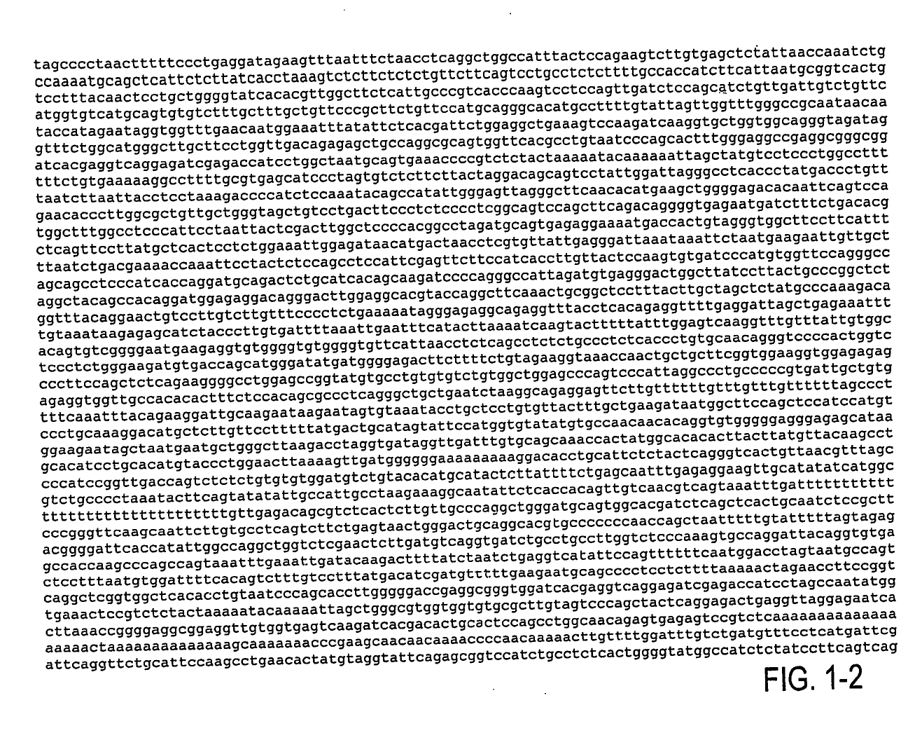

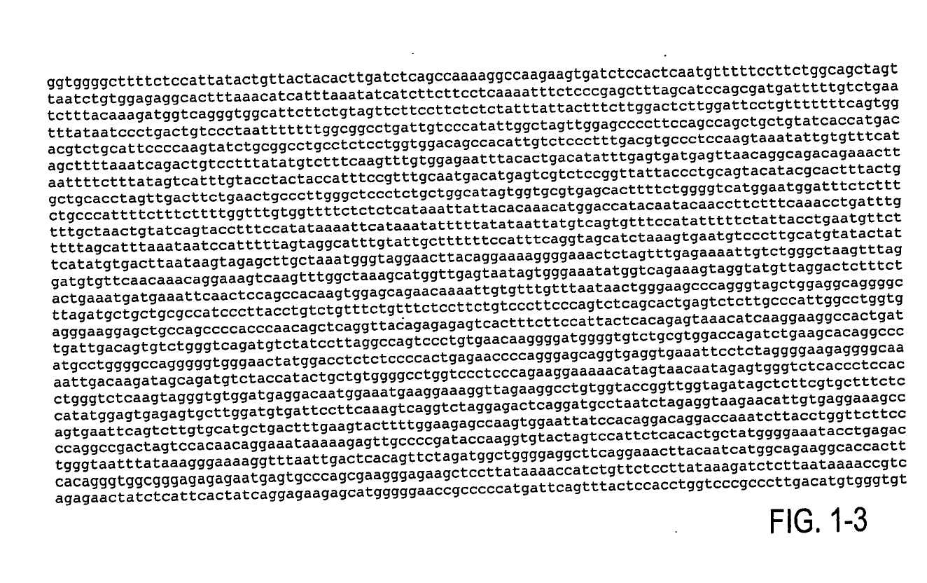

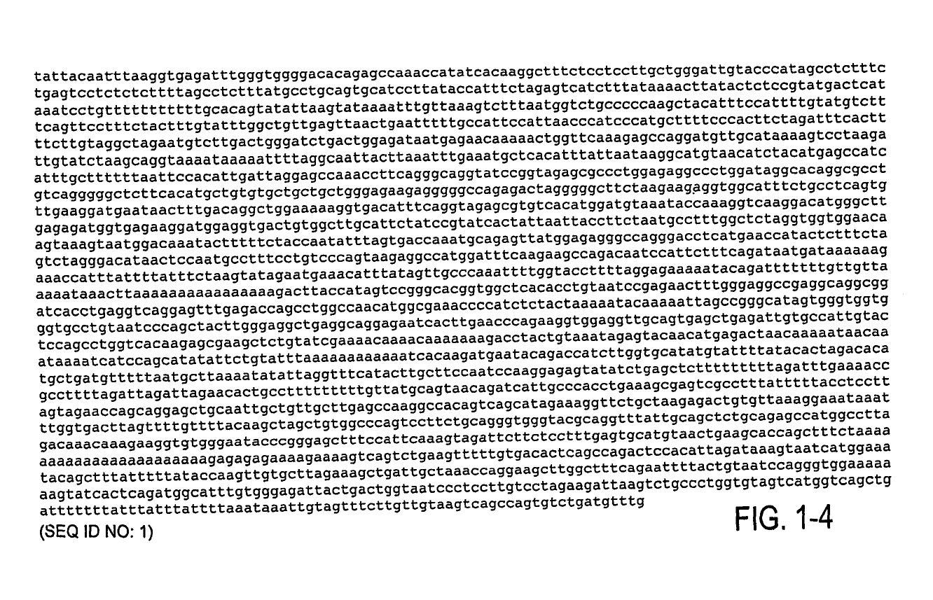

In particular embodiments, the invention features nucleic acid molecules that include at least 25, 50, 75 or 100 contiguous nucleotides of SEQ ID NO:1 (e.g., a fragment of SEQ ID NO:1 of 25-500 nucleotides); the contiguous nucleotides can include the guanine nucleotide at position −1 of FIG. 1 (alternatively, the contiguous nucleotides can include the adenine nucleotide at the opposite end of the sequence). The contiguous sequence can be longer however; the invention features a nucleic acid molecule comprising at least 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 10,500, 11,000, or 11,500 contiguous nucleotides of SEQ ID NO:1 (e.g. a fragment of SEQ ID NO:1 of that includes 25-11,500 nucleotides).

The nucleic acid molecules of the invention can be constructed using chemical synthesis and, where required, enzymatic ligation reactions using procedures known in the art. For example, an antisense nucleic acid can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used, and PrLZ nucleic acid molecules containing these derivatives are within the scope of the present invention). The antisense nucleic acid also can be produced biologically using an expression vector into which a nucleic acid has been subcloned in an antisense orientation (ie., RNA transcribed from the inserted nucleic acid will be of an antisense orientation to a target nucleic acid of interest, described further in the following subsection).

PrLZ antisense molecules can be administered to a patient in a variety of ways. For example, they can be injected directly at a tissue site (e.g., within or around the prostate gland or other PrLZ-positive tissue), or generated in situ such that they hybridize with or bind to cellular mRNA and/or genomic DNA encoding a PrLZ protein (and thereby inhibit expression of the protein by, for example, inhibiting transcription and/or translation). Antisense molecules can also be modified to more selectively target particular cell types and then administered systemically. For example, antisense molecules can be modified so they specifically bind to receptors or antigens expressed on a selected cell surface. For example, an antisense molecule can be linked to a peptide or antibody that binds to cell surface receptors or antigens. Accordingly, in the present invention PrLZ nucleic acids can be bound to androgens or any substance that specifically binds androgen receptors expressed in the prostate (other prostate-specific or breast-specific ligands can be used). Antisense nucleic acid molecules can also be delivered to cells using vectors, including those described herein. To achieve sufficient intracellular concentrations of the antisense molecules, the antisense nucleic acid molecule can be placed under the control of a strong promoter (e.g., a pol II or pol III promoter) (methods of treatment are discussed further below).

Ribozymes are also within the scope of the present invention. For example, a ribozyme having a specificity for a PrLZ-encoding nucleic acid can include one or more sequences complementary to the nucleotide sequence of a PrLZ nucleic acid disclosed herein (i.e.,SEQ ID NO:1 or SEQ ID NO:2), and a sequence encoding a enzyme capable of mRNA cleavage (see U.S. Pat. No. 5,093,246 or Haselhoff and Gerlach, Nature 334:585-591, 1988). For example, a derivative of a Tetrahymena L-19 IVS RNA can be constructed in which the nucleotide sequence of the active site is complementary to the nucleotide sequence to be cleaved in a PrLZ-encoding mRNA. See, e.g., U.S. Pat. Nos. 4,987,071 and 5,116,742. Alternatively, PrLZ mRNA can be used to select a catalytic RNA having a specific ribonuclease activity from a pool of RNA molecules. See, e.g., Bartel and Szostak, Science 261:1411-1418, 1993.

PrLZ gene expression can be inhibited by targeting nucleotide sequences complementary to a regulatory region of the PrLZ (e.g., the PrLZ promoter (SEQ ID NO:1) and/or enhancers to form triple helical structures that prevent transcription of the PrLZ gene in target cells. See generally, Helene, Anticancer Drug Des. 6:569-84, 1991; Helene, Ann. N.Y. Acad. Sci. 660:27-36, 1992; and Maher, Bioassays 14:807-15, 1992. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Any of the nucleic acids of the invention can be detectably labeled (e.g., with a chemiluminescent, fluorescent, radioactive, or calorimetric label). Moreover, a PrLZ nucleic acid molecule can be modified at the base moiety, sugar moiety or phosphate backbone to improve its stability, hybridization, or solubility (by adopting, for example, the methods of Toulme, Nature Biotech. 19:17, 2001; Faria et al., Nature Biotech. 19:40-44, 2001; or Hyrup et al., Bioorganic & Med. Chem. 4:5-23, 1996). PrLZ PNA oligomers can be synthesized using standard solid phase peptide synthesis protocols as described in Hyrup (supra) and are also within the scope of the invention. In addition to being useful in the therapeutic methods of the invention, PrLZ PNAs and nucleic acids can be used to analyze mutations in PrLZ (by, for example, PNA-directed PCR clamping) or as “artificial restriction enzymes” when used in combination with other enzymes (e.g., SI nucleases (Hyrup (supra)).

In other embodiments, nucleic acids of the invention can include other appended groups such as peptides (as noted above, for targeting host cell receptors in vivo or in cell culture), or agents facilitating transport across the cell membrane (see, e.g., Letsinger et al., Proc. Natl. Acad. Sci. USA 86:6553-6556, 1986; Lemaitre et al., Proc. Natl. Acad. Sci. USA 84:648-652, 1987; and PCT Publication No. WO88/09810).

The invention also includes molecular beacon oligonucleotide primer and probe molecules having at least one region that is complementary to a PrLZ nucleic acid, two complementary regions one having a fluorophore and one a quencher such that the molecular beacon is useful for quantitating the presence of the PrLZ nucleic acid of the invention in a sample. Molecular beacon nucleic acids are described, for example, in U.S. Pat. Nos. 5,854,033; 5,866,336, and 5,876,930.

Double stranded nucleic acid molecules that can silence a PrLZ gene can also be used as agents that inhibit expression of PRLZ. RNA interference (RNAi) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect persists for multiple cell divisions before gene expression is regained. RNAi is therefore an extremely powerful method for making targeted knockouts or “knockdowns” at the RNA level. RNAi has proven successful in human cells, including human embryonic kidney and HeLa cells (see, e.g., Elbashir et al. Nature 411:494-498, 2001). Gene silencing can be induced in mammalian cells by enforcing endogenous expression of RNA hairpins (see Paddison et al.,2002, Proc. Natl. Acad. Sci. USA 99:1443-1448, 2002). In another embodiment, transfection of small (21-23 nt) dsRNA specifically inhibits gene expression (reviewed in Caplen, Trends in Biotechnology 20:49-51, 2002).

RNAi technology in gene silencing utilizes standard molecular biology methods. dsRNA corresponding to the sequence from a target gene to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Gene silencing effects similar to those of RNAi have been reported in mammalian cells with transfection of a mRNA-cDNA hybrid construct (Lin et al., Biochem Biophys Res Commun. 281:639-44,2001), providing yet another strategy for PrLZ gene silencing.

Just as any of the nucleic acids of the invention can be detectably labeled, any of the nucleic acids can be expressed by way of inclusion in an expression vector, many of which are known in the art. Moreover, any of the regulatory PrLZ sequences described herein can be incorporated into vectors and used to drive the expression of PrLZ or any other protein. Accordingly, the invention includes a recombinant nucleic acid molecule that includes at least part of SEQ ID NO:1 or SEQ ID NO:2 inserted in a vector capable of delivering (and preferably maintaining) the nucleic acid molecule in a host cell. The DNA molecule can be inserted into an autonomously replicating vector (suitable vectors include, for example, pGEM3Z and pcDNA3, and derivatives thereof). The vector nucleic acid may be a bacteriophage DNA such as bacteriophage lambda or M13 and derivatives thereof (similarly, the vector can be any vector utilized in the studies described herein). More generally, the vector can be either an RNA or DNA; it can be single- or double-stranded; and it can be prokaryotic, eukaryotic, or viral. Vectors can include transposons, viral vectors, episomes (e.g., plasmids), chromosome inserts, and artificial chromosomes (e.g., BACs or YACs). Construction of a vector containing a nucleic acid described herein can be followed by transformation of a host cell such as a bacterium. Suitable bacterial hosts include, but are not limited to, E. coli. Suitable eukaryotic hosts include yeast such as S. cerevisiae, other fungi, vertebrate cells, invertebrate cells (e.g., insect cells), plant cells, human cells, human tissue cells, and whole eukaryotic organisms (e.g., a transgenic plant or a transgenic animal). Further, the vector nucleic acid can be used to generate a virus such as vaccinia or baculovirus.

As noted, the invention also features vectors comprising the regulatory or promoter sequences of PrLZ. A heterologous (i.e., not PrLZ) gene can be operably linked to such regulatory sequences. A PrLZ regulatory region (e.g., the promoter defined herein) can be fused to a reporter gene such as beta-glucuronidase gene, lacZ (which encodes beta-galactosidase), chloramphenicol-acetyltransferase (CAT), a gene encoding green fluorescent protein (and variants thereof), or red fluorescent protein firefly luciferase gene, among others. Any of these configurations can be used in the diagnostic and screening methods of the present invention. The PrLZ regulatory sequence can also be fused to a nucleic acid encoding a polypeptide affinity tag such as glutathione S-transferase (GST), maltose E binding protein, protein A, FLAG tag, hexa-histidine, or the influenza HA tag. Expression of the fusion gene results in translation of a single polypeptide that includes both a PrLZ-like region and reporter protein or affinity tag. The fusion can also join a PrLZ regulatory region to a fragment of the reading frame of SEQ ID NO:2. The fragment can encode a functional region of the PrLZ-like polypeptides, a structurally-intact domain, or an epitope (e.g., a peptide of about 8, 10, 20, or 30 or more amino acids). A PrLZ-like nucleic acid that includes at least one PrLZ regulatory region (e.g. a 5′ regulatory region (e.g. SEQ ID NO:1 or an operable portion thereof), a more minimal promoter (ie., a fragment of SEQ ID NO:1), and, optionally, 3′ untranslated region or a 3′ regulatory region) can also be fused to a heterologous nucleic acid. Vectors containing any such configuration are within the scope of the present invention and are useful in the methods described herein (e.g., the methods in which one screens for carcinogenic agents (an increase in reporter gene expression indicating that the test substance applied to a vector-bearing cell is a potential carcinogen), chemotherapeutic agents, or for patients at risk of cancer (e.g. prostate cancer by virtue of overexpression of PrLZ).

A wide variety of cells can be used in the methods of the invention, and cells bearing a nucleic acid of the invention are within the invention's scope. Suitable cells include any cell that can be transformed with a nucleic acid molecule of the present invention. A transformed cell of the present invention is also herein referred to as a recombinant cell. Suitable cells can either be untransformed cells or cells that have already been transformed with at least one nucleic acid molecule. Suitable cells for transformation according to the present invention can either be: (i) endogenously capable of expressing the PrLZ-like protein or; (ii) capable of producing such protein after transformation with at least one nucleic acid molecule of the present invention.

In another aspect, the invention provides an anti-PrLZ antibody or a fragment thereof (e.g., an antigen-binding (i.e., PrLZ-binding fragment thereof) or a phage peptide that specifically binds PrLZ. These antibodies are useful in the methods of the invention (as they can be used to detect PrLZ, they are usefull in methods aimed at identifying patients at risk for cancer, in methods in which PrLZ is assayed to determine whether a given test substance increases or decreases its expression; and in methods of treating patients by specifically binding, and thereby inhibiting the activity of, PrLZ protein). The antibody can be a classic tetramer (two heavy immunoglobulin chains and two light immunoglobulin chains, wherein the heavy and light immunoglobulin chains are inter-connected by disulfide bonds). The immunoglobulin can be a kappa, lambda, alpha (IgA1 and IgA2), gamma (IgG1, IgG2, IgG3, IgG4), delta, epsilon or mu immunoglobulin. Antigen-binding fragments of an antibody (or simply “antibody portion,” or “fragment”) are also within the scope of the invention (as used herein, these terms refer to one or more fragments of a full-length antibody that retain the ability to specifically bind to PrLZ or a fragment thereof). Examples of antigen-binding fragments of an anti-PrLZ antibody include, but are not limited to: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., Nature 341:544-546, 1989), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., Science 242:423-426, 1988; and Huston et al., Proc. Natl. Acad. Sci. USA 85:5879-5883, 1988; see also Colcher et al., Ann. N.Y. Acad. Sci. 880:263-280, 1996; and Reiter, Clin. Cancer Res. 2:245-252, 1996). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

The anti-PrLZ antibody can be a polyclonal or a monoclonal antibody (produced by, for example, the methods described by Yokoyama, Current Protocols in Immunology, 2.5.1-2.5.12, John Wiley & Sons, 1991 (see also the methods described below). The antibody can also be recombinantly produced by phage display or by combinatorial methods (as described in, for example, U.S. Pat. No. 5,223,409; WO 92/18619; WO 91/17271; WO 92/20791; WO 92/15679; WO 93/01288; WO 92/01047; WO 92/09690; WO 90/02809; Fuchs et al., Bio/Technology 2:1370-1372, 1991; Hay et al., Hum. Antibod. Hybridomas 3:81-85, 1992; Huse et al., Science 246:1275-1281, 1989; Griffiths et al., EMBO J. 12:725-734, 1993; Hawkins et al., J Mol. Biol. 226:889-896, 1992; Clackson et al., Nature 352:624-628, 1991; Gram et al., Proc. Natl. Acad. Sci. USA 89:3576-3580, 1992; Garrad et al., Bio/Technology 9:1373-1377, 1991; Hoogenboom et al., Nuc Acid Res 19:4133-4137, 1991; and Barbas et al., Proc. Natl. Acad. Sci. USA 88:7978-7982, 1991).

Anti-PrLZ antibodies can be fully human antibodies (e.g., an antibody made in a mouse that has been genetically engineered to produce an antibody from a human immunoglobulin sequence), or a non-human antibody (e.g, a rodent (mouse or rat), goat, or primate (e.g., monkey) antibody. Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, for example, WO 91/00906, WO 91/10741; WO 92/03918; Lonberg et al., Nature 368:856-859, 1994; Green et al., Nature Genet. 7:13-21, 1994; Morrison et al., Proc. Natl. Acad. Sci. USA 81:6851-6855, 1994; Bruggeman et al., Immunol. 7:33-40, 1993; Tuaillon et al., Proc. Natl. Acad. Sci. USA 90:3720-3724, 1993; Bruggeman et al., Eur J Immunol 21:1323-1326, 1991). Accordingly, chimeric, CDR-grafted, and humanized anti PrLZ antibodies are within the invention. These antibodies can be produced by techniques (e.g., recombinant DNA techniques) known in the art (in addition to the references cited above, see, U.S. Pat. Nos. 5,585,089; 5,693,761; 5,693,762; and 5,225,539).

Also within the scope of the invention are humanized antibodies in which specific amino acids have been substituted, deleted or added (in, for example, the framework region, which may improve binding to PrLZ). To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR.

Preferred epitopes encompassed by the antigenic peptides of the invention are regions of PrLZ that are located on the surface of the protein (e.g., hydrophilic regions), as well as regions with high antigenicity. For example, an Emini surface probability analysis of the human PrLZ protein sequence can be used to indicate the regions that have a particularly high probability of being localized to the surface of the PrLZ protein and are thus likely to constitute surface residues useful for targeting antibody production.

Antibodies most useful in the therapeutic methods of the invention will alter alter (i.e., decrease) PrLZ activity. However, antibodies without such ability are useful. For example, antibodies can be coupled to a toxin (including any of those that can be delivered by the nucleic acid molecules of the invention (e.g., by inclusion of encoding nucleotides in the vectors described above. Useful polypeptide toxins include ricin and diphtheria toxins or active fragments thereof. Antibodies can also be coupled to radioactive agents. Those that destroy the cells to which they are delivered can be therapeutic and those that do not can be diagnostic.

An anti-PrLZ antibody (e.g., polyclonal or monoclonal antibody) can be used to isolate PrLZ by standard techniques, such as affinity chromatography or immunoprecipitation. Moreover, an anti-PrLZ antibody can be used to detect PrLZ protein (e.g., in a cellular lysate or cell supernatant) in order to evaluate the abundance and pattern of expression of the protein. Anti-PrLZ antibodies can be used diagnostically to monitor protein levels in tissue as part of a clinical testing procedure, e.g., to determine the efficacy of a given treatment regimen. As noted herein, detection can be facilitated by coupling (i.e., physically linking) the antibody to a detectable substance. Examples of detectable substances are given elsewhere; here we note other prosthetic group complexes, which can include streptavidin/biotin and avidin/biotin, and provide examples of additional suitable fluorescent materials (umbelliferone, fluorescein, fluorescein isothiocyanate, rhodamine, dichlorotriazinylamine fluorescein, dansyl chloride, and phycoerythrin), luminescent materials (luminol, luciferase, luciferin, and aequorin), and radioactive materials (¹²⁵I, ¹³¹I, ³⁵S, and ³H).

Nucleic acid molecules that encode an anti-PrLZ antibody are also within the scope of the present invention, are are vectors which include the nucleic acid and cells transformed with the nucleic acid (particularly cells which are useful for producing an antibody such as mammalian cells (e.g., CHO or lymphatic cells). The invention also includes cell lines (e.g., hybridomas, which make an anti-PrLZ antibody and method of using such cells to make an anti-PrLZ antibody).

We raised polyclonal antibodies to PrLZ by immunizing mice with a synthetic peptide based on the unique N-terminal coding sequence of PrLZ. Antigen specificity was characterized by Western blotting against the endogenous PrLZ protein and by immunoprecipitation analysis of a FLAG-tagged PrLZ. One of the antibodies, M-50, was found to recognize a protein of about 28 kD, which is close to the predicted molecular weight of PrLZ (24.2 kD). We attributed the shift in molecular size and the doublet appearance of the detected PrLZ to post-translational protein modification, most probably glycosylation. In immunoprecipitation studies, M-50 specifically detected the full length PrLZ. When the N-terminal sequence was deleted or TPD52 was used in the study, which did not contain the antibody recognition sequence, M-50 did not detect any of these fusion proteins. Accordingly, anti-PrLZ antibodies, including M-50, are within the scope of the present invention.

We also assessed the ability of the characterized antibodies to detect PrLZ in clinical prostate specimens. We first used M-50 to detect PrLZ protein in prostate cancer cell lines (an immunocytochemical analysis), and detected strong cytoplasmic staining. This agrees with the results obtained using a GFP-PrLZ fusion protein. We then performed immunohistochemical (IHC) analysis for PrLZ expression in normal human prostate specimens. For this study, the Normal Tissue Microarray was obtained from the Cooperative Human Tissue Network (CHTN), funded by the National Cancer Institute. The arrayed slide contains 64 normal human tissues. M-50 immunostaining of these tissues revealed that PrLZ was distributed exclusively in prostate luminal epithelium.

We also analyzed PrLZ expression during prostate morphogenesis and development. Normal, healthy prostate specimens were subjected to IHC staining with the anit-PrLZ antibody. Included in the study were five embryonic (from 2 to 7 months of gestation) and six healthy adult (ages ranged from 24 to 42) prostate specimens. In addition, specimens were included from non-affected regions of eight patients with benign prostatic hypertrophy (BPH) (ages from 55 to 80). This analysis demonstrated that PrLZ protein is expressed at the sixth month of embryonic development. The expression peaked at age 24 and declined at 42. Of the eight BPH samples, low PrLZ expression was seen in both the non-affected “normal” regions and BPH nodules. The level of PrLZ expression appears to parallel androgen surge and the morphogenesis and development of the prostate gland. Since prostate epithelial cells undergo extensive proliferation during prenatal development as well as in young adulthood, expression of PrLZ coincides with the proliferative status of prostate epithelial cells. Accordingly, proliferation can be modulated by modulating PrLZ expression; inhibiting PrLZ expression inhibits cellular proliferation.

Several pieces of evidence indicated that abnormal expression of PrLZ is associated with the development and progression of human prostate cancer. First, compared with expression in the parental cell line LNCaP, the expression of PrLZ was enhanced in lineage-derived tumorigenic and metatastic C4-2 cells. Phenotypic transition of LNCap to C4-2 had been shown to recapitulate that of the metatastic progression of human prostrate cancers (Thalmann et al., Prostate 44:91-103,2000). Second, the PrLZ geen was localized at chromosome 8q21.1, a locus frequently amplified in clinical prostate cancers (Laitinen et al., Genes Chromosomes Cancer 35:66-73,.2002, Visakorpi et al., Cancer Res 55:342-347, 1995). Third, a closely related gene product, TPD52, is prevalent in many cancers, and it is associated with cell transformation (Byrne et al., Cancer Res 55:2896-2903, 1995; Chen et al., Oncogene 12:741-751, 1996). Last, as a prostate-specific gene, PrLZ was expressed in both AR-positive and negative prostate cancer cell lines, implicating that PrLZ might be involved in the malignant transformation of prostate epithelial cells.

To analyze PrLZ expression in clinical prostate cancer specimens, a prostate cancer tissue array was subjected to IHC staining with the MD-50 antibody. The prostate cancer tissue array used in this study consisted of samples from 100 cases of radical prostatectomy (the operations were performed at Emory University Hospital between 1995 and 1998). The results were evaluated by pathologists at Emory University Hospital. Criteria used to score the IHC staining were set as “normal” if 0-15% of the cell population stained more intensely than the normal level and “abnormal” when more than 15% of the cells were intensely stained. Expression of PrLZ was generally low in unaffected secretive epithelia (75.3% being low) and in glandular epithelia of the BPH (78.2% being low). In contrast, PrLZ was highly expressed in the event of high-grade prostate intraepithelial neoplasia (PIN) and in prostate cancers (75% being high). Gleason grade 4 tumors stained more than Gleason grade 3 tumors.

As described in more detail herein, PrLZ regulatory elements can be used to detect disease in PrLZ-positive tissues. Growth-related prostate diseases can have an early onset, progress slowly but irreversibly, and produce symptoms in later life. During pathogenesis, genes that normally function in embryonic morphogenesis and organogenesis may be “re-activated” and cause abnormal tissue and cell-proliferation in adult life (Weinhouse, Adv Enzyme Regul. 21:369-386, 1983).

The studies we performed on normal prostate specimens (some of which are descended below) showed developmentally regulated expression of PrLZ during morphogenesis and development of the prostate. Considering that PrLZ was also expressed at low levels in several other secretive tissues, such as the gastrointestinal tract, salivary and mammary glands, PrLZ may normally function in the process of growth, branching, canalization, and cytodifferentiation of the glandular ducts (Davies, Bioessays 24:937-948, 2002). Our results from prostate specimens of 100 clinical prostate cases revealed a prevalent expression of PrLZ in prostate cancer. The fact that PrLZ was not increased in all BPH samples, but was increased in high grade PIN and prostate cancers suggests that PrLZ is involved in malignant transformation of prostate epithelial cells.

Other TPD52 family members are associated with cancers in multiple tissues and organs (Byme et al., Cancer Res 55, 2896-2903, 1995, Chen et al., Oncogene 12, 741-751, 1996, Balleine et al., Genes Chromosomes Cancer 29, 48-57, 2000). Retroviral integration in avian TPD52 (R10) was accompanied with neuroepithelial proliferation (Proux et al., J Biol Chem 271, 30790-7, 1996). In leukemic cell differentiation models (using HL-60 and K-562 cells), expression of TPD52 members was inhibited upon treatment with 12-O-trtradecanoylphorbol-13-acetate (TPA). This indicates that TPD52 is associated with proliferation, but not differentiation (Byrne et al., Nucleic Acids Res 28, 212-213, 2000). These proteins interact with each other and with proteins outside the TPD52 family. Annexin VL for example, interacts with CSPP28, a TPD52-like protein (Thomas et al., J Biol Chem 277, 35496-502, 2002). Another interacting protein is MAL2, a novel MAL protein found in many secretive tissues (Wilson et al., Genomics 76, 81-88, 2001). As PrLZ may function by interaction with other molecules such as these proteolipids, the invention encompasses methods of inhibiting PrLZ function by inhibiting its interaction with PrLZ-binding molecules.

As noted above, PrLZ is expressed in cancerous cells other than those of the prostate, and it is expressed (albeit at lower levels) in normal epithelial cells, including epithelial cells in the prostate (e.g., PrLZ is involved in the growth and development of prostate glands in embryonic and adolescent mammals). Accordingly, one or more PrLZ regulatory elements can be used to direct gene expression in these cells and any other PrLZ-positive cell type. PrLZ sequences can also be used, for example, to express gene products that help maintain the health of healthy cells (ie., the compositions of the invention can be used in methods of cancer prevention) or to aid in the repair or regeneration of tissue.

As an accessory sex organ, development and function of the prostate gland are controlled by androgens, through the mediation of the AR (Gelmann, J. Clin. Oncol. 20:3001-15, 2002). To investigate the possible regulation of PrLZ by androgens, we treated prostate cancer cell lines with an androgen analog, methyltrienolone (R1881, 1 nM), after androgen deprivation. Under cell culture conditions for androgen deprivation (phenol red-free RPMI1640 for 48 hours), there was a marked decline in PrLZ expression in all the prostate cancer cell lines. However, androgen dramatically activated the expression of the PrLZ. This culture system, or another like it, can be used to identify agents that modulate (by either increasing or decreasing) PrLZ expression.

By comparing the patterns of androgen-activated expression of PrLZ and PSA, it became clear that PrLZ was controlled by a regulatory mechanism that was different from the one controlling the PSA expression. Upon androgen treatment, PSA showed an androgen-dependent up-regulation only in LCNaP cells, while the constitutive PSA expression in the lineage related C4-2 and C4-2B4 cells was largely androgen-independent. On the other hand, all the LNCAP lineage cells responded to the androgen with marked PrLZ transcription, with the C4-2B4 cells showing the highest PrLZ expression. Thus, along the transition from an AD to an AI state, there were heterologous changes in AR mediated gene regulation. While LNCAP derivative cell lines lost the mechanism of AR-controlled PSA production, they maintained the capability to regulate some other androgen responsive genes, including PrLZ.

We also examined an AR-specific antagonist, bicalutamide (Casodex), for an ability to modulate androgen-induced PrLZ expression. Casodex could block the effect of androgen on PrLZ activation in LNCaP and C4-2 cells in a dose-dependent manner. This confirms that androgen-activated PrLZ expression is mediated by AR. Accordingly, PrLZ expression can be inhibited by any compound that blocks the expression or activity of an AR. These results and our expression profiling studies revealed two important features in the regulated PrLZ expression: AR-mediated androgen up-regulation and constitutive expression regardless of the AR status.

One or more of the PrLZ regulatory elements can also be used to identify factors (e.g., tanscription factors and upstream signaling molecules) that mediate the expression of PrLZ and other genes involved in prostate cancer and other cancers. A PrLZ regulatory element (or a combination of elements, up to and including the sequences described herein that flank the PrLZ gene) can be operably linked to a gene that encodes a detectable product (i.e., a reporter gene). Cells expressing such a construct can then be exposed to any number, type, or combination of molecules, whether naturally occurring or synthetic.

Molecules that upregulate the reporter stimulate PrLZ expression (and may, thereby, stimulate unwanted cellular proliferation). On the other hand, molecules that suppress reporter gene expression suppress PrLZ expression (and may, thereby, suppress cellular proliferation). While reporter gene expression can be conveniently detected, one can also assess PrLZ expression per se.

In addition to the embodiments described above, the methods of the present invention can be carried out as follows. The “screening assays” can be carried out to identify modulators (i.e., candidate or test compounds or agents (e.g., proteins, peptides, peptidomimetics, peptoids, small molecules or other drugs) that bind to PrLZ protein, have a stimulatory or inhibitory effect on, for example, PrLZ expression or PrLZ activity, or have a stimulatory or inhibitory effect on, for example, the expression or activity of a PrLZ substrate. Compounds thus identified can be used to modulate the activity of a target gene product (e.g., PrLZ gene) in a therapeutic protocol, to elaborate the biological function of the target gene product, or to identify compounds that disrupt normal target gene interactions. These assays can include: providing a PrLZ protein or nucleic acid, or a functional fragment thereof; contacting the protein or nucleic acid with a test compound, and determining if the test compound modulates the PrLZ protein or nucleic acid. A test compound may modulate PrLZ activity by, e.g., binding to the PrLZ protein and facilitating or inhibiting its biological activity. The compound can be, e.g., an antibody, e.g. an inhibitory PrLZ antibody or an antibody that stabilizes or assists PrLZ activity. A test compound may also modulate PrLZ activity by binding to a PrLZ nucleic acid or fragment thereof. For example, the test compound may bind to the PrLZ promoter region and increase or decrease PrLZ transcription; the test compound may bind to a PrLZ 2 nucleic acid and inhibit transcription of the gene; or the test compound may bind to a PrLZ nucleic acid and inhibit translation of PrLZ mRNA. A preferred compound is a small molecule that binds to the PrLZ promoter region to modulate transcription. A test compound may also compete with the endogenous PrLZ protein for binding to a PrLZ binding partner. The test agent can be, e.g., a protein or peptide, an antibody, a small molecule, a nucleotide sequence. For example, the agent can be an agent identified through a library screen described, infra.

The screening assays described herein can be performed in vitro or in vivo. If performed in vitro, the assay can further include administering the test compound to an experimental animal, e.g., an animal model of prostate cancer.

The test compounds of the screening assays described herein can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g. Houghten (1992) Biotechniques 13:412421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J Mol. Biol. 222:301-310; Ladner supra.).

In one embodiment, an assay is a cell-based assay in which a cell, which expresses a PrLZ protein, biologically active portion thereof, or regulatory element thereof is contacted with a test compound, and the ability of the test compound to modulate PrLZ activity is determined. Determining the ability of the test compound to modulate PrLZ activity can be accomplished by monitoring, for example, binding to an endogenous binding partner, a nucleic acid, protein. The cell, for example, can be of mammalian origin, e.g., human.

In one embodiment a screening assay is for the purpose of identifying a protein that inhibits the proliferation of a cell. The method includes providing a cell (e.g., an epithelial cell; e.g., an epithelial cell from the prostate gland or a cancerous cell) containing a vector in which the sequence of SEQ ID NO:1, or an operable portion thereof, directs the expression of a test protein in the cell; and assessing the rate at which the cell proliferates following expression of the test protein. In such an assay, a decrease in the rate of proliferation indicates that the test protein is a protein that inhibits proliferation of the cell. The test protein can be an apoptotic protein, a tumor suppressor factor, a dominant negative oncogenic protein, a protein that inhibits the expression or activity of PrLZ, a toxin, or an unknown protein for which function is to be determined by this assay.

In another embodiment, the screening assay is for identifying an agent that regulates the expression of the gene encoding PrLZ. The screening assay includes providing a cell (e.g., an epithelial cell; an epithelial cell from the prostate gland; a mammalian cell; a mammalian cancerous cell; or a cell line) that contains a vector comprising SEQ ID NO:1, or an operable portion thereof, and a reporter gene, wherein the cell is maintained under conditions in which the reporter gene is expressed; exposing the cell to a test agent; and determining whether the test agent alters the expression of the reporter gene, wherein an alteration in the expression of the reporter gene indicates that the test agent regulates the expression of the gene encoding PrLZ. In this assay the test agent can be a member of a library of cDNA molecules, proteins, or small molecules.

In yet another embodiment, a cell-free assay is provided in which PrLZ protein or biologically active portion thereof is contacted with a test compound and the ability of the test compound to bind to PrLZ protein or biologically active portion thereof is evaluated Preferred biologically active portions of PrLZ protein to be used in assays of the present invention include fragments which participate in interactions with non-PrLZ molecules, e.g., fragments with high surface probability scores. Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turnis able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

Determining the ability of PrLZ protein to bind to a target molecule can also be accomplished using real-time Biomolecular Interaction Analysis (BIA) (see, e.g., Sjolander and Urbaniczky (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

The target gene product or the test substance can be anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein. It may be desirable to immobilize either PrLZ, an anti-PrLZ antibody or its target molecule to facilitate separation of complexed from uncomplexed forms of one or both of the proteins, as well as to accommodate automation of the assay. Binding of a test compound to PrLZ protein, or interaction of PrLZ protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/PrLZ fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or PrLZ protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of PrLZ binding or activity determined using standard techniques.

Other techniques for immobilizing either PrLZ protein or a target molecule on matrices include using conjugation of biotin and streptavidin. Biotinylated PrLZ protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with PrLZ protein or target molecules but which do not interfere with binding of PrLZ protein to its target molecule. Such antibodies can be derivatized to the wells of the plate, and unbound target or PrLZ protein trapped in the wells by antibody conjugation. Methods for detecting such complexes, in addition to those described above for the GST-immobilized complexes, include immunodetection of complexes using antibodies reactive with the PrLZ protein or target molecule, as well as enzyme-linked assays which rely on detecting an enzymatic activity associated with the PrLZ protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas and Minton (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: N.Y.); and immunoprecipitation (see, for example, Ausubel, F. et al., eds. (1999) Current Protocols in Moecular Biology, J. Wiley: N.Y.). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the PrLZ protein or biologically active portion thereof with a known compound which binds PrLZ to form an assay mixture, contacting the assay mixture with a test compound, and determining the ability of the test compound to interact with PrLZ protein, wherein determining the ability of the test compound to interact with PrLZ protein includes determining the ability of the test compound to preferentially bind to PrLZ or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the PrLZ genes herein identified. In an alternative embodiment, the invention provides methods for determining the ability of the test compound to modulate the activity of a PrLZ protein through modulation of the activity of a downstream effector of a PrLZ target molecule. For example, the activity of the effector molecule on an appropriate target can be determined, or the binding of the effector to an appropriate target can be determined, as previously described.

In yet another configuration, a PrLZ protein can be used as “bait proteins” in a two-hybrid assay or three-hybrid assay (see, e.g., U.S. Pat. No. 5,283,317; Zervos et al. (1993) Cell 72:223-232; Madura et al. (1993) J Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with PrLZ (“PrLZ -binding proteins” or “PrLZ-bp”) and are involved in PrLZ activity. Such PrLZ -bps can be activators or inhibitors of signals by the PrLZ proteins or PrLZ targets as, for example, downstream elements of a PrLZ-mediated signaling pathway.

The two-hybrid system is based on the modular nature of most transcription factors, which consist of separable DNA-binding and activation domains. Briefly, the assay utilizes two different DNA constructs. In one construct, the gene that codes for PrLZ protein is fused to a gene encoding the DNA binding domain of a known transcription factor (e.g. GAL-4). In the other construct, a DNA sequence, from a library of DNA sequences, that encodes an unidentified protein (“prey” or “sample”) is fused to a gene that codes for the activation domain of the known transcription factor. (Alternatively the: PrLZ protein can be the fused to the activator domain.) If the “bait” and the “prey” proteins are able to interact, in vivo, forming a PrLZ-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the PrLZ protein.

Modulators of PrLZ expression can be identified. For example, a cell or cell free mixture is contacted with a candidate compound and the expression of PrLZ MRNA or protein evaluated relative to the level of expression of PrLZ mRNA or protein in the absence of the candidate compound. When expression of PrLZ MRNA or protein is greater in the presence of the candidate compound than in its absence, the candidate compound is identified as a stimulator of PrLZ mRNA or protein expression. Alternatively, when expression of PrLZ mRNA or protein is less (statistically significantly less) in the presence of the candidate compound than in its absence, the candidate compound is identified as an inhibitor of PrLZ mRNA or protein expression. The level of PrLZ mRNA or protein expression can be determined by methods described herein for detecting PrLZ mRNA or protein.

In another aspect, the invention pertains to a combination of two or more of the assays described herein. For example, a modulating agent can be identified using a cell-based or a cell free assay, and the ability of the agent to modulate the activity of a PrLZ protein can be confirmed in vivo, e.g., in an animal such as an animal model for prostatic disease or hyperplasia, or susceptible to prostatic disease.

The chromosomal.region containing PrLZ, 8q12, is amplified in prostate cancer. The PrLZ gene family, which includes TBD52 and R10, is associated with cell transformation and malignant potential. The PrLZ gene encodes a protein having a molecular weight between 25 and 35 kD. The protein includes a leucine zipper region. This suggests that PrLZ could be a transcription as well as an intercellular docking site for other regulatory proteins.

The nucleic acids of the invention can be used in allelotyping studies and Comparative Genomic Hybridization (CGH) studies to detect chromosomal abnormalities associated with disease. Prostate cancer allelotyping studies (Carter, et al., Proc Natl Acad Sci USA, 87:8751-5 (1990); Kunimi, et al., Genomics, 11:530-6 (1991)) designed to investigate one or two loci on many chromosomal arms have revealed frequent loss of heterozygosity (LOH) on chromosomes 8p (50%), 10p (55%), 10q (30%), 16q (31-60%) and 18q (17-43%). Recently, several groups have performed more detailed deletion mapping studies in some of these regions. On 8p, the high frequency of allelic loss has been confirmed, and the regions of common deletion have been narrowed (nova, et al., Cancer Res, 53:3869-73 (1993); MacGrogan, et al., Genes Chromosom Cancer, 10:151-159 (1994); Bergerheim, et al., Genes Chromosom Cancer, 3:215-20 (1991); Chang, et al., Am T Pathol, 144:1-6 (1994); Trapman, et al., Cancer Res, 54:6061-4 (1994); Suzuki, et al., Genes Chromosom Cancer, 13:168-74 (1995)). Similar efforts also served to narrow the region of common deletion on chromosome 16q (Bergerheim, et al., Genes Chromosom Cancer, 3:215-20 (1991); Cher, et al., J Urol, 153:249-54 (1995)). Other prostate cancer allelotyping studies utilizing a smaller number of polymorphic markers have not revealed new areas of interest (Phillips, et al., Br J Urol, 73:390-5 (1994); Sake, et al., Cancer Res, 54:3273-7 (1994); Latil, et al., Genes Chromosom Cancer, 11:119-25 (1994); Massenkeil, et al., Anticancer Res, 14:2785-90(1994)).

Comparative genomic hybridization (CGH) is a molecular technique used to screen DNA from tumors for regional chromosomal alterations (Kallioniemi, et al., Science, 258:818-21 (1992) and WO 93/18186). Unlike microsatellite or Southern analysis allelotyping studies, which typically sample less than 0.1% of the total genome, a significant advantage of CGH is that all chromosome arms are scanned for losses and gains. Moreover, because CGH does not rely on naturally occurring polymorphisms, all regions are informative, whereas polymorphism-based techniques are limited by homozygous (uninformative) alleles among a fraction of tumors studied at every locus.

CGH can detect and map single copy losses and gains in prostate cancer with a high degree of accuracy when compared with the standard techniques of allelotyping (Cher, et al., Genes Chromosom Cancer, 11:153-162 (1994)). Copy-number karyotype maps have been generated for prostate cancer showing several recurrently altered regions of the genome (Cher, et al., Genes Chromosom Cancer, 11: 153-162 (1994); Visakorpi, et al., Cancer Res, 55:342-347 (1995)).

There is a high frequency of genetic gain associated with prostate cancer. Dominant oncogenes that exhibit increased expression with increased copy number are expected to be found in these locations in which genetic gain has been detected. The most notable of these is chromosome 8q, where the c-Myc oncogene is located. As described, infra, the PrLZ gene is found in this highly amplified region of the genome, chromosome 8q (specifically 8q12). Amplification of this region has previously been shown to be correlated with adverse prognosis in prostate cancer (Van Den Berg, etal., Clin Ca Res, 1:11-18(1993)). The frequency of gain of 8q detected by CGH is much higher than reported previously in smaller series (Bova, et al., Cancer Res, 53:3869-73 (1993); Van Den Berg, et al., Clin Ca Res, 1:11-18 (1993)) and may reflect the superior ability to detect gain using CGH.

One of skill can prepare nucleic acid probes specific to particular genomic regions of genetic alteration (e.g., genetic loss or gain) that are associated with prostate cancer. The probes can be used in a variety of nucleic acid hybridization assays to detect the presence (in particular increased copy number) or absence of the regions for the early diagnosis or prognosis of cancer. As noted above, the probes are primarily useful for the diagnosis or prognosis of prostate cancer. The regions can also be used for a large number of other cancers. These include, but are not limited to breast, ovary, bladder, head and neck, and colon.

The genetic alterations are detected through the hybridization of a probe of this invention to a nucleic acid sample in which it is desired to screen for the alteration. Suitable hybridization formats are well known to those of skill in the art and include, but are not limited to, variations of Southern Blots, in situ hybridization and quantitative amplification methods such as quantitative PCR (see, e.g., Sambrook, Molecular Cloning—A Laboratory Manual, Cold Spring Harbor Laboratory, Cold Spring Harbor, N.Y., (1989), Kallioniemi et al., Proc. Natl Acad Sci USA, 89: 5321-5325 (1992), and PCR Protocols, A Guide to Methods and Applications, Innis et al., Academic Press, Inc. N.Y, (1990)).

### EXAMPLES

The studies above, and the techniques that follow concern the isolation and molecular characterization of PrLZ in human prostate cancer cell lines and tissues. DNA sequencing analysis indicates that PrLZ encodes a 224-amino acid protein that is a new member of the TPD52 family. PrLZ was localized to chromosome 8q21.1, a locus frequently amplified in prostate cancers. Genomic cloning and DNA sequencing data indicate that the PrLZ gene contains six exons, spanning a 45 kb region of the genome. Analysis of multiple tissues revealed predominant expression in human prostate gland; PrLZ was detected in all human prostate cancer cell lines tests, and the level of expression was enhanced by androgens. PrLZ expression was not, however, androgen-dependent, since it was constitutively expressed in androgen receptor-negative human prostate cancer cell lines. Immunohistochemical analyses demonstrated a close correlation between enhanced PrLZ expression and malignant potential in human prostate cancer cells. The PrLZ gene was amplified in four out of seven prostate cancer cases. As our studies indicate that PrLZ is a prostate-specific gene and that aberrant expression is associated with higher-grade cancer, we conclude that PrLZ plays a role in the progression of prostate cancer.

Amplification of PrLZ in prostate cancer cells: Amplification of chromosome 8q21 is one of the frequent genomic abnormalities in prostate cancer cells (Laitinen et al., Genes Chromosomes Cancer 35, 66-73, 2002, Visakorpi et al., Cancer Res 55, 342-347, 1995). Certain genes in this locus may be involved in the intitiation and progression of prostate cancer, due to a gain of function (Porkka et al., Lab Invest 82, 629-637, 2002, Kim et al., Prostate Cancer Prostatic Dis 3, 110-114, 2000, Nupponen et al., Genes Chromosomes Cancer 2, 203-210, 2000). To investigate whether PrLZ gene is amplified in prostate cancer cells, we examined copy numbers of the PrLZ gene by PCR amplification of its first exon and the last exon. In this study, genomic DNA sample pairs were isolated from five radical prostatectomy specimens from patients diagnosed with prostate cancer. Cancer cells were isolated by laser capture micro-dissection (LCM), followed by genomic DNA isolation. For comparison, matched genomic DNA was also prepared from morphologically normal glandular cells, distant from tumor-inflicted area. Equal amount of DNA (100 pg) was used in PCR reactions.

Polymerase chain reaction (PCR) analyses: All the experiments involving PCR amplification were carried out on a Mastercycler Gradient instrument (Eppendorf, Hamburg, Germany). All the reactions were initiated with a two-minute incubation at 94 C, followed by 30 cycles of 94 C, 30 seconds; 55 C, 30 seconds; and 72 C, 7-minute extension. All the PCR reactions were performed with the GeneAmp High Fidelity PCR system (Applied Biosystems, Foster City, Calif.).

Primers used for detection of the PrLZ exon 1 were: cctgaactgtttgtacctctgggccatattgc (SEQ ID NO:10) and caaatttctgaagagtaggtgatccgggtggag (SEQ ID NO:11). Priiners used for detection of the PrLZ exon 6 were: tctaaagtagggggaaccaagcctgctggtggtg (SEQ ID NO:12) and actgatagatggaatttattaagcttttcacatg (SEQ ID NO:13). Primers for detecting the 3′UTR of the HIF1A gene were: caagttaactgagctttttcttaatttcattcc (SEQ ID NO:14) and gttggtacctccacagaagatgtttatttgatgtaac (SEQ ID NO: 5). Primers for amplification of the mitochondrial DNA (MTDNA) were: agtcaatagaagccggcg (SEQ ID NO:16) and ggggatttagagggttctgt (SEQ ID NO:17).

This study revealed varied levels of PrLZ gene amplification in four of the five prostate cancer cases.

We also amplified the PrLZ gene in serially diluted samples of normal genomic DNA. By comparing the amplified signals, we estimated that in these four prostate cancer cases, copy numbers of the PrLZ gene were 12, 3, 6 and 4 times more than the normal cells.

Molecular cloning: Total RNA from C4-2 cells was used as initial material in the construction of a cDNA library, with the ZAPExpress phagemid (Stratagene) as vector. Insert from the original EST clone (330 bp) was radioactively labeled and used as probe to screen 2×10⁵ pfu of the cDNA library. Positive clones were isolated after the third screening and rescued into pBK-CMV plasmid by in vivo autoexcision. The plasmids were subjected to restriction mapping, DNA sequencing analysis, and in vitro transcription coupled translation, which was performed with the TNT kit (Promega).

Gene-specific primers of gcctgaactgtttgtacctctg, and gagtaggtgatccgggtggagatg were used to screen a human BAC genomic DNA library by polymerase chain reaction (PCR). Agarose gel electrophoresis of the PCR product was used to identify the positive clone, which was then cultured for DNA isolation. Restriction fragments of the BAC clone were subjected to Southern blot hybridization with the PrLZ cDNA as probe. Positive bands were subcloned and subjected to nested deletions with Erase-A-Base kit (Promega, Madison, Wis.) for DNA sequencing analysis. Manual DNA sequencing was performed with the ATaq sequenase II kit (USB, Cleveland, Ohio), with ³⁵S-dCTP as labeling. Automated DNA sequencing analysis was performed on an ABI PRISM 100 Version 3.2 sequencer (Applied Biosystems, Foster City, Calif.).

Expression studies: The 502 bp PrLZ gene-specific fragment was subcloned after PCR amplification of the PrLZ cDNA in pBK-CMV, with primers: gcctgaactgtttgtacctctg, and gagtaggtgatccgggtggagatg. The amplified DNA was cloned into pGEM-T easy (Promega) and verified by DNA sequencing. The fragment was released from the vector by EcoRi and used in hybridization reactions. Standard protocols were used in Southern and Northern blot hybridizations (Sambrook et al., Molecular cloning, a laboratory manual, Clod Spring Harbor Laboratory Press, 2001), with α-³²P-dCTP used in the labeling step. For studies with the commercially prepared Multiple Tissue Expression array and the Multiple Tumor Expression array (Clontech), manufacturer recommended protocols were followed.

Polyclonal antibodies were produced by immunizing mice with synthetic peptides, with the standard protocol (Yokoyama, “Current Protocols in Immunology”, 2.5.1-2.5.12, John Wiley & Sons, 1991). Sera of the mice were tested for recognition of PrLZ proteins by Western blotting and immunoprecipitation. Subsequently, specific antibodies were used in IHC analysis with 1:5,000 dilution. Pre-immune sera with the same dilution were used as isotypic control.

Transfection studies with of recombinant DNA: To over-express FLGA-tagged proteins, complete coding sequence for the PrLZ and PTD52 was cloned in frame to the p3xFLAG-myc-CMV-25 vector (Sigma, St. Louis, Mo.) and transfected to different cell lines with the GenePorter reagent (Gene Therapy Systems, San Diego, Calif.). After 24 hours of incubation, transfected cells were harvested for whole cell lysate preparation. The lystates were used in immunoprecipitation and Western blotting with anti-FLAG and anti-PrLZ antibodies.

To study the subcellular distribution of the proteins, complete coding sequence for the PrLZ and TPD52 was ligated in frame to the pEGFPcl vector (Clontech). G418 (800 μg/ml) was used to select stably transfected clones, which were isolated by limited dilution. Successful GFP fusion of the proteins was confirmed by Western blotting. Subcellular localization of the fusion protein was determined by fluorescence microscopy on tissue culture dishes, and by confocal micrograph on cover slides.

What is claimed is:

1. An isolated nucleic acid molecule comprising at least 25, 50, 75, or 100 contiguous nucleotides of SEQ ID NO:1.

2. The nucleic acid molecule of claim 1, wherein the contiguous nucleotides include the guanine nucleotide at position −1 of FIG. 1 (SEQ ID NO:1).

3. The nucleic acid molecule of claim 1, comprising at least 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 10,500, 11,000, or 11,500 contiguous nucleotides of SEQ ID NO:1.

4. The nucleic acid molecule of claim 3, wherein the contiguous nucleotides include the guanine nucleotide at position −1 of FIG. 1 (SEQ ID NO:1).

5. The nucleic acid molecule of claim 1, wherein the nucleic acid molecule, when operably linked to a sequence that encodes a polypeptide, directs expression of the polypeptide in a cell under physiological conditions.

6. The nucleic acid molecule of claim 1, wherein the nucleic acid molecule comprises a TATA box, a CCAAT box, an Sp1 site, a TRE (TPA (12-O-tetradecanoyl-phorbol-13-acetate) response element), a CRE (cyclic AMP response element), an Ets binding site, an ERE (estrogen response element), a Myc binding site or a binding motif for Myc-Max dimers, an Nf-1 binding site (a binding site for NF1-A, NF1-B, NF1-C, or NF1-X), a GATA binding site (a binding site for one of GATA-1 to GATA-6), an E2F binding site, an LSF binding site, a Mef-2 binding site, a CarG box (also known as an SRF (serum response factor) binding site), a Myf binding site (a binding site for MyoD, myogenin, myf5 or MRF4), or a TEA/ATTS domain (a Tef (transcription enhancer factor) binding site).

7. The nucleic acid molecule of claim 1, comprising the nucleic acid sequence of SEQ ID NO:1.

8. The nucleic acid molecule of claim 1, consisting of the nucleic acid sequence of SEQ ID NO:1.

9. The nucleic acid molecule of claim 1, further comprising a sequence encoding a detectable protein, wherein the nucleic acid molecule of claim 1 is operably linked to the sequence encoding the detectable protein.

10. The nucleic acid molecule of claim 9, wherein the detectable protein is not PrLZ (Prostate Leucine Zipper).

11. The nucleic acid molecule of claim 9, wherein the detectable protein comprises a fluorescent or luminescent protein.

12. The nucleic acid molecule of claim 9, wherein the detectable protein is an enzyme that catalyzes a reaction that produces a detectable reaction product.

13. The nucleic acid molecule of claim 9, wherein the detectable protein is a metal chelator.

14. The nucleic acid molecule of claim 1, further comprising a sequence encoding a protein that inhibits the proliferation of a cell in which it is expressed, wherein the nucleic acid molecule of claim 1 is operably linked to the sequence encoding the protein that inhibits the proliferation of the cell.

15. The nucleic acid molecule of claim 14, wherein the protein that inhibits the proliferation of a cell is an apoptotic protein, a tumor suppressor factor, a protein that inhibits PrLZ expression or activity, or a toxin.

16. A vector comprising the nucleic acid molecule of claim 1.

17. The vector of claim 16, wherein the vector is a plasmid, a cosmid, a bacterial artificial chromosome (BAC), a yeast artificial chromosome (YAC), an adenovirus, an adeno-associated virus, or a retrovirus.

18. A cell comprising the isolated nucleic acid of claim 1 or the vector of claim 16.

19. A pharmaceutically acceptable composition comprising the nucleic acid of claim 1 or the vector of claim 16.

20. An antibody that specifically binds PrLZ.

21. The antibody of claim 20, wherein the antibody is a monoclonal antibody or a single chain antibody.

22. The antibody of claim 20, wherein the antibody is humanized.

23. An antisense oligonucleotide comprising at least 15 nucleotides having a sequence that is the reverse and complement of 15 contiguous nucleotides within the coding sequence of SEQ ID NO:2.

24. A kit for detecting PrLZ expression or altering PrLZ expression or activity in a cell, the kit comprising instructions for use and a nucleic acid molecule of claim 1, the vector of claim 16, the antibody of claim 20, or the antisense oligonucleotide of claim 23.

25. A method for identifying a protein that inhibits the proliferation a cell, the method comprising
(a) providing a cell comprising a vector in which the sequence of SEQ ID NO:1, or an operable portion thereof, directs the expression of a test protein in the cell and
(b) assessing the rate at which the cell proliferates following expression of the test protein, wherein a decrease in the rate of proliferation indicates that the test protein is a protein that inhibits proliferation of the cell.

26. The method of claim 25, wherein the cell is an epithelial cell.

27. The method of claim 26, wherein the epithelial cell is an epithelial cell of the prostate gland.

28. The method of claim 25, wherein the cell is a cancerous cell.

29. The method of claim 25, wherein the test protein comprises an apoptotic protein, a tumor suppressor factor, a protein that inhibits the expression or activity of PrLZ, or a toxin.

30. A method of determining whether a patient has a prostatic disease or whether a patient is at risk of developing a prostatic disease, the method comprising determining whether PrLZ expression is elevated in cells of a biological sample obtained from the patient or whether cells in a biological sample obtained from the patient contain an amplified PrLZ gene.

31. The method of claim 30, wherein the biological sample comprises prostate epithelial cells.

32. The method of claim 30, wherein the biological sample comprises blood, urine, or ejaculate.

33. A method of identifying an agent that regulates the expression of the gene encoding PrLZ, the method comprising
(a) providing a cell that contains a vector comprising SEQ ID NO:1, or an operable portion thereof, and a reporter gene, wherein the cell is maintained under conditions in which the reporter gene is expressed;
(b) exposing the cell to a test agent; and
(c) determining whether the test agent alters the expression of the reporter gene, wherein an alteration in the expression of the reporter gene indicates that the test agent regulates the expression of the gene encoding PrLZ.

34. The method of claim 33, wherein the cell is a mammalian cell.

35. The method of claim 34, wherein the mammalian cell is cancerous.

36. The method of claim 35, wherein the cancerous cell is a cancerous prostate cell.

37. The method of claim 33, wherein the cell is a cell of a cell line.

38. The method of claim 33, wherein the test agent comprises a member of a library of cDNA molecules, proteins, or small molecules.

39. A method of treating a patient who has cancer or a high grade dysplasia, the method comprising administering to the patient an agent that inhibits the expression or activity of PrLZ.

40. The method of claim 39, wherein the patient has prostate cancer or a high grade dysplasia affecting prostate cells.

41. The method of claim 39, wherein the patient has a cancer or dysplasia affecting breast cells, kidney cells, cells within the gastrointestinal tract, or any other cells that express PrLZ.

42. The method of claim 39, wherein the agent that inhibits the expression or activity of PrLZ is an anti-PrLZ antibody, an oligonucleotide having a sequence that is the reverse and complement of a portion of the PrLZ gene sequence, or an agent that inhibits an androgen receptor.

43. Use of an agent that inhibits the expression or activity of PrLZ for the treatment of cancer or high grade dysplasia.

44. Use of an agent that inhibits the expression or activity of PrLZ for the preparation of a medicament for the treatment of cancer or high grade dysplasia.

45. Use of an agent as described in claim 43, wherein the cancer or dysplasia affects prostate tissue, breast tissue, renal tissue, tissue within the gastrointestinal tract, or any other PrLZ-positive tissue.

46. A method of treating a patient who has cancer or a high grade dysplasia affecting a PrLZ-expressing tissue, the method comprising administering to the patient a vector comprising SEQ ID NO:1, or an operable portion thereof, and a sequence that encodes a protein that inhibits the proliferation of a cancerous cell, or a cell deemed dysplasic, within the patient.

47. The method of claim 46, wherein the cancer or high grade dysplasia affects cells in the prostate, breast, renal system, or gastrointestinal tract.

48. The method of claim 46, wherein the protein that inhibits the proliferation of a cell is an apoptotic protein, a tumor suppressor factor, a protein that inhibits PrLZ expression or activity, or a toxin.

49. Use of a vector comprising SEQ ID NO:1, or an operable portion thereof, and a sequence that encodes a protein that inhibits the proliferation of a cancerous cell or a cell deemed dysplasic in a patient for the treatment of cancer or high grade dysplasia.

50. Use of a vector comprising SEQ ID NO:1, or an operable portion thereof, and a sequence that encodes a protein that inhibits the proliferation of a cancerous cell or a cell deemed dysplasic in a patient for the manufacture of a medicament for the treatment of cancer or high grade dysplasia.

51. Use of a vector as described in claim 49 for the treatment of cancer or dysplasia in the prostate, breast, renal system, or gastrointestinal tract.


SEQUENCE LISTING (19 sequences)

SEQ ID NO: 1 - DNA, 12000 bp, Homo sapiens
atctcacctcttataattatgtgatctttttttttttttaaattgtggtagaatgtgtataacacggaatttgtgattttactctctcttcacgagatctacagtttttagctccacttgagtgagaacgtgtgacagttgtatttctgttcctagcttatttcacttaacataatgacctctagttccacccatgttgctgcaaatgacaagattcatttttttatggccaagtagtaataaattgtgtatatataccacaattttctttattcaactattaatgaatataggttgattccatatcattgctattgtgaatagtgcttcaataaacgcgcacgtgcaaatatccttgacatctgatttctttacatttgggcaaatacccagtagtaggattgctggatctattaatatgttaattctgtttttaattgttttgagaaatctccatactgtttttcacagtggctgtgctgggaagatgtgtcagtgggtggggggaggataaaacgaggttagttaatggttacaaacataccttagaaggaataagttctaatgttcgatagcagtgtaggatgaccatagttaacaacaatgtattgtagatttcaaaatagctagaaaagaggacttggaataatgccaacacacagaaatgacgaatactcgaggtgatagatgccccaaacccctaatttgatcattacacagtgtaagcatgtgatagagtatcatatttgtgcccccatatacatgtacacatatttgtatcaataagaaatatatacaaaaagccaaacctttttagctataaaatctgtctcttatgttcaaatgtatatagttagaaagtgctggagtcctgtaacatcagtattcagagtttactcaagctttcatcctcaggcaaacttagcagctgttatctaaagccagacttgcagcatcagaaatcacctggagcttgtccagaaagcaagagtcttgggctccaccccagactttctaactcagaatctgtagttccacaggctcccctggtggctctggtatgtaataaagttatttgagaagcactggctggagatcttttacctgagcctgtaatcatggaatcacccatcagtatgtggagtgaccataacatttcaactcaaactctatttctaaaatgaaaaaatctgtctattgtatctaattaagtatatgacaaatatcaagctctctttccactattttctggtgttctcatgatctgacaatgacagtggcaggaccatgtactgagatgaccgaagtgaaaatagtaatgctcttttgtgtattttttttttttttttttgagatggagtctctgtcgcccaggctggagcgcagtggcgcgatctcggctcactgcaagctccgcctcttgggttcaggccattctcctgcctcagcctcccaagtagctgggactacaggtgcccgccaccacgcccagctaatttttttgtaattttagtagagacggggtttcaccgtgttagccaagatggtcttgatctcctgaccttgtgatgcccgcctcagcctcccaaagtgctgggattacaggtgtgagccactgcgcccagcctcttttgtgtattttagaatccagtctgtgactccttgtgatagagaattcaggcattcttgagacttctcaacagattctgactttcccaggtacaggcttatccagacgacatgtttggagataggttgagagagggaatgacatcttatgcttattatatgtcactcttgctcttcagtatttgaaggaaacactgtgtgaagaacaaaaaccttctgtcctggctgctgacattaactcaattttagttattttgagagggcagaagaatattctaagtagactgttaggggttcctaacaagtgagaatttctttgcaagtcagtgggccaaataaacgatttctttaaaaatacaaatccattttatcacttaagagtatttacatttagttccaaataatttgcatatgcctgctaggttatgcctttttattctagagaagaggctccaaagaggatagtttttttttttttctttcttataatactgtgctataatagtcttttttcccccattgggatggaaaataaaggagaaaagaagtggcattcccatagggaatatttcctaagtttctaatgcaaagtttctaatctgaaaactgccacatatttttaaaagaatgccaacatgatttaatctagtttcattcagtttaggaactgaaatgcccctgggtgagggacagcatccctgaaccttccttggcccctggtctgccccgcccctccccatccccactgtgcttcctcttctgcattgtcttctttgctcttaaacacacacatttgaaaactttatttatttgttttttaaagaggtggcaccatcgtagctcactctagccttgaactcctgggctgaagtgatcccttctgacccagcctcctgagtaactggaactacaggtgtgcaccactgtgcctggctaatttttattttttattttttttgtagagccagggcctcgctttgttgcccatgctggccttgaactccaggcctcaagctatcctcctgcctcagcctcccagagttctgggattataggtgtgaaccaccatgcctggcctaaaatactttttaaaacttcttttcattcgcttcttccctttcctttcctccactgcctaccacttctgcttatctgacatctaccatcactctcactaaaacttcagtgcgagatcaccagaccccaagtcctgtggcttcttcttgcgtctctttagcatttgatcttatccagtttctctcctcccttgattcctgtgacattggtcactcaaggttttcttccagatgttttgcttcttgagagcaatggaattggaatcctaagttttaccactttctagctctgtgcctagggcaagacacttaactttgatgtgtctcagttttgttgtaagataagggtagggaaaaacttaccccataaaattgatgcaagtgcatagaagaatgtctggcacatagtaagggttccttaaggataagtcataattattgtcatcattaaaggtcattgcttgccctttttaggctctttggcctgcatgctgattgtaggtgttcactaatttttccactccttttcatagggcctctcttctcttcaaatttttttttgacgatctctgggtttcccacattttatatcttagcccctaactttttccctgaggatagaagtttaatttctaacctcaggctggccatttactccagaagtcttgtgagctctattaaccaaatctgccaaaatgcagctcattctcttatcacctaaagtctcttctctctgttcttcagtcctgcctctcttttgccaccatcttcattaatgcggtcactgtcctttacaactcctgctggggtatcacacgttggcttctcattgcccgtcacccaagtcctccagttgatctccagcatctgttgattgtctgttcatggtgtcatgcagtgtgtctttgctttgctgttcccgcttctgttccatgcagggcacatgccttttgtattagttggtttgggccgcaataacaataccatagaataggtggtttgaacaatggaaatttatattctcacgattctggaggctgaaagtccaagatcaaggtgctggtggcagggtagataggtttctggcatgggcttgcttcctggttgacagagagctgccaggcgcagtggttcacgcctgtaatcccagcactttgggaggccgaggcgggcggatcacgaggtcaggagatcgagaccatcctggctaatgcagtgaaaccccgtctctactaaaaatacaaaaaattagctatgtcctccctggccttttttctgtgaaaaaggccttttgcgtgagcatccctagtgtctcttcttactaggacagcagtcctattggattagggcctcaccctatgaccctgtttaatcttaattacctcctaaagaccccatctccaaatacagccatattgggagttagggcttcaacacatgaagctggggagacacaattcagtccagaacacccttggcgctgttgctgggtagctgtcctgacttccctctcccctcggcagtccagcttcagacaggggtgagaatgatctttctgacacgtggctttggcctcccattcctaattactcgacttggctccccacggcctagatgcagtgagaggaaaatgaccactgtagggtggcttccttcatttctcagttccttatgctcactcctctggaaattggagataacatgactaacctcgtgttattgagggattaaataaattctaatgaagaattgttgctttaatctgacgaaaaccaaattcctactctccagcctccattcgagttcttccatcaccttgttactccaagtgtgatcccatgtggttccagggccagcagcctcccatcaccaggatgcagactctgcatcacagcaagatccccagggccattagatgtgagggactggcttatccttactgcccggctctaggctacagccacaggatggagaggacagggacttggaggcacgtaccaggcttcaaactgcggctcctttacttgctagctctatgcccaaagacaggtttacaggaactgtccttgtcttgtttcccctctgaaaaatagggagaggcagaggtttacctcacagaggttttgaggattagctgagaaattttgtaaataagagagcatctacccttgtgattttaaattgaatttcatacttaaaatcaagtactttttatttggagtcaaggtttgtttattgtggcacagtgtcggggaatgaagaggtgtggggtgtggggtgttcattaacctctcagcctctctgccctctcaccctgtgcaacagggtccccactggtctccctctgggaagatgtgaccagcatgggatatgatggggagacttcttttctgtagaaggtaaaccaactgctgcttcggtggaaggtggagagagcccttccagctctcagaaggggcctggagccggtatgtgcctgtgtgtctgtggctggagcccagtcccattaggccctgcccccgtgattgctgtgagaggtggttgccacacactttctccacagcgccctcagggctgctgaatctaaggcagaggagttcttgttttttgtttgtttgttttttagcccttttcaaatttacagaaggattgcaagaataagaatagtgtaaatacctgctcctgtgttactttgctgaagataatggcttccagctccatccatgtccctgcaaaggacatgctcttgttcctttttatgactgcatagtattccatggtgtatatgtgccaacaacacaggtgtgggggagggagagcataaggaagaatagctaatgaatgctgggcttaagacctaggtgataggttgatttgtgcagcaaaccactatggcacacacttacttatgttacaagcctgcacatcctgcacatgtaccctggaacttaaaagttgatggggggaaaaaaaaaggacacctgcattctctactcagggtcactgttaacgtttagccccatccggttgaccagtctctctgtgtgtggatgtctgtacacatgcatactcttattttctgagcaatttgagaggaagttgcatatatcatggcgtctgcccctaaatacttcagtatatattgccattgcctaagaaaggcaatattctcaccacagttgtcaacgtcagtaaatttgattttttttttttttttttttttttttttttttgttgagacagcgtctcactcttgttgcccaggctgggatgcagtggcacgatctcagctcactgcaatctccgcttcccgggttcaagcaattcttgtgcctcagtcttctgagtaactgggactgcaggcacgtgcccccccaaccagctaatttttgtatttttagtagagacggggattcaccatattggccaggctggtctcgaactcttgatgtcaggtgatctgcctgccttggtctcccaaagtgccaggattacaggtgtgagccaccaagcccagccagtaaatttgaaattgatacaagacttttatctaatctgaggtcatattccagttttttcaatggacctagtaatgccagtctcctttaatgtggattttcacagtctttgtcctttatgacatcgatgtttttgaagaatgcagcccctcctcttttaaaaactagaaccttccggtcaggctcggtggctcacacctgtaatcccagcaccttgggggaccgaggcgggtggatcacgaggtcaggagatcgagaccatcctagccaatatggtgaaactccgtctctactaaaaatacaaaaattagctgggcgtggtggtgtgcgcttgtagtcccagctactcaggagactgaggttaggagaatcacttaaaccggggaggcggaggttgtggtgagtcaagatcacgacactgcactccagcctggcaacagagtgagagtccgtctcaaaaaaaaaaaaaaaaaaactaaaaaaaaaaaaaagcaaaaaaacccgaagcaacaacaaaaccccaacaaaaacttgttttggatttgtctgatgtttcctcatgattcgattcaggttctgcattccaagcctgaacactatgtaggtattcagagcggtccatctgcctctcactggggtatggccatctctatccttcagtcagggtggggcttttctccattatactgttactacacttgatctcagccaaaaggccaagaagtgatctccactcaatgtttttccttctggcagctagttaatctgtggagaggcactttaaacatcatttaaatatcatcttcttcctcaaaatttctcccgagctttagcatccagcgatgatttttgtctgaatctttacaaagatggtcagggtggcattcttctgtagttcttccttctctctatttattactttcttggactcttggattcctgtttttttcagtggtttataatccctgactgtccctaatttttttggcggcctgattgtcccatattggctagttggagccccttccagccagctgctgtatcaccatgacacgtctgcattccccaagtatctgcggcctgcctctcctggtggacagccacattgtctccctttgacgtgccctccaagtaaatattgtgtttcatagcttttaaatcagactgtcctttatatgtctttcaagtttgtggagaatttacactgacatatttgagtgatgagttaacaggcagacagaaacttaattttctttatagtcatttgtacctactaccatttccgtttgcaatgacatgagtcgtctccggttattaccctgcagtacatacgcactttactggctgcacctagttgacttctgaactgcccttgggctccctctgctggcatagtggtgcgtgagcacttttctggggtcatggaatggatttctctttctgcccattttctttcttttggtttgtggttttctctctcataaattattacacaaacatggaccatacaatacaaccttctttcaaacctgatttgtttgctaactgtatcagtacctttccatataaaattcataaatatttttatataattatgtcagtgtttccatatttttctattacctgaatgttctttttagcatttaaataatccatttttagtaggcatttgtattgcttttttccatttcaggtagcatctaaagtgaatgtcccttgcatgtatactattcatatgtgacttaataagtagagcttgctaaatgggtaggaacttacaggaaaaggggaaactctagtttgagaaaattgtctgggctaagtttaggatgtgttcaacaaacaggaaagtcaagtttggctaaagcatggttgagtaatagtgggaaatatggtcagaaagtaggtatgttaggactctttctactgaaatgatgaaattcaactccagccacaagtggagcagaacaaaattgtgtttgtttaataactgggaagcccagggtagctggaggcaggggcttagatgctgctgcgccatcccttacctgtctgtttctgtttctccttctgtcccttcccagtctcagcactgagtctcttgcccattggcctggtgagggaaggagctgccagccccacccaacagctcaggttacagagagagtcactttcttccattactcacagagtaaacatcaaggaaggccactgattgattgacagtgtctgggtcagatgtctatccttaggccagtccctgtgaacaaggggatggggtgtctgcgtggaccagatctgaagcacaggcccatgcctggggccagggggtgggaactatggacctctctccccactgagaaccccagggagcaggtgaggtgaaattcctctaggggaagaggggcaaaattgacaagatagcagatgtctaccatactgctgtggggcctggtccctcccagaaggaaaaacatagtaacaatagagtgggtctcaccctccacctgggtctcaagtagggtgtggatgaggacaatggaaatgaaggaaaggttagaaggcctgtggtaccggttggtagatagctcttcgtgctttctccatatggagtgagagtgcttggatgtgattccttcaaagtcaggtctaggagactcaggatgcctaatctagaggtaagaacattgtgaggaaagccagtgaattcagtcttgtgcatgctgactttgaagtacttttggaagagccaagtggaattatccacaggacaggaccaaatcttacctggttcttccccaggccgactagtccacaacaggaaataaaaagagttgccccgataccaaggtgtactagtccattctcacactgctatggggaaatacctgagactgggtaatttataaagggaaaaggtttaattgactcacagttctagatggctggggaggcttcaggaaacttacaatcatggcagaaggcaccacttcacagggtggcgggagagagaatgagtgcccagcgaagggagaagctccttataaaaccatctgttctccttataaagatctcttaataaaaccgtcagagaactatctcattcactatcaggagaagagcatgggggaaccgcccccatgattcagtttactccacctggtcccgcccttgacatgtgggtgttattacaatttaaggtgagatttgggtggggacacagagccaaaccatatcacaaggctttctcctccttgctgggattgtacccatagcctctttctgagtcctctctcttttagcctctttatgcctgcagtgcatccttataccatttctagagtcatctttataaaacttatactctccgtatgactcataaatcctgtttttttttttgcacagtatattaagtataaaatttgttaaagtctttaatggtctgcccccaagctacatttccattttgtatgtctttcagttcctttctactttgtatttggctgttgagttaactgaatttttgccattccattaacccatcccatgcttttcccacttctagatttcacttttcttgtaggctagaatgtcttgactgggatctgactggagataatgagaacaaaaactggttcaaagagccaggatgttgcataaaagtcctaagattgtatctaagcaggtaaaataaaaattttaggcaattacttaaatttgaaatgctcacatttattaataaggcatgtaacatctacatgagccatcatttgcttttttaattccacattgattaggagccaaaccttcagggcaggtatccggtagagcgccctggagaggccctggataggcacaggcgcctgtcagggggctcttcacatgctgtgtgctgctgctgggagaagagggggccagagactagggggcttctaagaagaggtggcatttctgcctcagtgttgaaggatgaataactttgacaggctggaaaaaggtgacatttcaggtagagcgtgtcacatggatgtaaataccaaaggtcaaggacatgggcttgagagatggtgagaaggatggaggtgactgtggcttgcattctatccgtatcactattaattaccttctaatgcctttggctctaggtggtggaacaagtaaagtaatggacaaatactttttctaccaatatttagtgaccaaatgcagagttatggagagggccagggacctcatgaaccatactctttctagtctagggacataactccaatgcctttcctgtcccagtaagaggccatggatttcaagaagccagacaatccattctttcagataatgataaaaaagaaaccatttattttatttctaagtatagaatgaaacatttatagttgcccaaattttggtaccttttaggagaaaaatacagatttttttgttgttaaaaataaacttaaaaaaaaaaaaaaaagacttaccatagtccgggcacggtggctcacacctgtaatccgagaactttgggaggccgaggcaggcggatcacctgaggtcaggagtttgagaccagcctggccaacatggcgaaaccccatctctactaaaaatacaaaaattagccgggcatagtgggtggtgggtgcctgtaatcccagctacttgggaggctgaggcaggagaatcacttgaacccagaaggtggaggttgcagtgagctgagattgtgccattgtactccagcctggtcacaagagcgaagctctgtatcgaaaacaaaacaaaaaaagacctactgtaaatagagtacaacatgagactaacaaaaataacaaataaaatcatccagcatatattctgtatttaaaaaaaaaaaatcacaagatgaatacagaccatcttggtgcatatgtattttatacactagacacatgctgatgtttttaatgcttaaaatatattaggtttcatacttgcttccaatccaaggagagtatatctgagctctttttttttagatttgaaaaccgccttttagattagattagaacactgcctttttttttgttatgcagtaacagatcattgcccacctgaaagcgagtcgcctttatttttacctccttagtagaaccagcaggagctgcaattgctgttgcttgagccaaggccacagtcagcatagaaaggttctgctaagagactgtgttaaaggaaataaatttggtgacttagttttgttttacaagctagctgtggcccagtccttctgcagggtgggtacgcaggtttattgcagctctgcagagccatggccttagacaaacaaagaaggtgtgggaatacccgggagctttccattcaaagtagattcttctcctttgagtgcatgtaactgaagcaccagctttctaaaaaaaaaaaaaaaaaaaaaaagagagagaaaagaaaagtcagtctgaagtttttgtgacactcagccagactccacattagataaagtaatcatggaaatacagctttatttttataccaagttgtgcttagaaagctgattgctaaaccaggaagcttggctttcagaattttactgtaatccagggtggaaaaaaagtatcactcagatggcatttgtgggagattactgactggtaatccctccttgtcctagaagattaagtctgccctggtgtagtcatggtcagctgatttttttatttatttattttaaataaattgtagtttcttgttgtaagtcagccagtgtctgatgtttg

SEQ ID NO: 2 - DNA, 191166 bp, Homo sapiens
gaattcattgagctacacactttacacaatggaatgtctgcaacaagcctatgaggtaggattccctaaactcagagagcttaagtaacctgcccaccatcacacagctaataaaatgtaaagtccacattcaaattcctgtctgcctgaattaaaagctcatgatttttctggaattcctttagactttataatagaaaatgaagcataacttctatcttgaaacacctgtttcttttttttaagacatattaatatatctttaaaaaaatgttttttacagagtcagggtcttgctctgttgctcagtctggagtgtggtggcttgatcgtagctcactgcaccctcaaactcctggtctcaagcaaccttcccacctcagtctcccaagcagctggggccatgggcccatgccactgcaccagctaattttaaaaaaaaaagttattttctttgcagagacagggtctcactatgttgcccaggctgatctcaaactgctggggctcaagcaatcctcctgcctcagcctcccaaagcattcttaatgtccctttactatcattttgtgcagaatcgtgtcccaagttcatcacacagtgctatggtgattgatacgctatttgcagggcaaataagagtagtagcggtagtagtaataataattcatttcttattataaagcatttttacagttttaaaataatttttaaaacatatatcttgatcttttcaggagtcttatgactcctgacaggcaagtctcactattgtttactgttgataagaactctgaagtttggcccagcactgtggctcacgcctataatcccagcactttgggaggctgagatgggaggctcacacaaacccaggaattcgagaccagccaggacaacatgatgagaccccatctccttaaaaaataaaaaaattagccagccatggtggcacgtactcctggtcccagctactcaggagaattaggcggtaggatcacttttgagcccaggaggtcaagactgctgtgaaccgtgttcatgccactgcactctaatgtgggtgacagagcaaaaccctgtctgaagaaacaaacaaacaaacaagcaaaaaaaaatcctgaggttcacagactgctttgcccaaggctcacatccaaatgacatgccagaaacacatactctgcttcaaaattttataccacatccattacactggtcttggggagtggttactccttatggtatggaaactgatttgcaaactatttccagcagagtgcctcagctgtggtcagttggcaagtatgcatttaggcaaacgaacccacttgcagtgggctgagccctggagtcaaagctgtatcttcttatgctgagaaggcttcttccccagagggaactagagattgcttccaaggggttgagagttaagtttttctgaaagtcattatgggcttggttttgtttttcattttttcccatacctccctcttcacagaaaagtcatagtgttttatagcctgcttaaaactctgtgttctctgtaatagctgtaaagtcttcacttaattatagtaggctcttctattattttttagttgcaatggtgtaaaagaaaataagccctgtttccctttgaagttattttgtgcaaacatagtagtttctcttctctctccaagtttggtagcttggattcatttgtttccaggaatttttctttatacaatataaatgtatgcccacagccttgagctaattacactgtagccaattcccttcctttgtcattatttaccagtcccctatctttttttctagaacatatatataattagttaaataagcacatttagtactttgcttctcatgtaagttcacgtttttctaaggtgtggataaattgtttagagaactaattttgcgttcacttgtttatgattacaggtgttatttttgtgccccaccaatgcttcttgtatgtttaaatcttcgttgttgtcgttgttgttgtttttaactatttgaaattcacaactcactgaattttgtgtctggttttattttgaaaaaaaagtcatatttccaattttttctttcgcacctatttgtatgctgagcattcattaatgcaaaccatctgatttctgagcatttttatggatagaaattttgattatattggtgatttaaaattgtagaacctgaaacaaatgatttttttactacacatacacacattttagctactcaaagtgataatataccattagtgaagcgtgttaaaagtatgctacttactttattcatttcagaccgagttttttttcacattttaccatctctgaaattgatatgtgtcttaaaatcaatgtggcaagaacacatgatttaattaactgcttcttctttcttggcagtatgacctaaaagaatgccaaattttcaatttggtagcatctcagatttgattagatacaatatgtaccaagactactattgtacatgttttacaaagtctctcatttaatcctctaaaaaatcccatgtcatagacacaattattcttcacatttcacagaagaagaaaatagatgatggaagttgctgccatcatgtatcagctacaccatcctcttgtagcatgttgatgactattagctctcatgggggtggaggaagccttgatttgtagcatttgttaatttccatggtgtaaatactctcactgtggccaatctcaagataccaagatgaagtcactgaacatagcattggaaagatatgcatgcagttcactgatgttaagcagatgtgagatgactgcagcatatcatatatgttacgtgttttttctgggttatctctcacaaatccttgtctttagtgccactcatatgccagaaatggcaatatccaactttctattggtcacctccactgggatgccctacttgttcctccaattcaatggataaatagtacctttcttactaccgacattctgcctaagaatcataattgaatgcaggaaaaggaaatctagaattgtaataatatgatgcctaagagctgtgaagggtgtgagattttattctacttgcaagctaacaaattagactgcctattttacggatattgtcacaacattccaaaatcaaaggactttattactcatagaaaaagcagtagccctagtattgacttttttcagtagcctgaatctcaattcctaccaggtgatgcaaagagggccttcatgtaagatggtggcattacaggagaggaaaccagagttcaaggcacctgaatcttttttttttttaatttttgaaatggggtctcgctctgttgcccatgctggagtgcagtggtgcaatcatggctcactgcagcctccaccacctgaactcaagtgatcttcccacctcagcctcctgagtagctgggatcacaggtacacaccactatgtccagctcatttttaaattatttgtagggacgaggttttgctctgttgcccagactggtctcaaacttctagcctcaagtgatcctcctgcctcggcctcccaaagtgctcagattataggcagtgccactgtacctggccctggctgaattcgcgaatgaaccattctttttttttttttttgagacagtcttgctctgtcacccaggctggagtgcagtggcacgatcttggctcactgcaacctctaccacctggactcaagtgatcttcccacctcagcctcctgaatagctgagattacagggcatgcaccaccatgcacggctaatttttgtatttttagtacagatggagttttgccatgttggccaggctggtcttgaactcctggcctcaagcgatctgcctgccttggcctcccagaagtgctgggattacaggcatgagccaccgtgcccagactgaataaccattcttgattgaactctttccatttgagcaactaagctcattgggctcttctcaccttgttagtctctgagttgatgttgacctgggcacatatgaccccagggttacctgagctatatcaaattatacaaatgttgatcagaaactcagatgctaaatcctggacagaaaaagccaattgaacttatccagcaaggtactgaggattcttctttcacaataaaccggaaggtcagaaaatagccccaaaagttgggtgaagtctcctaatagccatacctgaaatacttcccataagaattgattggatggcctggtgcagtggctcacacctgtaatcccagcacttcgagagggaggctgatgtaggtggattgcttgagcccaggagtttgagaccagcctgggcaatatagtgagacctcgtcactacaaaaaatacaaaaattagtcaggtatggtggtgcatgcctgtagtcccagctactcaggaggctaaggttgaaggtttgcttgagcccgggaggcagaggctgaagtgagcctagatcatgcctctgcactccagcctgggtcacagagggagacctcatctccaaaaaaaaaaaattaaaataaaataaaaattaaaaaaactaactggaccataactcaaacgtggagaatatgtgtatttgggataatgtaaaaaatatttttcaataacatttgagagctaaggaatgtgcaaatgtaaccgtggcatgagcaatctagacatacccttgggattttaactccatgggaaccttcaaaacccattgcttactgcagcctcatccttgacctggtcacagaagtttatcgcacttattatagggcaatagctgtcactgcaaaacttgtcaatacttctcctgaactagttctaggctccctatttgacctcatgtttcctcctgcaatacagacattactactgactgagaacatacagcaatttcatccagacaatgaagtgttctcactaaagaaataagtgtgtgaaataatgcatatgttaaatatcttacatataccaaaacttcacgttgtacatcataaatatatttaagttttgcttgtcaatttaaaaataataataataaataaaataaaactcagcagacagctttcgcacttggtagagctggtcaattaggtaccagaagtacctaattgaccaactaggtttgagcatgctttgcaaacaaaagatctcggatggaatccccatcaaaaatgaagaacaaattaaggaactttaggtgcagccctactatgtggaaagggagctactataaaggttgaggcccatgtagaaagagagactttgtaagctaaagaaatgctccagcagcatttcctaataagaagccttaatcaaggttatgattccaagtaaaccctcaaaagataaatctcaaagaggccattacaaaatatcaatttttagtttcttgttttaaagaggaagaatagaaaaaaatctggttacacccttcacacacataatctctaatccttccaaccaggctggtggcctcagatgatttcaaatgaacattagctacatttctccatgaaactactccttatggtacagaaaaattggttactatctcaaatcaacattgatggggaaaattttaaaagatggctgaagttatttgcaggtcatgtgtcacctgtcaacaacataattttagaaaaattgtaagggtggggcatagccaggaaccaaagcttcaagggccctttgagtgtcttcctgcatctatttcattctctccatggaatttgaatatattttaggtattttgaatctattttcaggatgggttgaagcatttctattctaaagagttacagcccttattgttgctaaggaaaataaaagcccacttgagtttaacctgaggtaaaccaaattttacatcaagtgactgagaaatgctttacaggaagcattgtataagaacctcgtaaggcattacctcttactcaaaaactatactcctcctaccacccacaatcgtgttctcttgttctgagttgagcaattgggattcctggttttgaagcattttacaggtaatgttaattgctcttgtttttgtcatagtggtcatcatgctgatctactgtattctcgtcagagtcttaaatgtatccattcggtcattttcttgctggacgatcacaatgatgatacaacaagtcaagatctctcaattagctatggagacaactggacaatctccaagaagtaaaaacctggatttccaacattttgtgagctggggaacttctcaaaagtgagagaccaaaatgggactgagagactgaatatagaaatagatagtgatccggctgggcacagtggctcatgcctgtaatcccaacagtttgggaggccaaggtgggtgcatcacttaaggccaggagtttgagaccagcctggcaacatggtgaaaccccgtctctactacaaatataaaaaattagccaggcatggtggagcgtgtctgtaatccagctactcaggaggctgaggcatgagaatcacttgaaccagggaggtggaggttgcagtgagctaagaatgcaccgctgcactccaggctgggcaacagagtgagactctgtctaaaaaattaaaaaaagaaatagataatgattagacatctatgatgaagaactctggcccacaacctgttcaggaagccaaatcacaacctctgcagcaatcagcacagaactgtcaggactgggtcaatgactgccagcttctgtttttttgtcctcacttctaattcacgaccaatcagagaaagtcaaatagactgaccatatcactcatgtgattaaaacccttcacctgtgactacacatgactttcaaggtttaaatcatacttcttagcacatcttgtaagtttctactatattcatcggcatttttttttctttttttttgagacggtcttgctctgttactcaagctggagttcagtggcacaatcacagctcactacagcctcaacctcctgggctcaagtgatccttccacaccagcctcccaagtagggatcacaggcacgtaccaccgcactaattttttaattttttgtaggggtggggtctccctatgtttcccgggctggtctcaaattcctgggctcaagtgatcctcccacctcagtctcccaaactgttggtattacaggtgtgagccaccatgctcagccatcagtagtcttttgattacaagtgatagacacttaactcaaggtagcttaagtaaaaaagggaattaccggttcacgtaactgggaagaacagaagtgaactatcattcaggcataagtggatctagatgaagctaataggatttggccatggttcaaggtggggagaagcagctaatgtgatcttctgggtgagacccttgggccagttcaatgtagtgttgttatggtttccactgatgatgtatcattggggttgatgtttcacatggttgtactgagaacggtcataaagttcagagggtctaaggtttaaattttacagttgcatgctggttcaagggcaggattgatgtttagagtcaactgatattttcaggtcttaggaaagttgttaggaaaggttggggagtctggatgtgggggaagcggagggaggagtctatggaggggcagaaggaaagagatcagaaggataaggcagaagagggagggctggatgtagggaagcagctgagaacaagcagtggggaggcatactagggaaatgagtctagattgttattgcttaagtttgtcaaattcctcattagctttggccatttagtttttcagtgaaccaattattgaatccaaatttcattcagaggcctccacataccaataaaaaattactgcccattggacatgagaaagtttatttctcttcttttctaaggttcctctcaaatgaacaatactgctgaagtcaaatgtttccccagagtggccattgaaggaccttggtgaagtcatgcatttagaaagaagtcataagaattagggttataatgagagttccagtaagaagtttttcctggaagaggagaagacctagacttacacaacccctggagagggggcaacaattacttaaaagtaatggttttgcactttgtgtctcaggccccaacctgacagtgggccacctctgaatgcagaccccacaatctgtgccccctagtatgcagaaaagcagagagaaaaacagcattctgtctggatcaaagccaagctcctgggagaaaaaatcatgacaaagacagaagtgacttattcagttttattggtaatccacaacaaaatttgtctgaatagctaccattttggtgagaaccacaaatgccccagtctgtgaggatgattaaaaaaaattggctaataggacctatgtctgggctataccttttgattctccttttatgtcaaataatccttttagacagcataatacaaatagaaagcacaaacaagagcaaaaaggatgaaaataaatggaactgattgggggccaggtgcggtggctcacgcctgtaatcccagcactttgggaggccgaggcaggtggatcacgaagtcaggagttcaagaccagcctggccaacatggtgaaacccccgtctctactaaaaatacaaaaattagccagtcatggtggtgcacgcctgtaatcccagcaactcaggaggctgaggcaggagaatcgcttgaacacaggaggcggagattgcagtgagctgagatcatgccactgcactccagcctgggtgatagagcaagactccatctcgaaaaaaaagaaaaaaaaaaaaaaaaaaagaaatggaactgattccatgaaggatgccaaacaaatggggatgagtaaattacctgggtaccaaacagaataaatggccaaagaactcaatgtaaaagagcagaatgtaagcctggtgctgatttaccatgtcatcatggactcagaggccacaagcagcaagacacaaggggccttcctgaagccacacctgactgaaggctggcgtctgcagcagcgggaagtgcagcctgtgtcttgtgaacctccaggaaaaatgtagaaataaacaaagatgcccaaatgagaacaatcagaggctgtttattcagagcctgctctatccaggttgtcagccaccatcatttgtgtttggcagagactcaaaggcagccaggagaaggggaagatttataatgaaaaaacaaaacaaaaaaaccaccaaaggcttccagtatgatttgattaggggttcttggcatgaggaagccgggagcaggccaattagcagtgagcatctcctatgattagtttgaggagcatatttggcttcctctggttggtcctgagttggaaataggcaaaaaataggaaacctgacagttatgggcaaagtcctaactattctgggttgattgctgcataggctatggtttggctttctgatctggttgacacaggttgtgggacagggttctatggtcatatacagtctggccattgtctggaattcctgcttcagcctcccaaaatgctggaattataggtgtaagccatcgcacccagccaccaatgtccatttgtatattcagtttcttttcctttttctttttttgagacagaatcttgctctgtcgcccaggctagagtgcagtggtgccatctcagctcactgcaacctctgcctcctgggttcaagtgattctcctgcctcagcctcccgagtagctaggattacaggcatccaccaccatgcccagctaatttttgtatttttagtagagatggggtttcaccgtgttggccaggctagtctcgaactcctcacttcaagtgatccacccacctcggcctcccaaagtgctaggattacaggcgtgagccactgtgcacagcctcagtttcttaatagtaatctgacgccccttaaactccactgggccaggcgttgtggctcatgcctgtaatcccagcactttgggaggctgaggagggaggtcaggagttcaaggcctaggtgacatagcagggccttgtctctacagaaaattaaaaaattagccagatatggtggcacatgcctgtagttctagctacttgggaggtggaggcaggaggatcactcaaatctgggagttcaaggctacagtgagctatgatcacgccactgcactccagcctgggtgacagagtggatccctctctcaaaataaataaattaaattaaaatttaaaacctcattgtctacacagaagtccactccacctactgagcaaatgtcccttcccgggctgcctgaaacatggccaaagtcagttcaaaatagtctggctgtttcctataccccaaagtcagttcaaaatagtctggctgtttcctataccacaaagtaattttgaggattcaactttggttgtctcttcatcaataaccataagccctgggcacttactgatcctctttggactcaactcctattccaagaaaatggacataacaataaagttatttccttgaaggcaaaggaccaaaccagatgttcacttacagaagtttctagccccaattcacatgactattccataattttttgattctaggtctgtcctttgggatgggaccaggctgctgggttaaataggaagtgttcagatgtggttgtttctttctcctgacttaagctgtgacaatgcattaattcaccctcagagccattttgtggtgtagtgagaagagctctaaacttgaagggagatctctggtgagttacagttctgacatttaagaggtatttgattttcagtaggtttccttatattctttgtgtcgcagtgtcctcatttgcaagatgtcattctgtcgccccaaggttgtgaagagagtcagaatgtgagcaggtatcctcacttagatctttggcttcattaaaattgcaatattgaatataatgaagtgtgaattggagaaaataattcaacttttttccagtgcagaattcaaaaccttaaagaaaatgtagagaacctgaggaaatatttttcaggcagttcatccataggccagcaagtgggatttcctaagtcccctgactaatggggagacctccgctggtttattggtagagactggagggagagtatttctttcccatatcagcagaagtagctctctggtaggtcatttcacatctgcatctagtcatttactttataaacctcctccttctttggcggtttccctcgaggggaagcaagacagaacataattgagggagtctctttaatgatgcatcatttttgttttaatgttttcataaagaaaaagcacttatactattgtgcatcttgggctttaggaataagcccatgaaaggacttcagtatttttattttccttttttagggctggagaaaaagagctgggcttaagctacaccatgagcaaagcttcctttggagctactttgaaaagacattggtgacagtaccatagtatatcattacagggtcatttggagtacatttttggctctctgaggaggctctggggtcaatgtcctaacacgtggggactggaacctttttctccttattgttaaactccaatttcactggaaggtaggtttagtctttaacttcctggttgacactccttagttctattctgattcgtatggatgatttatgatacaggtcgaaaagcccaggaatgttctagaatatatgattggcgaggaccctcactggtgccatccacatcttctcttcaggactgggaacgttggcagctgatgatcctctgggaagttccttggtgagaactgccatcaatcttgaaaagccaccttgcccaagatcatttcctgagggcagcccacttcctagggtggatcagcatggggctataaaggcagggccatttgcttcaatttgggacaactctgaagagacactgtggctccagagcttctggaggaatgggatgaggcctctgttgtgactgcatcacacacagtccattcttcctccattgaatccttctggcttctcttcccaacaggtcggatcccagggtgctcaccaatccacccccggcactcaaatctccatctcacaagctatgtgacagggaccctgactacaacaatggacaaaacattttggccaggcgtggtggctcatgcctgtaaacctagcactttgggaggctgaggcagacagatcacttgaggtgactgcactccagcctggcgtgatctttttttaaattttgactctatctcaaaattaaaaaaaattttttttttgcctgggggaaccatattactctatctttctcatgcccctactgagaatcaaactgcccctcgtttttatttttattttaattttttaagacaaggtctccatctgtcacccaggctggagtgcagtggtacaatcagggttcactgcagcctcaatctcctgggctcaagtgatccaccctcctcagcctcccaagcagctggcatcacaggtgtgcaccactgtgcctggcccaaacttctcttaaattcattttccttatggaacaacaacagcaacaacaacaacaaaaacttttgagagaaagttaggagttacatcccaccaagagagctgccttcagaaaatatcaattgttaggcaatttcacagtattcagtgtcttcttcaaacttaaaaaaaattatacaaattatttttaaaatatagagatggggtctatgttacccaggctggtcacatcttctttaaacttgtttgacattttttctgaccctctcaaacagttaactgttatttataataattaattggaagcgaataaagggcaagtattggataactctttttcgttgtgtttgtaacatatacacaagataaaatttaccactgtaatcatttaaagtgtatagcccagtggcattcagtacattcacagaactccagaactttttcatcatcccaactgaaactctgtacctgtttcttttctttctttcttttttgtgtgtgtgtttttttttttttttttttttttttttttttttttttgagacaggttcttatactctgttgcccaggctggagtgcagtggggcaatcatggttcacggcagccttgacctcccggggctcaagcaatcctctcgcctcaaccccacaagtacctgggactgcaggcatgcgccaccacacccaactactttttgtattttttgtagacatagggtttcaccatgttgcccagattggtctcggactcctgggttcaagcaatctactcgcctcggcctcccaaagtgttgggattatgggcatgagccaccatgctcagcccgtacctattaaacaatgtagattactgttaatatgataagctcagtgttaaaaaaatatcaagaacaaactaaagaggataataaaaataatctgggcctatcatcaagaagtaactattattaacatttgtcaatgtcttgccagatatctttctgtgcataagatcgaatatgcaaatctattttttccaaaaatgtgatcacaatctgctttgctttctcatcacgaagaatgttttgtccatcttcctacgcaccatagattttcagagctgaaaggaaaattggaaatagtgtcagtaagggtccttatatttcactggaaatgtgtggggcttgagagctctcttcaaagaacaaacacagagatttcatggctacttcaaggattcagggaaatagcaaataacttctccctaaaaaaaagtttgtgctacctagtatattcatgcaacctaatttgtttttttagcttattgtctcactaatttatcagtctttttatttttactttccagtttttagttttttatagttaaacatgtacatattttaaagaggcaaacaaggatttttgcaaaaaagcaaaaaacaaaacagaaaaacccagcagtccaccctcttaccctttgcattttcctccttgaaggcaaccactttcaaatcaaaatcatttaggttttctgtttgtttgtttttgaaagagtctaggctctgtcgcccaggctggagtgcagtggcgcgatctcggctcactgcaacctctgcctccctggttcaagccattctcctgcctcagcctccagagtagatgggattacaggctcccaccaccacacctggctaatttttgtatttttaatagagattggatttcattatgttgttcaggctggtcttgaattcctgacctcaagtgatctgtcctccttggcctcccaaagtgctgggattacaggcatgagccaccacacctggcctagttttttttttttttttttttaaataacttgcatatattgataattccagttttcggcattatccaccagttttccactatgaatgatgaggattcatcctttctattcacccctcagtattactcattcctaactttcttacctgctcccattctgtcttttcaatctaagaagaaaataaggtagaaatcacttttcttcagtaaattaagcctaatcctccattgtcttctagttttgttgtccatgactccaaacccattgttattccttttactttgaatgtgaccattttttttccatctctagaacttgtggggtctttatctccagtgttctgaactttataaatttgtgccgtgttctcagtgagccctttcaatctggaaatccttcagtctggggaaattccctgaattatttcactgattccttctttttttctgtttcttccatgtctttctcttggcctcttttttggatgttagacctctgggatcagtcttctgattttcttatatgttcccctttatttttcatacctttgttttttggtcttctctctagaaaatttactttatactccaaaccaaccatggagtggcttaaagtagcacagaggtattaacttatggttctgaggatcaggagtctaacatgagcctcacaggactaatgtcaaggtatctaggactaacaaggtatgttctttctagaggctcttgagaaaaatttgtttcttgcatattcaggttgttgcaattcggtgtcgcatgattgtatgctaaggtcctgttttcttgatggtggtcaactaagggctcattcagcttatagaggtcacccgtactccttggcccatggtccccttttctccatcttcaaagccagcaagagtgggtcaagtcctctcatgctttcgaaactctcctgcctctttttctgttgtggcatctctcagtcacaagattctcagctcttaagagcacatgtaggtggggtgcaggggcacacacctgtaatcccagcactttgggaggccaaggtgagcagatcacttaagctcaggagtttaagatcagtctgggcaacgtgacaaaaccccgtctctacaaaaacatatgaaaattagctgggtgtggtggtgctcatctgtagtcccagctacctgggaggctgaggtgggaggatttttttgaatcctggaagtcgaggctgcagttagccaagatcatgccactacattccagccttggtgacagagtgggaccctgtctcaaaaaacaaaaacaaaagagcacatgttattagagtgggtccatctgataatccaggataatcttcctctctcaagatctctaacctttatcaagtctgtaaagtccctttttgccaaaggttctgggattaggacacagacatccttggagatctttatcccaaatgatacctaccacaggagttttaatgtctgctataatataatttctaacaactggtttttctctgaatgctccatttttttatagagcaaactatttttgtctcatgtttaggatattttcacttatctctctgaatataataacaatagctttaaagatctgtttgtttccttctccttgtatagtttgtttcctgtaagttgcctttttatggtgtttattttggtctttatcttccattaagggacttgtattagttgggtcccacgctgctaataaaaccatactggagagtgggtaatttataaagaaaagaggtttaattgacacgcagtttcacatggctagggaggcctcacgatcatggtggaaggcaaaagaggagcaaagtcatgtcttgcatggcagcaggcaagagagagaacatgtgcagggtaactcccatttataaaaccatcacattttgtgagacttattcacttccacgagaaaaatatggggaaaactgctcccatgattcaattatctccacctggctccgcctttgacacatagggattattacaattcaagatgagatttgggtggggtcagagtcaaaccatatcagggtttttctctggatcatggctgcctgccctgagcatgaagcagctgactggtctctttaggtacgtgggcaagatttgtgaccatagatttgagggtaaggtgatctggtttatccaatgtcaacatttttggttctttactcttaggttggttaaattcctgagagatggatctttcagtctcctgctttcacagtatatgcctggctgtttgcatcctgagaaccaagcagagcaaggctggagaaacagcattcatgatgtgaactgctaatgaatttttctgcctgtattaggaaatcctcagcctcaaatgtacttagtcttctagtaaagagattgtcaaatttccttctccagagaagccactttagtattcttgcagaatagggaaggggatgggacccaacttcttaagtaaactttcaaccaatcctcttgttttgagcctcactttctttctacatccacagatacctgatgccaagtcctgaggtttttggaagtgctgcagtggaaattaggttcttcttggctttcccagtttagcatttggccttctcagattcacaagatcaattatcattacgtagacactcacattctctttgtccttgtgagtttatctctttactgtaattttagcagcatagagaggaagtaagaatgaatctatgttgagaatttacaagaacctcttaattttttacttcagaagggtgagctttatggtacatgagttatatctcaataaaaaacattttaaaaataaataaggctggatgctgtggttcatggctgtagtctcagctactcagtaggctgaaatgggaggactgcatgagcccaggaggttggggctgcagtgagctgcacatcagtgcagtgagctgcattgatcctgccactgtatttcagcctgggcaacagctgagaccatgtctcaatcaatcaatcaataaaaatgtgtctgatatgccatctttatcctaaagtatgactccatagtttagagatgaggacagtgaaccagagaagcttaagtgagaggcctaagatagtgatgttttcttttctttcttcttcttttcttttcttttttcttttcttttttttttttttgaggcagagtgttgctctgttgcccacactgagtgcagtggtgcaatcttgactcactgtaacttcaacctctcgggctcaagcaattcttctgcctcagcctcctgagtagctgggactacaggcacataccaccatgcttggctaacttttgactttttggtagagacaaggaggtcttactaagttgctcaggctggtctcaaactcctggactcaagcaatcctcccacctcaaccttccaaagtgctgggataacaggtctgagccaccacactcagatccatagtgatgtttttgtttgtttttgtttttgagacagagtctcactccatcacccaggctggagtgcagtggtgccatttctgctcactgcaacctctgcctcccgagttcaagctgttcttgtgcctcagcctcccaagtagctaggattacaggcatgtgccaccaccactcctggctaatttttgtatcttagtcgccatgttggccagcctagtcttgaactcctgacccccaatgatcctccctccttggcctcccaaagtgctggcattatagatatgagacactgtgcctggccagatagtttaaagtgatggtttagataaatccactccagagttttcagttgtttctggtcttgcagagttatattttggtgacagtggcacagaatttggctcttcatcaaagaccctgggcacttgccattctggcctacaccatttctgccagaggaccattggcattctggaaaactagctatctttgcaggggtgaaatggtttgtaagacactgagttgaacaccgtgtaaaaatttagaaatattgttaatcatttaattgttaaatatacatatctctaatgcattttataccctggagtaatcttagagagagatgaaaagattagaacggtgatggtggagactccaggagaaaagggacaatggctctcccagcactcccttccctacccagttcccatatgtaatttgataaatacacagaatccagagtggcttaaaaacatacaagaaagattaagtgtctatggcttttcttaatgtagccaaaatcaatagtgtgagatcgtttcctgttaatgtttaggtatttatgtgtccaacctccattagtgttaagtactctttcctgccaatacctgtttctacacatttttaaaagttgtttatttcatattcacagtcagtctcttacatggatggctaagagaatacaataagatggctattcaattatcttaagcaaaaaaatgttcattacctgacattaaatcaatttttctccaaaacttgttcttgatctcttgggcctcctaaccttcatccctttgtttactaccatgattcaagcaggaagagtctatctttctcctggatgatgatataattttcaaaattgtggtatcatatgccttcatttgtttgaacaatcagagagccagaactaccctttgacataggtagggtagtcagatttagcaaatagaaatataagacaacttgctaagaatttaagataaataataaattttagtataagtatgttgcaaatattgtagggtatacttttttgggttttttctgagatgaagtcttgctctgtcactcaggctggagtacggtggtgcaatcttggctcactgcaacctccacctcccgagttcaagtgattcacctgcctcagcctcctgaggagctgggattataagcatgtgccaccacacccaactaattttttgtatttttagtagagacaggggttgatcacgctggccaggctggtctcgaactcctgacctcaagtgatccacccaccttggcctcctaaagtgctgggattacaggggtgagccaccactcctggccagggatatacttacactaaaaaattacttgtcatttatctgaaattcaaattgaactgggtgtccttttatcttatctggcagcaaccctagacccaggctacctgaagcccctccactgggcgccctgggtcccatagagacttcccacttttgttctccttgatgagcagtccctagcatcaaatatctaccccctctttatcaacggtgcatctgatacctataactacagaatctcttgcctaaactggccaacatctgttgtcaaggccttcagcttttctcccaggacagtccttttgagtgcaaccctagaatggaggtatggccaaggctcggctgatgggaaagggtggatagagatgggatatgtcctaaggcatccacgcacaagggagtgggccagcctggtgcaggacagagtcaaagtgaaaggacagagccagaagagaaagccaggggctccatttgtatcctcgccccaggccccacaaatatgaggggtaagactacagaaagcatcccacttgccacacattctgtgtgatttctgctggctatcggtcaaggaagatacatacaacaagggcagagtatagagtttttccacttacataagtcggtttctatctatccttttcaataccttcatttgcttcaatgcatttgtttcaggggaagtttacaatttcttgtaaaaatttcaccaacaaatgagtattctgaaaaaagaaacttaaaaaaagaaacttaaaaaaaatccacgttcccatcaacaataacaccacaaagtaaatctagtgtttctttcctgtactgagccttttaaatgtggttcaactcaatgcacccaggtgctctgtttaagtgctagccattctccagagattcctcagagtgctaaaacataagacctgttcatttcagaagtaacacagcataaattgtaagcacagattctgtcagaagaaaacaaatgaagtagtacgaagacacaagagaaaagcaggcttttgtagaaatacatgtagtaggaagatgtaagagaaaagcaggcttttgtggaaatatcatgtactaggatatttatagtatgaacagtgctcatttaacctcaaagagaaaattaagttaatctatctctaaggctcaggattgcatgtatccacctacgtgggtgcatgccagagtgcattttctatatggcgcattctatcagttgggattgcatttggctccatctaacagaaagccaactacagtggccttacaaagtcaggcatttatttttttccagtaagaagcaggtctctgatgccatggattcttgggttctttctgccgtccttggcttgtgaatttcttcctcgtgtttgcaaaatggctattgcccctctaagcaggaatgggaagggtgaagggccaagtaatgctgccaagtctgtcttctttttaaatgctttcttggagattccaattagtacagtttgctcacatctcattggacattaatgagagagacccacctccagctgcaaggcagtttgaagtgcccagtgtttcagctgggcacattgccactccaaacaatatttaggttatcttaggaaggaaaacaggaaggctgtttatcaggtaggctatttgccatctctgctgcatgtgaactactctataagctcttcaaaagataagactttttttcctttcagcatacataacaagttcatcactatatagtaagttaaacagatacaaacaataaatatttgtaacggacactcacgttcaagtgctatttatttgtccacaaattagtacattgagagattgtctctccagatgtttatttctatacaatagatctcagcatcagctagtcaaaaattctttaccacatacagaattttctgctgatgccaaaatttacctttatgttacattaaaaattttttaaaaaatattgcacctttttgggctcaagcaatcctcttgccttagcctcctgagtagctgggtttacaggcaaacaccaccatgcctggtttaaaattacaatatttattaattgcatggtagaattaaaggatgcagagatgaccaaaataatctttgcccacctggaacttttttttttttttttgagatggagtctcactctgttgcccatgctggagtgcagtggcgtgatctcagttcactgcaacctctgcctcccgggttcatgccattctcctgcctcagcctgccgagtagctgggactacaggcgcccgccaccacacctggctaatttttggtatttttagtagagatggggtttcaccgtgttagccaggatggtctcgatctcctgacctcgtgatccgcccgcctcggcctcccaaagtgctgggattacaggcgtgagccaccgcgcccgacaatttttgtatttttagtagacacaaggtttcaccatgttggccaggctggtctcaaactcctgacctcaagtgatccacccgccttggcctcccggagtgctgggattacaggcgtgagccaccgtgccaggcctgttttttgatttttttaataggccattttaaggactttaccacctggagcttgtaacctaataggactgtgaaacatacactggtagcatcctggcaccagtgctggggtgagtgccacaggagaggagccaagtgcagtcagagtagaaggactcaaatagaagagatcacatctggttgagtcaaacagtaaacgtccccagaagacactggaagatgcaggctggagagagctacactttacatacagcacgacagtatagagatattaatagcatacagtcacttcaggtaactacaagtaattttaaaacatctgaataagtgcttctttgttgtctgtggagtgctacacaatgcttcggcctaaacgtcaagcaacacagaaaagaagccccacttcatttgcagcctcagatagcttacgaaattgagacaccacctgaagaccatgacccatacaatcaaagtgacttcatggaagacgagtaactgcaaaaccctgtgaaaacagattgttaacgctactaatgaaaagaactggattgttacgggaacctcaaactgtcctttgatcaacggacatgacgcctcaacacgcattttccaaagatctctgctgataattctgaccttcggttaatttaattaatttcatttgatttaattcattttagtgttgtcctcagtcccgcactcaaaacagatttttattatatgaaaatcttcctagtcacctaaaaaatccgcattctatggaaaagactatataatttttcaagagattctttgcaattttcacctatgctggtttatgacctagttagagaagacatcatagggcattatttaggtaataaaatacagagagaaaagagccactggcatttctaaataacgttcctgtgtttcctactgtaagtataggaaagtaggtgttcgcagatgcagagaaagggcatctccaaggcctcggaggaaacttttgattttataaataacttgatggtggaaggcaaacgccatcccggttcccaagcaggctccacgcagcctccatggccgagtccatctcctcgccgtcctcttccctcctcccggccgacttccagccggtgcgggagtcggggtgcgcccggccctcctagcgctgcgccgcccgcgccgacgtggcggagcgcacaggtcgaccgggtcaggtggttgggtgacgccccaggaaagcgctccaggccgagggcgggcggcgccgccgcgcccgacgcctctgggggtggggacttcgcgggagtcgccggcggaggcgaggaggagctctgcgcggcgcggcgggcgatccgagccgggacgggctgcaggcgggggtgctgcagaggacacgaggcggcgggctggagacatggaccgcggcgagcaaggtgagggcgcggacccagcgggcttgggctcgggcttggactcgggcttggactcgggctttggctcggcgcggggctgagcgcggcccggcggctcgagcaggtgccgagcctcctgggccggcgccgcaagttgcatctcccggatccggatctggcatcctggggccctggaaggagcggctctggcgggggcagatgggactgcatgtgtgagtgtgagtgtgcgtgtgtgggggagagggagagaaggttctgggaggtggcgaaggggctagcgtcaggggctggcggccgtgatgcgctttgggtggaaagccgcgtgaagggagcacggcgcagccagctcctgtaagtgactggaaccaggaacagaggacagtgagtcgctctcgtccctccaggtgggaaacccctctctccctcataaattaagactttctgccttcttctaagttcaggtggttaggatttttggtggcgtatgacccatggggggtttgggaggcagatatttatcatactagaaacccccagattgtggagtggtgtctgtgggtttgagggaagagagcttttcgtaggaagggtgaaatcccacaacacaaaaccttcagtctaatttaccatagcgttgggtgcacttgcttctgaaaacagctcaagtgaaattacccccaaatagagcaatagtaggccatggttttcagatttaaatagtttcatcctagagatctctgctgatcataagcctgtgactgtacgcagatgtgtgtggcacgcttttaaaaagatacataaatgctgaaaaaagaaaacaaggtaggtaatcttgggaagtgaggataggaatcactcctgttttgagccctgacacaagttttcatgtattgcttattttcctccagtatgtgccgtcatgtgtgcatttttatgtaaatcaaaccatattccaaatatactatcttgtactttatttttttcagactctggtgttttttttgtttgtttttttgagtctccacctgtagcccaggctggagtgcagtggcgggatctcagctcactgcaacctctgcctccctggttcaagtgattctcctgcctcagcctcccaagtagctgggattacaggtgtgcgccaccacagcccggctaattttgtatttttagtagagacagaggtttctccatgttgaccaggctggtctccaagtcctgacctcaggtgatccacccgcctcagcctcccaaagtgctgggattacaggtgtgagccactgggccaggcctgtactttactttcatttacttgtaagatagactctttggagtctttcagctctttcctcgctccccttcagattaatttcagacttaacacaagtgcttacaaacattacatacaatatgctttatatgtgttacctatgtgtatatatatattatacattacctgtataacgtatatatacacatacatattattatctaaattttaagcttcttgaggttagaaacatttcatctttggttgtcctgagtgggataacatgatgacttgtacatagtagacatgcaaatgtttgagttagaaagcttgtcacatgtcttgtatatattgttagccggcttaggggccaccatgtaagtcagcattatgtctgtctggctttcaatgggggcgacgcaggacgggggagacatgacgacaaacgtgcccatgtgtatttgtatactaatctgttacacactgtcgtttgacataatgatgtttttagaataactatgtttggtacttgtcaaagtaaattaaacggctgtgttttacttacgtgagtggagtaactagttttagtagtgtttgtgttctagcttgggtggtaagaatcaacagtcattggtagcactgcagtattccaacttcttgtgtagtttttataggactagtttaagtgtctaattctaagacatatttttttcaaatctttatcttgtaagatgagtcttttcgtgtctataattttcccatatgttctttgggtgaggtggaggactctgctccgaatgaaagaagaggcactgccttgggagggtcagttcatttactccaaagctttgctgtgttagaacaacgtggccaaggctgggtgcggtggctcacacctgtaatcccagcactttgggaagctgaggcaggcagatcacttgaggccaggagtttgacccctgtccaacaaggtggaaccctgtctctactaaaaatacaaaaattagctcggcgcggtggcaccggcctgtagtcccagctattcaggaggctgaggcaggagaatcactagaaactgggaggcagaggttgcagtgagcttagatcaaaccactgcactccagcctgggcgacagagcgagactctatctcaaaaaacaaacaaacacaaaacaaaaccacctggccattccttactcttgcttccacctgcctacctgccttttcatctttatgttaccagtgcacacagtaggtgcttagaaatatgtgttgactgcctaagatgagtgaggaaagtgtccaagagcaggccccagccatagtaacctctgtgtccctccaaatccatctcttgtttcccactttctcctgttctgttttccctgtctattctcaagaagcagaacttagccagagagttgctgataaggcagtgctgggcttggaggataactttacatggaagatcttagaactagtagagtggatgactcagtcttaagtgaatctggagcaagactcctgccttaaactggaaaaggcacaacctgggattttaaacctggtgttcactagcaaactgtgttggctgaaataaggtttatcaaactctgtgattgtctcctgaattgtgattttggcaacatacttaaaaccaaagcagaaaattttaggatttgttgaacttttaatagtttttaatgtacatggagttttcttatatttgtcccttaaaaaatccctaatgaaactctagttgtaaaattaaatgccctacctcttgtcttacatgcatttatctattgaaaatccctcttccaaccttcttatgtgaagttatcaaaaaatctgttcttgctactgattgtacttaacagagctgtaaatttttaaaataaacactgagttttccttatgttttcaaccacgagtattctaatgctctgtagtatcaggcccagattctgttgtttttctactttctctcatacctagggcttgaatacaatactctgcttacagtgagagttcagtgaacatcaaatgatttaaaatacgttaaattgcatcatgcctttcaagagtttccaagcattaagctcagcgcagcacatttactgtgtgtggtatttcctactttgatagagcagtgttgctaactagagcatacattgtgtagaaaaccctcctgactaccttttatggagctgaataaatatgtatacttgagtgctctcatgttagtcataacctttagtttagcactgttatatagaagtgacagtcacgtagcccactacacattatactagtttgaatttaaacaaagcaaaacaactttataattttaaggggaagaggaatggttcctgaggccaaaaaactagagaggaaaggcttcaaaaaaagaataacacaataatatgtcactatcattttaacattgtaattgtgccttagaaataaataacagaaatgtggggtatcatttatttaaaaataaagcaaatgtagagaataatggataataattttgagggtctactcccaagtctctgggtgtgacccaccccatccaggctgttaatttatttggtcaacagtttcgtgatttccatatggtgctgatgtctttgttagctcttcttcacttcctgcatttatttttatgtggcaggctgcctagtccctaggggaatagctgagtggtaggaggggttgtgatcttttagcaagatttcaaaacagaataatgtatgcatgtaataaaaacaaatacgctcactcacagtgcaaaaccaaaaagaacacccaaatccctggaacttctgtgctactaagtcttttgttgtaggctaagattattttccttttggggttaattatttctgccttaattattgactgttgtgaaatctctcttctgagacattcccaaattttatttaaactagtgggtaccagctgtactgaaaccataattttagccatgtatttttattagaaaaatgtttccaggggattaaggaattttaatatttagtggaagttttaaaaaagaatgtaaaaatgccttttggaacctaagaatagttgtctattttagaaaaatcagaaatattgatggagaaaaaagaaaattacccataatcctgtgcgagaatgagaaatcatagttagcatattaatttatacctgttcatgtatctatgctttaaacaaatcacaattttattattattttttttaacagtgtctcgctctgccacccagggtggagtgcagtggcgtgatctcggctcactgcaacctctgcctcctgggttcaagcaattctcctgtctcagcctcccaagtagctgggactacaggcgcatgccacgacacccagctaatttttttgtatttttagtagagatggggtttcatcatgttggtcaggctggtctcgaactcctgacctcaggtgatctgccctcattggcctcccaaagtgctgggattacaggcgtgagccactgtgcccggccaacaaatcacaaaatttcttattgaatattgcattttataacttgcccttttaaaaaaaccctaaatgtatttaaaaaatacactttaatggccgcatgtggtggctcatgcctgtaatctcagcactttgggaggccaaggcgggcggatcacctgaggtcgggagttggagacccgcctgaccaacatggagaaaccctgtctctactaaaaatacaaaattagccgggtgtggtggcatatgcctgtaatcccagctactagggaagctgaggcgggagaatcgcttcaacctgggaggcggaggttgcagtgagtctctctctcttgcccaggctggagtgcaatggcaccaatttggctcactgcaacctccacctcctgggttcgagtgattctcctgcctcagcctcttgagtagctgtgactacaggcgtgtgcctccacgccataatttttctatttttgtagagatgggtttcatcatgtagtccaggctggtctcgaactcctggcctcaagtgatcctcccgcctcagcctcccaaagtgctgagattacaggtgtgagccactgcgcctggcctagagcagctccatttttaatggccatttgtatgtatttatttatagatgtaacattttatatactgttatagacaatatttaacttgttccctgttgatgaacatttacattttgccttgtaggggaggaaaaatttctccctacccttttagggtttctggcgaggcctaggaattaaaccaacatgagatagattaacaggagaaaagcatgcaaattttatttagtgctttttatgtattacataccttttaccctcacaagaggaatgaagactcaaggaagtggcttggcctgatcgattatatactaggtcaaaaaaagagtggcagttgtggaaaagtaactaggaaggtaaggtttagtttagcaaggtttctttatgcagatttctttgggcttggacttcctgtgataagaatgtttctttcttcctggtagagggaggacatctttcatatgggagcttcatcttctgctttcaggaaaaagagaggaggtttgggtgcccttcttaacacctgctgtttttcaagtgcctttagcccaaaataatcagcatgctaaagtggaatgttttgggatgctgtgtcctgaactccttcaccctgatattttgctgttaaaacaaagctgcagcaaatgtatgtgtatgcaagtcattccataaaaatgccagtgtgtctgtaggaagaattcttagaagaatgtggctgttcaattttgatagtgccaaattctgctccatagaagtggtgccagttatacttccaccagcatgtgcgagcgtttgctttcccttgccatgtggccaggatgttgccacacttttccattgttgcttgtcttataggggaaaaatggtttctcagtataagtcttactatatatatttaatataaaggtagtttacttcttttgatatgtttacaatctacatggcattttctgtgaaatatctggccatatcttttgccaatttttctattgtattttatagaatgtaccccgtttatgagatgagttgttaattttttttctagtttgtcataccttttttgactttatagtgattttttttaaccaagaagaaatactaatagctctttttttttttttttttttttttttttttgagacattgtctccctctgccacccaggctggagtgctggagtgctggagtgcagtggcatgatctctgctcactgcaacctctgcctcccaggctcgagcagttcttgttcagcctcccaagtagctgggattacaggtgcgtgccagcatgcctggctaattttttgtattttttagtagagatggggttttgccatgttggccaggctggtctcgaacttgtggcctcaagtgatctgcccacctgggcgtcccaagctgctgggattacaggagtgggccaccatacccagcctaactaatggctcttttcatttatggattctagcttttgaatcatttttagaaatgcctccctcactttgagtttatgcttttatggttttattttttatgtttaaatctttcctcaatctggagtttgttttgataatgagatgtgaggtataaatcttaacacctccacctccaattggccacccaactgcctaacaccattcattgactaatggcttcttcctctattgatttgaaataccactgttgtcatgtactaaattcttacattcttttcttttctctttatctgtatgttcattgccagcagctaaacctagtttaagttattgtagcttcataaaatgtaaaaatgtctggaagggctagtttgctctgattactctatttttttttctgtcagtttttttctaggtattcttgccagactatttccaaacgaacattagaatcaacttatctaggtgaaaattcaacaaaaatcatgctggcagatctttttctccttctctctcctcccccctttcccctttctgtctgtctgtctctctctctctctctctctctctctctctgtctctctctcacagttagaatttggtctgtttctttattcaataccccaatatatgttcattagggttatactgtatacactacacataacagttttgttttttgttttggatattatttgataataagaattttaccacatcattaaaaaaagtttccccaagctataatttttgataattgcactcttccactattcaaatgtttatttaactctttctctcctggagtaggtttacattccattttagctatgatactgctttaagagaaattgttttaagataaatttccatagacaggtcaaaggaggtgaatatatgtaagcttttcgatgcctgttactgaatctcattctggaaaacataactgtcaatgccctctttttctcatggtaaaaaaatacataacaaaatttaccatcttaatcgtttttaaatgtacagtacgatagtgttaactgtatgtaccttgtgcaacagatctctgaaactttttcatttttcaaaatgaaaactctgtactcattgaacagcagcttcccaacttccccattcctccatcccctacccctggtaacagtcatagaaaccgtgattttatgagtttgaccactttagatacctcatataggtggaatcatgcagtatttgtcttcttgtgatgcttattttatttagcatgatatcggggctcatgcgtgtgtagcacatgacccaatttccttcttaaggctgaataatattccattgtatgtatgtactacattttctttattatttatctatcagtggaaattaaggttgcttccacatcttggctgttgtgaataatgctgtaatgaacatgagtgtgcaaatatctctttgagagcctgtgttcacttcttttagaaatatactcagagatgggattgctggatcatatgataattctgttttaattttttcagaaccttcatactgttttccataatagctttaccaatttacatttgcaccaacagtacacaaggattccagtttctctccatctttatcaacactcgttatttcccgttttttttgtttgtttgtttgttttgttttgttgtgttttgagacacagtctcactctgtcacccaggctggagtgcagtggctcaatcatagattaccataaccttgaactcctgggctcaagtgatcctcctgcctcagactcccaagtgctggacttacaggttgagccattgtgccaggctgttttgcctgtttttgatcagattagttgttgagttgtacgacttttttatatatatgttctggatattaaccctttattggatgtttggtttgcaaatattttttcttattacgtaaattgcctttttattgtcttttttttcttttctctttttaagagattgagtctttctcttttgtccagactagagtgcagtgatgcaattctagctcattgtagccttgaactcctgggctcaaggagtccttctgcctcagtctctctagtcatttccttaaaaaaaaaattacagagcttataggcttcaatttgtctgtttcctagagaggttatagatgttattatccatttgtatttccctctagagattgttacttatctactttgggatcttagtgtgtcttgccttctcattgttctctgtcaccttccctccttggcaagtatttactttttctattttgcttatcattttggttatgttggatctttcaatgttatttttattagcaaaatacagtattaggagcaaatattttccaggtgacattttgggggtaatttgcatggctttctgtggctttcagtacttccttcttggtgtcttttctgtttagacacttgtaattggtactaaaaatagtgatttcagacaggctattaatttacatgccttggctcataatcatggtgagtgtgttttatgttacagctatacaaatgtaaatgtcagttttaaaaattatttaaggccaggtgcggtggctcacacttgtaatcccagcactttgggaggctgaagtgggcggatcacttggggtcaggagttcgaggccagcctggccaacatagcgaaaccctgtctctacaaaaaatataaaattagcccggcgtggtggtgcatgtctgtagtcccagctactcaggaggctgaggcaagagaatcgcttgaacccaggaggtggaggttgcagtgagccaagatcatgcctctgcactccagtctgggtgacagagcgagactctgtctcagaaaaaaaaaaaaatatatatatatatatatataaatgtgttcttgataaaagttttggattgggaacttattaacaggatgaaatttcacccattgtatccttagtttatttggaggcattgtggtgtggaaaaggagcttatggtccaatcaggagggcttaagaaataaagatgatagtttatccaacccactagaatgtgtacttggctagaaatagtgtcactagaataggagactgccttacattttattcccagcttgcagttcaagtatgaatgtagtaggtactccataaatatttctcatattgcacatacatcaatcaaaaagacttctagcattaatagagtcagatggatttggatagaaaagactaagaattaaagaaggaacatggtttggcagggagataggaatatatgttctaaatatgcttatagtcttacatataatcactgaaaatctgtctgtgaatataagttaaattggtactttgctcctaaggcaggtatgtaagaatgtgtgtgtatacataggtagaagagacggtgttggttaaatttttattcttgaaaggatattggatatagtgctcaataaatacttcataaacacattttcagtgctgttctattgaagttagttatttacttctttttttctttttcttttttttgagacagagtctcgctctgttgcccaggctggagtgcagtggagtgcagtgattgaggctcactgcaacttctgcctcccaggctcaagcaattctcgtgcctcagcctcccaagtagctgggactacaggcatgtgccaccatgccgggctattttttttttttttttttttttttgtatttttagtagagatggtgttttgcctcgtttcccaggctggtctcgaactcctgagctcaggcaatccaccctccttggcctcccaaagtgctagaattacaggcatgagccaccgcccgtgtccagccagttatttacttctaatgcattctgagcctaggagacatggatggccttagcacatgttagtagtagtttttaaaaaacacatttcagaagggcaagcagtatgttgacagtagtggaaagtgcttgctactagtaaaccagaacaaaaattctgtaaagagggaaaaatgcaaacattttgcctgcctgagacatttggcaaatggatgattaatcttccatttcagtatggaagttggaatattccatatggaagttggaatattaagtatggaatattattttatgagaacccagaatgagaatgatgacgatgataccacatggcctttgagaactcttactaattttctacatattatcttgtttcagtctttcagtgctgcaggaaggtaggactggtacattttcattttatatatgaggaaacataatctgaatgatcacgacttccccagatgtactctgctggatgtggagagctctacaaaagcttcgttccaaatctgttgtctctaagagaataatctggagtccaagaaaaatttaaactactttttttttttgagatggggtcttgctctgtcacccaggctggagtgcagtggtgcaatcatggctcactgcagccctgaccttatgggctcaagcaatcttcctacctaagcctcctgagtagctggggccacaggcgcacaccaccatgcctggctaatttttttattgtttgtaaaggcaaggtcttactatgttgcccagactggtctcgaacccctgggctcaagtgatcctcctccctcggcctaccgaagtgctgggattacaggtatgagccactgcctggcctaaactacattttctaatcagtttatagggctaggaacatctcatactttttaaagaaatgttttctttccaaataattgaaacaaaattggaatcggaagtaacagagcttcagctttgcattacaattaagttttccagctggggttttactgggtcatttttagtaatacaactactgtggttaccagcaacaacatttattgttttctgtatgtcaggttctgtctaaatgcttttcatgttttatttttgaaaaatttcagaacataccctatgagcagatgctaacagtatccctacgttacaaatgaaacagggcatgaagggacagagtaataactttttcaggggcacacagcttggcgcagacaggtctgggattttgatctgtgatgtctggctagagagtacgtgtccttacccagcctatacctatactacataaaagttgcagacaatcttccttttccttgcaaaggtgttgttcaaaagcacagtacagctgtggtaggagccatggaaaggagcaccaagcagtgtatctataccagagtctggccagtgtgtctgatcttatgcatcagaattgattgtaaattagttcagaatactgaacctgaagtcatatcgaggttcaaaatttggcttcatcattttctggttgtgagacattggataagtcactaaacttctctaaggctgatatctcacatatgcatgaaaattaggtacaaatctgttactagtactggtgctcaatagatactgatttttgaaactgaatattagatacttctgtaaaatgctaactcagtggttcccagtttaaacgaaacattttatttctttagagacaagtgtgatattggacatagtgctcaataagtacttcataaacacattttcgatgcggttctattgaagttagttatttacttttttttttttttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcatcgttttagccgggatggtctcgatctcctcctgacctcgtgatcagcccgcctcggcctcccaaagtgctgggattacaggcgtgagccaccgcgcccggcctacttctttttttttttgagacagggtttcgctctgttgcccagcctggagtgcagtgatgcgatctaggctttgagcctgctctcaggctcataggccattctgaagaaacaaacatacaaataggctgctgtagtagagtagatgttgtgaaaagtttataaccctggctgaggaggaagtgagcaacgctggctggggtcttcagaaaagaattcccaaagtaggtgacctttgaacttggacttgaaagctgctattgactgaatgtttgcatctccctcacccccattatccaccactctggcagtggtagataatgctgaaagatttgggttccatggatgggggtggggggtggagttgaggagaattgggttggggttgaagaggagtggggtggggaggaggagaatgggagggctggtacaatggattgcaggcctgtgggaatgggggaatggtaaagggaaggcaaccggaaagggaaactgctctttggagaagtcatgggtgttttaaagggaaatttggattaagtgatgggaatttgcttggctattcccaagcaaatattcccaagcaaatagactatagatattaaatttgaatgtcgtttttagttcctcagtagagaggatgagtagtttggcatttgtgtgtgtttgtaaaatgagagcagaaactagaggcatgggagaataactggatttgctatgcctgtggatgccactggttttagttaagatgcttttaatgcaaaatgtattttaatagtgatatcgtagagtttgaccttcaagaagctcaagtctgtaagcaataattcactttaatgtgtaagagccttgcagagaataagaacatattaaatatttgcagaatgaaagtattatactttccctgggtggtatgatagccagagaagcagtttagtctagaggtgacatttgggtgaaattgtagtaatgctgtgattttagaattttaaccagtgatttctaagggccaacctgaagcttgttttcatcgtggagtgccttggctatgggactgatttgtgcagcttctagatttgttaactattctcgctcgggtttttttgagaaatgattttcattccagaacagcagttcctcgacctgagagtcggtcagtgcttttcttcaggtcattgttgagggggaaacctatcggccaagcctgcctctcctggtgactttatagtgctgtgactttatagtgactttacactgctgattgtgtcagggattcaatgaaaatcctgactgatggtaaagagggcatggcagatttctcttggtttaggagttttatttttcccagcattaccatcctttcccaaacttatttccttttttttttttagccttcaagaggggcctctggttccaggagccctgtcttcgccttggttgggctctgtctggggctgtggtcactgcagatgacattctcttgactggattattaatttcatgatgatgactatagatactaaatttgaaaagtagtgaagtctgatgaggcaaccagcttttcaaagtgcattttgtgaattccctccaatccaaagtggagcgggaacctcaaatcactggttagacttggtattattaaataatcagctccattttggcaaagagcagtctccctttctggttgccttccctttaccattcccccattcctgcaggcctggaatccattgtaccagccctcccattcttctccaccccaccccactcctcttcaacccgaccccaattctcttctactctaccccaatccacagagcccaaatctctcagcattatccaccactgcttcttatcaccaaatccagtatctactcttccctctcaacctacttggcatccccacagccttggaaagtccagcactgcatttcttaattcgtaccttcctttccccctgagatgttcacattccatgctcatttctccttgtttctcctattctttttcctgggccatctaaacagcttaatagcttcaaatatcacacgtcctcaggtctcctaaaccttgatatcctgcccagctctttctcttagaaatccatatgtcactcctgaatctacccacatcaacatcccacaaagacttggtgatggttttaaaacatgttcacacattcgttgatactcctccctccaaaaggtgttctgattcccctctccttgaatatgggctggcctttgtgacacatttctaactaatagaatgagacggaagtgacaccatgtgacttctaaggctaggccataaaaagccacacagcttgtacctgcctctcttggaactgctgccttgagaaccctgagcgaacatgtaagaagcctggacaccctgagtctgcatgctggaggccaggcagacagagacaggtgccaggaaacctcagttgctaggtcttcctggtgtcagtagcccctcatgtgagcgggtgcaccttcagatgcccctagcctcagtcttcaagtccccccaactgaggctgagtggagcagagaatagctgtcctgaccaagccatgcccaaactacagtagtgtgaccataataaatgctgttgtttaaaaccactaagttctggggtggcttgttatgcagtattggataactggagcagaccttaaatatgatgtaccccccaaacaaatgcatcatctccatcccttcatctgctcttctttctatattttcttttcttttctttctttttttttttttgagatgaagttttcctcttgttgtccaggctagagtgcaatggcacaatcttggctcactgcaacctctgcctcccgggttcaagcgattctcctgcctcagcctctcgagtcgctgggattacaggcacacgccaccatgcctggctaattttgtatttttagtagagacagagtttctccatgttggtcaggctgatcttgaactcccgacctcaagtgatctgcccgcctcggcctctcaaagtgctgagattacaggcgtgagccaccgcgcctggccctttccgtattttctatctcagtaacttctccattcaatctgaaagattttctctacttttccttttcctcagatgcctcgagtccccctccactgctcaacgtctgactattccaaccctgagccttgcagaaatccagcactcacaccccatgacaccccctacctggtttgaggattcataaatgcctttttgccctctcatacctctgcattgttgcacttgctgaatcttcttcgtaaatttctttccttgctgttctctttattctctcaccacctcaaatcactggaatagggagacccaaggaaagggagagagatggaggaacggccagtcagtggagcagtcagaacacacacaacatttattgattaaatttgctgttttatacaagcatggttatggtgcatgaaagcaattacaatagtaacatcaggcctggagcagttgtttatgcctgtaatcctagctacttgggaggctgaggcaggaggatcgcttgagccctggaagcggaggtgtcagtgagccaagactgtgccactgcactccagcctgggtgactgagtgaaaccttgtctcaaaaaaaaaaaaaacaaaaaaccaaaaacacaaaaagccaaccaaccaaccaaacaaacaaaacaaccccaaaaaacaattataatagtagcctcaaaggtctctcatcacagatcatcatatacataatttatgtatgatttatgtattatataacattttgttctgcaggaataatagaaaaaaaaccctgaaatggatttcctactttgtttatttattagttaatttcattgctacttcaaagctaatgtaggtgttttgtgtgtgtgtgtgtgtgtgtgtgtgtgtgtgtgtgtgtgtgtgtgtatgtcatgattcaaagcatagaagaaacattttgaattattaattgagaatttaaaaatcatacttcattagtttagctatttgagtttctctgtatgaattttgggctgaggagaaacatagaagccacagcagctttcccttttcttgattcaaaggtgtcataatttaaaaactttttagaagatgaagaggacatttgattttttgttatcacttaaggggagagggaagtaagcagtgagtcaattgcaattcaaatccggtgccatcccaggacatagacctctgtagtattgtcacgtccaccttgggctttgcgcacattggccaccagggagggttcgactccagagagcctcacctgtgtctagagcttggggaggctgcgggctgggcctgcctcttccacaggagtcctgggagccctcctgatactccccatgggggacaacagggcccgttgcttctctgtctctcttccttctcctgaccaagttgcctgcccatgcagtcaccctttagcttttgatgataatattattttttagaattttaaaggtgtgttcattttcccaccaacactcctacttcataaccttattttgtgcagtgaacaaaagaagatgtgattgtgtccttcacagtggggcaggttctgctcatcctttttgtagaggttggaaaaggtgagtgaaagggcgcatagggagggatggggatggtttgggcctttctgctgttgaagagtttgaaattcttagttcttttcaaccagttctatgtggaaagaaaggtattttatttgacagtgatactaacattgaaggagtgtttcctagggcctggctcccgccaagcatctatgggcagcaaatgccctgtttgtccctcacatcagccctgggcagatgtattttcccttttcctgttttactgatgagggagcttaaaatttagggagattaaggacctccccaagatcatagagtttatcaccaatattttactgactctccataatatagagagctcttatcttaaatgaattggttacagatgtaaatttgccaaaatggaggtgatttatttaataataccaagtctcaaatgttggaggttttaactttgaattattgacttaacctctagtagaatgttatgatgccagtgtagccaaaaaagaaaagagaaaaagtcttgcttttgttgattttaaaagtgcctgggacctaggaggggagagggcaatgatgagatgtggaaagtgctggaagggaagcattctgcttacccctgtccagccttcttgagggatcattgctccctttgaggaaatagtttttccttccaaggcaatatactctgcttattccaagggaagccaatggacagctcaccgtgcgggccccgtgtcttggaagcacctgtggtgccattaacttccttcagttcctggagcattgcagtcaaagcagctgtacttaatgtagttcgtattttcctgcacttgaagtaattctttatcattttttgcataagttatacataaccttagtggagaaacctgtgaaagcatagaagtaagacatttctttgtaataaaatcctagtaacctcagcactctcctcccacatttgttagtgcctaagagtcttcttttatttaatatacatataaattcagggtcaaatatgctatcagaaacaattctaatgaaggtaaaaactaattttattcacaaatgtttttgtgtaccctctctttctcccctccccagcacacaccccgtcaagttgatattcaggttcttaatcacaagcatttacttgccacataggctgtctttggcaggcctatcagtttcctcaattcgaaggaaacctgagatctgcttgtgcctggcattttttttttttttttttgagacggagtctcacactttcgcccaggctggagtgaagtggcgcaatctcggctactacaacatctgccccccaagattcaagtgattctcttgcctcagcctcccgagtagctgggattataggcttgcaccaccatgccctgctaatttttgtatttttagtagagatggggtttcaccatgttgacccggctggtctcggactcctgacctcaggtgatctacctgccttggcctcccaaagtgctaggattacaggcatgagccaccgctcccggctgcctgacatctttcatgtgagagatctttggagctagatgcaccagttactcttttggatattaaatggtagtgggtgttttttgttgttgggaatgagtgtttaggaggaagggtgttttcatcacagcatttcacacctcctttggtgctgccattcgagtttgcttctgcccatgtgattctgtcaggtactatcaagggttggcctggcttcttcatcctcctgctgaggatatggctggcctctctccccggtcagctctctacctgtgggtaggaaaaggtattcaagattcacctcctgctcctggggtcgggggggcatgaaaggcaggtctgatcttctccctttctcgagctgttactggctatttagaaaagtctgttctggaaggtgcgtctaaagcctgggtgttgcctttctagcgttgcagtattctatttttgaagtgatagagaggtgcagggctcttgtttatattgaaagctgcttaccaggaagtacttaggtctcttctgacaaaaatcaacataagcgcaattaacattgtcttgcttttaacttaatttgctttctaattcatttagcaatggatgtattactaaatgaccttgtaatatatcttacacctgtcaaaagacaaaattacaacaaatttagttatagatctaattggcatttattcattatttggtaagtgggcagcctccattctataaaagagactgagcttccactgggcaatgtcagaacagtgccttttataagggaacaaagaaacagaacaatagaaaaaaaaagctgattggttaacaacaagttacttcaggttacttcttttaaaggttaaagcagaagagactttcttattatgctgattctggtagtttggaatctcctgtttccagaaaaaactgatctttttttggggatctactgcttctttaaagtttcaatatgattatgtggcagtaagcatgagtgactcactttggtgtggtctggcttttgggtcctagtgcaggagcttagtccaaaaacagtggcctcccataatttttgtttaatatactttaacaatcacttcggctgtttcttcaagttttaagcctattctttttagaggtaaaatatacttttgttatttctctttcccattgtaatagtctgttctcacattgctaataaagacatgcctgagacagggtaatgtataaaggcaagaggtttaattgacttacaacttcacatggctggggaggcctcacaatcatggcagaaggtgaatgaggagctaagtcatgtcttacatggtgacaggcaagagagagcatatgcaggagaacttccctttataaaaccatcagatttcatgaaacttattcaccatcacaagaacagcacgagaaagacctgcccccgtgattcagttacctcccaccaggtccctcccacgacacatgggaattatgggagctacaattcaagatgagatttgggtggggacacagccaaaccatacctttcttcccctggcacctcccaaatctcatgtcctcacattgcaaagccaataatggcttcccaacagtcccccaaagtcttaactcatttcatcattaactcaaaagtccacggtctaaagtctcatctgagacaaggcaagtcctttccacctatgagcctgtaaaatcaaaagcaagttagttacttcctagatacaatgaaggtacaggcattgggtaaatacagccattccaaatgggagaaattggccaatatggaggggctacaggctccatgcaagtccgaaatccagtggggcagtcagatcttaaagctacaaaatgatctccatgtctcacatccaggtcacgctgatgcaagaggtgggttcccatggtcttgggcagctccacccctgtggctttgcaggtacagcttccctcccagctgctttcacgggctggcgttgagtgtctgtggcttttccaggtacacagtgcaagctattggtggatctgccattttgggatctggaggatgatggccctcttctcacagctccactaggcagtgtcccagtggggactctggtaggggctccatacccacatttcgctcctgcactgccctagcagaggttctccatgacagccctgcctctgcagctaacttctgcctggacatccaggcgtttccacacatcctctgaaatctaggcggaggttcccaaatcacaattcttgacttctgcatacccacaggctcaacaccaggtggaagctgccaaggcttggggcttgcaccctctgaagccacagttggagctgtaccttggccccttttagccatgactggagaagctgggacacaggacaccaagtccctaggctgcacacagcaggagggccctggacctggcccaggaaaccatttctccctcctaggcctccaggcctgtgatgggaggggctgccttgaaagtctcttgacattcccaggagacattttccccattgtctttatgtttgtctaacgtttgtctcctcattacttatacaaatttctgcagctagcttgactttcttcctagaaaatgggtttttcttttctactgcaaattttccaaacttttatactctgcttcctcttgagtgctttgccacttagaaatttcttctgccagataccccaaatcatctctctcaagttcaaagttctaccattatctctagggcaaaaagccaccagtctgtttgctaaagcacagcaagagtcacctttgctccagttcccaagttcctcacctccatctgaaaccacgtcagcctggactttattgtccatatcactatcagcatttagattaaaggcattcaacaaactttcccacattttcctgtcttcttctgagccctccaaactcttccaacctctgcctgttacccaattccaaagttacttccacattcttgggtatctttacaagcagcacctcactataccagtgccaatttattagtcttttctcacactgctaatgaggagacatactggaaagtgggtaatttataaaagaaagaggcttaattgactcatagttccacatggctggggaggcctcacaatcatggtggaaggcgaatgagaagcaaagtcatgtcttacatggcaacaggcaaggagagcgtgtgcaggggaacgggcctttataaaacctttataaaaccatcagatctcatgagacttactatcacaagaacagcattggaaaaatccgcccccatgatttaattacctcccactgggtccctcccacaacacacgggaattatgggagctgcaattcaagatgaaattcgggtggggacacagccaaactatatcacctatgtagtggcaaatctttttttttgtttggttttgttttaataattggatatttcttttatatgaggctgtaacaacactttttgcttcatcttctctgtgtaaataacttaagctttttaatggatacagctaaaaatttggccttgttaaaggaggaaaaaaataggctgaatagtgggaagtgagcagaactgtgtttgcttttaggttaaaaaaagaaatgatttttctctgtacaacagagaaacatggcccagaaagtggaatggcatacagctgtttctttgtagttcttcagtcttcaagcatgggcttttgcttgtatcaaaatataagtgggttatgatgacctcctagaatccaagagaataaagcttgaaattggagtaactgatgttccccaaaccccagagtgagaatgtgcttttatttagcattttctcttcagcaaaatctgaagagtgcttgttacagagagcaaaacttttacttgatcttttgtcagaaagttaaagaattaaaaaaaggctgttttagatattggctttaaaactgttgttttatgttgttatgtcaagggtgatgccacacactgttgacatcaaatccaccaaaatctcacttaaaaacaatttagggccaggtgtggtggctcttgcgtgtaatcccagcattttgggaggctgaggcaggaggattgcttgatcccaggagtttaagaccagcctgggccacataggaagattctctctgtacacacacacacacacacacacacacacttagccaggaatggtggtgcacttgtaatctcagttacttgggaggctgaggcaggaggatcacttgagcccgggaggttgaagcttcagtgagctgtgattgtgccactgttttccagccggggtgacaaaatgagaccttgtctcaaaaaaaaaaaatttagaagtggaaaatttgtaagttttaataaaataccacttactttttagctctggtttggaacctgacttacagcaatagtagggagattaataaggttacctcttaaggcagtctctcaaatgaaaattgagtatagttatcttatttttagatttatagatgtctctaaaccttattttaaagtgagttcctgactactcttgtggcgtttattttatctttacaagcattggcagtaatggttgatttcacttctgattatgaagttagtaggaaataaagtacagggggtgtggggggtgtgtgtgtgtgtgtgcgtgtgtgtgtgtatgtatgtgtgtgtgcacattttccaggccccactgtcttgaagaggcttgattagggattagaatggtagtggctagagggcctcactaaggtttgttccataggtctcagagctcccttttttgtcgcctttgccctgctgccctgctcaacaacccctgcaaatgtaaggatgatggtgggggctgggagctggggtggccgctgccacatgtagtgctaaagaatcaacaaaacaggacagcaaaagactatgcaaatagtttagctttctcaaacaaggtcttcattgttcattctttttcatcctttggtagctagctgtccttcctactagtgttgactgtttgtcatgtactctctccaccggagtcaccagtaagaccagctgaactctgttgagagttttggagtaaaattctgattggacgtggttttttgtgctttttgtgatcggcagtgtttcagtagctcttagttcccggaacctcattctcctatcccgtcagcgaggtctcccttatttttgctgagatgcatggttaaccatttccccaggctcctccctagcacctgcagtatcttaaaaatggtacttttaacttccaggagcaatacatgaacacaagagtatagataaagataggcaagagtgtagataaagagagttgtgcagagacttcatctggtgcaagtctggccaaccttttcttagtgatttttatgcctgcccatgtaggccagtggttctcaagcttgttaaaacaagacctcacaccctcagagggcttgttaaaacagactgctgaccccacccacagagttctggttcagtaggtctgcggtgaggcctgagaatttgcatttttagcaagttcacaggtgatgctgatactgctggttcagggaccacatttgggaatgctatatagcatgggatttttttgtgtgtgccttttagaaaatcaaatgctgtcatgcttgtgtttctgtatttttcatcagctccatgtcatggagcactttctgtgctagtacatattgatcacttcattatttttaactgctacatagtgtacccacatataaatgtgccttaattctattaatggatacttgattctttccagtattttttgttaactattataaataaatcctgcattgaacatctttgtttattcctcctttatgcagctatgtttgtctagggtaatatggagaagtgaaattgatgaacatgtgcactttttgttctatttttgttttaatagataatgggaccatcactctatgatgctcaccacattaatgcccggccttaggtagtttgttttattctctgcctaagctgaggctaacctggagttgctaggaaaatcataaatgtgcttactagaccctttataaactgaacctgaaatgggctttgggctgctagccagtttttctaagtctttttgtagactaactaaaagcagtgatagtcaactgtttttttcccctaccagcacacctggtaaataagaattgcccttagcaacaatggctcacttaggtgaacttccaggggctatacatccaagtatatttttttgcacgacttcagtgctagacttttccaatctggatgttcttacctcatgttagattccgtgtatgtatagccaaatacagcattctttgtcccctctgccttctaaaccagtttgtcctgtttctgttctgcacagtgatgtgtgttaggtgagttgggctgtaggcagggcaagtgggtgttacgtcctagcattaccctttttaattttttggaattcataaattaaaattgggtgggggagagctcacactgagttgtctactccttgttttgccaagttattaaaatcaactaacaaaaagccaccatgtttttatagatatgagatttttaacactcctcctccccttccaaaaaatcaggaaaaacattatctcagaataaagggcattacttttaaatttagtttcaggaacactgtgggttttttccctcctcctttcatctcggacaagtgaaaatgctgctattgtctagagttgccctaggttgtagagaaactgagtcatttcttgaagaaaagatttgatgtctatattacatattcctaattagttccctattagtttgtcatactgctgatgatgttcctgtctaatagtaacttctggggcatgaagtgggcccggaatgtgcagtactcctgaaagaagaaaaattcttatgattcctacagtcacccattcccattcccggagcacctgctatgtgcccagcactgagctgagtcctggatacagagaagtaggggaaggtgttcttgcatttcggctgctccttcatagtctagcttcttcagtagcacataggcaactttcccgtgaaaatgtgttcgaccttttgtggtgcaactgccatgcccgctcaaagatcacctgccatcaaaatcttcacatagattcatcacttctggccacatgctaggattaaacaagattactgttatatctttgagccttgtgggagaatctgcttttcacttagctgttcatgcagggcagggtgaagcaaagaaatcctttctcttgtaaggaaactttgatatctcttacctacttctgtgcttctgctttgggatgtgaaaatactctactaagtggcaacaaaatttgcaccagtttttaacaccagtgcttcccagataaagagtgaccctccaggagttcttcagctgagcctgagttagttgctcattttgaacctcatgtggtgtgtagaaatgaattaaaaaataacccacagagtaactcattataatcttaatcctttccacacaaccaggatataagggcttattaggctttgaatagatactatgcttattaggtttttttttttggttgttataacttactggacaaggtgctacagggaatgagatgacctacaaatttagacaatccttgagtttggttagaatgcacaccgaataccatgtaaaaaatgcacacaaaaagccatgtcaaggagacagtccaacacctgtctgccccaaagaactggttgtggtctccctttctgggtgtatttcagtgactctgatagaaaaggtgtttttgaaggagggatgggtggctgtgagttgtgtgtgcctcccatgaacttccctgggaagaccactattcagaaacctaataaatatttaatgtaagaggaggcacctatctgttatctttcatagcgctgaagggcatgctgcagttgctatggagaggcaggtgctggctcagctgtgtgacaaagacaggccctcaggaacaggagcaggagcaaaaggtcatttaacctcagctggaagtgcctagttggttctgatgagtattgattagtttttttatgggctgtcccatattctaatatatgtaacatttttaattgttcagtgttttttaaggcactgttttattttgtcagaatttggcttctaattgcaaaatcctagccttgtatgttggtaatattaagatgtaatttatgtactaattggaatattgtgatattcttcatagtaggaaaaagaacaaaatattatgatatatgtaatattacaaaatattacacaaatttttcctgacagagattactttcatttgaagaaggtgaaagttctggttgattctgattataccttcaacatttttagaagctgattacagaatttgtcatataaaattaaaatttttgttgtataagaggtatcctttggggaaaaaatttaaattttagagtattttacatggatttcaccttacagattcatcaacaaacaaactaatgtgacttttatgattgtttaaaatgttaatttttatgtttcatgaacattacaagaacataaatggcatttttctgtaaagtgttttgcatatttgtctgctttgatgtttaatatggtggaatgtactaaaatcttaataaatgtttagcttcctgttgcaatggaactatcaaatcattagtaagtttttcattgtttctactttcttagttccctaagttagggttatgtgatactaagttctagaaatgataatttttgattgatcagttggtcgcttttaaaatgatggacattttgttaaactatgaagcttaaaaaaagagagaaatgaatccatggctcctgcacgtaaggaaggtgtagacaggtggtggatgctagcatatgagttagctacagtaagctattaatatatagggtggtgctgtgtataattagctcactgtgggtggcttcctgccaagttttaatgagttttggtcaaagagaaatgtatgtcccaaaggagccaaacttttaattggggttatacgtaagtctatcagtgaagttgagggttatgttttcaaccctggaccctcattcgggatttgtagcagtaaaggcaaataaagatgacttgactttgggtccttatgttagggtttagaccttctcattctggatacaacttctcgaagcctggctccagagaggggaaaagaagtgagtgatttgacaagcattgttcttggtggtatatacaaactcctagattgatcatatttcatcttttcagcagttgccttaataatttgtagtaattttcagatgggagatgtgagagtccatgaagtaggtactttcctcctagttcatatggttcatagtggagtcaagatccagaccccatctatttaattcccaagttcatgctttgtgaccatggtcaccagatacttaagacatggcagctgatttggagggtgttgctctgttgattggtgaactacctacttttacctcacttcccctgagtgtgtgcatccttacatgcacatgaggaaatacatgaatgtgtgtgcgaggtcctagctcttgctggattctgggcacaacagtcagcagtgtcgtcaagtcagagtttctatagattattcctccttccctattctcttttaatgggactgtaagggagagtgtaggggtaaggaagggaaggttgagttctgcccaggaccagtctgattgtgctttctttttgtgacagagcctcactccgttgcccagggtggagtacagtgacatgatctccactcacttcaacctccacctcctgggttcaagcaattcttgtgcctcagcctcctgagtagctgggactacagttgtgcggcacaacacctggctcatttttgtacttcaggctggtctcgaactcttggcctcccaaagtaatccaaagggattataggcatgagccactgtacccagcctgattgtaatttttgtcttcccctttttgctgacctgctttattttccagttgtggtccttgactagagacagcgtcttcacagttaagcctttattactgtggaccagcccaaccttaatttaatgtccaacaggatcacttcccagtttttcctgtacacaggtacggggtagatgagaatctctgatatgactaggctaactgctctccatgagaagatacagacccgatttgtgcctttgggagctcatggttagtatggaatgcaggcatatgagccttaattgcaacacatgacagaataacgtcactgttagagactgacccatgccatttaagagaaagaagtgatgctggtgagtgagattccaaagcattttcataaaagacaagccactttccttggatctcaacttttcagaattttgatagacctagggaagaacattccagatgaagatggtgtggctattatcttagtatctattgctacatattatcccaaaacttagcagcttaataaaatatttttttatttttatttttattgtttttttgagccagagtctggctctgtcgcccaggctggagtgcagtggtgcgatctcagctcactgcaacctctgcctcctgggttcaagtgattctcctgcctcagcttcccaagtagctgggacaacaggcccgcaccaccatgcccagctaatttttgtatttttagtagagacgggatttcaccatgttggccaggatggtctcgatctcctgaccttgtgttctgcccacctcagcctcccaaagtgctggcattacaggcgtgagcccccgcgcccagccacatttatatctcacagtttctgcaggtcagaaatctggggctggccaagaaagtggttctggctctgggtcttccatgaggtcgcagtagagttgttggctggggcatcagtcatctcagggctggaggatctgcttccaagcttacttgggtggtcattgacagcaggtgccagttcctcaccccagggacctctccataaggctgttcacaacgtggcttccctcagagcaaatgatgcaagagaaagagaacccaagagctgtattattctgttggtcacacagaccaaccctggtgccacgtggaaggggcctacccagaggtgtgaatgccagtgatattgggggtcatagagatgacccactgggtaatgagtaaggaaaggtacagggcgtctgtagctggaatttaggggattaggagtgatgtctgggaggtgaggctgatgtgtcgtgagttgcatggagtatgaatgtgatgtggaattaaccttcaaacagcttagtctagtagtggagttacattttgtatgtaactgtggaaagtgacacatgcacagagccacagatgatccacgtgttgctaacactgggtggtgtggttcatccatgcctcataatgtcatgattgatactctttctacacacacacacacacacacacacacacacacacgagttgtattgcccctgaaaatgctcctcttggtctgttcttcctgccggtatgacagaacttcctgagattgttgagtttagggttaaatcaaatctgaggtgacccactcgttagtttggtagtgtttccccatcgttttcctgttgctaagtggtgcaggagtagaagctggtgcaatagtttcctagagtgcggtgcagctaacccttttacgtacatcatattgtttagaatttgttaccaccttcaaccaattcatataaaaatagattacatgtgggagttgtatttgtttgtgtgtggtgtgcatccaagtcagtgttaattttaagtgagtgtcctgcactctgggtgtttttcgtagctacaaggaagccttaaccgttgttacttacaatggattacttttcaaaagttgtttaatctctttcaggtcattaatggcttggagactaataagcaaaaccattaaatactactttatgtctgaaaaaagagttaatgttcctggaaaaagcgagctgttttctttagttaacaaggtggagctggtgttcccagtggttccggttctaataagacttgatgagttggaaatgatgttattctggagacaactgtggagtaattagaattttacctcattttaattaaaagattctaactctaacctaccttcccaggcatttgggaaaggcacaggtaggtctccttgccagtgaggctccactactcacttgtggggttgacatcaggagaacccaggacttgggccttagtgaggtctcacgttctcaccactaaacaaagagaaagcatttatgcaggcatcgaaccagcgtctgtgtgtcacagctggactggtgctgaaaaaatcaccagggccttagcataaaacaaaggctgctttattatgttgagagcataaataggcttcttttcttttttcagtcagatatgaaattagagttgcttactgctaacatggagcctgctggattgagctgttttgctcacttctgccagataatcagaagagccgggtggggtcccctagctgggtgactgggagtcaggcatccatcccccgaacatcaggctccccatttgtaaatgtcttgtattcctgtcaagatggttaggaggattgaaaaacgccgttgtattttatttgattttgataatacacttaacatgagatctaacctcttaacaaatttttaagtgtataatgcagtattgttagctatagtgttcaagtgcattgggaggcgctatccaaatgcaaccccttccccgccctcacccccaccccccgtttattgctacttgttattttcatggttgtgctgaatttttccccaaggcaaatctttacctctgcccttgaagttgcatatgccccagagtgggtaattattaactgtattaatctggaccatatactgtacagtctttggataacctggacagtttataattttaaatgttttaaaagtttgcaagtctgaaactctgaaaaaattttttttttcataaaaatactagcctaggctgctgatggatgtgggtttctttttggggtatgaaaattagattgcggtgatgtttgcataactctgtgaatatactaaaaaccagtgaaccatactctttataaaaaaatagtggattttatagtatgcgaattatttctcaagctgctattagaaacaaaacactgtcctaattgttactgtaaaaatacccttattttctcccctccctgccagtgtcttaggcagagcagccattcagtaaatacaggtggctgagtgttgaataatatagttgaaataatccaagtggcacaaatagatgagagtggaagtgggggctgcagtgtcagttgtagaaatctcacgggaggcagactcatggggtggctgagacggtggctcctttgtcagtggaggcactggaggaagaagaccccactagcaggagaggctctgggtcggaggggctgccatgatcctggaaacaggtcatctggttggctcgtgtttgaaagttgagaatactaatattttatggtccaaattttggttaccttggccttgtaaatcaggaagccgtctgatgacaggatgagcctaaggtggggaggggaggatgcggtgagccacaggttctcaatgttcagagagccagtgtccttaaatagcgaggtgaagagctcaattaggaaccaaagccaaaaatgagctgaggcaagatggtagaacccaggcctggatctgagcagttgtcaaaagtctggttacaacatttgagagtgtggtgttcagctacctttttggattcagttttagttttttatttgcatattctttttctttttgataaagctctatgaggactatgttttcaactccctagatgcatatagtcctgtactcagatataatgtatctaatctcttcccagatttataattgcaaaccataagccactttgagttgtgttccagcacctgccgaaggagacgactaaatgatatgaaccccattttcctcctaagagggcattcaaattctcttcctcttggattttatgaaatcagatttcagttggaagattctgtcacttggcctcttattgtcaaacacaggtaccttttaacgcagacttctactgagaaataattagagacagggtctcatggtcaggcgcggtggctcatgcctgttatctcagcactttgggaggctgaggcgagcggatcacctgaggtcgggagttcaccaccagcctgaccaacatggagaaaccccatctctacaaaaaatacaaaattagccgggcgtggtggcgcatgtctgtaatcccagcaacttgggaggctgaggcaggagaattgcttgaacctgggaggcgaggttgcagtgagccaagatcgtgccattgcactccagcctgggcaacaagagtgaaactctgtctcaaaaaaaagaaaaaaaaaagaaaagagacggggtctcattctgtcgctcaggctggagtgcagtagcacaatcatgactcactgcagcattgaactcgtgggctcaagtgatcctcccgccttggcctcccaaagtgctggaattacaggccaccctaccttgctgaaatatttaaattctgaaggaccaaactatccccttctcaaagaacaatatgaactagcaccgttataaacttggtactgctgtaagagtcctactcctgagggcttccacccatcttattttcatacttatgaaaaataatacatgttcaaagcaaatgaatccaaacagtatgcaattaaaactcaaactctcttagcctagacccatttctcccagagcggctgttatgatcagcctctgggtgtccttccagaaatgtctaggcaggtaatgtatacaatcactttaaaagttcaaatgggaacatgtcatacacattattctgtactttgcttgttttaactgacacgtatcttggatattaaaaaaaaaatctaacatatagtttccattttttaagaaaaaagctttattgatatataatttgccataaataatcaccatatagtttaccttaaaattcacttgttttaaatgtacaattcagtgatttctagtaaacttacagagttgtgcagtgatcaccacaatctaattttagaatattttcatcaccctgtgtctatttacagccactccacatcccgtgcccgcaggcaaacacttatctatttagtttctatagattgattttattactctttttaatagcaacctgtggtccattttggaaaaatacaaacatgtatttaattgtgctttatccaagcacatttagcttttgctgatgttttattttgctgcctgcctactttaaaaaaaattggaaaagattgaaattttgtctttgagccatacttgttaagagtgataagggagttttagctgaagttgtttgtagattttccagtcattacctgtggaactaggagaagaattttggggcctggataattatttatatttgaatagaaactgcctgaggttgttcttgattagatgaaagccccagcatttattgtgtattttatggatgtgggtgaagtggaactaagtatttacacatgattggggtgaagggggatttattatttcagttttttgcctttgtctctacctaggaggattttaggagctgttgagtgttcagaagtatgtcttcggatatattctccatactgtttcttgggaccttttactgggttgtggattttgaccaagtgtttttttaagaagaacaaagtagtagaacagtatgagaccaaaatgtccttcatgttctaatggtgagcactgttttcttttttttttttttttttttttttttttgagacagagtctcgctctgtcgcccaggctggagtgcagtggcgtgatctcagctcactgcaagctctgcttcccgggttcctgccattctcctgcctcagcctcctgagtagctgggactacaggtgcccaccaccacgcccggctaattttttgtatttttagtagagatggggtttcaccgtgttagccaggatggtctcgatttcctgaccttatgatccacccgtctcggcttcccaaagtgctgggattacaggcgtgagccaccgcgcccggccattgttttcttaaatgtcaacttttgtttaaatgtgtgtgtgagtgtgcttgttttttttttaacctcaaagactgaaatttttttttttaattaattaatttattttttattatactttaagttttagggtacatgtgcacattgtgcaggttagttacatatgtatacatgtgccatgctggtgcgctgcacccactaactcgtcatctagcattaggtatatctcccaatgctatccctcccccctccccccaccccacaacagtccccagagtgtgatattccccttcctgtgtccatgtgatctcattgttcacttcccacctatgagtgagaatatgcggtgtttggttttttgttcttgtgatagtttactgagaatgatgatttccaatttcatccatgtccctacaaaggacatgaactcatcattttttatggctgcatagtattccatggtgtatatgtgccacattttcttaatccagtctatcattgttggacatttgggttggttccaagtctttactattgtgaataatgccgcaataaacatacgtgtgcatgtgtctttatagcagcatgatttatagtcctttgggtatatacccagtaatgggatggctgggtcaaatggtatttccagttctagatccctgaggaatcgccacactgatttccacaatggttgaactagtttacagtgtgcttgttaagtagtaattatcagaaatgttggaaaaccagtgccttaagagtaagggtatattcaggcttcctctttgtcagagctctaactctcctggggggcaggtaggctggggcaggtagggtgagaagactcaggtagttcaccgagataaaactttgttgaggttggctggggaagtgagccagttgtcttgggaagtgggaggtgaaaatgaaactccctgtgtgaccacttttctctatcaaaaataataaattatttcaaatgttctaaataaggaggctttggatagattttagtcagcttgttagctattcaattctggcaacaaccgtgtgtacatctagactagggagacgtttacaattaaattcactgggtcttgtggttggaagtaacctgaagggatatctatgcaagtttccatcctgtgtgtgcatcccttctgtaatacctctgcctggcagttcagcatctgcacttgcgtacgtctcctggaagggaattcatgaactctcttgggatcccagttcttgtcagcttcttcatgttgaaaccctcaggcttcatatatctcagggtttggattggcattagttatctatcgctgtgtaacaaattacctcatcattaaggggcttaaggcaacaaatatttattatgtcaggaacaagtcaggagtcttggagcagctcatctggatggtgtggctcagggcctctcgtgagactgtggtcaagttgacagttggggctgtcgtcatttggaggctgaatggactaacggatccactttcaacacggctcctgcacctagttgctggcaggaggcctcagttcctactgtgtggacctccacttggagctgcctgagtgtcttcatgacacactctctctcggatcagaaaggagacattagtaatgccaaggatactcctttactcccttcacaagtgggcacagatatggtcctttttccaccagctctagaaagaaacatgcagagatgtttgaaggcaagaagacagtggcctgcatgacttggagcttttcttaattgtaagtcagagataataattaatgtcatgcctagttcagagaattattgtgtgactcaaatgagatgatgagcattaagacattggaaactttaggtactatatgaatgttttcttattggggcaaaccattttcctcttgtaaaggtgagcccagggacatgtctctcccccatctctatactatcagttctgatctttgaatgattgcagtgcccctcatagggttgggtaaggaccaaggcagagaatatgtagataatgcagcacagtacccggcacatagtaagcattcagttaacagctacaattacttaatggttttgaaatagatatgccaggcactaaagaaaattaattgataaatgaaataactactgctcctaagctgtggccgtggattagaatcaccgtgtctgccatgtcactgactctacagaatactgtatatggaaaaatggtttttattatttgttattttctatatttggatgacatttctgatctgaatttcaaccgtgacttctagtcctctgtatcttcttcttattctgacttgctacaatataaacttcatcctttaaagaaaaagtgaccctgttattcttccttggggtgttttgaatatcctccccctccttgcccagcttccctcaaatgtgccaacaatttttttcttttcttttctgtttttttttttttttagcatcagcggtaccatcaagttcctggactcttgaacttgatgaaatcgagatagtttctgtagggctccatttggaattgaggagcaggtgtggtctagacctaaggtccagtggtgggaggaatggaaagaagaaatggagccaaaggaaactgagggaaggattcatgtttctctggctcagtgagtgggtgggtcaaagaaataaaatgtttgtaattggttttaagcctgggcaactggagaaagtggtggtagtgttaataagagaccaggaggaacacaaagaggaaagaatatctggttttggagggaagaaggaaaattcagttttgattctgaatttgaggggggttggcatccaccagacagctaaacatggaatatggcccatgggtggtggtagcagtccttgctgctattcctggaacctctcagagggtcaatcacaggctgtcaccgtttgcctaagtggtatcctaagaaaatgtctgaagtggtccaggaagaggggccgataattattgagtaaaggtaaagggaacacacattattagactggtgcttttgaatgtcatatttattcttaatagcaactatatgagttatatgtgcatacctggttttacaaatgcaggaaaaaaggaccggagtgaccaggtaagctatccaaggttatgcagctaagaggtgttggagccaggatttgaactctacactcagttttcatcagtgaacagattttccttttacatcagcatcagaagcattatggctggctgaactgatagagaactagcaagtatggactcaaaagtggcatggggaccccttagtaattaattactgagggtaactaaactccaggtaaccttttgccccttcctttcttccagccattggaagaacgtcctcatgtggtacagtgtcaggcaggagacccatgtttctttctctgaatcaatagtgggtggcacagaacaacaaaaggacgtctgcttttagatcctgggcttccaaatcttctggcgccatcagctccccttccctcttggcacctctcctgtcctcaccctggtatgttgtaaacccaagaagtactattctctgggtccacctgcccataggatcctgaatcttcacacaaacaatagtagtcacaattctaacaaccagttctgaaacatttactatatactgaatgatttgcaaacatagaaaagttaattttcacggcaaccgaatgaaatagattttcggcctggcatggtggctcacacctgtaatcctagccctttggaagcctgaggtggatggatcacttgagtgcaagagtttgagaccagcctggacaacatggaaaaaccacgtctctaccaaaaaattaaaaaaattagctgggactacaggtcctggtatattgtaaacccaagaagtgctgttctctggggccaccagcctttgtattatgttggtgtggaggagggaggattggtatacagataaatttgcccataggctcctaaatcctcacacaaacagtagcaatcacaattttaacaacacaactttaacaggtgctacaggtgctacctgtagtcccagctatttgagaagctgaggtgggaggattgcatgagcccaggagattgagactgcagtgagccaagatcacaccacagcactctatcctaggcgataagagtgagaccctgtctccaaaaaaaaaaaaagaaagaaagaaaaatagatttttattatcctattctcattgatgaggaacttcaggttcagtgagttgagatttcaccatttcatacatctaatcagtaggagagttgggtttgcatccctagttcttctgacttgacagctgcgttcttaactaggatgctgaattgcgtcttcatagaaaatctgtctccaaaccaaaaccttttccacccacatctattaatagttttacgctattcagtatagatggctgccacgtggagcacctgtaaaggggtgtgtggctgtgagcggccactgagtatcttaaagtgtgtgagctcataggtattattgtagggtatctggtcctaaggtatcttggaagactaaatgtggccaaatttcaacaatgttggaatggaatgaagggtagaggaagtgaactgttagtagtggcccaggactcttgctacaaaagccaccaggcataactggttccacctataactgggaaattcagctggagtcttgccaaggagtagctagagtggagggattggaagacatactgatcttttgaagatacctggatacagttgataccagattagcacttaaaagggagtcaatattcctctgagtaccaattaatatttgagggtatataatatgctaggtgttttcctttgaaatatgtatacagtgattaataaagcagatttattagaataatagtttattaattcctagtagaagtataacaggcagatggaatcgttggactgactggtcagtttgtttcctttcaagtagcttagccattctggatatttcaaagagttgtctttgctgatttttttctcctgatctcaacaccagcctttcttcttggctctcctctctattctgaccacactggctctttaggtcaccttcctcaggaccaaagctttaaataccattcagtcactgactcctgaagctaaacctctacccctcaccccacccacattttaacaatttacggaagacactgtgtgaaaaattaccctatgcacttgagaaaggtctgggtaaattactttcttcctagattgttctctttaaaaatttgttttaagcatttgggttaaacagtaattttgtttgaatggtgaaaatagtttaaatacgttaaatgattcccctttttgttaacggagccctgaaatcaggtattttatttcccagcctttagttgatattgtagtgcattgaagactcttactgaacagaaaatgccaggatttatattcctggtgagttggattgagggaaggttggtcaccaaacagtccagctggtagctctgcatcttaatttctaagtcatgccgcagaatgttattttaagctatacacttatgaaatatattttctagtatcttgttgtgatttcttgactctgtcttgtccatctcaatttaagatccaatttaactaaaaattcctgctgtgggcaagaagagtgctctgggtggagggcacagcatatgaaatgtcctgaaggttaagtagctctttggaaaactgctcatcaaccagatggttaccatgtagaatatgatgggactggtaggaagaaatcagagggctaaaaaatctgctacatttaggagtaaacgtggtcctaagagcaatcggtccttaaaaccactgaaggattttaagccgtgtaatgacataatttgggttttgtctgcaaccagacattgcaaagtaagttagaagtttttaatggtggtttcatcaagcctgaaatactgacttctgtgattttgtctgtgtctgttccctttttcatcctttccaccccatgtctattccaaacttgcaaaactttccctccacctcctcttcatctcagcagatgctcttgtcttctctctctgagcaatctgagccaattcggtgtgacctcttgggcttcttgtcactttccttcttcagaatataaaactcgtgacctggctgacctcttatccaggatgatcatttagaaacaaaggagagtggccgggagcaacggcttacgcctgtaatcccagcactttgggaggccaaggccagtggatcatgaggtcaagagatagagaccatcctggccaacatggtgaaaccccgtctctactaaaaatacaaaaattagctgggtgtggtggcgtgcgcctgtagtcccagctactcaggaggctgaggcaggagaatcgcttgaacctgggaggtggaggtttcagtgagctgagatcgcgccactgcactccagcctgggtgatggagactccgtctcaaaaaaaaaaaaaaaaaagaaatgagagttaggatttgagagaagtcgggggaggatgccacggttctcctttctaattgctaaagtcagtgaaatcagctaaacaaactatggaacatacattgtggttctcagggaaatataaaacaacaacatgcaggtagaatacaagagcaaagaagagtgatgagcacttagctatggctcagctgacaaattccagtgctgacgttgctatccggccagcattctacgtcccgtgtagaaggatttgttgttcgtgtcaatgtcttagcagatttgtaatgcttgtatgggaattttaaatgtaaatttcatgagcatacaacttttaatataatcatgctgttaacttaatataaattttaatacaaattgtatgcctgtgtaaatatactattttataattaaacatttgttcaatactttgtgactggttgtgatttttaaaataatagcaattattttttaaaaattttaaaaagagtttatgttctgaggcaagcagatatgaatagtggggaacaaccaataattttttcactattaataaattcatgtatatctaatatataggagatgagaactaggaagtatcaagcttgaatcttgcttatccaccaaattcttgaaaataagtgaatgactatgtcaatcaaaatttatcttcaagttagaagacattcaagcaaacttcaaaggcttaatgttttttaaaaatcttgtaatttatatttttgtaaatagttgaaatgtaacattacagagaatgaaatcattctggtactaatctagcatttcttcctgaaaaaaaaaacccagatgtaaaaatgatcaagagaaatttagaaaacatgaaacaaatgaagaagtgaattctttaaaatttactgcaaattttgatggaaatggtaatactgtctgatacagtattacatcttaatatctaatattaccagtattagtatctgtctgatactgtttattaatttttttaatatttaacatttaaatttattttattttattttattttatttgagacagggccttgctcagttgcccaggctggagtgtggtggcgtgataatagctcacagcagccctgacctcgtgggctcaagcagtcttcccacctcagcctcctgagtagctgagaagtatgggtgtgtaccaccatgcccagcattttttttttttttttttgtagagacagggtctccttgtgttgctcaggctaggggtcttgaactcctgagctcaagcagtctttccacctcggcctcctaaagtgctgggattatacgcatgagccactgctcccggccaatgataaattattatctattcactcaagatagacagaaaaatagaatgtacttgaaatttttagtaaaatatcttacttattaaagagtttaccattatgaggtgatatgttgttttccttctatgaataaaagtaaaaatcagaataaaaatatacaaaaataaattttaaaaatatacaaaatgaacagatttcaagtgagataataaaggttgaaatggacattatgagataattgtttcttttgtttacttagtaatgtgataagacagtctatagtactgcggacagctggcaatttaagatagcctttgacattttcagcagctatctaattttcattagctgcttgttagaaaatgtaaaaaaaaaaacaaaacagaaatacaaaccaaaaccaagcagcacagaagagtacaaagtgaaaaacaaaaccattgctatctacttccctgatttttgagtcctatttttgagagagaaccactataatgttttagtatttacctttatattttttattatgtatctctgtgtgtgtgtgtgtgtgtgtgtgtgtgtgtgtgtgtgtgtgtgtgtgtgtttatttagaaatcacaagtgggacctgtatttgtcaggttctagattataggataattcctgtaaaaatctcaaagtttctgtttctttgcataataaaatttgaacacttgctggtctgatttgcagtgtctttatttgggtttttcttcttggcagtgctccattaaatggaaactcaggaacccaggatctgtccacatggtggctttactcccacctgtttggatgtgaaaaacagaattcgaattatagaattagagaaaaagaaccaagagctctatagccaggctgccatctttaccttttgagtttgtaagtaattagatttgtgaagatatgaacatcctgtcaattttttgtcattctgtcagtttcactctatagttaacattcatgcatttatacaatgattgtcaaattatcaaaatatacatgaacgtgaacgaagtgaagttataatacacaggaggatgataatccatcaaaaatattcaggccatcagtttggatgagagagctggagagtgttgaaatgtttcaagttgtatcaaaagattctttacatttatagttagctgatgagtcaattgattgtttgacagtggtttctaaacctgaatttcttttgtacaaaatttcttttaacatggtatctttctcttcttttttttttttgagacatcatcttgctccattgcccaggctctagaatacagtggcatgatctcagctccctgcagccttggcctcctgagctcaggtgatccttccacctcagccttctgagtaactgggaccacaggtgtatgccaccatgcctggctgtttaaaaaatttttttgtagagacagggtttctctatgttgctcaggctgggctcaagccaccctcccacctcaacctccaagtattgggttaaaggtatgagccattgtgccctgcctgatatgctattgtaaatgaaactgttgtaaataatttaaaatcaaaattataaacttttggctgggcgtggtggctcatgcttgtaatcccagcacatagggaggctgaggcgggtggatcacttgatgtcaggagttcaagaccagcccggtcaacgtggtgaaaccccatctttaccaaaaaatacaaaaattggctaggcatggtggcgtatgcttgtagtctcagctacttgggagcctgacgtgggagaattacttgaacccaggaggtggaggttgcagtgagccgaaattgcaccactgccctccagcctgggtaacagagtgagaccctgtctcaaaaaaaaaaaaaaaaaaaattataaacttttatcttcaaaagcaaaagcattgtttactgtttcagtcagagagaaatgatgcgtaattaaaatccatttgggatgaacagagaatgtttgcaaaaaaaaaaaaaacctcaatgagtatttcaaaatgaaaagaaaaataaaaaagcaaaaatatttttccctgtgaaagaaaagaataaaaccagaataagttatagaatttgagataatacattttcccccttgctttctcaaaatagtatgtaattcataatcttatgcttgaatacaaatttatgcttagtttttcagaaattaattggagcaaaatcaaaataaatgaaacaaaatatagatattaaataaagcaaagatcaaatatgcaggtgaattttacagactccataaatctgagtgtggacatttatacagactgcagcttatctgtgattgtgtcaccatattaatgctggcttcttatttccctgagtgaacatacaccccttgctgtgagcattcaggacagcctgttgagagtgctcacgtcctcctcacatactcacttaggccgtgtgtgatgattctggggtgggatttcataacaaaagtgttagagcctacagctgtcagtggttttatgtacaactttgttgcgttaccttagagactgacaaggtttaatagtttatctctagaaattgtattcttaggtagctgaattgtcatatttcctatttaaattttaatcacttaggagtctttaggaccttaaaattgggtgtgatgttgacactattgggttgacataatgaatgttaatgaattataaaaatactgtattttattgattctataatcaagacacatcctgtagtcagtggcatcttaggttgaatgaactgcagtagtctctctttttttttttaaatcaaaaggccaaattaattgtgaaaaatgttaacttaaaatttctcagaacttttggcttcttaattgccactggaaaaagaagtatcagtatcatcgccagctgtgcaaggcttgcatgtttgtttgtttgtttgttttttaatgggacagtgtctctctgtcactcaggctggagttcagtggcaggatcgtagctccctgtaacctcaaatgtgccaccacacctggctaactttagtttttgtagagagggggtctcgttattaatattttggttaggctagtctctccaactactggccttaagcagtccttccaccttgacctcccaaagtgctgggattatggcgtgagccactgtgcagggctgaggcttgcatcttcatatctgttggactctgtagtccttcatgctcttgctccttggctccctctttcttgaatctccactcttcccatcttgataaaccattacctctgagtctagtggtttcttaactcacttgtacattagaatcatcagggaagctttaaaaaaagaaactgatgctgactgggtgataatgatgtatcagtgtaggtttatcgattgtagcaaacattattgtggcatgggatgtcaatatgagtaagggaggttgtgcatatatggggacaggggctatatgggaactctctgtactttctgctcaattttgctgagaacctaaaatactctaaaaaagaaggtggtttttttgtttgtttgtttgtcttaatggtcaaatggtcctgggatcagaatacgtgctaagcatggcaaaaaagatgatcaacatcatttagatagtgctattcttaaaaaaaaattctttctcccctagagtgatgttatttttatgctagtgaaattcatataaacatacagctagcatatatcatctaatttaagacactatcaattgtaagccacactactaagatagaaaattgctaattaaactatgccatactgtgtgtgtatgtgtatgtgtaaacgtacgtatacacactactgatgtctaggtccagactaagaccattgcctttagtctatgaaacttgccaggctgggcagggtggctcacatctgtaatcccagcactttgggaggccgaggagggtggactgcttgagcccaggagttcgagactagcttgggcaacatggcaaaaccccatctctaatctccacaaaaaatacaaatattagccaggcacggtggtatgcactggtagtctcagctatttgggaagctgaggtaggaggatcacttgagcccgggaggtcgagattgcagtgagcaaagattgtgccagtgcactctagtctggatgacagagtgagaccctgtctaaaaaaaaaaaacaaaaagagagagagagaaacatgctgataaccccttctttggctacaggtccctctctagtattgcctaacatgttacctccctttttcctgcacagttctccaaagagggatgtgttctcagtcttcactttcttaccttttagtcatgttcagctcaggtaattaggcttttcctttgtgagtttcctgacatgctgtcgctgataatcaccagtaaccacctgcttgcctttccttccttactagacattctgttgcactggacgctgttgtccattccttttcttgaaactctatttccaaatctttccagatgttgaactctcccagtgtttttcatacctctttgaccagtaccaagaatgtagaaccaagactacattcttggtccatcctttatgacagtttccctgcctctctgtgccctccttcagagagcttatacattttcaagttttcagctacaattctgtgctcatgatttataagccgtttctttggttattcccgccagcatcagaatcttatttccaactgcccacgcagcacatctacttggctatcccataaacttgaccaagatggaattattagtagtatttcttttcatagagatgaggtcttactgtgttgccaggcttgtctccgactcctgggctcaagcaatccttctgccttggcctcccaactgttgggattacaggcacaagctgttgtgcccggctggaatatatttttctttaaacctacattctctttcttggtcggaggtaccatcgcccaccttgtcttgagagtcatttttccctttttttcagcctctacatacactcatttaataagacctacacattttattttgaatatttctcagcatttacattcccctctaagccaccagcatcctagctcaggacctcatcttcacatagattctgcagagccctcctgcctctgcttttggccctttcaaattggtcctccacagtcaccagagagactcatctaagatgcagacctgaactgtcacctctctaccttatcaccagcacttacctcctttcctcccacccactgtccttgtctcaagtcagctggtgcctttctgctcagaggacctcagtagtgctcctgagcatgcttgcagttccggctttcttcctactccatgccccactgctgacctgcttctgccacggcatgcccttgctggcctagaggcagacgtcagaattctcagcctagcagtgaaggtttcccaaactattgtctgtggaattgccatgtccttgttcattaccatgcattcagcctgaatcctgcttctgactgaacaccttcttccctctgtgtactccaactgtcactggatggtgccgtgcagttccaggctcttggtgtcctgaacaaagaattggacgtgacacacacaagtagcaaagcaagcagcaaaagtttattaagcactgtattacactcttggagagaggagagtggactgatctctgtgaaatgagatcagcatcagtttgatgtactttgggtatttgtatgtgtttttttgtctcttctcttcccaaggctgcctaatctttagccagactctgccatttaattgataggtggactgcttactttggcccttatgtgcttgtgtgttgcctccatcccataatatacagtccatatgcatgagctttaatgagctgattatcatatggggtcatgttaaggatactttttctctctgatgcgcatgcccatctctgaggagccagctatggggtctctttactcactttttaatcttgtttttgttttggctgctcagcttctgtctcttgtttcttactcagctacccattcaccttgcttctgctctctgcttttactcattctgccctttatccaacttccagttccctctgttttcctgcatcacagcttggtttgtcttactttctgttttgttttgagatgtgacgtgtcaccatagagaaagcccttatgtagtaagacctgattccttgttctcttgagagaacttttgtgagattttagctaaattaaataatttgattttacctttctcatctataaaatgacaataatgataaaacatcattgcacaggagtttttttttttcttttgaaggtgtaagtaagcaaagcaaaggggaagtagaaaaaaatgtgaagtagaaataatgataaacataatttctttattaattacataattataatttttgaaaaacccttttactgtgtcttattactactagctgtaggaactcagctggaaatcaatgaaaaaaccttgtaaatgaataaagaaagggataaagccagactggagccctggtttataaaccaggctacctgtggatgaaaggagataaaagaggaccatccgatgcaggttttgcgtcgtgaaggccacttggttaggcctgggcttggattgttttggaattaattccaggcatttgcactaagaatgtaatcacagtaggaactccctgtaacagatgtacagagtttagtgggaggtaattatattagattttccacgtggctcctgaggtttcaggggattagtttggatggtctgtttgggcttgactacttttacacaatgtatttgttagtttgctgtacggtattcatggggattaatggctgcattaagtaagagtaaccctaaaactctctgagagctcgttataaaaacattcagtgaaaattctgtctccatggccataatcttttaattgaattgaattatgtggcctcttatcctgcacggagaagggtcttgtcactttcatgtttactgggggctcctgtcacatcttgcctttgctggggctgtgaaagggcagcagttttgctgtgttcaccttaaataacccttagtgttaacatgattatttgttcttggtggaaaatgttagagtgttgacttttgacgtcttgataatgatcttggatatgttcacaaatttctgtgggtagaagaagggaaaaccaacataaataagtttgtatttcaggctgattttttctttttcctttttttttttttgagacagagtctcactctgtcgcccaggctggagtgcactggcgtgatctcagctcactgcaacctctgcctccctggttcaagctatcctcctgcctcagcctcccaagcagctgggactacaggcacatgccaccatgctcagctaattttttgaaattttattagagatgggggtttcaccatcttggcctggctggtctcgaactcctggcctcaggtgatctgctcacttcagcctcccaaagtgctgggattacaggcctgaaccaccccgtccagtctgattttttgttttaaatgacaaatgaatattatctctatgttataagacataaggcaactgaagaaagccttcttagtgtgaaatatttatcttagatattttttagccaaaatgattttagtaaacattctgtttatattgctaactgaagaaggtcaaatgacaatgttgataaaagtgcaagccatttcaaagattaaagcagcacttcacttctatgcatatattgtgttttagtcccatcgatagcttttacaggatcaacctgtcattcaatccatccattcttttctttttttttcttttcttttttttgaaacagagtcttgctctgtcgctcaggctggagtgccgtggcagctcactgcaacctctgtctcctggattcaagtgattctcgtgcctcaccctcctgagtagctgggattacagacgtgaaccaccatggccagctaatttttgcagtttcagtagagacagggttttgccgtgttggctaggctggtctgaactcctgacctcaggtgatccacctgcctcggcctcccacagtgttaggattacaggcatgagccaccgcgtccagcctattatttttttttagaaatattcatttaattccaattttgttgtgcttagataccatctactgaatccttatatatatacatggtacaaggtatgtaaacctctgtctgttataaatattttctctcaggctgtcacttgtcttttaacttgtgctattctatcataacagagttttaatagtatatagtcaaatttgtcagtctcttagtaacttttgagttttttatcccagagagatcccatcagtacccaaattattaatgtcctgtagtttttctaacattttaattgtgtatattttctaatgttttctgctatagctttttatttcatctggaatttctggcttgaattagggaaagatttatcttttctccactcatataaaatgtaagtattttcatacattaaattcctttatatttgtggatctatttctggacttgtttcaaatgttttgtttattcttatgccagtactgtgtggttttaaaggccatatattcttttaattttaaagaagcttatattttctttcacatggtgtgaaacaaattttacatgaacagcaaggtataaagtaaaaaattgaaatccctttttcttgaactcattcactatagtttcccttcccaaagtaaactactgttaaatttcttgactttttttcccccagaaaactgtccatgtactagcacatacacaaagccacgggtagaagtttacaatgggatagtatacacattattctttgctttgctttttaaaaaaataacatctttaagatataattctgataaccatacagctcattcaatctataattcattggtttttagtgtatccagatacttgtacaacatttaccacaattttagaacatcaacccaaaaggatactccttacccattagcagtcactcctcattcctctccccacttccttactcccagccctaggcaattactgatctactttctgtctataaatttgcctattttggatatgtcatataaatgggtttatacatatgtggtcttttgtgtctggcttctttcacgtcacatgatgttttcagggttcatccatggagtagcatctgtcattacttaatttccttattgctgaataatattcccttgtatacatgtaccatattttatttattcagtcatcagttgatggacatttgggttgtttttactttttaactactttgaatacttgtgtatatttgtgcatgtttttttgtacagacatattgttttcatttatcttagatatttacctaggagtgcaattatggggtaatacagtaatttctatatttaaccttttgaggaaatgctagacggttttctgaagtgactgtactgtgttacatacctgttaggagtgtatgaagattgttgtttctctggatcgtcaccaacacttattattgtcttcttgattataaccattctagtgggtgtgaagcatgaggtggttttgatttgcatctccctgatgtctaatgatgttggccatcactgtgtgtgctaattgaccatgtgtatgtcttctttggagaaatgatttattcagattctttgcacacttgaaaatttggttgtcttttcattattgaatggcaagagttctttttattttctagatacaatgccatcatcagatagatgattttacagtatttcctcacattctgtgggttgtcttttcacttcgttgacggtgtgcttgaagcataaaagattttaatgttgatgatgtccaatttatctatctttttttgtgcttttggtgtcctgtctaagaaaccattgcatgatccaatgtagagttatatctgtgttttcctctaagagttttatagtttaaacccttacatttaggtctttgattctaagtgtatggtttgaggtaggggtccagttttattcttttgcatgtggctatccattttccccagcaccatttaatgaaaagactatttttccatgtcagactgtcttggcaccttgtcagaaatcaattaaccttaactgtgaatgtctatttccgaactctgaattctattccattgacctatatgtctattcttatatctataacacattatcttgattactgtagtgttgtagtgaagtttgaaattagaaggcttgagtctgggttgggtgcggtggctcacacctgtaatcctggcactttgggaggctgaggtgggcggatcacgaggtcaggagatcgagaccatcctggctaacatggtgaaaccccatctctactaaaaatacaaaaaattagctaggagtggtggcacgcgcctgtagtcccagctactcgggaggctgaggcaggagaatcgcttgaacctgggaggtggaggttgcagtgagccaagattgcaccactacactccagcctgggcaatagagcaagactctgtctcaaaaaaaaaaaaaaaagaaagaaaaaaaaagaaagcatgagtcctctaagttgttctctctttcaagattgttttggctactctgggtccgttgaatttctgtgagaattttaggatcagcttgtcaatttttgtcaagaagtcagctgggattttgataggcattacactgaatctgtaaattgattgagaggtattgtcatctccacaatattaagtcttcagacccatgaatatggatgtcttcccatttttttagacttgccatttatttaaatgtatttaatttctttcagccatattttgtagttttcaaagtataaattctcttattaaatgtattcttttcaatgctgttgtaaatacaattttgtcttaatttcaattttaggttattcattgaaattgtatagatacgtgataagttttgtgaattgatcttataagcctgcaaagttgctgaactcatttatgagttctaatagttttttaatggattccttattattctatacatgagattatgttactgcaaatggaaattgttttactttttcatttccaaaacggatgccttttagtcctttttcttgcctaagtgtcctggctagaacctccagttcaatgttaaatagaagtgataagaacaaataccctttctgttcctggtcttaggggaaaaacattcggtctttgttaactaagatgttagctgcggttttccatagacaccctttatcaaactgaggaagttcctttacattcctagtttgttgagtgttttgatcataaagggtgatgaatcttgtcaaatgctttttctatgtctcttgcatctattttgatgatcatatgttttagtcctttatctattgatatgattacattgattgatttttggatgttaaaacaaacttacattcctgggataaattctacttgctcatactaaataaactttttttttttttttaacattcctagataatggtttgctagtattatgctgaggactttcgtgtctgtatttattagaaatactagtctgtcgttttcttgtgatatctttgtccgattttggtagcagggtaatactggcctcatagactgagctgcatagcatttcctcctcttctattttatgctgaaagtatttgtgaataattggtattcattcttctttaaatatttggtagaattcaccagtgaagccatctgggagtaggcctttgcatgtgggtggtttttgaatatctttgcttgttagaggtcttttcagattttctgtttattcttgagtcaatttcaggattttgtgtctttctaggaatttgtcatttcatctaagctaaccaatctgttggcacacagttggtcatagtattactttataatcctttttattctgtatggttggcaataatgattgctgttttaattcaggattttagttctaccttgctttttaatgctttatattttagagatctttttgtaaaaattgaattcatcttttagtgaccacatagtgttccataactaacctatttatggtcattaatgttatgaataatgccacagttaatatcttttgatatattgtagggtacttttggtgatagtgtgtttctgagggtaaattcttagcagttgatttgttaggtcaaagggatataatgttcaatattagtagatattgacaaattatcctttatagagtttgcactatttacatttatttttctatactatgatatcctttacttctttgtcttttattgggttatttattttaacaagtttttgttatattaagaagaaattagcctttgtcatatgtcttccaaatattttttccttgtttatagtttcttttgaccttgttgaacattttttgccatacggaagttttaaagttttgtgtggtcagatttatcctccatttccgttctggcttctggactggtatcactgccatgtatcacacggtattactgccatataaataggctctgttaggcttcaagaaacaagtgatggattttattgaattcatagagaataagaaaagtaggactagccacacagttaagtttatgttttggggatacagcagtcttccaggagttagtctgtaaggtctgccctctgcagggagcttgattgaaaatctagtagttggctgttcagagatgacattccattcctctcttgcctcccttccttctctctaggtgacattctactgttatggttctgctgccttttccgccgcttgtatgactcttgatctttcttgtttattgaagccttatagcttctgctctcttgctctgcttttcgtcatctctctgtaggtcattgtggcttctgtgcccattggtaagtactgactcttggtatctccattttagactcctgttcagaggagagatcagattggtttgtcaggtcactgttttttattcattgtagaccactgtagacgggcggagactttctgccaagcctgtcttcagtctgcagattggctgctctttgcactctccagtcggcctgggcttcaaaagtgggcctaagtggttgaatgtagggcagccctctgtgtttaaaaaagtcaaaaagcttaaaaaggtgcatgtggcagaaattttcaaaaaacaaaaggagcattataagcaacaggccccttccattgtctgacttgtaaaatgtgcaacgcaataggcagctattctttgtgtacagatgtacctcaaatcccagaccatcagggccctcagttagctagcgtcagtggaggggtacgactcagacacaattaaccacaacatatggaagcatggaagatggcacttggcagaaactaagccttaggagtggagcctgtcacgccactgaattcagggatgtgtttgaaacagacaccgtttcagagaccttggagagtagtttctcattcacaataggcatcacgttgatagataaagatattgtggtataggcaaggcagccagaaagggctacatcaacagaggcacaggggcaggaaagcaggaagtaaggaaatgtcaactcttggacttctgagtctgacttaggaaaaattaaggatggaaaggtcagttagggcctaattttggagtgtccagattccctgagagcgtgcttggattttatttgattagtagtggggagctattgaaagttttgagctggacaatgacattaattaactaattaatgcaacaaatatttatgtgtgtcaggcactgttctaggcattttggggtataacaatgagcaaaataaaccagaaaaaaaattcctgcttctaaaacttatgttttagttgaggaggcagtcaatattactatgtgtaataagtatatcatatagtatgctaggtaatatattctgttgaaaaaatttaaaaagtagggtagtggggaatgggtgtataggtggaggtttagggagaaaggcagagttgtgattttattttattttttgagatggagtctcactctgtcgcccaggctggagtgcagtggcacgatcttcactcactgcagtctccacttcctgggttcaagcaattcccccacctcagcctgctgagcagctacaggtatatactgccacacccagctaatttttgtatttttggtagagacggggttttaccatgttggccaggctggtcttgaactcctggcctcaagtgatctgcctgcttcaacttcccaaagtgctgggatcacaggtgtgagccaccatgcccggcctgtgattttatatatgcctcttatttttacttgacacataattgtatatatttattagatacagaatgatactttgatatcgggatacaatgtgtaatgatcaaatcagggtaattagcatattcttcaccacaaacatttatcatttctttgtgttgtgaacatttaacatcctctcttttagccttttgaggatatagaataagttatagtttctactctctacttccatgaggccacattttttttagctcccacatatgaatgagagcaggtgctttctgtgtctgacttgttttgcttatcataatgtcctccaggctcatccatattgctgtgaatgacaggatttcattcttttttatggcagcatagtattccattgtgtgtatatgccacattttcttcatctcttcatcttttgatggatgtttaggtttatttcatatcttagttattgtgaatagtgctgcagtaaacatggggattcaggtatcattttgaaatactgatttccattcctttggataaatagtagtagggttggtagatcatatggtcattctatttttagttttttgagaatcctccacactgttttccatagttactgtactaatttacagtctcacaaacagtgtatatgaattcccttttctctgcatcattgccagcacttgttttttttttttttctttttgacaatagccattctaactggggtgagatgatatttcatttcatagatgtgattttaaataggtggtcaaggtagggtttagtgaaaaggtgccagaagatagaacctgctgcaggttgagcatccctaatctgaaaatttgacatctgaaatgctccaaaatttaaaaggttttgagagctgacaccacaaatggagacttccacacctgaccttgtgacaggttgccgtcaaaacttggtttcatgcacaaaattattaaatatagtgtataagtttaccttcaggctatatgtataaggtgtatatgaaatataaatgaattttatgtttagacttggttcccatccccaatatatctcattaggtatatgcagatatttcaaacctgaaaaaatcggaaatctgaaatacttctgaccctaagcatttcagataagggatattcaacctgtagttcattagggagaatattatagaaagccaggtcgaatattgttgaatgggttggggggttgctgatgaagagtctggaggcaagaaaaccactgaagatttgtgtgaactgataagtcgcttcagtgttggaagtgaaatttttttccacttggtttatatttttctaccttagcaaagaagtccagcagttaactagcccaataaactctacattgtttttttagacttgcgagatacgatttctaaactattggtattctttactaccaagagtctagtgtttctttatttatggactactcataccagaatcacttgggggtactagttaaacatagagatgcctgggacctgccccaaatcttggaggaagagcaagatagtggccaagaacacaggctcaaggtaatcagtctgggttcaaatcctggatctgcccttacttgactgggtggtctcgagcaagatacttacttaccttttctgtgcctcagtttcgccatctgtaaaatgggaataatgataatatctacttcatagaattattgtgaggattaagtagattaatttatgtgaagtgctagagcactgcttatcacctggtacacacttagtgttagctacaattgtttgtattcaatcataatctctgaaggtgaggcaagtaatcttcaaattcaacagtcaccctgggtattttatttgtccttgtgctgtgggaaaaactgttattacttctggagccatggtgaattctgtgagtggtccacacatcaggtatggtgacttagttagaatatgagccaattaatgttttgatttcacacatacaatggtttatttagcaacaaatgccactgtttactgtacatattgcatgttaagcatattctgttgccattcacaacaaccctatgatgtaagtaaatatcactcagattttacaggtgagaaaactgaggcttacactgatgaaaactttccctaagtgacacagcctgtgagctgtgcagttgtcattgatccttggctctgcctgacccaagagtacttgttccttctacatcttctagttattcaacctttttatcttgcaaacatatatattttatagtcagatgcatttgtacaaattctttacaactattggataaaaacctcactgggcatgatttattaatggtaagctagttgcatcattctatatgatcaagatcttaaaaaattttgtctttgtaagtgtagagatataggaaaaaccaaagttcttttcccattctaacatagtcactcagtaccacacagaatgcttctgttaccagtacatgggggtttttccctacacactaaccaaacagttctgcagcagacatcagctctggtgtctcctctaattcaatttaattttgactctatctagaggtagtgtcagatcccactgcttgagagctcagtcccataagactgtcccccacttcagatgccaatcaaagtcagggcctccagaatatggaatttctgactgaccagtggttaaagaaaaacttaagcacttttaaaccatttatttttattttttatttttatttttagagacaggctcttgctctgtagcccaggctggagtacagtgatgatatcgtagctcactgcagcctcgaacttttgagctcaagtgatactaccacctcagcctcccaagtagttgaggttacatgtgcacatcacgatgccctgctaatttttatgtttttatttttcttagagacaaggtctcactatgttgcccaggctaatcttgaactcctggcctcaagtgatccttccacctcaccctcccaaagtgttggaattacaggcgtgagtccccacacccagccgaaacagcttgagcattcagcagttgatggatagggtagcagcagaccacaagggagtgtcactcctccaggaggggcaagaggggaagcttttataaagtatttgcagaagcaagacaaataaaccaattttgattgattagagtggaaagttcctagttagagattagttggtagtttctgattggtgcagtttctggttttaatttactgtttacattgggctttggtttgttcgattagggatttaaagtgacaagagctaccctaccctagtctaatggactctcaattggaatggttttttttaagtttcctcctttttttttttcatttatttatttatttattattattatactttaagttttagggtacatgtgcacaatgtgcaggttagttacatataagtttcctccttttggtcagcctctcatttataaacttggacgtaagtgccactctctgtcaccatcatggctaggttgtctttgtcttagtgtggaactcataaatcatgacaacaggctcaatgaaaaacaattccttttgttgtcaatctcattttttttctaatcacagtgagacaatctggtatgctgttgatgtttttgaatatacatttaagacccttgagagaatatagtgcaccaggaagattataatgatgactaccaggaggataatactgagagacttagcttcttagttaaagtccccacgaaccaaactaactaatatttctatggaagcaaaagaaaaacaaagcttaatgataagaacaaatcataaattcggtctctgagtccagagggtgattagtcaggaagatttctagatttgagctctaagcatattttcctggtttgcagtttgaatgcccctagttatggcagtgggcattttggtgaactccctgagtggtccacacatcaggcatgagggttgtcctctgaacttgtatcaggtatacatctccatcttgcagggtttcaggagcagagcagttcttgttcagagtgattccaagtcagaaaagtgggagaaaattggatggaggaacctagaagaactgaggattcagtgcaaactacaggtagataataaaacttgaaaacagttaatagggctgcagtctaataacaggtgtactattgtttctttctgaaacataattttctctctgtagtcatccttgtttctaccaaagataaatgggccaggcgcggtggctcacacctgtcatcccagcactttgggaggccaaggcgggtggatcacctgaggtcaggagttcgagaccagcctggccaacatggtgaaaccctgtctctactaaaaatacaaaaattagccaggcatggtggcaggtgcctgtaatcccagctactcaggaggttgaggcaggagaatcgcttgaacctgggaggcggaggttgcagtgagccgagattgcaccattgcactccagcttgggggacaagcttgagacttcgtctccaaaaataataataataatcagtgtaagactaatttgattgcataataaattttgtctcatcagacttggcctgattatgtaagtgcagtaagaatagtgattgaccgcacaagttctttttaaagtttgttttgctggaacctttgatagggaatcagattgaacttttaaaaaaacctcttaaatctaggaagccaaaccaaggcaaattttagacttcacctgcagtacctatatatttgggtgaattcttctcttctcgtaaggtcccccaaatatcttgaggttcctgggtctgctgggaactgatagcttttactcactcactgtgagactgggaacacttttagccaggcattctatgcacactgtcaaatatgacccttggtaatataaccaatgtttccaattgtgtcttgttataaagagaacagattctttttgaacttatgcaaatagttatattgccataaaaaaaatcttcacaagtagctttccaagttcccgaggatcaggtagagaggaaaagtaaatacttcaatctttattcacaaaagtatacttttccaaattgctgcaagcagtagatagcttaaaaggtaaaatgtttcttaaaactttaaagaaccagcaaaacttccacatataaagtcataaaaacattattttcatcagttattcagtcttatgtaattcttgttctgcatgatctcgggatagcagtttcacaaactcaccagtttctttattagcgttctgagaattcttacccactttcatggcattatcttaatttaaagttataagaaacctgtatatccagagtacttgtcagagttctttccgtgaatatctttgaagaagaagcaattttggactatagctgattgcaaatgcttttagagaaaaatcaaaacaataactgcctatggatgacaagacttaagatggccatagttaaattctaatgaggtttcattataataatgcaactgacaaaaaaatctggttattttctgtgacatacaacattttaacataataatcaagattatgactgtagacattttataaaacataatttcaaaaagttttaaacttttattccatttatatttaagtcactccttcttaaacaaatatacttgaattgctcatgaaaaatgtcatgagatattgaacagagttagccattgaaagagttcaggaaatgccactccaaaatttactgctttggaatattgattgctttgagccgaaagcatttgaaaaataacaaatgcagggaggggctttttgggactcctttgtctgcctaaagacaggtcctccaaagaaattcagctatcatccatgccttctcccacgccatcatcttaatgttatttaatttttcttgacaaatcaggcaggtatcacagttttttacttccatgactgacgtgcatcaagatcaagcaattcatttttactgcacattttcctcttaggttggatttatggttttatgatcttaaacatctagcagagccaacaccaatctgtctaaccagcaacccaggaaaaacttgagaattctgagaaactcccattttattttaccgacaatttaaaaactagctttatttaccacagacttatgtaaatcatgtgaacttattaaaaaaaaaacacacatttgggtcagtttctgcatttctgagagttttagaaataatttacatgcatgctcatttatccccaaaccaatttgaatagatctccattaaaggattttataaattggtttagtaataccatctgaaaatatcacatatgtgtgtgtatatatatagacacacacacacacaaacattcatacaaagacagacagaaggagataacgtctggtttgagtcacttattaaaagtagtccaaaggtccttcttggttgttggttgctccgcagttgctataattacaaggccattaatttggaaaccttttgtttctcatcttgctctgaggtcctcagctaacttacttaaccctgtagcttccttatccttttcctctctctctttcttgtttggtaatgttgtggggagactccatttacaaaaaggacagctaggattagctgaattgatgtaatgcttggaatgctgtaggaaaaacaaaaggtccaagtgggctttgaaattagtaacagattcatttttcttttgtctgcaggtctggtttagtgcaggtgggaaacactttaggaatggctttcaaaagcttatttatggccgggcatggtggctcacgcctgtaatcccagcactttgggaggctgaggtgggcggatcacgaggtcaggagatctagacaattctaggtaacatggtgaaaccccgtatctactaaaaatacaaaaaattagctgggtgtggtggcaggcgcctgtagtcccagctactcgggaggctgaggcaggagaatggtgtgaacccgggaggcagatcttgcagtgagctgagatcacgccactgccctccagcctgggcaacagagcgaaactccgtctcaaaaaaaaaaaaggttatttatgtttttttggggtttttttttgctttttgtggtttatatatgaaccaaattgggtctcatggccacagatatcatcttcagagtgatacccttctgccttttgcgtccattttggctttcctcatgtcctatccacatgagtttactgggtacaaaaaaaaaaaaatagaacgagtaagacctactatttgaggctggacacagtggctcatgtctgtaatcccagcactttgggaggccgaagcgggtgggtcatgaggtcaggagatcaagaccatcctggctaacacggtgaaaccccctctctactaaaactacaaaaaattagccgggtgtggtggcgtgcacctgtagtctcagctactctggaggctgaggcaggagaatggcgtgaacccgggaggcggagcctgcagtgagccaagatcgcaccactgcactccagcctggtgacagagcaagactctgtctcaaaataacaacaacaaccacaacaacaacaacaataaacctactgtttgatagcacaacagggtgactacagtcaacgataacttaattgtacatttaaaaataactaagaatataattggattgtcctttgtgttatacaaaggataaatgcttgaggggatggacaccccattcttcatgatgtgattattatgcattgcatgcctgtatcaaaacatctcatgtaccccataaatatatatacctactttgtacccaaaaaagctagaaaaatctaaaaacccacaaaaaaataggaaaaaaatccatgtttacctaggttgccaagttcagagcaaaacccattcatgaatagggcagacaaagcattttctctgccagacagggataccttttattattgctctgagattaagattttgatcaagacataagggtacatggcgaaaccccacctctactaaaaatacaaaagttagctgggcgtggtggtgagcacctataatcccagctacttgggaggctgaagctggggaattgcttaaatccgggaggcagaggttgcattgagccaagattgcaccgctgcactccagcctgggtgacggagccagactccatctcaaaaaaaaaaaaaaaaaaaagcagacctgggatgggtgtaatcctagcattttgggaggttgagacgggaggattgcttaagtccagaaggtttgtagcaggacgagccgcagacaaaacccctcagacactgagttaaagaaggaagggctttattcggctgagagcatcagcaagactcatggctcaaaaaccgagctccccaagtgagcaattcctgtccctcttaagggcttacaactctaagggggtccacatgagagggtcgtgatcgattgagcaagcagggggtacatgactgggggctgcatgtaccggtaattagaaaggaacagaacagaacaggacagggattttcacagtgcttttccatacaatgtctgtaatctatagataacataaccgattaggtcaggggtggatctttaactaccaggcccagggtgtggtgctgggctgtctgcctgtggatttcatttctgccttttagtttttacttctttctttggaggcagaaattgggcataagacaatatgaggggtggtctcttcccttaggttgagtctacagtgagctgtgttcatgcctctgcactccagtctaggtgacagagggagaccctatcacaaaaagaaatgaattaagtaaaaaattttcttttaaaaaatttttcttattttttatttttttgagacagtatcttactctgttgcccaggctggagtgcagtggcacatctcagctcactgcagcctcagcctcccaggttcaagtgattctcctacctcagccacccaagtagctgggattacaggtgtgcgccctcatgtctggctaatttttgtatttttagtggagatggggtttcgccacgttgcccaggctggtcttgagctcctgagctgaagtgatatgcctcccttggccttccaaaatgctgggattacaggtgtgagtcagcacgcccagcctaaaaatgttctattaaaaagaagcagacctggttaatctgagaacctaactataaatactcatttagttctttttccttcagggcatgcagataaacaagcaaaaaaaaaaaaaatcagcagactcagagtagacgagaggttagaaacctctacatgccagtaaaaaatacttctcatttctgttttttttgttgttgtttttttttttttaaatagagatggggtcttgctctgttcccccggctgatctcaaactcctcagcctcccagagtgctgaaattacaggtgtgagccaccgtgcttggcccttcccactttctgagatgttcaagatcgtgtgtgtacgtgtgtatgtaggtgaacaattttgtttaggtaaaaacttccactttgtagccactaaaattctaaattgcttttggtaagatttgcccattcttttagaagagcttaggttctgagatgctattatgagaagcacttgagactataacatccttctccccacacccccaaactgggaaggagctgggagaccaaagaataacttggataaatctagcttgacggctagatgagcttgttaggacttaacttcaggacactcctggatggtggctggacagctctagagatccacaccacctcccatttctaagctgcttttaagttaattttctggccctttgtcaattttgtgtggttttccttgatatgtccccagctatgccccaggatgtttaggttctcagggacacgtggtccttggctgggcgcagtggccttggttcactgcctagcttttggggttcaagcaatacacatacactcttaagtaacctggtaggggaccagtcacactatagatggagtcccagactctggatttaaataaactcattatttccagtcttcgtggaacctttcctagtggaggtcttttgcttaacccagtttggtttttgtctaacctagttggacacctgaagcctccctgcaaaacccagtcttgtttctgctgtgacttccaaacctagtctggagcagaaatgttaaaatgaacttggagaacttgacgctagtgttagaatgcacgtatctaagtctacttactggtgatgacctccaaatgtgcagggagtaagcacaaggggctctgtggggtaactgtatctgtttgcttgttgctcccagaggtcattatcatgtagtgtccattggatcccacttctgatatcagatctgataaaagaaaatcttaagcacatttacattttcagcagtttagttgagcattcagcagtttgtgaatggggtagcatcagaccacaaggggctagcactctgcccacaggggcaagaggggaaacttttataaagtatttgcagaagcaacacaaatgaaaccaattttgattggttagagtggaaagtttctagttagaggttagttggtgatttctgattggtgcagtttctagtttcagtttactgtttacattgggctttggtttgttcacacacgaatttaaagtgacagagctagtcagtcagactcaatcagaatttttttttaacattgcctgtaaattggggtttccatgactcacctccttgggtttgatcagtttgctggagagtggttcacagaactcagggaaacacttttctgatatttacccatttattaaaaaggatatgccagaggataaatatgcacagtttgatggaagaggtgcatagggcgaggtgtgggagaaggggcatggagcttccatgtctcctttgagcatgccgttctccaggaacgtccatgggttcagccatctggaagctcccagaactgttttgttgttgttgttgtttgtttgtggaaactttattacataggcatggttgattaaatcattggctgttggttaatcgattcaactgtaagcccctctcccctcccaacaagttgggagtgggactgaaatttccggccctctaatcacatggttggttcccctggccaccagaccccatcttgaggctatctaggaacccccagctactaattatctcattaatacgcaaagacactctgatcactctggagattccaaagattttaggagctctatgtcaggaaacggggacagaccaaatatatattttgcaatatcacaacacgatgttctaaaaggccttttctatttgcgaggtatttttctgctaccctctgtagccagcctccagcatggtttgtgatgatctggtcttcacactcgtgtagttgcctcccacatgttaccaggtttagtctgtgtgaccagtagaatatagcagaaatgatatgtcacttctaagataaaggacagtggcttccatctaggggtctctttctctgtcaaccactttctctggaaaaccagctgacatgtagtgatcaggtccataaagaggtcaacatcgagaggagaaaaaggctcctcagtagctacgtgaatgagcttgaaagtgagtgagcttggaagcaagcctttggagattacaaccctgcctgagggcttgcctgcaaccccaggggaaatccggagccagaaccactcagctaagctgctccctcatgcctgaccttcagaatctgtgagatggtaaatgttcaatgttttaagctgctgagttttggagtaatttgttttagataactaggacatgctgcaattcctattgtgagtcgagttgtactccttcattcccaatgccctggagcccctcagtgtgtgcacacaggcttagttgcatgcactctctttctctgtctgccttcctcctctgtttctctttctctttgcctctctctcacacacacaatttcacttggctgctgacacggtttgggtgtttgttccctccaaatctcatgttggaatgtgatactccccggtgttggaagtgtggcctcatgggaggtgtttgggtcatgggggggatccttcatgaatgactttgtgccctccccttggtaatgatcaagttctcacccggttggttcacacgagagctggctgtttcaaagaacctggtccctctcttgttccttctctcaccatgtgatgcgcctgccttcccttcaccttccactatgagtggaagcttcctgcgaccctcacagaagcagatgctggtgccatgcttctgtacagcctgcagaaccatgagccaaatataaacctattttctttataaattacccactctcagttattgctttttaataatgcaaacagattaacacagttgcttaattctttttttttttttttttttttttttttttgagctgggagtctcactctgtcgcccaggctggattgtagtgttgccgtttcagctcactgcaacctccacctcccgggttccagtgattctcctgcctcagcctcccgggtagctgggattataggcatgtgccaccacatctggctaatttttgtatttttagtagagacggggtttcaccatgttggtcaggctggtctcgaactcctaacctcaggcgatctgcccgcctcagcctcccaaagtgctgggattacaggcgtgagccaccgcgcccggccttaattctttattatgtttatctgttatctacattatttctcttcccatacatcttttcttctcttttaaatatgttgttgtctgggtccctgctgctaatacgttcaatagtaaatatctcactgtaacacttggtatggcaacactctgagctcaggaatgatgtaacggttaaatgtatgtgtggacttgactgtgccatggggtgcccagacatttggtcaaatattttgggtgtgtctgaggctgtttttggatgaaatcaacatttgaattggtagactgagtaaagcagattgacttctccgaatgtggatgggcctcatcctatcattgaaggcctgattggaacaaaatggctgggaaagagggaacctctctctgactgtcttccagctgggacattggtcttctcctgcctttggacccaggcttgaactagaatctaatgccacagtttcttctgggtctccagcttactaactgcaggtcttgggacctcttacttagcctccataattgtgagatatgagaaccctgactgtacaaatgacctaataaactttggctagctaggtgactggaaagagctgacttagtaagtaatttctgagatgccacctgctgaaaagtcctttatccagcagccagtatgaacctggtttcaggtagggtttttcagtgaggcagattctagtttctgttccgaaaacaattggtttttagagagaaatgctgaaggtgctgttgcctttttgcttctctttgtctgcttgttcataagcagcagagtacaatctgtaaaggggagtatgaatagtgtttctgtttttgtttaagagtaagaaattgccagagaaaataattagggcttggatcagcttatttaaagcaatcatcttcacactaagatattactttggtttatttcagagcttgtttctgtgtgtcactttgcccccttctgcaatttgtgccctttgattttcagttcttaaaatttggaggcgagatttccccagaggattatctttctaaatcaaatcctctttatcaagaatctaaatttaaaattgcgtgttttgataaacatgcaggttaaatgtttagaacactacttagagaagatgctggggcccatgcctgccttcttagaaatccacagaaaggaactagggctgccagtatttgttgtatcattgggccataataggtttgtcatcttgataggctgttttcaccaaatatttgccaagaaaagagactttaagaattaaatgtaggccttaaacttgttctacttctgcattcaatagatatctgtttctgtttcattttaaaaagatgttagaccagctgggcgcgctggcttacgcctgtaatcccagcactttgggaggctgaggtgggtggatcacttgaggtcaggtgttcaagaccagcctggccaacatggtaaaaccccgtctctactaaaaatacaaaaattagctgggcgtggtgtcacgctcctgtagtcccagctactcaggaggctgaggcaggagaatcacttgaacctgggaggcggaggttgcagtgagccgagatcacaccactgcacttcagcctgggtgacagagcgagacttcatctcaaaaaaaaaaaaaaaaaaagatgttagaccaccatccagttttgtgaacctccctcacctgtggtggaaggttctttgcagatggtaataccctcccatcatttgcagcatctctagcttggccagatattcaaaacattgtgttttttgtttgtttgtttgttttttcataaatgccaagaatttagctacagttggtcaggcgattgtctactttgcattaataatggcatttgatattataatagggccgttatttcctttattatgagcttagcctttccattggaactgctggaataccttgggagatattgtgggtttgattccagacagccataattaaagcaaatatagcaataaagcgactcacacacattttttggtttcccagtgtatgtaagagttatgtttacactatactgtagtcttttaaatgcacaataggcattatgtctaaaaaattgtgtatataccctcgcccccaaaacaaaacaaaacaaaacaaaattgtgcacttacctcagtttaaaaatactttattgccaagaaatgctaacggtcagtgagtcttcagtgagtcctaatgttttttgttgctggagggtcttgcctcaatgagaatggctgctgactgatcagggaggtagctgctgaaggttgagctggccgtggcaattgatgggctcttcctttcacaaaaaaatttctctgtagcatacaatgctgtttgatagcattttacccacagtagaacttctttcaaaactggagtcaaaactctcaaaccctgctgctgctttatcaactaagtttatgtaatattctaaattctttgttgttatttcaacagtgtttacggcatcttcaccaggagtagatttcatctttgctcatctataagaagcaactccttatcccttcaagtgttatcatgagattgcagctattcaatcacatatccaggctccacttgtaattctgtaatagttcccttgctatttctgccacatctgcagttatttcctccactgaagtcttaagcctctcagagtaatccatgaaggctggaatcaccctcttccaaactcctgttatgctatgttttaacctcctcccatgaaacatgaactttcttaatggcatctagaatggtgaatcctttccagaaggtttttaatgtactttgcccagacccacccgagcaatctctggcagattggccacagtagccttacaaaatggatttcttaaataataagacttgaaaataggaattactcattgatccatggtatgcagaatgtatgttgtattagcaggcatgaaaacaacattcatctccttgtacatctccatcggagctcttgatgactaggtgcattgtaaatgagcagtaatatttggaaaagaaatctttatctgagcagtagctctcaacagtgggctgaaaatattcagtaaaccatattataaacagatgggctgtcatccaggcgttgttattccatttctagagcacaggcagagtagatttatttagcatcattcttaagggctttagaacttttgaaatgataatgagcattggattcaacttaaggtcactggctacattagctcctaagaagagagtcagcctgtccattgaagcattgaagccaggcattgacttctcctctctaactttgcaagttctagagggcattttcttccaataaaaggctgtttcatttacatagacaatctgttgtttagtgtagctaccgttttagtttgttcttgcattgctgtaaagagctacctgagactgggtaatttataaagaaaaggtttaattggctcacagtttcacagaccgtacaggaagtatggctggggaggcctcaggaaacttagaatcatggtggaaggcaaaggggaagcagggacgtcttagatgggtggaacaggaggaagagagcaaagtggggaggtgttacacacttttaaacaatcagatctcttgagaactcactattgtgagaacagcaagggagaaatctgcccctatgatctaatcacctcccagcaggcccctcctccaacactggggattacaatttgacatgagatttggatgggaacacagatctaaaccatatcagctaccttcataaattatcttagctagatcttttggatagtttgctgcagcttccctatcagcacttgctgcttcaccttatacatttatgttatggagatggcttctttccttaatcctcatgaaccaacctctgctagcttcagacttcttctgcagcttcctcacctttcttagccttcatagaattgaagagaggtaggaccttgccctggattaggctttggcttgagggagtgttgagctggtttgaacttctatccagagcattcaaactttgtccatatcagcaataaggctgtttttgccctcttatcattcatgtgttcactggagtagcacttgtaatttcgaagcttcaagaactttttctttgcattcaaaacctggcaggtgcaggcggtctggtttttggcctacagagactattgacatgcctttctcgctaagcttaatcatttctagcttttgatttaaagtgaaagatgtgctactcttcctgtcatttgaacacctagaggccatcgtagggacattaattggcctaatttccatattgttgtgtctcatcaaatagggaggcctgaggagagggagagagatgagagaatggccagtttgtggaacagtcagagcacaaacatttatcgattgagtttgccatcctgtatgggtgtcgtttgtggcatcccaaaacaattacaatagtaacatcaaaggtcactgatcacagatgaccataacagatataataataatgaaaaagtttgaaataatgagagaattagcaaaatgtgatgcagagacacaaagtgagcatgtgctgctggaataatggcactaatagatagactttctggacacagtgttgccataaaccttcagtttgtaaaaaacatactgtgaagtgcaatgaagtggagtgcagtaaaacaggagtatgcctgtacatgtaaacatgctttctttggtgctacttcagatacttaggaagccttgaccatagtccaagcagatgagttttggtgcttgagtattttaccccaggcagactaggagaagtttgtgtgtgtgtgtgtgtgtgtgtgtgtttgtgtgtgtgtgtttgatagtctttatttattttgagacaagttcttgctcttgtcactcaggctggagtacattggcacaatcatggctcactgcagccttgaccacctgggcttaaacaatcctcccacttcaacctcttcagcaggtgggactgcaggtgcacaccaccatgcccaactaatttttgtatttttcatagagacgggtttttgtcatgttacccaggctggcctcaaattcctgagctcaagcaatatgcctgctttggcctcccaaagtgttgggcttggaggcgtgagccactgtgcctggccgatagtatgtatgtatgtatgtatgtagagatggagtctcactctgttgccaggctggagtgcagtgtcgcgatctcagctcactgaaacctccgcctcctgggttcaagcgattctcctgcctcagcctcctgagtagctgggaccacgggcgcatgccgctacactcagctaatttttgtatttttagtggagacagacggagtttcaccatgttggccaggatggtctcgatcttttgacctcatgatcctcccaccttggcctcccaagtgcttggattacaggcatgagccaccgcacccggcctagtctttgtattttagggcagttttgggtttacagaaaattgaacaagtagtgcagagttccttcacgcccctagtttcccttattattaacaccttaaattcatatggtacatttgttacatttggttaatgctgagacattattattaactaaagtctgtagtttttattagtgttgtccagttcaatggattttgacagatgcataatatgtagccaccattgcagtatcatacagaatactttcactgccctaaaaatcaccagcaccccaacctagtcattcctgcttgcctccccctcagcccttggtaaccattgatctttttactgtctccataattttgtcttttccagaatgtcacatagttgagatcttacagtattttggcttttcagattggcttttgtcaataatatgcatttaaagtttctttgtgttttttcacagcttgctagttcatttctttttagtgctgaataatattccactgtctggatgtaccacagtttattatccattcacctactgaatgacatcttggtggtttcttgtttttggtgatcatgaataaagccactatgaccattcactacagggttttgtgtggtcataagttttcagctcatttgggtaaatacctgggagtgcaattgaaggatctgtgatatattcttggtcccagcatctctcctcctgctgacaattaccctccttattgtgacaacctaaccaggcctgcctctcccagggcttcctcttcttcacttagagcattttctgaacaagtctggacacaaaggtcttgcttatattttcaggaaagagtatttaaaatacttcttaatcatggttccaacttatttcaactcttataattaagtgatcattttttttttaaattatggtagaatatgtgtatataagatataatttgtgattttaaccatttttaagtatacagttcagttgtattcatcatattcacatcattgtgtaaccatcaccactatctgtttccaaaactcttacctggggatatgatcattttaaaagaggaatttgaagaaatacatctgaagctatatttggcaatatgtttccatcagtaactcccaaaatctcagtggctattggcaacaatctttatgtctccctcatgttatatatacatacatatatatatatatatatatatatatatatatatatatatatatatatatatataaaattttcttttgtctagaacttagtcacatggccacatctaactgcaaaggagcctgggaggtgtagtctagctacctgcgtaagaataagaacaaattggcttggcgatttgttagcattcctggccatattaagtaagattattaaatcatacacattgttaacttgatctttcagagatgacatgacagtaattgtatggtttcatagatctggctgtgtttcaggagagcatttttccatcccatgctctctttccatctaaacgttaattatcttatccttttaacactgagagagcttcctggaataaataaaattatatgcctattttgataaatacctgtttgatagcgatgttttataaaactctgagaaaagtcaacaaggagccttcacagtgattaatacagcagttattatggtcagatgttaaaaaaaatctcatgtcttcagtcttgtgccaaaaggcaaagaaagttttgaagctttccacacactgatttggtttcaagataatgggctactattgagtttacatgtagcattggtcttctgctattgtctttataaaggaaagtatgtgaaatttggaattggaaatgtcatttcttaaccagagtagcaggttcagatggtgaagctatcacttttggcacaggactcttgtttcttaataaaatttaatgaaaaattgacaagatttgtgcagtgtatattttttttaatggttgacccactgaatgcatctaattagcttattacagtttaaaagcaagtttaactgcctaaatgtatgacttggtattttgatcttgtttgttaggtctcagcttgctgatgatagaagtttgtggtccaatattaattttaaataagtgcctactgttctctagaaactcccattcccttaaggaaaagtgtttcatatgagcctctcattgccaggaatagctgtgtaagcattgtggaagttttttccacattaaaatgtagtccaccagttcatttattccttttagggttgtgacagtcgcttgtgtttatgcacccctgtagagctgtctttaaactcattcagcaaattggaaacatttagcttggatgacacctctttaatcttccagtaatctcaccatagagaccactgtactctcaaatacattgttaatacttttagtaagaatggcctgctttcattctcttattactgagccattattctggactctgtttattattattattattattccagagtcgaaataattatcccgttgaaaagaccaagcatgtgcacaccctgttgtgacacagccggttttgtcctatctgaacttgcctgctttgtgaactgttagggtctcagaaaaacaaaaaacaaaatcttttatttttaacatttaattttttatagtttttatattttttaattgagacaaaatatatgtatataatttaccatctgtaccatttgaaatgtacacttccttggtaataaatatattctttattcccccttccacctttattccatcctttattcctacccccacccctgctcaactcctcctcttcccagcctctggcagtaatttaaagtgtgagtgacaatctcacctcttataattatgtgatctttttttttttttaaattgtggtagaatgtgtataacacggaatttgtgattttactctctcttcacgagatctacagtttttagctccacttgagtgagaacgtgtgacagttgtatttctgttcctagcttatttcacttaacataatgacctctagttccacccatgttgctgcaaatgacaagattcatttttttatggccaagtagtaataaattgtgtatatataccacaattttctttattcaactattaatgaatataggttgattccatatcattgctattgtgaatagtgcttcaataaacgcgcacgtgcaaatatccttgacatctgatttctttacatttgggcaaatacccagtagtaggattgctggatctattaatatgttaattctgtttttaattgttttgagaaatctccatactgtttttcacagtggctgtgctgggaagatgtgtcagtgggtggggggaggataaaacgaggttagttaatggttacaaacataccttagaaggaataagttctaatgttcgatagcagtgtaggatgaccatagttaacaacaatgtattgtagatttcaaaatagctagaaaagaggacttggaataatgccaacacacagaaatgacgaatactcgaggtgatagatgccccaaacccctaatttgatcattacacagtgtaagcatgtgatagagtatcatatttgtgcccccatatacatgtacacatatttgtatcaataagaaatatatacaaaaagccaaacctttttagctataaaatctgtctcttatgttcaaatgtatatagttagaaagtgctggagtcctgtaacatcagtattcagagtttactcaagctttcatcctcaggcaaacttagcagctgttatctaaagccagacttgcagcatcagaaatcacctggagcttgtccagaaagcaagagtcttgggctccaccccagactttctaactcagaatctgtagttccacaggctcccctggtggctctggtatgtaataaagttatttgagaagcactggctggagatcttttacctgagcctgtaatcatggaatcacccatcagtatgtggagtgaccataacatttcaactcaaactctatttctaaaatgaaaaaatctgtctattgtatctaattaagtatatgacaaatatcaagctctctttccactattttctggtgttctcatgatctgacaatgacagtggcaggaccatgtactgagatgaccgaagtgaaaatagtaatgctcttttgtgtattttttttttttttttttgagatggagtctctgtcgcccaggctggagcgcagtggcgcgatctcggctcactgcaagctccgcctcttgggttcaggccattctcctgcctcagcctcccaagtagctgggactacaggtgcccgccaccacgcccagctaatttttttgtaattttagtagagacggggtttcaccgtgttagccaagatggtcttgatctcctgaccttgtgatgcccgcctcagcctcccaaagtgctgggattacaggtgtgagccactgcgcccagcctcttttgtgtattttagaatccagtctgtgactccttgtgatagagaattcaggcattcttgagacttctcaacagattctgactttcccaggtacaggcttatccagacgacatgtttggagataggttgagagagggaatgacatcttatgcttattatatgtcactcttgctcttcagtatttgaaggaaacactgtgtgaagaacaaaaaccttctgtcctggctgctgacattaactcaattttagttattttgagagggcagaagaatattctaagtagactgttaggggttcctaacaagtgagaatttctttgcaagtcagtgggccaaataaacgatttctttaaaaatacaaatccattttatcacttaagagtatttacatttagttccaaataatttgcatatgcctgctaggttatgcctttttattctagagaagaggctccaaagaggatagtttttttttttttctttcttataatactgtgctataatagtcttttttcccccattgggatggaaaataaaggagaaaagaagtggcattcccatagggaatatttcctaagtttctaatgcaaagtttctaatctgaaaactgccacatatttttaaaagaatgccaacatgatttaatctagtttcattcagtttaggaactgaaatgcccctgggtgagggacagcatccctgaaccttccttggcccctggtctgccccgcccctccccatccccactgtgcttcctcttctgcattgtcttctttgctcttaaacacacacatttgaaaactttatttatttgttttttaaagaggtggcaccatcgtagctcactctagccttgaactcctgggctgaagtgatcccttctgacccagcctcctgagtaactggaactacaggtgtgcaccactgtgcctggctaatttttattttttattttttttgtagagccagggcctcgctttgttgcccatgctggccttgaactccaggcctcaagctatcctcctgcctcagcctcccagagttctgggattataggtgtgaaccaccatgcctggcctaaaatactttttaaaacttcttttcattcgcttcttccctttcctttcctccactgcctaccacttctgcttatctgacatctaccatcactctcactaaaacttcagtgcgagatcaccagaccccaagtcctgtggcttcttcttgcgtctctttagcatttgatcttatccagtttctctcctcccttgattcctgtgacattggtcactcaaggttttcttccagatgttttgcttcttgagagcaatggaattggaatcctaagttttaccactttctagctctgtgcctagggcaagacacttaactttgatgtgtctcagttttgttgtaagataagggtagggaaaaacttaccccataaaattgatgcaagtgcatagaagaatgtctggcacatagtaagggttccttaaggataagtcataattattgtcatcattaaaggtcattgcttgccctttttaggctctttggcctgcatgctgattgtaggtgttcactaatttttccactccttttcatagggcctctcttctcttcaaatttttttttgacgatctctgggtttcccacattttatatcttagcccctaactttttccctgaggatagaagtttaatttctaacctcaggctggccatttactccagaagtcttgtgagctctattaaccaaatctgccaaaatgcagctcattctcttatcacctaaagtctcttctctctgttcttcagtcctgcctctcttttgccaccatcttcattaatgcggtcactgtcctttacaactcctgctggggtatcacacgttggcttctcattgcccgtcacccaagtcctccagttgatctccagcatctgttgattgtctgttcatggtgtcatgcagtgtgtctttgctttgctgttcccgcttctgttccatgcagggcacatgccttttgtattagttggtttgggccgcaataacaataccatagaataggtggtttgaacaatggaaatttatattctcacgattctggaggctgaaagtccaagatcaaggtgctggtggcagggtagataggtttctggcatgggcttgcttcctggttgacagagagctgccaggcgcagtggttcacgcctgtaatcccagcactttgggaggccgaggcgggcggatcacgaggtcaggagatcgagaccatcctggctaatgcagtgaaaccccgtctctactaaaaatacaaaaaattagctatgtcctccctggccttttttctgtgaaaaaggccttttgcgtgagcatccctagtgtctcttcttactaggacagcagtcctattggattagggcctcaccctatgaccctgtttaatcttaattacctcctaaagaccccatctccaaatacagccatattgggagttagggcttcaacacatgaagctggggagacacaattcagtccagaacacccttggcgctgttgctgggtagctgtcctgacttccctctcccctcggcagtccagcttcagacaggggtgagaatgatctttctgacacgtggctttggcctcccattcctaattactcgacttggctccccacggcctagatgcagtgagaggaaaatgaccactgtagggtggcttccttcatttctcagttccttatgctcactcctctggaaattggagataacatgactaacctcgtgttattgagggattaaataaattctaatgaagaattgttgctttaatctgacgaaaaccaaattcctactctccagcctccattcgagttcttccatcaccttgttactccaagtgtgatcccatgtggttccagggccagcagcctcccatcaccaggatgcagactctgcatcacagcaagatccccagggccattagatgtgagggactggcttatccttactgcccggctctaggctacagccacaggatggagaggacagggacttggaggcacgtaccaggcttcaaactgcggctcctttacttgctagctctatgcccaaagacaggtttacaggaactgtccttgtcttgtttcccctctgaaaaatagggagaggcagaggtttacctcacagaggttttgaggattagctgagaaattttgtaaataagagagcatctacccttgtgattttaaattgaatttcatacttaaaatcaagtactttttatttggagtcaaggtttgtttattgtggcacagtgtcggggaatgaagaggtgtggggtgtggggtgttcattaacctctcagcctctctgccctctcaccctgtgcaacagggtccccactggtctccctctgggaagatgtgaccagcatgggatatgatggggagacttcttttctgtagaaggtaaaccaactgctgcttcggtggaaggtggagagagcccttccagctctcagaaggggcctggagccggtatgtgcctgtgtgtctgtggctggagcccagtcccattaggccctgcccccgtgattgctgtgagaggtggttgccacacactttctccacagcgccctcagggctgctgaatctaaggcagaggagttcttgttttttgtttgtttgttttttagcccttttcaaatttacagaaggattgcaagaataagaatagtgtaaatacctgctcctgtgttactttgctgaagataatggcttccagctccatccatgtccctgcaaaggacatgctcttgttcctttttatgactgcatagtattccatggtgtatatgtgccaacaacacaggtgtgggggagggagagcataaggaagaatagctaatgaatgctgggcttaagacctaggtgataggttgatttgtgcagcaaaccactatggcacacacttacttatgttacaagcctgcacatcctgcacatgtaccctggaacttaaaagttgatggggggaaaaaaaaaggacacctgcattctctactcagggtcactgttaacgtttagccccatccggttgaccagtctctctgtgtgtggatgtctgtacacatgcatactcttattttctgagcaatttgagaggaagttgcatatatcatggcgtctgcccctaaatacttcagtatatattgccattgcctaagaaaggcaatattctcaccacagttgtcaacgtcagtaaatttgattttttttttttttttttttttttttttttttgttgagacagcgtctcactcttgttgcccaggctgggatgcagtggcacgatctcagctcactgcaatctccgcttcccgggttcaagcaattcttgtgcctcagtcttctgagtaactgggactgcaggcacgtgcccccccaaccagctaatttttgtatttttagtagagacggggattcaccatattggccaggctggtctcgaactcttgatgtcaggtgatctgcctgccttggtctcccaaagtgccaggattacaggtgtgagccaccaagcccagccagtaaatttgaaattgatacaagacttttatctaatctgaggtcatattccagttttttcaatggacctagtaatgccagtctcctttaatgtggattttcacagtctttgtcctttatgacatcgatgtttttgaagaatgcagcccctcctcttttaaaaactagaaccttccggtcaggctcggtggctcacacctgtaatcccagcaccttgggggaccgaggcgggtggatcacgaggtcaggagatcgagaccatcctagccaatatggtgaaactccgtctctactaaaaatacaaaaattagctgggcgtggtggtgtgcgcttgtagtcccagctactcaggagactgaggttaggagaatcacttaaaccggggaggcggaggttgtggtgagtcaagatcacgacactgcactccagcctggcaacagagtgagagtccgtctcaaaaaaaaaaaaaaaaaaactaaaaaaaaaaaaaagcaaaaaaacccgaagcaacaacaaaaccccaacaaaaacttgttttggatttgtctgatgtttcctcatgattcgattcaggttctgcattccaagcctgaacactatgtaggtattcagagcggtccatctgcctctcactggggtatggccatctctatccttcagtcagggtggggcttttctccattatactgttactacacttgatctcagccaaaaggccaagaagtgatctccactcaatgtttttccttctggcagctagttaatctgtggagaggcactttaaacatcatttaaatatcatcttcttcctcaaaatttctcccgagctttagcatccagcgatgatttttgtctgaatctttacaaagatggtcagggtggcattcttctgtagttcttccttctctctatttattactttcttggactcttggattcctgtttttttcagtggtttataatccctgactgtccctaatttttttggcggcctgattgtcccatattggctagttggagccccttccagccagctgctgtatcaccatgacacgtctgcattccccaagtatctgcggcctgcctctcctggtggacagccacattgtctccctttgacgtgccctccaagtaaatattgtgtttcatagcttttaaatcagactgtcctttatatgtctttcaagtttgtggagaatttacactgacatatttgagtgatgagttaacaggcagacagaaacttaattttctttatagtcatttgtacctactaccatttccgtttgcaatgacatgagtcgtctccggttattaccctgcagtacatacgcactttactggctgcacctagttgacttctgaactgcccttgggctccctctgctggcatagtggtgcgtgagcacttttctggggtcatggaatggatttctctttctgcccattttctttcttttggtttgtggttttctctctcataaattattacacaaacatggaccatacaatacaaccttctttcaaacctgatttgtttgctaactgtatcagtacctttccatataaaattcataaatatttttatataattatgtcagtgtttccatatttttctattacctgaatgttctttttagcatttaaataatccatttttagtaggcatttgtattgcttttttccatttcaggtagcatctaaagtgaatgtcccttgcatgtatactattcatatgtgacttaataagtagagcttgctaaatgggtaggaacttacaggaaaaggggaaactctagtttgagaaaattgtctgggctaagtttaggatgtgttcaacaaacaggaaagtcaagtttggctaaagcatggttgagtaatagtgggaaatatggtcagaaagtaggtatgttaggactctttctactgaaatgatgaaattcaactccagccacaagtggagcagaacaaaattgtgtttgtttaataactgggaagcccagggtagctggaggcaggggcttagatgctgctgcgccatcccttacctgtctgtttctgtttctccttctgtcccttcccagtctcagcactgagtctcttgcccattggcctggtgagggaaggagctgccagccccacccaacagctcaggttacagagagagtcactttcttccattactcacagagtaaacatcaaggaaggccactgattgattgacagtgtctgggtcagatgtctatccttaggccagtccctgtgaacaaggggatggggtgtctgcgtggaccagatctgaagcacaggcccatgcctggggccagggggtgggaactatggacctctctccccactgagaaccccagggagcaggtgaggtgaaattcctctaggggaagaggggcaaaattgacaagatagcagatgtctaccatactgctgtggggcctggtccctcccagaaggaaaaacatagtaacaatagagtgggtctcaccctccacctgggtctcaagtagggtgtggatgaggacaatggaaatgaaggaaaggttagaaggcctgtggtaccggttggtagatagctcttcgtgctttctccatatggagtgagagtgcttggatgtgattccttcaaagtcaggtctaggagactcaggatgcctaatctagaggtaagaacattgtgaggaaagccagtgaattcagtcttgtgcatgctgactttgaagtacttttggaagagccaagtggaattatccacaggacaggaccaaatcttacctggttcttccccaggccgactagtccacaacaggaaataaaaagagttgccccgataccaaggtgtactagtccattctcacactgctatggggaaatacctgagactgggtaatttataaagggaaaaggtttaattgactcacagttctagatggctggggaggcttcaggaaacttacaatcatggcagaaggcaccacttcacagggtggcgggagagagaatgagtgcccagcgaagggagaagctccttataaaaccatctgttctccttataaagatctcttaataaaaccgtcagagaactatctcattcactatcaggagaagagcatgggggaaccgcccccatgattcagtttactccacctggtcccgcccttgacatgtgggtgttattacaatttaaggtgagatttgggtggggacacagagccaaaccatatcacaaggctttctcctccttgctgggattgtacccatagcctctttctgagtcctctctcttttagcctctttatgcctgcagtgcatccttataccatttctagagtcatctttataaaacttatactctccgtatgactcataaatcctgtttttttttttgcacagtatattaagtataaaatttgttaaagtctttaatggtctgcccccaagctacatttccattttgtatgtctttcagttcctttctactttgtatttggctgttgagttaactgaatttttgccattccattaacccatcccatgcttttcccacttctagatttcacttttcttgtaggctagaatgtcttgactgggatctgactggagataatgagaacaaaaactggttcaaagagccaggatgttgcataaaagtcctaagattgtatctaagcaggtaaaataaaaattttaggcaattacttaaatttgaaatgctcacatttattaataaggcatgtaacatctacatgagccatcatttgcttttttaattccacattgattaggagccaaaccttcagggcaggtatccggtagagcgccctggagaggccctggataggcacaggcgcctgtcagggggctcttcacatgctgtgtgctgctgctgggagaagagggggccagagactagggggcttctaagaagaggtggcatttctgcctcagtgttgaaggatgaataactttgacaggctggaaaaaggtgacatttcaggtagagcgtgtcacatggatgtaaataccaaaggtcaaggacatgggcttgagagatggtgagaaggatggaggtgactgtggcttgcattctatccgtatcactattaattaccttctaatgcctttggctctaggtggtggaacaagtaaagtaatggacaaatactttttctaccaatatttagtgaccaaatgcagagttatggagagggccagggacctcatgaaccatactctttctagtctagggacataactccaatgcctttcctgtcccagtaagaggccatggatttcaagaagccagacaatccattctttcagataatgataaaaaagaaaccatttattttatttctaagtatagaatgaaacatttatagttgcccaaattttggtaccttttaggagaaaaatacagatttttttgttgttaaaaataaacttaaaaaaaaaaaaaaaagacttaccatagtccgggcacggtggctcacacctgtaatccgagaactttgggaggccgaggcaggcggatcacctgaggtcaggagtttgagaccagcctggccaacatggcgaaaccccatctctactaaaaatacaaaaattagccgggcatagtgggtggtgggtgcctgtaatcccagctacttgggaggctgaggcaggagaatcacttgaacccagaaggtggaggttgcagtgagctgagattgtgccattgtactccagcctggtcacaagagcgaagctctgtatcgaaaacaaaacaaaaaaagacctactgtaaatagagtacaacatgagactaacaaaaataacaaataaaatcatccagcatatattctgtatttaaaaaaaaaaaatcacaagatgaatacagaccatcttggtgcatatgtattttatacactagacacatgctgatgtttttaatgcttaaaatatattaggtttcatacttgcttccaatccaaggagagtatatctgagctctttttttttagatttgaaaaccgccttttagattagattagaacactgcctttttttttgttatgcagtaacagatcattgcccacctgaaagcgagtcgcctttatttttacctccttagtagaaccagcaggagctgcaattgctgttgcttgagccaaggccacagtcagcatagaaaggttctgctaagagactgtgttaaaggaaataaatttggtgacttagttttgttttacaagctagctgtggcccagtccttctgcagggtgggtacgcaggtttattgcagctctgcagagccatggccttagacaaacaaagaaggtgtgggaatacccgggagctttccattcaaagtagattcttctcctttgagtgcatgtaactgaagcaccagctttctaaaaaaaaaaaaaaaaaaaaaaagagagagaaaagaaaagtcagtctgaagtttttgtgacactcagccagactccacattagataaagtaatcatggaaatacagctttatttttataccaagttgtgcttagaaagctgattgctaaaccaggaagcttggctttcagaattttactgtaatccagggtggaaaaaaagtatcactcagatggcatttgtgggagattactgactggtaatccctccttgtcctagaagattaagtctgccctggtgtagtcatggtcagctgatttttttatttatttattttaaataaattgtagtttcttgttgtaagtcagccagtgtctgatgtttgcctgaactgtttgtacctctgggccatattgcagaaccctgcccttctttgttgactgaggaaagctcgctccctgcccaggtttttcattgttgatcgaaattaacaccaggtggtgaatagagcccctcctaaggttgctcaggataaatcatttattaaataggtctgcttatcaggaggggcgtgaaggctcccaaaaggaaatgctggcacctgggcccagaagccagggcctctaactcctggggttgatttcttcagtgaagttgcaccctacaaagggaatatggccaaagcggcactcaactgaaggctgatatcaggcgattagacagccatgcattctgcgtttgtctggaatggattgtagagagatggacttatatgaggactaccagtccccgtttgattttgatgcaggagtgaacaaaagctatctctacttgtctcctagtggaaattcatctccacccggatcacctactcttcagaaatttggtaactactaattttgtaaagttaagtttgcttggaataaaacctagacataacttggggtttgcctggctttaaaattgttagtgtaaagatgtaccagggtggtgttgggttgtaactagctaataagctattctagattcaaagtacagagatagatggctcattaaatatttagaatatgtgtatacttagaagttagattttaaagttagtactatttatgcgatcactagacttctccattgatagatattccaagctcactttgagtttgttgagctggtaaataacaaaaaaggaggaatctgaagtccttggtttctagttgcttaggtaatttacgacaactttcccgatttttctctttagtaaaatgatggttaagcactgactcagttcaaggtcttgctaatgactgtcctgggttttgttatggaatattgagagcaacttacagaaattgctaggcaagagatttttttttttttaaagaaaaactagaaattgctgtacctttagaagcagttattccttcatacttaccagtatgagattgctttgaaggccaggagcaccatcagggaggcccttcctttattggcacaaggcagcagaaagccagaagcagcttagccatcctcccggctgggtccctcatctcatagccaatgctgggaactggttaaatgaccttctctgtaatttgccaaataagcaatcaggtttttccagaagttaattttttccttattaatgaagaacaggtcacattttgttcattttaagctgaaaaaatgggtaacctagaaaaaaacatggtaaaggtgacagcatgaaacttagttttaagtattttaggtttgtgtatttgccgacagtagggatgtgtgaagcagcattcctctcttggttgcacctggtagccatctttccccaaaagtggctgggatttccaggaacaggaaaatgttgaatataacagagaaggggtagagctggtctgtagaagttgggggcaggaggcagcagcttgatgtctgctgggggattctgattgccaggaaactttctccagtgctgaccttgccttgcccaagtggcccccctcagacctctgtaataaaggggcctgggctccccctttgcccagcccccttctaacttataaattttatgtataatttaataggggccacacagaatggggaagctagagcttgataatggactgaaactcaagtgacttgcgggggagggagggaacctttgagaagctggggctgtggttttgtgacttcaggtggaccttgatcggtgtgcaagcatccagattcccaggaagattgacactgcttgcttcttttagttcccttctacccttcttgtctcttggtttcacctcctttcattttccaagttatggtcatggctgccttgggatgttttctgtcaaaaccagggcagttgtagggttgggtgtacctctaggcagattctgaaagattatcttcacaattcctgttagctgttcgaaggaacctggggttgtgagatttaaaaaattattaaaaagcaaatgaataacagtaatgaaaaacaaactcatgtagctaggtttagtagtttgcattaattgaaagaaaagagggtgaacaagcattaaaacaagcagaaaccaccctcagcagtcatctgtaagaacacagtgattttaaaatggccaaataaaagtgggggcctggaagagtgattactcatctctgtgcccatgacccctcagggcataagaagtctgcacacctgtctgaacatcaaacatggttggtcgatggaatgaataatgtgtcctccgtgtgtttgcactctttgtatttctacgtagaagcttttaggaccaatacattatgaatccattaaaagttcttgtggcttttggccactatgtattttcttaatcaccagacaagtaatacagacaagaaggactgatataaaaaggaatcctacccagagaggttgggaatactgatttgaaaagagtgaaaaatatctctgatatttatccaaaatgccatcatggggcaaacgggaaagtaatgagattttatctatgggcaggatgttcctaagaagtaattaaagcgaaggttaggatgaggacagtggctgaaacaaaatgaagccttttattagggagttttaaactcctttgacacaaccaacccatattcagaaatgccttatacctccttacctgcaatcctcacaaatataagaatctatcaataactgaagcaaaattttatgaaccagaacttctccttactatatgtgatgtactctgacattttatactatttgatgccatttcttcttttaaagtgctcatctggttctgctaactggattttacaacccatagtgtgaaaacaccaatttataatagtttcagaatctggaacataagctttgtttgtctttttagttcacctctgtttccccaagcaccaagaatagtgcttgacattatatggctcaaaaactttgtggaatgcatgaatgaatgaatgacactggtaaggagtcgttcacactccagttcattttacggatgtgaagattgaggctgagagacatatgtgatgttctcaactcacaaagtttagttaggctaagataggactagcactcatctcttaattcttaagctagtggtttggttttttgttttttttagaccgcaacatctcagcctctctggtaatggatattatagaacagattaactattcaaggcttaagttctcctttggtcaagtgttttaccgtgggaataatttgttgatactttatacgccacttggatctcaacattctattccatctttgggctgaacattatgaggaccagggtctcacagctggggtcttttgttgcattatctcttccattctgtgaacacccttaaggataatatcccaaatgttatttacatagcacctatcttcctcttaagaattttttttttttttttttgagacagagtcttgctctgtcgcccaggctggagtgcagtggtgcaaccttggctcactgcaacctctgcttcccgggtttaagcgattcttctgcctcagcctcccaaatacctgggactataggtgcgcgtcaccacatcgtgctaatttttgtatttttagtagagacggggtttcaccatattggcgaggctggtcttgaactcctgacctcgtgatccgcctgcctcggcctcccaaagtgctgggattacaggcgtgagccactgggccgggccagaattttcttttttaaaaaaaacttttggctgggcgcagtggatcatgcctgtaatcccagcactttgggagtctgaggcgggcagatcacttgagctcatgagtttgtgaccagccatcatggtgaaaccccatctctaccaaaagtaaaagaattaatcgggcatggtggcacacgtctgtggtcccagctacttgggaggctgaggtaggaggattacttgagcccaggaggggattgcagtgagctgagatcacaccactgcactccagcctgggtgacagaatgagactccatctcaaaacaaacaaacaaaaaacccttttatttgaggttcaggggtgcatgtacaggcttcttatatgggtaaacatgtgtcataggggtttgatgtacagattctttcatcatccagatactaaacctgatacacagtagtgattttttttccctttttttgctcctcttcctcctcccaccctccaccctctggtaggccccagtgtcatttgttcccctctttgtgtccatgtgttctcatcattttgcccccacttataagtgacaaggtgccctgtttggttttctgttcctgtgttagtttgctaaggataatggcctccagctcatccatgttcttgcaaaggataggatcttgttcttttttatggctgcatagtgttccatggcatatatgtaccacattttctttattcagtctatcattgatgggcatttaggttgattccaccctttgctattgtgaatagtgctacaatgaacttacgcatgcatgtgtctttgtgatagaatggtttatattcctttgggtgtatacccagtaatgggattgctggggagaattttctaatgtacaggaagatgcagacttggcttatggcacctttggttatttctattgacttaaggaattatgagcaatggaaaaatctgacattttagtttaacacgatcatatttgatactctagaaagactgaaaaatcaaactgaaattccttagctcatagttcttttcattctgttttaccctttttgattttcactctgtgtcattctcacttctttttagagaagtggttaccacttcttaaaagtttttcatgattgcatcagtcttaaatagcaaacatggctgtggtttacaaaataaaaactggagtcaaattgaagagatgtagaatttccttattcctagagaggaaatttaaggatggaagagacttcctctctggggattctgagatttctgcatggctgacctttgtcactcttttctggaatgtcccagttactagttttatactcaattttggggcaggggagcagagcagtggggagaggagatggctctgaagtaattgactgaagattttttcttttaaaaaaaaatgaaagagatttaagggtccatttagaaatacctggaaggaccaagtaaagcatttgaaaatactgtgttttaccttgaggatcgtttcttctttgtccactctttctgtgaagtgaagctttgggaggtaaccttctgtcttgggtggggacattgaccactgttcagggtcctgctttcagagctgagtcccaggagcactttgcacccgatgtggctcccaagcgctcctttccctttttgtcttagtgcagcccctcccctcctgctctgtttcctttactctttaataacccagacattggccaggcgtggtggctcacgcctgtaatcccagcactttgggaggccgaggcaggcggatcatgaggtcaggagattgagaccatcctggccaacatggtgaaccctgtctctactaaaatacgaaaaattagccaggtatggtggtgggcgcctgtagtcccagctactcgggaggctgaggcaggagaattgcttgaacttgggaggcagagattgtagtgagccaagatcgcgccactgcactccagcctggtgacagaccaagactccatctcaaaaaataaaaaaaaataaaaaataacccagacattgtccccttacgcttgtgaggagcttgggtcttcccttaaatggagtggatcaagttttctcagtttttttcttccctgctaccttgactacaattacccaactcgttttccccctaaggttttactgttcttgccatgggcacacatgggaaaattccagccttaagaatctaaagttgtcacggcctgagctgctgcatccctctcttctgaaggcactcagtggcagacagcatcctcttagatgctccctcgccttaagtcagtggttctcaaccacagttttgccccactgtgggcatttgaccatgtttgtagatactcttttggttgttacaactgggtgtgggtgtggggctgctactgacatctggtgagtgggcctgagatgctacacaggaagatgcccagggcagtccctgccacagtcatccaccgcatgacgcccgtggtgccgtgcctgagaagcgctgccttaactggatgcttaatcttttgggtctcccgaagacaaccattgtggtgtggggacttgcaaatattcctcccacttctgggattttattacatccagaaacaacaaggcacagagaatgggagccccaggcacagacttaacatggggaaaaaacatcctgtgatctttctggatgttaccactaaagatactttgaattatatgtttaaatctctttttatgcctctaggtttaaaaagggggatgcatatttcctcttgtttgtgcttaaataaaacagtcatagatactgaagtttcttaccgcgactccggtcttaaagttattggtcctgctttgaagacttgtgtaggaaaggcctgtgcagctttattttccagagacgcttccagggctcaaatatatgttaacagattctttcttgggtaggcagtgttaacattcaggctaaatagcagatatcttagactccattttggcatctttttccctcctagttgaatttttttgggggcagctgacatattctttctgtgatatcatttttgtcccacctatgtaaccatggtacttttgactgcacactgttttcctgtaatatgaaggtaggtgacctgcccccaaaagtgacaatctttggacagattatgtatgagatggaagtcatatttctttaggaaagtgacatttcacatctgctttaaatagaaaaacctttctaggtaaaaacagtgaagtgagaatgcaatatataaaataaaaagctcgagataaaaggagagagaatgaagggagactgggttcttatctttccttctaacttgactgtataacttcctataatccatttatccacagtgttgacacgcatctgctgtgtgccagacactgtgcatcagagatcctgctaaggctaaagacacaggattggagcatgaaggccaagtattgcccagctgcagtctgtattctatacaatagactggaccgtaggttcttattcccagagccaagaactgtttctcagccaaggtcacggggtccaggggagatcccccaagagtaggagacacttttgacttatttaagctaatgggacctggaaatagggatggggtttcaggcagagaaaataatatgtgcaaagacacacgaaaaatagtgtgttttagagatagcagagatggctgtacaacattgtgaatataattgatgtcactgaatgacacacttagaaatggttaaagatgatacatttttgttgtatattctaccataataaaaaattaaatgggccaaaaagtatgggaggtacatttaaacaaaaattttttttgagttaatacctttatttctttccttctttctttctgtatttctttctttctcgctctcgctctgttgcccaggctggagtgcagtgggtgatctcggctcactgcagcctccacctcccaggttcaagtgattcttgtgcctcagcctcccaagtagatgggattacaggtgtgcgccaccacacctggccaatttttgtattttttagtagagttgaggtttcaccaggttggccaggctggtctcgaattcctggcttcaagtgatccacctgcctcggcctcccaaagtgcagggattacaggcatgagccaccatgcctggccaagtttatatatatatatatatgtgtgtgtgtgtgtatgtgtatatatatgtgtgtgtgtatatatgtatatatgtgtgtgtatatatatgtatatatgtgtgcgtatagatgtatatatgtgtgtatatacacacatatctatgtgtacatgtatatacacacatatctgtgtacatgtatatacacgcatatctatgtgtacatgtatatacacgcatatctatgtgtacatgtatatacacgcatatctatgtgtacatgtatatacacgcatatctatgtgtacatgtatatacacgcatatctatgtgtacatgtatatacacgcatatctatgtgtacatgtatatacacgcatatctatgtgtacatgtatatacacgcatatctatgtgtacatgtatatacacgcatatctatgtgtacatgtatatacacgcatatctatgtgtacatgtatatacacacatacacacacacacacacacacacacacacacatatatgttttttagatggagtctctctctgtcgccaggctggagtgtggtggcgcaatctcagctcactgcaacctctgcctcccgagttcaggagattctcctgcctcagcctcatgagtagttgggactacaggtgcacgccaccacgcccagctaatctttgtgtttttagttgagacgggtttcaccatgttggccaggatggtctcgatctcttgacctcttgatccgcctgcctgggtctcccaaagtgctgggattacaggcgtgagccaccgcgcccggcctacatttcttaaaaaacaaaaaaagcatgtgtttctggaggaactgcaggttggagggtatagtgggagtgttgtgctggtgagggcagtagcaggagatggggcaggtgctggggagtggggccagattgtccagagtgaggagctttgcaagaagctgggaagccatcaaaggacttaaggcacgggagtggcgtcgtcagatatgcactctagaaagctctttgttggggcaggttgaggaatatttgcagatgtcagagactggcaaggagcttaggggtaggaatctaagcaacacatagtgacagctcaaactaggcaggagggcaggattggaggcaaggagtgaagtggatgtccagagagtgagatggagccatgacgtctcagagaggtaggaagagaaaaaccaggagggaggagaggaatggagatttcagactgaagtgcaatgtcagatggccttggcgtggctagatgagggtagtgacccagaattgtccttagtggaagggaggtggaaggggcagccacaaggttgcagggggttgaagagtagtgggcagtgaggaagtgagacctgagagccaagactcagggaggacgaaaggagagagaagcaattgccaggacaagttatagggcatggaacctgggggaaacagcaaatccttgtgtgtacccattttttgcaggtaaggggcttggggctctttgaagataaatgtgattaaagaagatcaaccaactagtcaatggctgaatcagtgtttatgcccctttccatcggaattattctcctggggctgcggtagccattctgggtggcttatcacccggttctggaggttagaaatctgaaatgaaggcgttggcagggtcttgctccctgtgaaacgtgcagggcaatgaatccttgctgcctcctcgcttctggtgtttgcttgcagaccttggtgttccttgttctgtagtgatgtcaccccaatctctgcgtgttctctgtgctttgcgctctgtatcttcacataatctcttctctgagtctctgcgttcaaatttccttcttataaggacatgaaccattggattagagttcactaatccagtgtgacctcatcttaacttgattacatctgcagaaatcctgtttccaaataaagacacattcattgttgtcaggggttaggaaattgaagacatctttaaaggcagcagagttcaactcacaacaccatctaatttcaaagctcatcttgtcttttcctattcttgtaaaaatctgtcctcagttttctcatctttatataatgaagcatttgacttaaaccagggatccccaacgcccaggtggtggactggtacccatctgtggcctgttaggaaccagtgagtggcaggcgagtgagcattaccgcctgagctctgcctcctgtcagatcagtggtggcattagattctcataggagcatgaaccctattgtgaaccacacatgcgagggatctaggttgcacattgcttatgagaatctaatgcctgatgatctgaggtggaacagtttcctctcaaaatcatcaacccccaaccgccgtggaaaaattgtcttccatgaaaccagtccctggtgcaaaaaacactggggactgctgacttaaacgattcagaattctttcagatctaaaattgtatgacaatggccaaaaagcttttaatcaataatcttagaaagtcttgaagcacttggtcataattggaataaaagtcaaagacgaagaactagagggaagataaccatttaagtgggtgctccaaaggggggttggggctgcacccaagcagagagaggcgctcctcaaactcttatttttggcttaagtgtaggttctacatttttagtaaaaacgcttttgctgctttccccagttgaaatttgtgaaagtatgtggcaaatgggtggttatgcatgggaaagtaaagagagagctactttaattaaatctttaaagaataattcagttcagatgcaatattaacattgactgttctaggcactgtgagttctgagatggagagataaagtgacatcaggactttatagtctagaactagagatgtgttcaggactaagctcttaccggttcttggccactctgctctgcctttccctgctctgaccacactcgcacacacacatttccactttgtcttccgcacattattcacaaggactctgaagtcttgaagtgttgtatttcatcaaatccaagacaccacagattgtaagatgttactattattttttgtttcattaagtataaaatatgctaatagttaaactaaaaaaaaacctgctttatcataacttaagttttaaatcttttttaaaaattatttttagtagagatggggtttcaccacgttgcccaggctggtcttgaactcctggcctcaagtgatctgcctgtctctgcctcccaaagtgctgggattacaggtgtgtgtgagccaccatgcccagccccctaaaaatttttattaaaatttttgttgttgttgttttgagacagggtcttgctctgttgcccaggctggagtgcagtggcacgatcacagctcattcagcttcaacctcctaggcccaagcgatcctcccgccacagcctcctgagtagctgggactacaggtgcaggccactatacccggctaatggtttttgagtttttgtagagacaggatctcactttgttgcctaggctggtctcgaactcctgggctcaagtgatcctcccaccttggcctcccaaagtgctgggattacaggtatgaaccactgtgcctggccaaaatttttacatttattgaagttcttttttagatttattcagacgtttacactgatcataacactcctgtgcatattttctaaaataaaaaagaacatttcctaaaatgtcttcacacccataatctaacttatttgaattgctattcaacttggagtaatctatgtctattctattgatttattttcctcacataaaatgtctgtggtgcagcctttcttaagagacgtctgtaaccggacctggcattttctaagccattttgcaaattttgaagcatgtttgcaggtgataaccatgtcacaatgactggctgattaattgtacagtgcatctcaaagatgtaaaatgtaaaaagtatcttcagattgatgggatgtgtctctgcttgtcttctccatccttacccctgagacactagaactctcagctgggaatcagcgagatgcattgccctgccagtggactccagacccttcccaggggaggccacccaatacccagcttgctggaaggacttttccaaagacaagtctagacttgtcactcccccacctaaagctcttcagggatcctatggatggaactgaagacaggctgacaatctgtttgcacagctagatgaagagaactaaggcaatggctaaaatgttaataacctgatggaagaaattatcactcattttcagtcacttgtgtttgataatgttatctgtgactttcaaaccagggatccccacaggttcctagaccctcaggaaagtaaaacgtttctatggagaaagactaggccctgtttccttgtcctgccatgatcattgatgtttctctaagatgttcgtgatggagctgagtcgagagacagaaagacccaagtattaatcctgcctctgtaactctgggcaaattatatagccttagaggaccgcagtttcctggtggaaaaaaaatactcctctcggatggtggttatggggtgtttttatttttataagataacatgtagacttcccctcacagtggctggtgtagtagatgtcagtagatattaattgtattttccccaccatctttgtcttttaaaaacagctttccatcagattctgagaaaaaaaaaacaacattaaaattaatgaacaaataagttttggattatggttttcaataatctatacttttaaatgttcttattttccacctttcatgttgtgaatgagaaattaatcttttctcccaaaagccagactctattttcatattaaatcctataccattactgaacatacaagaaacaggatcatatctagacgaatatataaggcaaaatttggattcccaggtttggggaaactttagttggaattaatgaagactgcttttgatctagtctgccagattctttcactttttagggtcttattattgcttctagatacctttcaggtctgaccatcagcctcctacttttgtctttgttttcagatcagtgatgtgaagcatttgtttttacatcagaccgtctaggtttaaaatctgacctccaccaaagatttaggtgtgttaacctgggcaagttgcttaatgtctgtaagcttgaggttcctgatctgtaagtggtgacagggatccccacctcacagaagtgttgcacataagtatttaaaacacttaatttggacttgtctgtaaatggaatgcactctgtcatggtggctgtggttaataattttctcaaggcatgactgtgatgttttgctggtgattttggtatctaccattctgttcttccttggtgttctttaatttctaatttttttttccagactgcaccttttaattttaatgataatgatttgctgccacctacgggctaatgtggaaagagcagaacatgtcattaccaactttcttagatgctgaatataaaatctcaaggtcatcctggcagacttgaagccaactggacaacccgtcatcctcttttctgagagatttattttccttgatagcctagccgcatacttgaatagaaccagaatccccttggattctttttggaattgggcagtatacataaagcattgtgttagcagtcagatactgtatttctccccttgtttgagtagatttgcaaaacagtgtgtatcaaaaaagcatgaaagagtctttgctatttaagtaatggtttggtacctgagttttgcttgttttgttttggagatctgttgtagttggagaacctgggagttctgtacaaaacattaaaaataaaataacgtttaacaggtagctctgttatggttcttactgtgttgccttatatgattgttacttgaatgcttgactctcagtttcaatctcatacaactttcttcacataatctgacatagagggtagatttacaagcacacacagatgctgggaaatggtttctgacagtacatagtacagcacgttgttcacggtgtggtttggcaggagttggagtaagaggtgccaatgccagctagcaggagacgggtccagtaagggctacctgccaacctcaggggtcacactttgctttgaaaacagtaaggaatcaccaaaggattttaagctgaagagtgatacagtcagatttctattatagaaatattatttgggcagtctgttcacaaatattttagaggaagacctgcggaagcaattatgcattgagcttttctacttgctgattgtacctattaaatattttatattcagtcaagagcttaatcatattttttgccagtccgatttgtggtttttgaattttttgtgttacttttatgcctttaacagttctgtgctttccaggtaggcactattttggtgctgccatgctgatgattattattataattattaactgctattaatcagtgagtgtacagtagagcctgtaccaagtgcctttgtaacttgtacaccatgtacataagcatcatctcattgactcatgtaattctctaatgcttgctgcgacgctggctgcttgttccacagtgtggttattctccttcctttttcttctgtgtctgctccctggcgccttgcattgggcaggatgccacattgctgctgtggctgaactggcagttctcagaaatggtcctggggggtagcagggaacaactgcagtggctgtgatgctcactggcaggaagagcaggcagaggagtaaaccttcccctggtttctttgcaaaaaaaaaaaaaaaaaaatctgttgccagaggagactttgtaatcccaaatctgtaaactgaactgaagcctagggtaaggcagctgaggccaggaagggggtgacaaaaagcttagtcgtcaaataatatacaaatacaatatttttaaaaattaatgtaaaagaaaatcatgaataaaatatcacaattttaaataaagatgagattaccaatagtgctgttcatgcagagccattttggagtttgagacaaagtatcagcaatactgatccttcaatttaaaattttggcatttttgttcatcatgggttctttgaatttgatttttaaaaaatactgcattaagatatttatattgagtgttttagtggcctcttcaattttgcacccaaggttggggtgcctttctggcctcatcttagcctgtgggtgagggttgggcaggaaaagaggggagatctgaggggcagctgcctcctggtcactctccagttgttcttcctgacaggctccgttccaaggggaatgggtgcggtgtggttggtcttcagtagtgtagaatgtctctgtgtggctgttggtcatgtctctgaacatataccttagtattcttttataagggtatctggatgatatatatatatatagatagatagatatagatatatagatatagatatatatagatagatataatcatctatatcatcatatatatatatgatggcttgaagccaggagttcaagaccagcttgggcaacatagcgacaccctgtctctacctcctaaaacaaacaaacaaacaacaaaaaaagaacttacaagaagatgttttctgcctccactaatggaaagaaacagaataaaatttacttaggacatttagttcattcagtggttactcctttactgagtttatgaaatgttggctttgattaccacagcaagctgaagagatatgcaaactcctgccctgggttactgggaaggagctaaaatctttcatctaatttatactgatttacttttgcagcaatttaaagtctcagcaagatctcattttgggcccttcactcttctgccataactatattgtgactgagactatgaacagaagctaagagaatgctatattttctaaattcatattttggcagagtctacttggtagtttccggtggttagctcagattttcttttgtcctttgctgagaaagccaggtcatttgaatatccagagatgagatgtgtggatctactctaattggctttattctggctaggacagtggaggagcttaaatagggattttacttagattttgcactttaaaaaatggtttaaatccccaaccaggtctgctgagaacagacccagtccctgaggaaggagaagatgttgctgccacgatcagtgccacagagaccctctcggaagaggagcaggaagagctaagaagagaacttgcaaaggtaaagaaccattccttttgcaactttatttttgcacttaaaatgtactgtcatttgtgattctaaagtacatggtgatatgtttatagttagttccaggggagagaaagcatatgtgtttacttctccagcaggtcttgagtaggggagaggagatagaagagggtgacatcggttcgtttttatatgagaaaaaagtatatatgggtaaaatagatgtgaaaagtagagtgtgaagcaggaaatgaatgagaggagtagtagtgaatgtcttctaagggacttcactgacatctgatacacttgctcttcagtgcatcagtgactttgttctacccttggctgtgttgcagagatggccactttccaacttccttccgcctttccttccttccagctccctccttccctcccctcccttcccttccctaccctgccttccctcccctctctcccttccttccttcccctctctttcttccttcccctccctccctcccctccttcttccccttctggcttctcttttttctctttccgccccccctccccctccctccctctgccattcccccccattcctcctctcctttccctcccttcttacttttttctttcctaacttcttccatgaaattcatttaacataaaaattaaccattttattatattttatttttcgagatggagtctcgctctgttgcccaggctggagtgcagtggcacaatcttggctcattgcaacctctggctcctgggttcaagtgattctcctgcctcagcctcctgagtagctgggattacaggcgtctgccactgtgctcagctaatttttgtatttttagtagagatggatttcgccatgttggccaggctggtttcgaactcctgaccttaagtgatccacctgccttggtctcccaaagtgctgggattacaggcaaaaaaattaacgagtataattcagtaacatgtaacattcacagtgttgtgcagctgccacccctttcaagttccacacattttcttcacctccaaagagatccctgtattcattaagcattcaatcctgactactcccctccttcagcccctggcaatacctaatttgctttgtgactctatggatttaactgtcttctggttatttcatataaatggaatcatacaatatgtaaccttatgtatttgggttctttcccttcatgtttttgaggttgattcatggtgtagcacatatctgtgcttcattatgtttaatgactgaataatattccattacatgtatctattgtattttgtttatccatttatccattcatagacatttgggtgatttttcaccttttgactatggtgggtagtgagttaggaacattttatgaacaaatacttgcttgagtacctagtttcagttctttttcatatatgcctagatatggaattgcttcatcatgtggtagttccatgtttaatgttttaaaatttttttaactttttttttaagatacaaggtcttgctctattttgtgggttggtgattgtgcagtggcatgattatagctcactataatgttgaactcctgggccaagcaatcctcctgcctcagcctcctgagtagctgggactccaggtacatgccattatgcctggcaaattttttaatttttatttttcatagagatgagattttgctatgttgcccaagctggtttcaaactcctggcctcacgtgatcctcccacctcagcctcccaaagtgctgggattacagatgtgagccactgttcctggcccatgtttcaagtttttttgggaagcaccaaactgttttcacagtattaacaccatttaatattcccatcagcaatttccccacatccctggcaacatttgttattttctgtgttgttgattatagtagtcctagtgggtgtgaagtgatatctcattatggttttggtttgtgcttttggtgtgatatctaagaatccatttctaaatcctagatgataaagatttacccctatgtttttgtctaagacttttatagtgttggctcttaagatttaagcccttgatccattttgagttaattagcgtatatggttttggtagggatctaacttcattcttttgcatgtggctagttttccccagtgaatgctcgtggcatccttgttaaaaaagaaaaaaaatcagttaaccatagatgtataggtttatttctggactctggattcagattcattcatctatatgtcttttcttttgacagtatcacactattttgattactatagctttgtagtaagtttgaaatccagaaatatgcgttctttacctttttcttctttttaaaaattgttattcaaggttccttgcaattccatatgaatttcagaatctgcttttccacccacacaaaaatgctgttggcattttgatagggctcgcgttcagtttatagattgacttggtgagtattgccatcttaacaatattaagtcttccagtccatgaacatgaaatgtctttccatttatttagttcttattttactttagcaatgtacaaaacatttttctatgtacgagtcttatacttccttagttatatttattcctaagtgttttattcttgttgatgtgattgtaaatggattttttttaaattttcttttcaggtggtttattgctattatgtagaaacataactgaatattatatcttgatcttatattttgcagctttgttgaatttatgagctcaaatatatttaaaaaaatttttttgagacaggtattgctctgtcacccatgctagagtgcagtgatgtgatcatggctcggggcagccttgacctcccagctcaagtgatcctcctgcctcagcctcctgagtagctaggactacaggcgcttgccaccatctgcctggttaatttttttatttttcagtagaggtggggtcttgctatgtttctggactcaagtgatcctcctgccaaggcctctcaaagtgctgggattacaggcataagccaccacaaccactcttgaatagatttttttgtggattcttttttttttggggggggcggtgggggggaaggtatggagtttcactcttgttgcccaggctggaatgcaatggcatgatcttggctcactgcaacctccacctcccaggttcaagcgattgtcctgcctcagcctcccgagtagctgggattacacgaatgtgccactacgcccggctaattttgtatttttagtagagatggggtttctccatattggtcagctggttgtgaactcccaacctcaggtgttctgcctgcctcggcctcccaaagtgctgggattacaggcatgagccaccgcacctggccttttttgtggattctttaggattttctatatatgaaatcatgtcatctgtgattaaacatcgttttacttttcctttccaatttgaatgcctttaatttatttttcttgtctaactgctttggctaaactcttcagtacaatattgtgtaaaaatggtaaaaatgggtatccctgtctttttttctgatcttatggggaaagctttcagtctttaaccataagtatgatattagctgtcatttttttcacaaatgcacttgcgttgaggaagttctcttttattcctagttttagtgggttttttttttcatgaaagagtattggatttttatgaaatgctttttttttcacctattgaaatgattgtgtttttcccctttactatattaatgtggtatataacactggttgattttcatgtgttgaatcatccttgcattcctaggatatattccacttggtcatgttttataatccttttaatatgctgctggatattcataggagatatgggctgctgttttcttgtgatatcattctctggctttggtaaatagggtaatgctggcttcttagatgagttggaaagtgttctaccatcttctggttttttagaagagtttgaaaaggtttgtccattctttaaatgtttggtagaattaaccattgaagctatctacttttacacttttctttgggagatttttgatacttgttcaatctctttacttgttcagatttcttctattttgttgggtcagtttagtcatttgtatatttctatgcatttgtccatttactctagactatctagtatgttggtgtacaattgctcataatattcttttataatcctttctgtttctataaggttagttaatgtccccactttcatttgattttagttatttatgccttctctctcttttttttttttcggtcagtctagctataggtttgtcaatttcactcatctttttcttttcttttttttttttgagatggagtcttgctctgtcacccaggtggctggagtgcagtggcagcagtcttggcacactgcagcctctgcctccagggttcaagcaattcttgtacctcagcctccctggtagctgggactacaggcgtgcaccaccacgcctgactaatttttgtatttttagtagagacgaggtttcaccatgttggccaggctggtcttgaacttctttttttttgtttgttttgagatggagcctcactccgttgcccaggctggagtgcaatggtgcgatctcgtctcactgcaacctctgcctcccaggttcaagcgattctcctgcctcagcctcctgaatagctgggattacaggcacgtgccaccactcccagctaatttttgtatttttggtagagacggggtttcagtgtgttggcatgactggtctccaactcctcacctcaggtgatctgcccgccttggcctcccaaagtgctgggattacaggcgtgagccactgcgcctggctgtctttttcttttttttaacccttaatgcactgttgatctttttaaagaatgaattattgcttttattcattctctctattgtttcctcttgtccattttatctttactctaatatctaatctcttccttattcccttccttctcttagctttgggtgtagttttctattttttaaatttttctctaattccttaaggtgtagagttaggttattgagattttttttaatgtaggcatttataactatacatttcttactgacagttcttcaactgcatcccaaaagttttaatacactgtttttgttttccttcatttcaaggtgttttctaatttcctttgtgacttcttttttggcccattggttgtttaaaagtgtgtggtttgattattacatagttgtgaattttctggttttcctcttgtttttgatttccagtttcatggtggtggttggaaaaggtgctttgtatgactttaagtttattgagacttgttttgttgcctaacgtatactctgtcctggagaatgtttcatgtgcatttgagaacagtgtatttctactaatttggggtgagctgttctgtacatatatgtgttaggtccagttgatgtatggtattgttcaagtcgtctctatgatcttctgtctgtttgtactactcattttttcgcccaaagttttataaacaaggtggggatagaatgggtaaggcagatgggtgataaaccatttgtgtggttgtttttttgttttgttttgttttttgagacagagtttcactctgttgttgcccaggctggagtgtggtggcataatcttgactcactgcaacctctgcctcccaggttcaagcgattctcctgcctcagcctcctgagtagctgggactacaggcgtgtgccaccatgcccagctaatttctgtatttttagcagagatggggttttgtcatgtaggccaggctggtcttgaactcctgaactcaggtgatccacctgccttggcctcccaaagtcctgggattacaggcatgggccaccatgcccagccatatatttttatatttgtagagggcattagaatacagataaccttttcccacttaactgattttcaaaaacatttttatgccaatatactcttgtgcaacctcattttaaattgctgcgtagtccattttgaatttccgcaagctcatttttaatttatattatttataaacatttggattgtttccattttttgctattatgaataatagtttgatgaaaataccttcatgtaagtttttatgcacttctttaattagcgctttaggataaagtcctggaagtagcattatgcctgggtctaatggtttttaagagtatatagcaacatggccaaaatgctatccagcatatttcttacagtgttttgaaagagcattttttttcaaatccttgacagttctcagtattgttttattatcaacaaaataaaaataatagtaatagcacctaatgtctatagactacatgctgtgtgggaggcgttaaactgagctctttacatgcattatttcattaattattacaacagtcttacggggcaggtgctattatgattgatggaagaggaaacatagtctttatttaacaaatgaagttaccagctatatgatttgtaaatattttctcccattctgtggattttccttttcattttctttatagtcccttttaaaacatttttaaacttaaattttttatttaaaaaattttagattcagggggtacatgtgcgggtttgttacatggtaatgcctcatgatgttgaggtttgggcttctaatgatcctgttgcccaagtagtgaacatggtttttcagctctttcccccaactctccctctccacttctggaatccccagtgtttagtgttcccatctttgtgtctgtgtatacccaatgtttagctcccacttataagtcagaacatgtggtatttggttttccatttttgtgttaatttgattaggaagatggcctccagctgtatccatgttgcagcaaaggacatgattttgtactttttaggggctgcatagtattccatgatgtatatgtaccacattggacagtgggcaccggggttggtttcatgtctttgctattgtgaatagtgatgcaataaacatatgagtgcaggtgtttttttggtagaacaatttctatacccagtaatgggattgctgccattgaatggtagctctgtattaagttctttgagaagtatccaaattgtgttccacagtggctgaactttaggactgttatgaataatgccgctgtaagcaattgtgtacagctttttgtatggatgtgtgttttctttttattgttgcttgtttgttttttgagaagagtcttgctctgttgcccaggctggagtgtagtagcatgatctcagctcactgcaacctctgcctcctaggttgaagtgattctcctgcctcagcctcctgagtagctgggattataggcacaccaccaccatgcctggctaatttttgtgttttagtagtatggggtttcaccatgttagccaggctggtctcaaacccctgacctcaagtgatctgtccaccttggcctcccaaagtgctgggattacagccgtgagccattgcacccagcctggatgtgttttcaattctctttgttatatatacctaggagtggagttgctgggtcatatggtagctctatgtttaactttttgaggagctgtcaaactgctttgctgcccaacttcattttacattctcaccagctgtgtctgagggttccaatttctccacatccttgccaacacttattaatctctgttttttttaattgtaaccatcctaatgggtatgaagtggtatctccttgtggttttgatttgcatttctcaaattactaatgaatggtgttaagcatcttttcatgtgcttattggtcatttgtatatcttgggagaaatgtctatttaagtcctttgcccattttctaattggattgtctttttattgagttgtaagtttctttatatgttctagatactagactcttattaggtatgtaatttgccaatattttccccattctgttggttgtcttttactttcatgacagtgtcgtttgaagcacaacctgtaggcatcctccctttcccaacctctaacacccacaattttactctctacttagatgggttcagagttttttcttggcgtccacatgtgagtaagaacatgcagtatttatctttctgtgcctgatttattctgcttaacgtattgttctctaggctcatccatgttgctgcaaattacaggatttcattcttttttatggcagaattgtattccattgtgtataccacattttctttatccattcacctgttgatggacacttgggtattgtaaatagtgctgcaataaacgtgggggtgtaggtaccctttgatacactgatttcctttcctttggataagtaacccagtagtgggattgctggattatgtggtatttctattttaagtttttctgagaaacctccatactgtttttcatagtggctgtactaatttatattcccagcaacagtgtataagagtttcatcttctctggatccttgccagcatctgttattttttggttttttgataacaggcataataacaggtgtgaggtgatacctcattttgattttgatttgcatttccctgatgattagtgatgttgagcatttttaatatatttgttggccatttatatgtctcttctgagaaatgtctatttagatcctttgcccactttttaaatgggattgcttgtttgttttgttgttgaattgagttccttgtatattttggatattagtcccttgtcagatgaatagtttgcaaatgttctctcccattctacaggttatctcttcactctgttgatgatttccttgaatttagtcccatttgtctatttttgtttttgttgcctgtgtttctgaggtcttagccataaaatctttgtctagaccaatgccctgaagtgtttctcctatgttttcttctagtagttttatggttttgggtccttagtccattttgagttgatctttgtatatagtgatagacacatcagagattcttaatcatgctctttatgcttttaaagtcactctgttttacatgtatatattgaattaaggaatcgcagccatgatgtgctgggctaggtgtgttagcagcacaagctaaccaacaaaggtggggaatatactgtgttgtgcattgttcagaaagatttagagtggtttgtctttgcagctttcattagaggaagtgagtacctcagagaagtgctaaggggtgggaaacttaccatttaagtctattcttagaagtgtctgaagctttggagcctgaagtagagacagaggaagtgggaagcttaagagagtgtacatgagaattcagccagtaaactgagacgtgcattcagaaaacctagataggaattctggttcctacattatttgtttgatgactttgggcaaatcatttaatttacttaattttgaaccttattctttcctctttttttgtccttttcttctcgtattccacttcgtcaatgtacatcttaaaatgtgacttacaaaactgggtccagtacttgtgactctgacaagcacaggggattatttatttgttctgagggacctctgttccagttaatcttatctgagagtggtgttgtaggttctttttcttctttttttttttttcttggcagctgcatcacatttgatttatccttttctccgttgcactaattttctttgttttgtgttctctaaagacttaaacatctgtgaaaacatttgactgagcaatactctatgttatttttaaagggcatgttgtctttgaaatcatagaaataccgtgcaggttggggctgaaaatatggctttgcaacaagccaggagacgcatttctctgagggtcttgaatgttaatggatatactttagggacctagattttcaatttcctgggacctaaagtctcttccacatggtgtctatttctctctgaaataggcagtccacccttttccctgtttctgaaatagagagtctaccattttccctgttttcctggattagccttttgttctactctgtccattttttccttttcttcatagcttctgcttacagttttagattcctcaagcatatttaagctttccatacctctcataatctcactgtagcctacctatacctcaaggcttcttcaagttttttcagcttctacttgcaatgtcatctcacttctcaaactagattcctgagtgagagagacgaatagttgagcacctcttttcacactgggctcatccactggctcttgatgggtggctgtccatgggtcagggcccacctctaaccacctagtcaagtctgggcagtcagcatcaaggggttcacatgaaacccttgagagcagggatctcctgaggaggcggtttgatttattatggccatgactttgccagacacagggactgagtatatgtatccagtatatatactcatatcaaatatgcaaggagattggatttgggtttggcttggttgtgtgtgttttggtaggggatggagattattttgatttagtatacaaatacaagttttgtaggcattcagatagaaatgtctaggctgggaaggcagacccacaggtcgggagagtggatgttagagtgctttattaagtattatctgtgtagaaatttgagtcctaaacctacatgagatagctgagtaaggaagtatagggagagaggagaaggccaaaaacagcttaagatgggattataaagagctcccatggaggaggaaagagaactctacaggagagatcagcaagtaaccagtttactcaacaacattatacattgtttttagagagaaagataaatgattcaaaaaaatgtttaatgactggaattttggatgtagttctgtggtaataatcttaacttactgaatgacttttgctatattcttaccccaaaccccttcctcataggtagaagaagaaatccagactctgtctcaagtgttagcagcaaaagagaagcatctagcagagatcaagcggaaacttggaatcaattctctacaggaactaaaacagaacattgccaaagggtggcaagacgtgacagcaacatctgcgtatgtttccttgatcatacactttccttgggagtgtaaaggacactgttgtgccttttgtctggagaggggaagaaaaagaggataaggatgcttcagcagcttttctttggacgacacggcaccctttttttctgattttcaagtgtgcctgcctttttttgtaacttcaatttttccagaccacttattctaaggaatagaaagaaagctagccagttggagaaataaaattcaaatcccataagtgttatgtattgacttgccacaaatatgtacattttgtcctttagtactcagaataacccagtaagaatgggtggcaagataaccctggataaggaaacaggctcagacaaattaagttttcatgtcacaaacctctcaaaatgtggagtgggaattcaaactcaggtctggagccagtctttattattattattaatgtatttttttaaccttgttgtgtaggtatcaaaagattctcaggtcgtagtctcagatatatgctcttggcagaggtcaatcccttaggctttttgtctttttgaccacacatgctatgatatagcattatcataatgcagttttgtttcccggcacaaggaggagcaagcttctagcagcagaaaccgaactgctctgtcttctgtattgagagccatctgcagagctggtgagcacagaagttgttttacaatttaactatcagaagtgaggccaactgcatttaacatgacagtacttgagtccccagtcttttggtccattttctacccatctaagttggcagtgggaaaacatacaggaaactattttttcaacagtattaccatgaaccatgtttttttattggtatttgatcttgacagggtaagtaaaaatgcctgagcattctttcccataatgtgggaattgttttggtaggtacgatattattttgttttttgttgttgttgttgtcatcatttttttgtttttgcttttctcttctctaatctccaggtagctgggaaggaggtggtaaattatagctttgcgtgatagcagataccacactagaataattttaagcattgcatattttacatactttgttgaaagtccatcatgtccgtatttggtagctctctaaagacatgcaaaaatttttagaaagtatgcccataaaatgtatttggataataaaatacaaatgggtcctacttacgccagctactaattgcttctggcccttttttaggaaatcaagttgacaaaggcatctgaggaatttgatttcatttttgttttgtttggattatgtgagaaaagaacaataggtatgagttctttcaggcctttgcttattttcatcttggtttgatgattatttttagcccttaccttgacttctttggccctgcctttataggactctgtctttctggctggactagggcactgcagaagattgtgcctgagcttagggttgagcatacgaagatgtggtgatgccgctctgctggttatgaaggattttagaagttctgaaaaatgttttctctttttgagagttctccagcaggtggtgaccactgcacttgaaacaattgatattagaaaagatgagctgacccttcttttgctctgtgtgtttataatttaatatagttacaagaagacatctgaaaccttatcccaggctggacagaaggcctcagctgctttttcgtctgttggctcagtcatcaccaaaaagctggaagatgtaaagtatgtcctcatttttccttgctcattaagtagctgacgcaaattaaaatgttattgttgttgcatctgtttcctgaccttggctctagttgatgctagctactcctgttagagtgagggagggcactccttcaaaccagtacctggaaggctcatctaaggtcttctaggcctagcattctaacatcaaatgatcatctgatgatcatttaaggttgttgaaaaattctggttgtgaacttctttgtctttttagttcttcatgatcatgcagggtaagtatgtctttggttgcaaatttgtgattgtgggcagagcctggtgtgttactaaggatgactacaggagcactgaagtgcacgcttcgattcagatttggcttcacgttctatatgtgacattttatatcatggtgtgattacaacatgctcatttgtgcactgttttattctaactggtaagctgtaactatgttcatgcttaggccaaaatcttgattttgagttgttgctttttttttttttcatttagattagtctgcagagttttccttgccatttttataaaaccacttcacagtgcttttgtgcccccccttcactcatgtggattaggatagtgatgaaaatgacaaatgtggtttaagtcagctgtgcttctgttggcatgcaagatttgtgtttaaaaaaaaatgctcagattgcatggcttcgtgttttgcatttgtggggaaagtgtatatcttacaaccagaatttcttggttgaatagatttggtcaatttatgtgttatactttacaccaaaagaacaaattcctcgctcattatatttagactgtcattgtctaagcataatgtaataaatatagctttcttgaactcttttgacaattatgtatgtatctattaggtgttcattttctctatgctgactgaattgtgtagcagtttagagcccagggaaaacattatatgtttaaaacttaggttatttaattgttttagatttatcagaacttctttcttttttaatgcttactctggcttcttacagattgcaagccttttcacattcctttaggtaaggagagtttgaaccatcactgcagtccagcagttgtataagaaaagtgtgctgagattagattaccatgctacgttggatgatcgtgtttgtccagtacataattttcttgcgtttagagtatctttttctctagtgcattcctaatttcaagggagggtttcttgtataccatgggtagttgataaattaaaagaagtatttgacactgtggttcaagggatatggaaatgattttgacatttcttttttacttttttctttggtaaaaagaagcagctttaccatggcatgcaaaattcctcccttttaatgtctgactttgtttgttttgatgcttttgtttctttggtacttttgtaaggatgttggaattcttcacctgggtttcattacacaattgtaacagactgcatatgtactgcctcctgatgttagtcttccatcttctattggttactctacctttttttttgttgttgtggtaaaatatacataaggtaaaatctgccactactctgcactttttaatacttgttttactagcttatagtttttatgattttattctttctttacacttgttatattctttatgtacacaaggcaacttcatggtttgaaaaagggaaaagaatattaagctaaaaaaagcacattaaaaagatgttagctcaaaccatgccttatagaaagagactttgtgatgttgaggaaacctttctctttcccaggcagactctttgattatctgattagttgttggaggaaactaaggtggaggaaagtctttagagtataagcaggaaggatgatatagtagtcttcatctctgttcctatggatgcagattattttcttaagcaatcactcatggtagtctagctgcttgcctttttaagtaagcaaacaggtacttaaagctcctatcttggtgacacttacccctggaagtcagagtaaattatagggatatataaactctaaatgttgaactatttaaagctaaaattatgcttaaaaaatcaagtaaccaatgataggtgattgtgtatcgttggcattcagttaaaaagtgctataagtaactgaatacagagccagtcacatcacccattctgctaacatttagagtgtctactgtggacaggttccatgggcataaccataaagaacgcatggcttttattagaatgaagcttaaaatcttagagtggagtaggggaagagtggggacttgggataagttaagaatggaagagccttatcagtactggttataaaatgttcgatgtgatgcaaacatcagtgtaagatataaaagtaagggctattgaggatgatgtttcttcagtacatatcactagacttttctatacctttaaagaatacctttctataccttaaagccatacctagatatggctcagtgtgtgctgtcttattaattcatcagcaacaaattatgttttgcaactgttttctagctcctttcacagtcactttgtccattttgtttaaaaaatataattaagattatgctgtaccattttcagtcttgtttttaataaatattaagtttttatgaatatttaattgcccatcagtatgtaggggttaaagtaaggtttttgattaaaatttggaaatgaaagaaatgagattatcttttgaaagttaaactttgtaattcttgcaaatcacaggttaggtaaaggcatttaaaaaaataatttcaaggccaagaagtcctgattgtgttctgatttaatgtgttgacacatataaagaaaacctacatcttttagattatgtgagtcacagaggcaaatattggccacgttccctcaagaactacccaaggaaagggagcttaggctgtagatttgagtgttacaattccagcaataagtccttttctcccatggaggtggatacagtaggctccttttgctaagtgctatgaagtaggtggttagtccttaatcccactcactgtttgttctaggtccattttacctggcaaacttatgccggtgattttactccttaatgtaattatggatatagaaggagaaaatgcagcagttttgccaacctgaatgtgttagactcagcaacagtttttattttacatagtcagttgataaatcagcacttgggatttagatatttgccttatgccttgattgaggggagggagatgtataaggaaatagagaagagagagagagagctgctcttgatgtcctgagaatgaggtgtggtggtagcaaggccaaggaggacgtggtcatgagtgctgtcctgggaggccctctggggcagcctgcttttccctcaggtgatatgcttatatttcccctctgtttgcatatgttctatgcctatgtgaaagtattgactcatgcctttagcattttgagggaaactttaaaagaaccttctcattgcagtcagtgccagggttaaacataaaattttagtgttattaaaagcatactataatattcagtagacacatttgtctcatatttttggtatatttaagaggaagtgctaattaattttgcctttatttccaaagaaaatttctcctttttaaatgtgtgtttcttcatgtgcccctgtagatatttatttggtttgggctcattaaagtcctgctagacaattcccttttgctgaaaacttccaccacttaatgggaaaagaaatgatctctttcctcgtgtcagaaaaagaatatcttttgattagaatttgaaacactttaatgaacttttctctgtttcatgcccatattcccaattttagttatcaaaattgtttattcatttattcattcattctttcactcaaaagtatttattgagtgctaactatgtgccaggcattgctgtagactataggggtggagctaaaaaccaaaataatgctgaaatttgaaagcttcaatgtgaaatttgaaagcttcaacgtgatatgcttatatttcccctcctgtttgcatattttctatgcatatgtgaaagtataatatacagtagatgctaattcatgtttcaattaaagcatgtgttataataataattaacatttataagcccagcgagacaatagtacctcagaagcagatgctattgtttttgatattacagatgaggaaactgagccatagagagattaagtgacttccccaaggttgcacagcaggtataagtggctgatggtcaggactggaaccgagcacacctagtcctggagcctgcactgtcctcctgggcctccccagagcagtggtggtttaagacagtgcccctctaggcactgtccagcactttgaacaatagctgctgtgatcaccaacatcaaaagattgtgtgagggcaaagtggaataatagtacatcaatagctgtgaaaagcatacgtggtatatagtaagcagtccaacatgttagccattaactttcccttctcctcctcccgctgctgctacttccttccttatcctccctctaccccctcctctcttattattattgagcatgaaagcatcagataactgttagaaacaaggaatattttttaaaaatcaattcagctgttttaaaaaaatgactttttcaaagtaagagtaaatgcagtgtgagattgaaaagtacttagtgagaatcagattatcctgattttttttttcagtgttttgaggagcccatgtttgcattcctagtttgattacagattaatgtatttgggggttacaaataagatgcaacatttttaacaaaatgtgccttatagatggtaaccgattcttacaacctatctattagagatgattctggtcccgtagcaacatgactttttacttttgaaggtttccatattacatttaagcttgaatgaaactgaaaatgatgagcaagttgaagctttgtgtaacttgccatagagaagccagaaatgaaagaacagccaccctatagttagattacaactagttaccaacaaaattgaatttcatctaactatggattatggggaggatccagcaaccagatagaattgaagctgatgtgtgctccttttcttcctactataaaatagctaccataaaagcagagagaccttgactttgactctgttaactgattttattctgtttaatgggtatatttatcttcaagtgtgatataatgcatacagaaaagtgctcaacaccaaattcatttcagtgatttttttcacaaaataaacagtctagtcacttccttgcctttccttatatattaccactgaaacatatttagtaggtacatgctaggtaaagtggtatagagcccaaccgatccactgctggaggaagtgatattgtacatgggttcttagggacagtgactagaattagctgtgaaattctgaaatttgcacttacttatccacctgtttccacattttgttgatactaccaaattcaatgtgtgggtccccgggacaaggaatttatcacatatttggccattaccttctttttcagcgatttctgccaaaatgaatgtcctaatgaagagttttgattattaggaacagaaagctatgtaacttttagctcacctaaaaagaagaatgtgttgcacaaatacaaagtttttttgtaattcaaggagagaaaagaaagcaaggccaggctccacccaaaccagctccatcaactgggaaattgaagtagcttctattattgaaaccaagaaacctattcctcttccatcctgctggattccctgcatgatcacctctcctgacctcatctgctctacctctctgtgtgcttatattcgattctttctttgggaactggtttcctctgtgttctgtcagctttcacctggtcccaccagatcctcttcaacatctaagtgtccagcagcttagtgtcctaattacaatatgctaggataagagtcttaattgtcttcactccttcatttttcatacattccaaacaatttgtaggccactgggaggttagcagtggtgggtggtgctccactgatccacctatcatatcagttatggtagaaggtggtttgtggcaatgtgactattccaggcagcttcagcagaagggtctgaggaagaacaggtgatacttagagttctggtgttgtaaatttagagattgggaccagcacactttgatagatccaaagtaggaaaagaattatcaaattcctcccagaatagattattatactcttataccctaggtattgaccaccattgtaggatagagagaatactgagttacatgcatcttttcctgttatgtaagacatcaagtacagaggtgtttaaatatttcattgataatcctgattacacagattgccagcatatttgacatatatcacaatcattctccatgttatcagatattaatgcaaaattagatttgaggacctacattgaagacacgttatcatttgtaccaaatgagtctttaagaaaaggaaatcataagcgcacaaaatatttaaatataactgagaattaataccttgaacctatacactttacccaaaggaaagtttatttaccaaagtcgtgccagtggtcaaactattataagcatagtaacaatattagcagaatacaatcattttacgagcctaggaatgaaggcactctcatctcatgaggtggtctttatgggagatggctaagagtaggtaaccttgaacctgagtggtcagtgaactgcattgtccttgagtgggcagttgcctccttgtcttactaagagcccttccctggccttgcttgtgtgtgtatgtgtgtatgtgtgtgtgtgcatgcttcatttttgaggggaggcgataaacactgaattagacccaagttctattcttgattctgctgtgacctacggtaactcatttaacacctatatgccttacagctccgggtgaaagcactttagttttcaaagtgctgtaaaaatgcaaggtggtattgttagctctagtaactcctgggaattaattggctcctgaataaaagtaaattatgttggaataactttgatcccctccacctcaaaaagtaggtaattcttcatggcgccaagagaataaagctcctggggagctaagttttgtcagcttttaataatgttgaagcaccactatactaaccttatttctatctctttttaatgctgcctcttagtatacgttccattcagcattcaattagcatgcctgctatgaggtaaagtatatttttatgaaatgtagttggtcttatgctttatttttctttatttagatgtttaacttgaactgttctgaattagggaactagaattccccttgaaacaagctgtgtgtttaacactctatcagttttcagctacacttcctctgatgttgggatgggtgcaagagatccgttaaatgttttgattttgtttttaaaaaaattatttctattttattttgtaaaacctttgtttcctgtctgaaatctgttcacaaagtgagcactacttgttaacaaaccttcttttgtcccgccacctctgtctgcattcgctttagttacattctccttttcttcatccttcccattttcacttcccccatcccactgtgacatttggagccaactatgatttaattctactatctgctttcattagcacttgcaagtgatattacctcacctaacctctctagagagttagaggatttttaacaatctgtgcttggcttaaacttaagtgtccatcaaaatcacttggagtgcttgtgaaaccgctgcttgctgggccccacctctagagcctctgatcctgtagagttggggtgggacctgaaaattggcattgcttatgacttcctgatagctgcagcagcagctggtctggggcccacactttgagagctgttggcttaactgttctgccatcaccaccgtgggaaggaagaaaccccttttcctttctttgcatttttcatcttggtagtaggaaggatattcaaggtcagttggataggataacagcattggaaaccctgtttccaaagcgcctttgtgagagacaaaggcgctatgatcctccttaattaatatggatagatgtgaatgtaacatggatgaatgaatgtcctgtgggctcagcgtgctctcctcatcctttgggacagagttgtgaagtaggcaaagccgtttctatgcttctcccccttctttttggtccctgctttcccagtctattatgctgctccctgatattgaacctcataagcttgtcccacttaggtgatgtgtgttcatgtagaaattctgccagggaagatgtgaatcaaaatagggattttagagcgtagtaccttaaccaaaattaaactgatctaatggtggcattttcttgtaccatgacaagttggttggggaagtgacagaattgctattttacccggcttcgttacctctccttacacacgtacttgaccatatctatattacttatgtctgagactggccctgttcatggaatcaaatgcatactcttaaaatattagatcagaaattgagaatcttacattataaatacttgtatgtacctgcacatatatggtacagattatatctgttcttttgccgattgagaataatgttgaagaggaatcaattatattttcaaactagttggattttttttcagtatttgtatttactatttgtttttcttttctctccatagaaactccccaacttttaaatcatttgaagaaaaggtcgaaaacttaaaggcaagtagagagatgaacagggtctttttgatacattgcctggtgtcttaattaactttgctgtgcgaaaatcattgacattaatatttctttctaaaaatatctaaagaatgtaaataactttactccttatgttactagtggacatttaaggtgttatgtacttaatatctttttagtgtatgctgtagtgtggcagcatgccacatgctgaaaggaaatgtacaatttacaaagaaatggcactttagaaatttgtgctgctttttgctttattcatttcaagtacctttgtaggtggacaaaatttaatataactttcttgtcaacaattaacaagaatataggaagcacaagaaagaaaacaaaaaattggtcctaccactcagacagtgatagatgtatgtgctattatgctgttacgctttaaaaaagaagtagccatacatcataatcattttcctgtatcagtaagtattctctttcaatattatgtgcaatggctgcttgtatattactttttttttttttgagatggagtctcactctgttgcccaggctggagtgcagtggcgtgatcttggctcactgcaacctccacttcctgggttcaagcgattctcctgcctcagcctcccgagtagctgggactacaggcgcgtgccaccacacccggctaattttttgtatttttagtagagatggggtttcactgtgtcagccagaatggtctccatctcctgacctcgtgatccgcccgcctcggcctcccaaagtgctgggattacaggtgtgagccaccatgcctggcctgtattaccttataatggatattatttgttcagtcctcaattgttggacattttttttattataatgatgccatgatgatcgtttatgattcactgtttttattttttgatttctttgattttagtggaggtgaacatttccccataataatgagctttattgatttgtaaattatattatctttgagattatattcaaagctataaaatcatcaactcttaggatcacaggcaacctcaaaagttgtctagttcccatctattcctcgaccctctctacggagtttgtataaaacccatattttcattacttcctaactctgataattataggggatatattttaaggattaaaactagtatcttaaatgtttttatatcagtcagtttaaaaactaatattcagtttagtctttcagaactttgagtcacgaaatgcatctttaaaagcagggtacatttattgaaataaaacactctacagtgatctggatctttttttaatttatttttttattatacttttaagttctgggatacatgtgcagaatgtgcaggcttgttacataggtatacacgtgccacggtggtttactgcacccatcaacctgtcatctacattaggtatttctcctaatgctatccctcccctaccatcccaccccctgacaggcgccttggtgtgtgatgtttggatcgttttttttcttgaacatacaaacgttagtacataccttttctgtttgtatgcagacatatgaatgttttttcagaagaggagttacactgtacacacacagtcctacagcttattcattcagccctcactagcagggtgctttgtgagccataaatgcagggacctgttctttttggtcactgatacttctcaaaggcttagaacagttcttggcacattgctcaatatgcatctgatgagtaacaatatatataagagtcattcttctatgtcatttcaagagacctgctttatttttcttagtgccaagtcatatatttgaaagcaaactgtagttgcaggaagtcaaggattactaaaattcagttctcaatattgttaggactgttatagcctttccttgaaatctgccccaagtgttaatttttgaaatggcaaattggaaaaccaggaatcttatgtgtttttgacattggacatgaggagaaacttccaggtcattcactatatactctcctgaatatatttagattctataaattgtaatgaagtctgctttaaaatatgactaggcaccattcagattaaaaagtgtaatttaaaaacatacaatatgttggctgggcgcagtggcttacacctgtaattccagcactttgtgaggccaaggcaggcagatcacttgagtccaggaatttgagaccagcctgggaaacatggcaaaaccctgtctctaccaaaaacgatagaaaaattagctgatgtgttggcatacacctgtagtcccacctactcgggaggctgaggtaggaagatcgcttgagcccgggaggcagagtgagcagcgattatgccactgcactccagcctgggtgacagagccagaccctgtctcaaaaaaaaaaaaaaaaaaaaaaaaaaaggatagcgtattaaacagataccccaagtatcctaaggaagctgtaaaattacatgctggggatataattttgttaaattacatttatatttaattaaacatgaggtccatttagattttgggttgattcattcatctgtgattactgagcatttgccatgtgccagtcaatcagtattaggcaccagagttaccacagtgagcaagagagacatggaccccttctgcaagatgcctagacaattaaatggccaatgaaactaaagtgaggtaagggttctgatgtgaaaagctgagggtgtgatgaggacaagtggcagaaagttcacctggcttggtggaagggaaggtcagagaagacctcttagaggactcagtaatgtccattgaggattttaggttgaagggaatgtttgaagtttgagggcccttttgaggaccaccaagaacttcactgtgctgtaggatattgagcaggaggtgaagctagagagatgtgtaggccacattgcagagtttgaacttggggcaatgggaagccattgaagggttttaaacaacccaagtggtgcattcttctgtaaatgttagagagatcagtggctgccctgcacatcaaaggggagaacgattggagacaggcagattagatggaaggacggagttgtactccaagccaaagatgatggagagtgaattaagttagtgcaatgggaccaagtctagagctatttagaagtgagaagtgacacggaaaaaaaaaaagattgattggatgtagaacaagttggataagaatccaggacaaccccaggtgtcaggctggggcagctaggtaggtgctattgccatataaagtgttaaaaaagaatagagaaggggctagttgtcgggggaaggagttcagttttagacatgttgaactgcaaggagatttgatactataattaaggaatgtgttttaataaatttaataaacaatgaacacacattattcgcatttcccaaacaatttggatggtagatcaaaaagaatgagagtgaaaataaggggtctgtgaccactcaattaagattgagctaagagcaaggtcaagttcaaacccactataagcttcttgcatccatggtcactgactgttttcttactgttagctgtcctgttggacagtgtaacagtgagagagcatatacagattagcattgaaaaggataaagtgacatgtgtaagggttttgaaaaagtattatacaaatgcaaggcagatatattgaaataaatgcttattggcaaaatttcaggccagataggttggagtagtgcttcacaaactataatgtgcacatgaacaactcagggattttgttcaagtgttgatttcaattctgtagctggagttgggtggggtagaaaggatatctgagatcctgcatttctaataagctcccaggtaatgttgagtctgtaggtccttgaaccacactttaagtattaaagggttagaaagcttatactgtaactgagaaaaaaacatatatttctttcccatgttgtattcaatatttcttgaatgagtgtagctaaatgagcaattcgatgcaatacaaaatgttatgcaagtgtagagacagacagttcactaggtactttaaaataatagaccttacaaagttttctagacagacttcctgtgaagaacaaggagcagggggaaaggggaaggggtagaatttaccaatctgaatatttcagcaagggagaatgcatgagttttactataaaaccacactgaacattgcaacaatactaaattccatacatatataataatcactgagctataaggttgctgatgtatttgttcttgaaagtgaattagagttactttatgcagaatcagctagttttatgtttcataatgtctttcccttttttgaacttttttttaagtctaaagtagggggaaccaagcctgctggtggtgattttggagaagtcttgaattcggctgcaaatgctagtgccaccaccacggagcctcttccagaaaagacacaggagagcctgtgagattcctacctttgttctgctacccactgccagatgctgcaagcgaggtccaagcacatcttgtcaacatgcattgccatgaatttctaccagatgtgcttttatttagctttacatattcctttgaccaaatagtttgtgggttaaacaaaatgaaaatatcttcacctctattcttgggaaacaccctttagtgtacatttatgttcctttatttaggaaacaccattataaaaacacttatagtaaatggggacattcactataatgatctaagaagctacagattgtcatagttgttttcctgctttacaaaattgctccagatctggaatgccagtttgacctttgtcttctataatatttcctttttttcccctctttgaatctctgtatatttgattcttaactaaaattgttctcttaaatattctgaatcctggtaattaaaagtttgggtgtattttctttacctccaaggaaagaactactagctacaaaaaatattttggaataagcattgttttggtataaggtacatattttggttgaagacaccagactgaagtaaacagctgtgcatccaatttattatagttttgtaagtaacaatatgtaatcaaacttctaggtgacttgagagtggaacctcctatatcattatttagcaccgtttgtgacagtaaccatttcagtgtattgtttattataccacttatatcaacttatttttcaccaggttaaaattttaatttctacaaaataacattctgaatcaagcacactgtatgttcagtaggttgaactatgaacactgtcatcaatgttcagttcaaaagcctgaaagtttagatctagaagctggtaaaaatgacaatatcaatcacattaggggaaccattgttgtcttcacttaatccatttagcactatttaaaataagcacaccaagttatatgactaatataacttgaaaattttttatactgaggggttggtgataactcttgaggatgtaatgcattaataaaaatcaactcatcattttctacttgttttcaatgtgttggaaactgtaaaatgatactgtagaacctgtctcctactttgaaaactgaatgtcagggctgagtgaatcaaagtgtctagacatatttgcatagaggccaaggtattctattctaataactgcttactcaacactaccaccttttccttatactgtatatgattatggcctacaatgttgtatttgttatttattaaattgtgattgttttattattgtttatgccaaatgttaactgccaagcttggagtgacctaaagcattttttaaaagcatggctagatttacttcagtataaattatcttatgaaaaccaaattttaaaagccacaggtgttgattgttataaaataacatgctgccattcttgattgctagagtttttgttagtactttggatgcaattaaaactatgtgctatcacatgtgaaaagcttaataaattccatctatcagtagtataggtctcaatatttattatgagaccagtggtctggaaacagcttgttgtaccgaatcaactggagtctatgcttaaaaaaaaaaaatttttttttaaccatccttaaattattgcttaatggtatcatattaacatattctaaataagggctttaaggcacaggctgttgaagcattttctcagaggagtggatctgtagaagtctgtctttctatagaaatattgtgcttactcaagtgttaaattattttttctatgaactagtctacttcttaaaattcaaacatattcttttgatcacattgtttcttgagcatcctgccctgctactaacttttcaacaaggcaaaatggagtaaagtggcaatttctttagatgagtgaaataccctcaagtctcttttctgcccaaaaagggaaaagtgatagaaatgggggtggcaagtggggtgagtggatgaaggtgggtattgggggtggctgtgaaagaaaataatggagaatcacttttctagacatctacctatacttaatctaagaaacaaagtaatctactgtaaagtactctgccccttgaaagaagtattaaaaagagtgaggatggatttagaaaaaaacatgaatttagaaatattcaaaatggtttttgtggcagattcaatattatgaattcacagatatttaaagaatgagaaacatagtaattagtagaaatgccagaaacagttcctggttcctcttgtgtttgacactaagaaaatagcaagagtgtgaaatctcagatacttatgaaatctcacagatgtaaggactcaagtgtagaagaaaatatccccttcttacaaaaagaaatgtcaatttatggagtttgtgggaaatagggcaagaattcttatgcttatgagagccaagtagtcagtggaagagagtagagctcaaaactggattatcaccttagcaacttagaatagtttgaaatagaaaaaaagtatttaatttggatctggatctgttaagatatgcacagtctattttttgtatagtattggaaaataaaaatgctataatttgggtatgggttcttttgtatacattacctgcccattgaggaaaggggcaagtccattatgcaacttctctccaaacccttcatattctggatatgatacttaaagagccatgagcagctacattctgcctatcagaaggcatttattaaatgactgcttgctgaagatgtgtccaaagagatcctacattaaggtcatttgtcagaatgatgtttgtgtttgtttagagttggctgacctccaactcctggggtcaaggaatcctactgccttagcttcccaaatagctaggactataggcatgcacccggccatgtgttttatttatagctcttaaagcccagatgaagaaatcacatttttgcccatagtgaagaaacatttggccattgattagtccttattttcagtgactgtcctgttttcattagattagagagaccctgtgtgggccacagttaatataaaccattatcacttttaagtaaacctgcacatcttagatttcataatttccttattgttctgactcaaaatgaactaagagcttttcactttttgtttgtaagttctcagagagtcgggtctgcaagtgcttttgcctgccaggattttgtactcaaataaatgctatttggcaactaaaattcttgccagctctcatgtaattttgatgttactgaggttggttggtagtttattttattaattttgtggttcttctatcatatagaaaaagccaaatacagttttgtttgaatatttgtaaatggacgtgatctttcactttaccactctccagcatgctttaaagtttctccttggtttctagaattgattctctgttcttattttccattttgactattttttagtattagaattcttccagctcctaaatgctattgtctatctgttgttatcttctacatgatccccttaatgatcttatccatttctatgggtgaaatgactatttttagagtgatactaatagcttcccaaccggtatctccagacttgttgatggtggtggccctccttgagcagctgctgcaaggacaccagctgcagcaagggaagcatggctggggggttgcaggctctgcagaagtggcaggggccggcaataggtgatcccagcaggaaccctgccttaccatgttggcagggcaggagcttgtgctcctgggtgcagctgcagccacccagctgcagctccagatccaggcatctgtgtgctctcgggggcctggaaagtcccctgcccatgcaggcctggaagtgcctgctccagttcggagcaaagttgtggctgagcttgggtgctgtcacaacctggccaggtgtgtgcatcctcagggtggtgctgacatgtcaggttcctgctacctcggccccctcccaactatgggcaccagtgagcatgggagggtggccaggggctgagggtagcttggtgtggacagcctgggtgctctggatggcatgttgatggtggcaggaggcaggttcctaggagggaaggggtgggtccctgttgaaaccccaccctcaagccagggacagcctgaagcctgggggccaggctgccagttttaggtagagcccacaacccagagcgagaactctattaatgcatttcggctaatcggatggtgttttttccaggcccacccttcctccattctgagcacataaaaaccccacactcagccagactcaacactcatcaggacaacctgcctgcagaaaggaccactttgggtctcctgagtgctgttctgtcattcaataacgctcctctctgccttgctcaccctacggttgtccatgtgacttcattcttcctggacacaggacaagaactcaggacctgctgaacagcgggagcaaaaggaactgtaacatgttcctggctggcttgccaagctggggcggtgacacgctcccagactgcaggagagaagagtggtgacccttctggggacccagaccttgggattccctgagccagagctgctataacattatagccttcccacctttcaccggcaccagacagctgccccacatgacaggaggcagcagcagagccaggccagctgaggagccatgggctggagtggggtggtgggactgaaagagctgtaacacaaacgggctgaaacaaccccccaacaatgtcccccccccaacccccctccccgccccgccggtcaccacattgcaggcaacgagaagagttgagcccttctgggagcccacaccttggggttccccaagccagggctgtgacatgctgtaacaccctctttgggactctgaggttgctggcatctctgagcttttgggttttaccacgtttcccttgcccagacactggtgcctgcagaagctggttgtggtccatctggtccagctgcagccttgcacagagccggtgcctgtagctgctcaccctgctgcagcagctggtgtgcctggctgggcgcagtggctggaacctgcactcacttgctcacgcacccctcaccagcccatgcctggctcacccttggcaggtgtgggatctgggccagcagcacaagatgagcacagcctactgggcagagtgggcagaatgagcccagcaggcatgagcaaaactccagcagaggcgctgcaggccatggaagtttccagctggtgaagcgacatcctaagaactttgtgacactatcctttcttcttggtttgcacacatggttgaggcatgaatttctagtaaactcatggtcacctccacctagccgtcaatagctactattcattaagtgcccaggcatcttacactcatcacctttaactctttactcaggcatgtggcagtgtcaccattttatagataagaccaaagccagtagctgtaaagtattaggcttgaacttagcagtagaatgaaactattttctgttcactttccttggcttttttgaggtgggggtctcactgtgttgcccaggctggccctaaacttctgggctcaagcgatgttccctcctcagcttcccgagtagctgagactaaaggtgtgcactgctgcccagccaggcctgtatttaataaagcaaggagtggttacagcaggatcagaatccttaagatggcgtatgtgaaaacatcactattacagaggatgctactcttggcacttgtataggcaaggagaaagttatgcggacatccccctattgacttgttcagttctttacagtccctctcatttgatgcttctgatcctctaccaaagacagaactaaggccaaatttgaagtcatttgatacaaacttactcattttttttttttttttttttttttgagctggagtctcgctctgtcacccaggctggagaacagtggtgtgatcttggctcactgcaacctccacctcctgggttcaagtgattctcctgcctcagcctcctgagtggatgggattacagccatgtgtcatcacgcccagctaatttttgtatttttagtagagacagggtttcaccatgttggccaggctgatctcgaactcctgacttcaggtgatctgccctccttggcctcccaaagtgctgggattacaggtgtgagccaccacgccaggggtgtgtttttacggatacttgagttttgcacttttcacccatgctggagtgaagtggcaggaacatagctcactgcagcctcaaactcctgggctcaagtgatcctcctccatcagcctcccaagtagccgggactacaggcatgcaccactgccccaggctgatttttctattttttggtagagactggttcttgctatggcccaagctggtctcggactcctcatctcaagcaatcctccctcctcaacctcacaaagtgctgggattacaagtgtaagccaccctgcccagtcagactgagggttcttgaagaccactggccatgctctttacaatttaccacactttctcctgaaaaaaagtggaagtagcactatccgtagaaaataatgttctaggcaagagtccattaatagtctcgggagataaacagttttaactgccaagagggagaactcacctggagaatttaatgaatttgtaaggactaccatacagttagctagtaataattttatgtgattcatacaattgtgcaccattctgagatcagtgtacttcgtgttcttaacactatctgtaccattagagagcatcagaaaagcttaaaacccatagtcaaggcattcttaaaatagggtctgtgatcataaaaattctttctctcgaagtacttgcaaatgaactggaaaggtgtgtgcccccaaaataaaactaactgtggattaatattaaactgctgggtcaaagatagacggacaactaattaataaacacttaagaatgatgttaggctgttttgaacagggtgggtaaattttgggaattaacactgcaggagaaaagggaaagaaaatccagagtgagggtgggggcctacggcggtgcaatacttagggacattccaaaacttctgaactcctaagttagataataggtatctagaggtgtcaaaacatttcagaaaaactatttttgtggcgtgagtggactggctgcagaagtggcaaaggacaaacttggatgcagggtttggcaaatgttgggtgagccctaatctgtgcctggtgctagggaccctacagcaaagttcctcttttcaaagtttaaaaagttgggagtagacactaaatattattaaatatttaatattaatatgtggcatattagaaagtgctacatggtactaagaaaaacaggtgccagagagtctgcatttgggcttccacttctaaatagggtaatcaaggagaccctcatatttgagtgaagaccccaaactcgagtgctggcggccatgtaggggggacggcagggcgggagaacgagcgtcgcgttagcagaatttacagtacaatggggtccaacggatgcccaggaaggcgaaattcctgcaagttaaacgcacttctaacgcaaaacttaaccaggcccctttccaactatttagcccaccaattttagcttaagtctcttcttccccacgccactcccagatgaaaggaagacacgcctcgttggcctccagagctctgcgtccggggggcggacctctcatagctccgccccgtgacccgcgcgcttgcggaggggaggtcaaaggtcagaggtaaagaagtcatggcggcgctgtgtcggacccgtgctgtggctgccgagagccattttctgcgagtgtttctcttcttcaggccctttcggggtgtaggcactgagagtggatccgaaagtggtagttccaatgccaaggagcctaagacgcgcgcaggcggtttcgcgagcgcgttggagcggcactcggagcttctacagaaggtggagcccctacagaaggtggagcccgggcgggtgagagggagtcgcgggaattacgccaacgacgcggtgggcggggtcgggaataggggcggagcctggcttaggaaggggagctgaggtccggggcccagttttccggagttgcggtgcgttgtggctcagccctcggcccctagaatctgtgcttggtgtttttcttcctttcgttgtccttttgtgtggcatttgcgtactgggccgggtctgtccacactgcgcttgttttcctgggattgaatgctggtgggaaggtaaccagcggggagcgctgctggggaggcgatctagggcgattcctgggacgctaaggagaatgggactcacacggagaggcagggctctgctgggtcttgttctgaacagtagctctagcatggagcttgctgtctggagtagaaggcccttcataaacatttgttaatttaatgactgggtgatgaatcactttctgaacttgggcaagtgatttaacttgtcagcgcttcacctctcttttgtaaatgtcttctttttttaaaaagtaaagtaaaatctccgtttgtctcagccagtacttttgtaaaataccataaatttatagataaatgaaatggaagaaataaaattacaagcctaagtttttaataattaaactcagcagacatgaaattgaattatatacaaataaactctcaatttctgtatttttcttcctagggagtagtaccaaacagttcacaagactggcagtgtcctgggttatagggtcattttagtagcaattactgatgtcaaaatttttctgaaatccaaggactagaagaagcctggtatattagtttattattttctgttgtaacaaattactacaaatttagtggcgtaaaacacaggaaatgtgttgttgttagggttgtgagaagtcagacgggatctcactggtctaaaatcaaggtgttggtgaagttgtgttcttttccgtagcttctaggagaatctcttcctggcctgtcttttctgcctctgttttctacttttttgttttttggatttttttgtttgttttgttgttttttgagacagggtctcgctctgtcacccacgctggagtgtggtggcatgatctcagttcactgctaaaccccctgggctcaagtgatcctcctgcttcatagctgggaccgcaggtgtgcaccaccactctgggctaattgttttttctgtttttttttgtagagatggggttttgccatgttgcccaggctggtcttgaactcctgggttcaagtgatcctcctgcgttggcctcccaaagtgctgggattacagatgacagatgtgagccacctggcctggctgaccccgcccggccccgccccccccgcccgtcttctgcttttgagatccctgtgattacattggcctccctagataatctattttaagatcagctgattagcaatcttaattccacctgcaaagttaattctcctttgccacggaaggtaacatattcacaaattctagagattaggatgtggatgtctttgtagggggagggggcattattctttctaccatatatggagaagtcataaaatactagcagaatagcgatgattgacctgaagccacctaagggatggattttagatccatccttaaggtaaggccataagagttaagtgcagtaaggttggaaacatagagttcagtgctcccttccaaatatatgtataggaaaggagtttttctatttcttacctaatatttctcactcacgcttcgctcccattcataccacattctaagccccttcggaaaccccccaaaataattctcaattgctgagttaactgtggtaagagtgaaaattgtagcatcacatgtatagtaagttaagtgtttgaaactaaaaatgtactgtttatttaatgaaggaaacaaccaaggtgttttagagtccccagaattccctttcctttccttttctctttcctttcctttccaaggcagttctaattgggctgttcagtttttccaggaggaggctggggtgtttcctggcagtcccagcacatccgttagattttctcagtccagctcaggctcagcagcatctctgcaccctgcgtggttgttctccgtctaacacagtttagaaaacattattctactagatgatgggtaaatgtgtttggttgtgatgtacagttcaccaatttcttattcacaagtctttgacatcttggtttaatggaaataaaattccctggggaggaggatatcaggaggctgtgttgtagtctccgttcagcaagttcttagctttgaagcatttgaagtttacgtgaacccctttactccttcttaattgataccactgacttcagttgctccctggttcaggtcattacacacactgtggatcaagttatcttcctaaagttgggctctataatcagacttaatgaggttatttacttattgctctccttgagaatcttctaaacctccattcacactgcctgcaagattaaatatacaatggtgcatctctctactgtggcttcagaggcctactgccattcaagaccctgcttgggttaaaagcttcggctcagttcaggtttccagtttctgccatctttcaactgagtgactttgggcaacttacttagcctatctaagtttgctgtcttatctcaaagtggagataatgcttcaactggagttactgggaagtttatgtgaaataatacatttaacgtgctctcagtacattgactggtacagagtaagctttattcagtgcattttagcttttatgtggccccagccgtttcttctccagccttctgccttcctgagaatagtttcacagagatgcttccacaattacacagttctttgaagtcccattgttttcttaataatgaactttaatttatcaaaccgtgtagattcatctcttgtaactgatgtaccgctctggtgggggatgttgacagtgggggaagctatgtgtgtgtcagcacaggaggggtatgggatatgtctgtactgtactctcaattttgctgtaaacctaaaagtactctaaaaaataattttttttcttaatgaatgaatataaaatgctatttaaaaacataaggcagtatatgctctaactagaggttgagctgtgtcattcaaagagacaataaaattaagtttttcatggcaaaaagcccaaaaccaaacaaaaaacaccagaaaacaaccatgcacatgccattttttttaatcccaaatgtaaggacattcgtcatcactaatatgttaacttgtggtgccagagtaacacatgacacatcagaataaaatttttactaacttttttcccagtagaaaatgtttcaggaggtcagagaccttgtctttcttattctgttgtatttccagtgcctagcacagtccttgacccatagtgggcactcggtaaatatttgtcaaatgtgtgaatgaatgaatagtacatgtagtgcatatttgaacttcagttatacctggcctgtcgttgaattgtaatatatgtgtcactgtccacaaacaattgtacaagaatagtacagtcttgagcatgtcagttgaatgtgcctgtccagaataattgagtaaaaataattttaagagtagtaagaaattgaaaataccacattagttttagttttagtatttactggaagttttcagtgtttttctaacatatttatggtaaggaatatgaggagttgttgggtattgataggaattcaacttgtgaaaaaacttgctttacctcttttgaacttactggacctatttaattgaacagtatcgtgagattacaagatgaataaacgccaattgtgtattcagggtttttgaaaaaatattgtttacaaattttacaaaagtaatatttggttactttagaaagaaaatttacatagccctgcaggttattatgtgaaatgtaaaagtccctttttcttactttctcaaggctggagagtgtgttttttatgcatagctgcagagttttatattacacacgtaagtataagctaagtattgctatatatatttaaaaacattaattggttcgtagtatgaatactgttttaaaatatattacagttaaagagtgtaaattctgcagtcatgcaatcttggtttaaatcctggttcctctaccatttactagctgtgttgccttggaaaattacttaatatctgtgtatctccttttccccctatgtcatgagatttttatgagattcaatgaattcattatttaaaacacttaacacaagtgcctgatacttaataagcacttagtatgttttagttgctgctgctgaaaattgcagtttttacctaagtatgactatttttccttatcattacataatatttgcttggtattccatggtgtggatgtagcataatttatttaagtaggaaggccttaattattgaacttttaggttgtttctgtttgtttttgttttctaacacagtgttgcagtgaacatttgcattttttgcgtacttacaagagtgtatttgtaggttatatgttgggtaaaagtgtacacacaatttgagttacagcctgtttgtactgaactcccacctacagtgcatgacaatgtttcctcacccccgtatcaactcatggggtgtaataggtggattttcttgatattcgacaattaaagcaaaatatagaaattggaaactggggttatcatctttcattcctgatttcagtttttgatttttatcataactgcatgttgacagtttttgactttatagtaagtgctatcaatttgaactcataaaatgcacttaaaataacaaaatactacatttacactatatatattgggattgtatgtaagatttcatttgtaaaatggtttctgcttaaaatattctttttgaagatgaattttgctgttccctaaacaaatcattatttctttaccttttaccctggaattagcttccctgctgcaaattctgttgtaaaaatcacctcctctcttagacctttgtgatctcaccaaacagatgaaactgtttttgaatgctctggagttttgtgttttactacctttatggtatacccacagctctcatagtaaagtcatttacaaacctgttttatcctgttttctagctgtagttcctgaagaacaaggggccattttcagtccaccctcttcatcctttgcataatgctttacacttaagtagggattgagtatatatttgctgatttaagttggaatgaaatttaaacagtttgatggttgaataattacaaatgaaaaatagcatctcagatggcctattattagacttaaaaatatgtatatggtgaaagtccaagttttacctattgatttagttcttatagtcattaaagtgtagacttacttacatgtggacctcagaaataagcccatacacattatccttacaaagaactttcccctgcagccaggaatttaatcaagagcaatgcatcatgaagttcttatcctattacacactcgggttactagttaaagggaaccgaagttaaatggcattaagtgtaatgccattgttctgtatggtggttgtaggttccatactcatcccatgctcacttacctacttttcttttactttgcttcattagctttaatattggtttatgcatctttcccttcaactagttttgttttgtttttagagtagcaaccaaatcttttttttttttccagacagggtcttgctctgttgctgaggctggggtgcagtggtgcaaacttggctcactgcaacctctctcccttggctcaggtgattctcccacctcagcctcctgagaagctgggaccatagctgtgcgtcaccatgcctggctgatttttgtatttttttgtagagatggggttttgccatattgcccaggctgctctcgcacttctgggctcaggcaatttgcctgccctagcctcccaaaatgctgggattacaggtgtgagccaccacaccaggccgtcccattcatttttataaccatttaagtcaggtaccattttccctccatttttaaaaccttaatccttcttatgtcacttagacaatataaatttgtagaatctgttaaggcaattgggagctgtgctatggcatataactcatctcgtagccacaaggtggcaatagttgagcaaagaattggtttttctgctttatgactcttagcagtagagggtggcatgctacttttcagaacattggaaaaaatgtttttcagagagttttagggctgcacagtaaaaattttactcaaattttaaatttatttaaaatactgatgttacatgacagtttatagttttaccttattttagcaatatattagatttcatgcaaatatttttaaatgtaattttttacttttttgtgaatatttttattagtattttttatttcaaaatattcgaatattccagaagacagaatatctaaaatcagatgttcaatcctttcagaatactggaaaaataaaaatatatagatttaaagggcttatttcaatataatagggaaattaatttatttttagtataatttttaagtccactaaattcatatactgtaatagtaaaattatatagttttacttaaccctttgttttcttatcagttttactgacaactttaagaattataccagtgttttaaaaactatttgatatgtaaaggaatgatggaaaatgtttctggttttaacgatggtgccaacctgtttttacattatagaattctaggagaattacctctgagtaataaaaatcttttgaaatgttgtatgtctcacattttcaaagcatgtttagaggaaatgaccagttaatatataaacaatgaaatagtgaagaagtacatagaattctctggggaaagtaggtttttgttaacacctgtaacatgcttataagcagtactatacatttcttaaaagaaaagatgtatatgttctgtatgtattttgtttttgggtggtcatatgatctgattatacccaatcagactcatgacttcccaagccaaccttaggtttttcactatttggagatcgtaactctcatttttactcagaaatttcacttccatcttttaatgtgtctaagtttgcttctaaggctacacttaaatttttctcttttaatcactttaatttttataacctgattatggcccttaagttttttatctattggcattaaatatcaggaaataagagcaattaagcacattcaagtggagcctggtttgttgggatttgggaataaaactaaatagtcttgattcagtttgggttttgcctacctttttaaagctttggagattccattgttgacaactgccaggaaatgagtggtggactcagtctaggagttggcagctgaacaagttgcctggcctctggccagctacccaaattgagatggttggattctctgaagggacataaatgtacactgagaactgggtgtcactgggacacacagtatttatcccaaagaaatgaaatgttcctaacagcttcatttgtaatagccaaaaactgaaagcagcctagatgtccttcagctgttgaatggttaaacaaactgtggtacatccttaccgtggaatactacttagcagtaaaaagtaatgagtgtttgatacacaaaataatttgaatgactcaaaggaattatgctgagtgaaaaaaaaaaagccagtcccaaaaagttacatatggtgtgatttcatttatacaacatttttgaaatggcagaattatagagatggagaacaggtcagtgcttactaggagttaggaaggagggaggtaaaagggatatgtggagttacaaaaggacagcacaaggaatccttgtggtaatggacttgttctgcattttgactgtgatggccagtacagaacctttacatgtaataaaattgcattgatgattacgtgtataactggtgaaatctgaatattggtggattgtgtcaatgtcaatttcctagttgtgatatcatactatagtcttctaaaatgttaccattgggagccctgggtaagggtgcaggggctctctatgtattcttggttacaattgcatgtgaatctacaatgatttttaaatagaaagttaattttttttaaagggagcatttcactggtctctcgggcccagcactgataagaccattgaattccataaacatgtgacagttatcccacattatttcctgtaaggtaattttttgttgttagaagcaatgcagtgtagaataccagaaaaatccaagaatagttagaagtaaagttccacatgcctacctgcataccttagcctttagtcagacaacttctgtcttactagaaaccttaaaagcatcccttggtcccagtctatgcatctgtcagcatctggcttacagctctgctcataaaatgagagagagactgactgtccagtctccttttgtaactgtttttccctatctagcataatgtttctcctgctcccacgatataccactattacagagatttctaaacttttttgatcatgtcccttcattaataaatttttgagcagatgttccagtatttacttaaaaaatacatttgtggctgggcgcagtggctcacacctgtaatcccagcgctttgggagaccaaggtgggcggaacatgaggtcaagagatcaagaccatcctgaccaacatggtgaaaccctgtctttactaaaaatacaaaaattagccgggcatggtggctcatgcctgtagtcccagctactcgggaggctaaggcgggagaattgcttgaacatgggaggcggaggttgcagtgagtcgagattgtgccactgcactccagcctagcgccagaaagagacccagtctcaaaaaaaaaaaaaaagtgtgtgtgtgcgaatatatatattcgtactgtagtttcttttgttatatacattttaaagcatttacacataaaaacattttgaaggtgaagttaaaaaaaacaaatagaaattccagtgttttcttcttctgtcctatgggttatgatagagatcactattctttgttacaacctgacctttttccttcaggcctgctctgtaaatgtgtgttcgttggccacaaatttcctatgtctttttacgaacaatctcccctttaaccttctctacaagtatttcttgcttgctttcttggtttcccttgataacactgcttcccttctggtcttccctagtatagattgcaacgggtctcaaggctaggaggaaaaaatgactcatcttcgttctcgtttctcagtaccatttcagactactgttctatcagaaatactttaaaaaggctccttgataaaaatcttttcaattctttccatttttcgtcagcatctggctcacagcttttctcttttcttcatacttaccattttcattgaccagcatttacatttgtaaaccaaaaataaaattctaaagccccctcaaccatctgaatggacccctcctgtcagccaagggcgttccaaagttaacctgaaaaactagtgcaggccgtgatgggaagggggagccagacgtgcctcattatacctccctcctttcagaattagtgatagaaatgactctttaagtctagtaagaaacatttacaatctgttttctctgaagcctgctacctggacgcttcatctgcatgataaaaccatactctccacaatgccttatggtcacgcagacattcctttctattgataataaccctttcaaccaattgccaatcagaaaatctttaaatctacctatgacctggaagccaccctcgcaacctgcttctagttctcctgcctttctggactgaaccagtgtaaccaatgtacatcttacacgtattgattgatgtctcatgtctccctaaattgtataaaaccaagctgtaccctgacttccttgggcacgtgtctcaggacctccttaggctgtgtcatgggcgtgtccttaaccttggcaaaataaactttccaaattgatttagacctgtctcatactcttggtttacacattaatgactaccaatactttggccacatagttttttgtatattaaccttatattgtgtaatgttgctgtaattacttattccaggaggtttttttcttgttgattctttttgattttctacataggtcatcatgccatccatgatcatacacagttttatttcttccttcccaatctgtatacttttattttttctttttgtcttattgcattagctaggacttctagtacaatgttgaaaagcagtgatgagaggggacatccttgccttcttcctgtgcttagtaggaaaagtttcaagttacttaccattacatatgtaggttttttgtagatattctttaggaagttgaagctttctctagttttctgagagttactattatgaatgggtattgggttttgtcaaatgcttttccatttgacgaaatgaaatacgaccatgtgatttttcttctttagccaattgatgtgatggattacattttttttctatttttaatttttgtgggtacatagtaggtgtatatgcttagggggcacacgagatactttgatacaggcatataacatgtaataatcacatcatagtaaatagagtatccattccttcaagcatttactctttgtgttaacaatcaaattaaactcttttagttatttttaaatatacagttaaataattattgcttatcaccctgttgtgctctcaaacactaggtcttactcattctttctaactactctttgtacccattaaccatccccacccccttatatactataagtatactatatcacatataggggagtggggatggttaataggtacaaacccccaatacccttcccagcgtctgggtaaccatccttctactctgtctccatgagttcaattgtttaaatttttaactcccacagataataacatgtaaagtttgtctttctgtgcctggcttatttcacgtaacgtaatgacctctggttctatccatgttgttgcaagtgacaggatctcattcttttttatgactgaatagtagtactgcattgtgtatatgtaccacattttctttatccattcatctgttgatgggcacttaggttgctttcaaattttggctattgtgaacagagctacagcaaacatgggaaatgtctcttcagtatactgatttcctttctttggggtataaaaccagcagtgggattgctggatcatatggtggctctatttttagttttggttatttgttttttgtggaacttcaaactgtttttcagagtggttgtactaatttatgttcccgctaacagtatatgatggttcccttttctccacatcctcatcagcatgtattactgctggtcttttggataaaagccattttaactggggtgagatgatatctcattgtaggtttgatttgcatttctctgatgataatgatgttgagcactttttcataagcctgtttgccatttgtatgtcttctttcgagaaaggtccattcagattatttgccttttttttttttttttttttttgagacagagtattgctctgtctcccaggctggattgcagtggcacgatctcggctcactgcgagctctgcctccttccgggttcatgccattctcccgcctcagcctccccagtagctgggactacaggcgcctgccaccatgccctgctagttttgtttttgtattgttagtagagatggggtttcaccatgttaaccaggatagtcttgatatcctgaccttgtgatccgcccgcctcggcctcccaaagtgcagggattataggcgtgagattatttgcctattttaaagttggattattagaatttttttcctatagagttgtttgaactccttatatatcctggttattaatgctttgtctacattttgatttttgaatgttaaacctgtcgtaagtctcagttgtggtatataatttttatatgttgttggatttgatttgctaatattctgttgaggatttttgcatctacatgcatgagaaatattggtctgtagttttcttttcttgtaatgtctttgtgtggttttgatattaagatattactgtcctcataggataagttccactcctatcttctgaaagagattgtagagaattggtttgatttcttctttaaatgtttggtagaactcaccagtgaacccatatggacctggggctttctgttttggaagtatattaattactgatttaatgtctttaatagacataggcctattcagattatctgttcttcttacttgaattttggcagactttgtctttcaaggaattaatccatttaatttaggttatcaaatctgtgggtatagagttgtttatagtatttctttaatgttcatgggatatgtagtgatgtcctctctttcatttctattattagtaatttgtgtcctctctttttgtttttcttagtctggctagaagcttacctcaaataaccaacttttgctttcgttgattctattgatttcctgttttcagtttcattgctttctgtgctaatttttattttagataagttgtgttttcattttcatttagttcaaagtatttttaaatttctcttgagatttgtttgactcatgttatttagacgtgtatttgtttactctaagaattttgggatttttgagctatctttctattcctgatttctagttaaattctagtgtggtctgaaagcagacattgcatgatttctattcctttaaatttgttaaggtgtgttttatggtccagaatgtggtctgttttggtgactgtttcatgtggcatgagaagaatatatattctgctgttgctggatgaaattgtctacagatgtccatcatatccagttgatttatgatgttgttgagttcagctatatcctgagtgattttctccctgctggatctgtccttttctgatagagggacattaaagtctccaactgtaatgattcatctatttttccttgcagttttgtcagtttttgcctcatggtttttgactatttttaggcatgtacacattaaggattattccgacttcttcagataattaacccttttattattagcaaattgtttaacatgaacaaactgttaattgttagatcaattactaattaaaaacttaaaagttttaattttaccttcacatattccttctctgtgttctgcctttctttatgtaatgtagatctgagtttttgacttttataattttcttctttctaaagaatttctttaaacatttcttgcaaggcaggtctacttgcaacaaattccctcaatttttatttgtctgagaaagtctttaattctccttcatttttgaaggataacttgtcagggtacagaattctaggttagtattttttttttttgtttcctcttaacactttagatatctcactccactgttttcttgattgcatgatttatgaggagaagctggatataatttttatcttttctcctctgtaggaaagctgttttccccccaagcttcttttagaattttttcttcatctttgatttttctgtagtttgaaaatgatactatatgcctagatgtagtgtttttggtgtttattctggttgatgtcctctgagcttcctggatctgtggtttggtgtctgatattaatttgaaggaaagtcttagtcattattgtttcaaatatttctgctgtatttgtctttcttctccttctagaattcccattagtttacaccttttgtagttgtcccaaagttcttgaatattctgttcaatttttccctcaatcttttttctctttgcttttcagttttggcagtttttattgaggtatcttcagagtcacagtttttttttttcctcagctgtgtcatctacctgtaagcccatcaaaggcattcttcatttctgttacagggctttcgatctctagcatttctttttgattcttatttaaaatttccatctttctgcgtacattgcttatctgttcttacatcttatttcttccgttagagcccttagtgtcttaatcatagttgtattagatgcctggactgataatttcagtatccttgctgtgtctgaatctggttctgatccttgctctgtctcttcaaactgtgttttttgcttttttgtctatcttgtaatttctttttgatagctgggcatgatgtattgggtaaaaggaatggctgtcaatcggcctttagtaatgtggtgatagggtgtgtggggagggaaagtgttctatagtcttatgagtgggtctcagtcttctagtgagcctgtgcctctggactatgaactgcacatttgcttctcagtgtttccctctcttttggtaggacagaatgactagtgccagctagaattggttatttcccttcctccaggacacttaggctctgacaagtgtttaggctttggttgaagagtttccttgaggacacatgtcatatgcatgatagttttatatatgtgcttaatgtatattaaaaagacaacacctgtttgataaatgaattgttgaataagagagagtgctctgatgtatttcaaaatagttctattccccacagaacttgccgtctgaagtacaaggggatttttctcctatattcactgtaaggaccaggttgagctcctggagttaaaactcacaaaagtgtgagagtccccaataactgagtctctctggagtttttaactttcagacttgtcccactgagcatccaccaattcaccagttacagctcgggtttccctacctcggcactggttcccacagaggtttcttcttggaggcttcttttcagtagttctctgcattcacttgtctctccacatttgagaacagcagttttccctgtgacccccacctctcagatgcatctaagaagagttgttgatttttcagtcatgacaatcaaacacatctccagacattgccaaatgtcccctggagtgggggtggcaggggggaaatcatcttggttgacaaatactagattagtcaataaaacttgatggtggtgactgagtctataagaaattaatttttaaagaagctaaagtttttgacagggtctgtttaccttcagacactatttgagtgctttacatatggccacacatttattccttacaacaatcttgtaaaatagatagatataattacaaacaccattttataagatgagtaaacagcagcacagagattctaagtgcacagacctgtccccagagctggtaagcggtagagccaaggttcaagccatgtggccagacttcccagccttcctttgggagccctcgtatttgaatcatcacaaattgttcatatgcttgctgcacagctgtcatttggagtcttttctttgctgcttctataggttatagaaacagtataagttactgctgcataacaaaccatcccaaacttggctctcctctgtacaatttgtttgatttttttaagggaagagaaaaaatatatttatttttgacaggataatacatgcctattaccagatattatgcacttgaggaattttgtgcttgacagaatattgactgccctgcctctgttgtttttagctattctccttgttttcagcttgtcttctcactgcagcctggcatgactgagctccatacctttatggaatacttcaggatagaatagctttctttttttcacagcttcttcttcttgaatttcactctgcttcattaattatcactcttccacatgctttctgtccactagaaagtatgtttgtatacttttgttctccatctgttctcttcaatattgagagtttataatttttacaattttttaatttattattttaccatcatttaatgggatttcaggagggagagagataaatgtaagtaccttatcaccttgagccaccaatacgtttatttcaataaaagaactctgttgcgagcgtaacatttattggttagtgttaaagaaacaaaagttattatagcacggttaatagaagtgggtcatttttggagatgcatgttgaaatttttttcttgctaaccagtatgaagtagttaaagtatatataccatttcattacttttattgttattgttttttttcttttttcttttctttttttttttttttttttgagatggagtcttgctctttttgcccaggctggagtgcaatggcgcagtctcagctcaccgtaacctccacctcctgggttcaagtgattctcctgcctcagcctcctgagtagctaggattacaggcatgcaccacctcacccggctaattttgtatttttagtagagacagggtttccccatgttggtcaggctgttctcgaattccggacctcaggtgatccacctacctcagcctcccaaagtgctgggattacagggtgagccactgcacccagctgtttttattgtctacattaaaagaggtaactgggtaggatgttgtctcccatttctaatttgtattgttttataaaatcctttttatattttagaatatttaagaattcctactaccttatagaattggaagaaaatttatagttatccaatcctttaatttacaaatgagaaaacagatctagagaagttaaatgacttattcagggtttttttttcgctgtttattggctgagttggtcatagaagtcaggtttcgtatttgttattcttctgtactgttttcttttaagtttgctatgatgtttttatctattttctatcttctgcagcaagttttatttaagaaccactgttcagtatcccatgtaactttgaatagcatgtatggtactcatgtaaaacttgaggtagttggattagttgatgtctcatattcttttagcactagttgcccctgtttccaagctgtatattaaatggtcctcatatgcatgataggtatatatatgcttaatatatattaaaaagacaacacatgtttgatgaatgaatgaatgcatgattgaataaatgaaagtgatactgttccatatgtctgtaattcctttaccttactcctttctaccttgacggtcctgtcaactttcaggcttcaaaggagaaaatagtacagcagaatagcaggagatctgatctctatgaccaactgttaaaggaaatagtcaactccaagttagtacctgtgacagctcagttagtgagctttattgtaacattttccagggttaggcactgagctctatgatacatatccagcggtgacagtatgcttccagtctctgcagtctatatcagcatgagaggagctatcaatgttagtttagagactttataaacaggactccctgtagtgaacatggattgtgggagttgctacatttactgtttttaggagtcatttgacaagtaaatgagtacattttggcataagccctaggattcagagatggttctagactatgatttttgaagcaagggtttgtctgattgtttgatgtagcttcccaaaatcaatgatgcaatatcaaaaatcaaatgaaataataagctacaatttaaaagcaaattcaggtgttaatccagggttcttatgcttgtggtaaaattatttcactcgccaaaaacacactgacttccttgcaaaatataatagaggcaaaaaatggaggagaaatgttctacttccacttgattttaaagttacctataaaaagtaactataactttataaagtttataatgtaataaatatttaactgcagacttactaaaaatctcaagagaatctttttaaacagttgagagcactcaataattttctaaaaccaaggggaaaataccacttactattatactaatcttagctcaaacatctcttcacaattgaaaaaggacattgtgttattttaggaacaggtcctagtgatggctgaattatctccagtaaaagtttaactactggatctgctactactgtatcttagtgtcatctggttttttgttttgttttgttttttgtttttgttttttgttgtttttttttgagagtctcactctgtcgcccaggctagagtgcagttgtgccatctcggctcactgcaacctccgcctcctgggttcaagtgattctcctgcctcagcttcctgagtagctgggattacaggcacatgccaccatgcccgactaatttttgtattttagtagagacagggtttcaccgtgttggccaggctggtcttgaactgctgacttcaagtgatccacccaccttggcctcccatagtgctgggatcacaggcgtgagccacaatgcctagcccctgtcatttagttttaattggactatggcttcaattgaaagtcagccaattgaaaaaaaaaaatagccaaagcgaaaaaagaagaaaaagaacagaggaactctggtagatatcaaccataaaagtggagtaatgggaaagcagatcatagggaaagtgggcaggacctgccctggttacaagcagagacccccaagttgatactgtttgctcttctcttgagcactgctattctcatctgctttagtggcagctactatggatggaataatggctcccagcaaaggattatgtagctgctcttgtcaagaatggcttacaaaaaaaaataaaaataaaaaaataaaaaaaaaagaatggcttacttagaattactcttttacactacctctaaagggcaagtcagctgtgtctgtgttacctctgtaggatttgttttatctctggctctgcaagaaagaactattttcaaagtgcttgtttaaggacctttaatgttgtattttgtggatttataaaactagtatgattgctcatggcatacattcctggttaccttttcagctctagttggcctctaggcctactgacttgtctttagtttagattgacccttttgtgctaaggattttttccctgtgtgtttcagttcaccaagtaaattcatcatcataccctccctctagcacctgagggacttggcataagccaaatgtcgtgactttctttttcctgaagcagaggtcataaacttgtggttcgtgggccaaattcaacacctggatgtgttttgccgggtttgtaaaatggaaaactttatccccctgcatataagtatttaaaaattggaaaatttacattaatatgtggatttctggcttttcttggaaaattaaaacattaaaaattaaaacatggggacttgaattgcagctgcagctggagctgagtagtgtctacaccttttaaacttgggcacatatcctgattatcaagccctcacctggcctggcatttgagtttataatctttcaagtagtacatgcattaaacttaagtcacaaatatagttgtgtagctttaaaagtaaatgtgctttactattgaacttttagtagttctccccgaataacaaatgtaaaactcattccaaatacagcatttaggtcgtcagatcaaattgttaatgtccttccatgatttgctctcacagatagaaacttagcacttcctttagtttgtaccaaattgcacacaccactccatacacaaaaatgagtagaaggcacaaatctgtgttcacggatttggaaaaaatgtgtttggaaaaaatttttgtaagaaaaaaagaataattagtcatacctcaagtgaattcaatatgtaaaaagattcccggaaagtgaatagcacaaaacaagctgtttttcttttggaatttactttaggagaaagaaaagtataacaaattcaagttgtttaaaaagaattatgtatcaggaactcatgttcagactgatcattgtgatgaaaggctcttaattgtttttatcttaatgtatatttacatttgtattaaaagaatagtgactttcactaaacttgatttagaaggcatttttttacagatggtcagtgctaggaataaagtcaattgcttcttggatgccctcctcacgtgctcatactctgaccactaaccaccccccaccccattctcagagacttgatatacaggcactcttctactctccctaccctgcctgggctctcgagacctcaccctggactatcacatctctgtcatcctccaccaagtatagatgcctaatttctttcttttttttttcttttttgagacagtttggctcttgttgcccaggctggagtgaaatggcgtgatctcggctcaccgtaacctctgcctcctgagttcaagtgattctccttcctcggcctcatgagtagctgggattataggcatgcaccatgatgcctggctaattttgtatttttatttttatttatttatttatttattttttattatactttagattctagggtacatgtgcacaatgtgcaggtttgttacatatgtatacatgtgccatgttggtgtgctgcacccattaactcgtcatttacattaggcatttctcctaatgctatccctcccctctgcccccaccccacagcaagccctgctgtgtgatgttccctttcctgtgtccaggtgttttcgctgttcaattcccacctatgagtaagaacatgaggtgtttggtttttttgtccttgcaatagtttgctgagaatgatggtttccagcttcatccgtgtccctacaaaggacatgaactcatccttttttatggctgcatagtattccatggtgtatatgtgccacattttcttaatccagtctatcactgatggacatttgggttggttccaagtctttgctattgtgaatagtgccacagtaaacatatgtgtgcatgtgtgtttatagcagcatgatttataatcctttgggtatatacccagtaatgggatggctgggtcaaatggtatttctagttctagatccttgaggaattgccacactgtcttccacaatggttgaactagtttacagtcccaccaacagtgtaaaagtgttcctatttctccacatcctctccagtacctgttgtttcctgactttttaatgatcaccattctaactggtgtgagatggttatcccattgtggttttgatttgcatttctctgatggccagtgatggtgaacattttttcatgtgtcttttggttgcataaatgtcttcttttgagaagtgtctgttcatatccttcacccactttttgatggggttgtttgattttttcttgtaaatttgtttgagttctttgtagattctggatattagctctttgtcagatgggtagattgcagaaatgttctcccattctgtaggttgcctgttcactccgatggtagtttctttggctgtgcagaagctctttagtttaattagatcccatttgtcaattttggcttttgttgccattgcttttggtgttttagacatgaagtccttgcccatgcctttgtcctgtatggtattgcctaggttttcttctagggtttttatggttttaggtctaacatttaagtctttaatccatcttgaattaatttttgtataaggtgtaagaaagggatccactttcagctttctacatatggctagccagttttcccagcaccatttattaaatagggaatcctttccccatttcttgtttttgtcaggtttgtcaaagatcagatagttgtagatacacggctttatttctgagggctccattctgttccattggtgtatatctctgttttggtaccagtatcatgctattttggttactgtagccttgtagcgtagtttgaagtcaggtagcttgatgcctctagctttgttcttttggcttaggattgacttggcaatgcgggctcttttttggttccatatgaacttgaaagtagttttttccaattctgtgaagaaagtcattggtagcttgatggggatggcattgaatctataaattaccttgggcagtatggccattttcatgatattgattcttcctattcatgagcatggaatgttcttccatttgtttgtatcctcttttatttcgttgagcagtggtttgtagttctccttgaagaggtccttcacgtcccttgtaagttggattcctaggtattttattctctttgaagcaattgtgaatgggagttcactcatgatttggctctctgtttgtctgttattggtgtataagaatgcttgtgatttttgcacattgattttgtatcctgagactttgctgaagtttcttatcagcttaaggagattttgggctgagatgatggggttttctaaatatacaatcatgtcatctgcaaacagggacagtttgacttcctcttttcctaattgaataccctttatttctttctcctgcctgattttcctggctagaacttccaacactgtgttgaataagagtggtgagagagggcatccctttcttgcaccagtttttaaagggaatgcttccagtttttgcccattcagtatgatatcggctgtgggtttttcatagatagctcttattattttgagatacgtgccatcaatacctaattgcttgagagtttttagcatgaagggttgttgaattttgtcaaaggccttttctgcatctattgagataatcaagtggtttttgtctttggttctgtttatgtgctggattacatttattgatttgcatatgttgaaccagccttgcatcccaggaatgaagcctacttgatcatggtggataagctttttgatgtgctgctggattcggtttgccagtattttattgaggatttttgcatcgatgttcatcaaggatattggtctaaaattctcttttttggttgtgtctctgccaggctttggtatcaggatgatactggcctcataaaatgagttagggagggttccctctttttctattgattgaaatagtttcagaaggaatggtaccagctcctccttgtacctctggtagaattcagctgtgaatccgtctggtcctggactttttttggttggtaggctattaattattgcctcaatttcagagcctgttattggtctattcagggattcaacttcttcctggtttagtcttgggagggtgtatgtgtcgaggaatttatccatttcttctagattttctagtttatttgcgtagaggtgtttatagtattctctgatgttagtttgtatttctgtgggatcggtggtgatatcccctttatcattttttattgcgtctatgtgattcttctctcttttcttctttattagccttgctagcggtctatcaattttgttgatcctttcaaaaaaccagctcctggattcattgattatttgaaggtttttttgtgtctctgtctccttcagttctgctctgatcttagttatttctgcgttcattttgttatgtacccagtagtcattcaggagcaggttgttcagtttccatgtagttgagcggttttgagtgagtttcttaatcctgagttctagtttgattgcactgtggtctgagacagtttgttatcatttcttttttacatttgctgaggagtgctttacttccaactacgtggtcaattttggaataagtgtgatgtggtgctgagaagaatgaatattctgttgattttggttggggagttctgtagatgtctgttagatccacttggtgtggagctgagttcaattcctggatatccttgttaactttctgtcttattgatctgtctaatgttgacagtggggtgttaaagtctcccattattattatgtgggagtctaagtctctttgtaggtctctaaggacttgctttatgaatctgggtgctcctgtattgggtgcatatatatttaggatagttagctcttcttgttgaattgatccctttaccattaagtagtggccttctttgtctcttttgatctttgttggtttaaagtctgttttatcagagactaggattgcaaccgctgcctttttttgttttccatttgcttggtagatcttcctccatccctttattttgagcctatgtgtgtctctgcacgtgagatgggtctcctaaatacagcacactgatgggtcttgactctttatccaatttgccagtctgtgtcttttaattggagcatgtagcccatttacatttaaggttagtattgttatgtgtgaatttgatcctgtcattatgatgttagctggttgttttgctcattagtttatgcagtttcttcctagcatcgatggtccacagtttggcatgtttttgcagtgtctggtaccggttgttcctttccatgtttagtgcttccttcaggagctcttttagggcaggcctggtggtgacaaaatctctcagcatttgcttgtctgtaaaggattttatttctccttcacttatgaagcttagtttggctggatatgaaattctgtgttgaaaattcttttctttaagaatgttgaatattggcccccactctcttctggcttgtagagtttctgccgagagatccgctgttagtctgatggccttccctttgtgggtaacctgacctttctctctggctgcccttaacattttttccttcatttcaactttggtgaatctgataattgtgtgtcttggagttgctcttctcgaggagtatctttgtggcgttctctgtatttcctgaatttgaatgttggcctgccttgctagattggggatgttctcctggataatatcctgaagagtgctttccaacttggttccattctccccgtcacttttgggtacaccaatcagacgtagatttggtcttttcacatagtcccatatttcttggaggctttgttcatttctttttacttttcttcctctaaacttctcttctcacttcctttcattaatttgatcttcaatcactgataccctttcttccggttgatcgaatcagctactgaagcttgtgcattcgtcacgtagttcttgtgccgtggttttcagctccatcaggtcatttaaggacttctctgcattggttattctagttaaccatttgtctaatcttttttcaaggtttttagcttctttgcgatgggttctgacttccttctttagcttagagaagtttgatcgtctgaagccttcttctctcaagtcatcaaagtcattctctgtccagctttgttccattgctggcaaggagctgcattcctttggagggggagaggcactctgatttttagaattttcagcttttctgctctattttttccccatctttgttgttttatctacctttggtctttgatgatggtgacgtacagatggggttttggtgtggatgtcctttctgtttgttagttttctttctaacagtcaggaccctcagctgcaggtctgttggagtttgctggagatccactccagaccctgtttgcctgggtatcagcagcagaggctgcggaacagcgaatactgatgaacagcaaatgttgctgcctgattgttcctctggaagcttcgtctcagaggggtacccggctgtgtgaggtgtcagtctgcccctacttgggggtgcctcccagttaggctactcaggggtcagggacccacttgaggaggcagtctgtctgttctcagatctcaaactcagtgctgggagaaccactactctcttcaaagctgtcagacagggacatttacgtctacagaggtttttcctgccttttgttcggctatgccctgcccccagaggtggagtctacagaggcaggcaggcctccttgagctttggtgggctccacccagttggagcttcctgactgctttgtttacctactcaagccttggcaatggtgggcgcccgtcccccagcctcgttgctgccttgcagtttgatctcagactgctgtgctagcaatgagcgaggctccgtgggcatgggaccctccaagccaggcatgggatatagtctcctggtgtgccgttggctaagaccattggaaaagcgcactattaaggtgggagtgacccgattttccaggtgccgtctgtcacagcttcccttggctaggaaagggaattc

SEQ ID NO: 3 - protein, 251 aa, Coturnix coturnix
MVAVLSELPGMDLYEDYKSPFDFNAGVDRTYLYLSPGINLTPPGSPTLTKSGLLRTDSIPEVGEDAAATVGMSETLSEEEQNELRKELAKVEEEIQTLSQVLAAKEKHLAEIKRKLGINSLQELKQNITKSWQDVTSTAAYKKTSETLSQAGQKASAAFSSVGSVLSKKFEDVKLQAFSHSFSIRSIQHSISMPIMRNSPTFKSFEEKVETLKSKVGGSKPASGDFGEVLNSAANASATETIAEETQEETH

SEQ ID NO: 4 - protein, 224 aa, Mus musculus
MECRDMELADDYQSPFDFDSGVNKNYLYLSPSGNTSPPGSPTQNVGLLKTEPVAEEGEDAVTMISAPEALTEEEQEELRRELTKVEEEIQTLSQVLAAKEKHLAEIKRKLGISSLQEFKQNIAKGWQDVTATNAYKKTSETLSQAGQKASAAFSSVGSVITKKLEDVKNSPTFKSFEEKVENLKSKVGGAKPAGGDFGEVLNSTANATSTMTTEPPPEQMTESP

SEQ ID NO: 5 - protein, 224 aa, Homo sapiens
MDCREMDLYEDYQSPFDFDAGVNKSYLYLSPSGNSSPPGSPTLQKFGLLRTDPVPEEGEDVAATISATETLSEEEQEELRRELAKVEEEIQTLSQVLAAKEKHLAEIKRKLGINSLQELKQNIAKGWQDVTATSAYKKTSETLSQAGQKASAAFSSVGSVITKKLEDVKNSPTFKSFEEKVENLKSKVGGTKPAGGDFGEVLNSAANASATTTEPLPEKTQESL

SEQ ID NO: 6 - protein, 184 aa, Homo sapiens
MDRGEQGLLRTDPVPEEGEDVAATISATETLSEEEQEELRRELAKVEEEIQTLSQVLAAKEKHLAEIKRKLGINSLQELKQNIAKGWQDVTATSAYKKTSETLSQAGQKASAAFSSVGSVITKKLEDVKNSPTFKSFEEKVENLKSKVGGTKPAGGDFGEVLNSAANASATTTEPLPEKTQESL

SEQ ID NO: 7 - protein, 248 aa, Homo sapiens
MTPRESAPGRGRAAPPRPTPLGVGTSRESPAEARRSSARRGGRSEPGRAAGGGAAEDTRRRAGDMDRGEQGLLRTDPVPEEGEDVAATISATETLSEEEQEELRRELAKVEEEIQTLSQVLAAKEKHLAEIKRKLGINSLQELKQNIAKGWQDVTATSAYKKTSETLSQAGQKASAAFSSVGSVITKKLEDVKNSPTFKSFEEKVENLKSKVGGTKPAGGDFGEVLNSAANASATTTEPLPEKTQESL

SEQ ID NO: 8 - protein, 184 aa, Oryctolagus cuniculus
MDRGEQGLLRTDTVPEEAEDAAATISATETLSEEEQEELRRELAKVEEEIQTLSQVLAAREKHLAEIKRKLGINSLQELKQNIAKGWQDVTATSAYKKTSETLSQAGQKASAAFSSVGSVITKKLEDVKNSPTFKSFEEKVENLKSKVGGTKPAGGDFGEVLNSTANASATSGEPVPEQTQEGL

SEQ ID NO: 9 - protein, 185 aa, Mus musculus
MDRGEQGLLKTEPVAEEGEDAVTMLSAPEALTEEEQEELRRELTKVEEEIQTLSQVLAAKEKHLAEIKRKLGISSLQEFKQNIAKGWQDVTATNAYKKTSETLSQAGQKASAAFSSVGSVITKKLEDVKNSPTFKSFEEKVENLKSKVGGAKPAGGDFGEVLNSTANATSTMTTEPPPEQMTESP

SEQ ID NO: 10 - DNA, 32 bp, Artificial Sequence
  Synthetic Primer
cctgaactgtttgtacctctgggccatattgc

SEQ ID NO: 11 - DNA, 33 bp, Artificial Sequence
  Synthetic Primer
caaatttctgaagagtaggtgatccgggtggag

SEQ ID NO: 12 - DNA, 34 bp, Artificial Sequence
  Synthetic Primer
tctaaagtagggggaaccaagcctgctggtggtg

SEQ ID NO: 13 - DNA, 34 bp, Artificial Sequence
  Synthetic Primer
actgatagatggaatttattaagcttttcacatg

SEQ ID NO: 14 - DNA, 33 bp, Artificial Sequence
  Synthetic Primer
caagttaactgagctttttcttaatttcattcc

SEQ ID NO: 15 - DNA, 37 bp, Artificial Sequence
  Synthetic Primer
gttggtacctccacagaagatgtttatttgatgtaac

SEQ ID NO: 16 - DNA, 18 bp, Artificial Sequence
  Synthetic Primer
agtcaatagaagccggcg

SEQ ID NO: 17 - DNA, 20 bp, Artificial Sequence
  Synthetic Primer
ggggatttagagggttctgt

SEQ ID NO: 18 - DNA, 22 bp, Artificial Sequence
  Synthetic Primer
gcctgaactgtttgtacctctg

SEQ ID NO: 19 - DNA, 24 bp, Artificial Sequence
  Synthetic Primer
gagtaggtgatccgggtggagatg